(19) United States
(12) Patent Application Publication
(10) Pub. No.: US 2012/0329111 A1
(43) Pub. Date: Dec. 27, 2012

(54) Microorganisms for Producing Cyclohexanone and Methods Related Thereto

(75) Inventors: Anthony Burgard, Bellefonte, PA (US); Robin E. Osterhout, San Diego, CA (US); Jun Sun, San Diego, CA (US); Priti Pharkya, San Diego, CA (US)

(73) Assignee: Genomatica, Inc., San Diego, CA (US)

(21) Appl. No.: 13/528,541

(22) Filed: Jun. 20, 2012

Related U.S. Application Data
(60) Provisional application No. 61/500,125, filed on Jun. 22, 2011.

(51) Int. Cl. C12N 1/21; C12P 7/26

(52) U.S. Cl. 435/148; 435/252.33

(57) ABSTRACT

Provided herein is a non-naturally occurring microbial organism having a cyclohexanone pathway and comprising at least one exogenous nucleic acid encoding a cyclohexanone pathway enzyme. Also provided herein is a method for producing cyclohexanone, including culturing these non-naturally occurring microbial organisms.

### CROSS-REFERENCE TO RELATED APPLICATIONS

The present application claims the benefit of priority to U.S. Ser. Nos. 61/500,125, filed Jun. 22, 2011, the contents of which is herein incorporated by reference in its entirety.

### BACKGROUND

The present invention relates generally to biosynthetic processes and organisms capable of producing organic compounds. More specifically, the invention relates to non-naturally occurring organisms that can produce the commodity chemical cyclohexanone.

Cyclohexanone is an important chemical precursor of Nylon 6 and Nylon 66. Oxidation of cyclohexanone with nitric acid results in the formation of adipic acid, a key building block for Nylon 66. Cyclohexanone oximation and subsequent Beckmann rearrangement forms the basis for the preparation of caprolactam, a precursor to Nylon 6.

The cost of cyclohexanone is mainly subject to the raw material cost of pure benzene. Cyclohexanone is chemically synthesized by oxidation of cyclohexane using a cobalt catalyst, resulting in a mixture of cyclohexanone and cyclohexanol called “KA oil”. Alternatively, cyclohexanone can be produced by partial hydrogenation of phenol.

Thus, there exists a need to develop microorganisms and methods of their use to produce cyclohexanone from inexpensive and renewable feedstocks. The present invention satisfies this need and provides related advantages as well.

### SUMMARY

In some aspects, the present invention provides a non-naturally occurring microbial organism that includes a microbial organism having a cyclohexanone pathway having at least one exogenous nucleic acid encoding a cyclohexanone pathway enzyme expressed in a sufficient amount to produce cyclohexanone. In some embodiments, the cyclohexanone pathway includes a PEP carboxykinase, a 2-ketocyclohexane-1-carboxyl-CoA hydrolase (acting on C—C bond), a 2-ketocyclohexane-1-carboxylate decarboxylase and an enzyme selected from the group consisting of a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase.

In other embodiments, the cyclohexanone pathway includes an enzyme selected from a PEP carboxykinase, a 6-ketocyclohex-1-ene-1-carboxyl-CoA hydrolase (acting on C—C bond), a 6-ketocyclohex-1-ene-1-carboxyl-CoA synthetase, a 6-ketocyclohex-1-ene-1-carboxyl-CoA hydrolase (acting on thioester), a 6-ketocyclohex-1-ene-1-carboxyl-CoA transferase, a 6-ketocyclohex-1-ene-1-carboxyl-CoA reductase, a 6-ketocyclohex-1-ene-1-carboxylate decarboxylase, a 6-ketocyclohex-1-ene-1-carboxylate reductase, a 2-ketocyclohexane-1-carboxyl-CoA synthetase, a 2-ketocyclohexane-1-carboxyl-CoA transferase, a 2-ketocyclohexane-1-carboxyl-CoA hydrolase (acting on thioester), a 2-ketocyclohexane-1-carboxylate decarboxylase, and a cyclohexanone dehydrogenase.

In further embodiments, the cyclohexanone pathway includes a PEP carboxykinase, an adipate semialdehyde dehydratase, a cyclohexane-1,2-diol dehydrogenase, and a cyclohexane-1,2-diol dehydratase.

In yet further embodiments, the cyclohexanone pathway includes a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from the group consisting of a 3-oxopimeloyl-CoA synthetase, a 3-oxopimeloyl-CoA hydrolase (acting on thioester), and a 3-oxopimeloyl-coA transferase.

In some aspects, the present invention provides a method for producing cyclohexanone, comprising culturing a non-naturally occurring microbial organism having a cyclohexanone pathway having at least one exogenous nucleic acid encoding a cyclohexanone pathway enzyme expressed in a sufficient amount to produce cyclohexanone, under conditions and for a sufficient period of time to produce cyclohexanone. In some embodiments, the cyclohexanone pathway includes a PEP carboxykinase, a 2-ketocyclohexane-1-carboxyl-CoA hydrolase (acting on C—C bond), a 2-ketocyclohexane-1-carboxylate decarboxylase and an enzyme selected from the group consisting of a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase.

In other embodiments of the method a set of cyclohexanone pathway enzymes are selected from (a) PEP carboxykinase, 6-ketocyclohex-1-ene-1-carboxyl-CoA hydrolase (acting on C—C bond), 6-ketocyclohex-1-ene-1-carboxylate decarboxylase, cyclohexanone dehydrogenase, and an enzyme selected from 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; (b) PEP carboxykinase, 6-ketocyclohex-1-ene-1-carboxyl-CoA hydrolase (acting on C—C bond), 6-ketocyclohex-1-ene-1-carboxylate reductase, 2-ketocyclohexane-1-carboxylate decarboxylase, and an enzyme selected from 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; and (c) PEP carboxykinase, 6-ketocyclohex-1-ene-1-carboxyl-CoA hydrolase (acting on C—C), 6-ketocyclohex-1-ene-1-carboxyl-CoA reductase, 2-ketocyclohexane-1-carboxylate decarboxylase, and an enzyme selected from 2-ketocyclohexane-1-carboxyl-CoA synthetase, 2-ketocyclohexane-1-carboxyl-CoA transferase, 2-ketocyclohexane-1-carboxyl-CoA hydrolase (acting on thioester).

In still further embodiments of the method, the cyclohexanone pathway includes a PEP carboxykinase, an adipate semialdehyde dehydratase, a cyclohexane-1,2-diol dehydrogenase, and a cyclohexane-1,2-diol dehydratase.

In yet further embodiments of the method, the cyclohexanone pathway includes a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from a 3-oxopimeloyl-CoA synthetase, a 3-oxopimeloyl-CoA hydrolase (acting on thioester), and a 3-oxopimeloyl-coA transferase.

In some aspects, the present invention provides a non-naturally occurring microbial organism that includes a microbial organism having a cyclohexanone pathway that includes at least one exogenous nucleic acid encoding a cyclohexanone pathway enzyme expressed in a sufficient amount to produce cyclohexanone; the non-naturally occurring microbial organism further includes:

(i) a reductive TCA pathway comprising at least one exogenous nucleic acid encoding a reductive TCA pathway enzyme, wherein said at least one exogenous nucleic acid is selected from an ATP-citrate lyase, citrate lyase, a fumarate reductase, and an alpha-ketoglutarate:ferredoxin oxidoreductase;

(ii) a reductive TCA pathway comprising at least one exogenous nucleic acid encoding a reductive TCA pathway enzyme, wherein said at least one exogenous nucleic acid is selected from a pyruvate:ferredoxin oxidoreductase, a phosphoenolpyruvate carboxylase, a phosphoenolpyruvate carboxykinase, a CO dehydrogenase, and an H₂ hydrogenase; or

(iii) at least one exogenous nucleic acid encodes an enzyme selected from a CO dehydrogenase, an H₂ hydrogenase, and combinations thereof;

wherein the cyclohexanone pathway comprises a pathway selected from:

(a) a PEP carboxykinase, a 2-ketocyclohexane-1-carboxyl-CoA hydrolase (acting on C—C bond), a 2-ketocyclohexane-1-carboxylate decarboxylase and an enzyme selected from the group consisting of a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase;

(b) a PEP carboxykinase, a 6-ketocyclohex-1-ene-1-carboxyl-CoA hydrolase (acting on C—C bond), a 6-ketocyclohex-1-ene-1-carboxyl-CoA synthetase, a 6-ketocyclohex-1-ene-1-carboxyl-CoA hydrolase (acting on thioester), a 6-ketocyclohex-1-ene-1-carboxyl-CoA transferase, a 6-ketocyclohex-1-ene-1-carboxyl-CoA reductase, a 6-ketocyclohex-1-ene-1-carboxylate decarboxylase, a 6-ketocyclohex-1-ene-1-carboxylate reductase, a 2-ketocyclohexane-1-carboxyl-CoA synthetase, a 2-ketocyclohexane-1-carboxyl-CoA transferase, a 2-ketocyclohexane-1-carboxyl-CoA hydrolase (acting on thioester), a 2-ketocyclohexane-1-carboxylate decarboxylase, and a cyclohexanone dehydrogenase;

(c) a PEP carboxykinase, 6-ketocyclohex-1-ene-1-carboxyl-CoA hydrolase (acting on C—C bond), 6-ketocyclohex-1-ene-1-carboxylate decarboxylase, cyclohexanone dehydrogenase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase;

(d) PEP carboxykinase, 6-ketocyclohex-1-ene-1-carboxyl-CoA hydrolase (acting on C—C bond), 6-ketocyclohex-1-ene-1-carboxylate reductase, 2-ketocyclohexane-1-carboxylate decarboxylase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase;

(e) PEP carboxykinase, 6-ketocyclohex-1-ene-1-carboxyl-CoA hydrolase (acting on C—C bond), 6-ketocyclohex-1-ene-1-carboxyl-CoA reductase, 2-ketocyclohexane-1-carboxylate decarboxylase, and an enzyme selected from the group consisting of 2-ketocyclohexane-1-carboxyl-CoA synthetase, 2-ketocyclohexane-1-carboxyl-CoA transferase, and 2-ketocyclohexane-1-carboxyl-CoA hydrolase (acting on thioester);

(f) a PEP carboxykinase, an adipate semialdehyde dehydratase, a cyclohexane-1,2-diol dehydrogenase, and a cyclohexane-1,2-diol dehydratase; and

(g) a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from the group consisting of a 3-oxopimeloyl-CoA synthetase, a 3-oxopimeloyl-CoA hydrolase (acting on thioester), and a 3-oxopimeloyl-coA transferase.

In some embodiments, the present invention provides a method for producing cyclohexanone that includes culturing the aforementioned non-naturally occurring microbial organisms under conditions and for a sufficient period of time to produce cyclohexanone.

### BRIEF DESCRIPTION OF THE DRAWINGS

FIG. 1 shows the transformation of pimeloyl-CoA to cyclohexanone. Abbreviations are: 2-KCH-CoA=2-ketocyclohexane-1-carboxyl-CoA, 2-KCH=2-ketocyclohexane-1-carboxylate.

FIG. 2 shows the transformation of acetoacetyl-CoA to pimeloyl-CoA.

FIG. 3 shows the transformation of 3-hydroxypimeloyl-CoA to cyclohexanone. Abbreviations: 6-KCH-CoA=6-ketocyclohex-1-ene-1-carboxyl-CoA, 6-KCH=6-carboxyhex-1-ene-1-carboxylate, 2KCH-CoA=2-ketocyclohexane-1-carboxyl-CoA, 2-KCH=2-ketocyclohexane-1-carboxylate.

FIG. 4 shows the transformation of adipate semialdehyde to cyclohexanone.

FIG. 5 shows the transformation of 3-oxopimeloyl-CoA to cyclohexanone.

FIG. 6 shows the enzymatic activities of A) 3-dehydroquinate dehydratase, B) 2-hydroxyisoflavanone dehydrogenase, and C) 2-cyclohexenone hydratase.

FIG. 7 shows a route to pimeloyl-CoA from 2,6-diaminopimelate.

FIG. 8 shows the reverse TCA cycle for fixation of CO₂ on carbohydrates as substrates. The enzymatic transformations are carried out by the enzymes as shown.

FIG. 9 shows the pathway for the reverse TCA cycle coupled with carbon monoxide dehydrogenase and hydrogenase for the conversion of syngas to acetyl-CoA.

FIG. 10 shows Western blots of 10 micrograms ACS90 (lane 1), ACS91 (lane2), Mta98/99 (lanes 3 and 4) cell extracts with size standards (lane 5) and controls of M. thermoacetica CODH (Moth_{—}1202/1203) or Mtr (Moth_{—}1197) proteins (50, 150, 250, 350, 450, 500, 750, 900, and 1000 ng).

FIG. 11 shows CO oxidation assay results. Cells (M. thermoacetica or E. coli with the CODH/ACS operon; ACS90 or ACS91 or empty vector: pZA33S) were grown and extracts prepared. Assays were performed at 55oC at various times on the day the extracts were prepared. Reduction of methylviologen was followed at 578 nm over a 120 sec time course.

FIG. 12A shows the nucleotide sequence (SEQ ID NO:1) of carboxylic acid reductase from Nocardia iowensis (GNM_{—}720), and FIG. 12B shows the encoded amino acid sequence (SEQ ID NO:2).

FIG. 13A shows the nucleotide sequence (SEQ ID NO:3) of phosphpantetheine transferase, which was codon optimized, and FIG. 13B shows the encoded amino acid sequence (SEQ ID NO:4).

FIG. 14A shows the nucleotide sequence (SEQ ID NO:5) of carboxylic acid reductase from Mycobacterium smegmatis mc(2)155 (designated 890), and FIG. 14B shows the encoded amino acid sequence (SEQ ID NO:6).

FIG. 15A shows the nucleotide sequence (SEQ ID NO:7) of carboxylic acid reductase from Mycobacterium avium subspecies paratuberculosis K-10 (designated 891), and FIG. 15B shows the encoded amino acid sequence (SEQ ID NO:8).

FIG. 16A shows the nucleotide sequence (SEQ ID NO:9) of carboxylic acid reductase from Mycobacterium marinum M (designated 892), and FIG. 16B shows the encoded amino acid sequence (SEQ ID NO:10).

FIG. 17A shows the nucleotide sequence (SEQ ID NO:11) of carboxylic acid reductase designated 891GA, and FIG. 17B shows the encoded amino acid sequence (SEQ ID NO:12).

### DETAILED DESCRIPTION

This invention is directed, in part, to non-naturally occurring microorganisms that express genes encoding enzymes that catalyze cyclohexanone production via fermentation from a renewable sugar feedstock. The theoretical yield of cyclohexanone starting from glucose as a raw material is 0.75 mol/mol glucose (0.409 g/g) as shown below in Equation 1:

4C₆H₁₂O₆→3(CH₂)₅CO₂6CO₂+9H₂O Equation 1

In accordance with some embodiments, a cyclohexanone biosynthetic pathway involves a pimeloyl-CoA intermediate. This pathway uses channeling of flux towards the synthesis of pimeloyl-CoA, an intermediate of biotin biosynthetic pathways in bacteria, archaea and some fungi (168). Although pimeloyl-CoA is a widespread metabolite, the pathways involved in producing this intermediate have not been fully elucidated. In some embodiments, the present invention provides energetically favorable routes for synthesizing pimeloyl-CoA. The routes disclosed herein for the synthesis of pimeloyl-CoA can be applied to produce cyclohexanone from central metabolic precursors. In additional embodiments, a route for synthesizing cyclohexanone via enzymes in a benzoyl-CoA degradation pathway is disclosed. This pathway does not proceed through pimeloyl-CoA as an intermediate, but does pass through a potential pimeloyl-CoA precursor, 3-hydroxypimeloyl-CoA. In a further embodiment, the present invention provides a pathway from adipate semialdehyde to cyclohexanone. This pathway relates to Applicants previous disclosure related to routes to adipate as disclosed in U.S. patent application Ser. No. 12/413,355, not yet published. In still further embodiments, a pathway to cyclohexanone from 3-oxopimeloyl-CoA via the intermediate 4-acetylbutyrate is described herein.

For each pathway, enzymes are identified with their corresponding GenBank identifier. The sequences for enzymes listed in this report can be used to identify homologue proteins in GenBank or other databases through sequence similarity searches (e.g. BLASTp). The resulting homologue proteins and their corresponding gene sequences provide additional DNA sequences for transformation into Escherichia coli or other microorganisms.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a cyclohexanone biosynthetic pathway.

A metabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the ten is “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

The non-naturally occurring microbial organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having cyclohexanone biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% can represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan.-5-1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep.-16-1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In some embodiments, the present invention provides a non-naturally occurring microbial organism that includes a microbial organism having a cyclohexanone pathway having at least one exogenous nucleic acid encoding a cyclohexanone pathway enzyme expressed in a sufficient amount to produce cyclohexanone. The cyclohexanone pathway includes a PEP carboxykinase, a 2-ketocyclohexane-1-carboxyl-CoA hydrolase (acting on C—C bond), a 2-ketocyclohexane-1-carboxylate decarboxylase and an enzyme selected from a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase. Such a microbial organism can also include two exogenous nucleic acids, each encoding a cyclohexanone pathway enzyme. In other embodiments such an organism can include three exogenous nucleic acids each encoding a cyclohexanone pathway enzyme. In yet further embodiments such an organism can include four exogenous nucleic acids, each encoding a cyclohexanone pathway enzyme. Any exogenous nucleic acid can be provided as a heterologous nucleic acid. Such a non-naturally occurring microbial organism can be provided in (and cultured in) a substantially anaerobic culture medium.

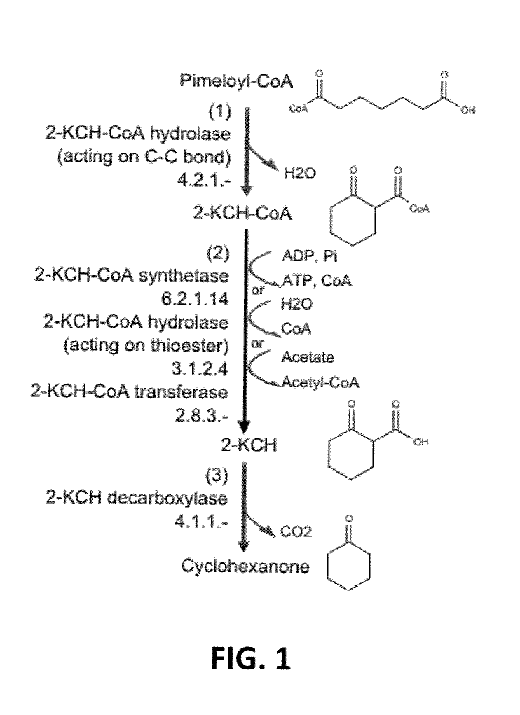

Organisms having a cyclohexanone pathway for converting pimeloyl-CoA to cyclohexanone can include a PEP carboxykinase. The PEP carboxykinase can be encoded by one or more genes selected from PCK1, pck, and pckA. Organisms having a cyclohexanone pathway for converting pimeloyl-CoA to cyclohexanone can include a 2-ketocyclohexane-1-carboxyl-CoA hydrolase (acting on C—C bond). Such an enzyme is run in the reverse direction to cyclize pimeloyl-CoA as shown in FIG. 1. The 2-ketocyclohexane-1-carboxyl-CoA hydrolase (acting on C—C bond) can be encoded by one or more genes selected from badI, syn_{—}01653, syn_{—}01654, syn_{—}02400, syn_{—}03076, syn_{—}01309, and menB. Organisms having a cyclohexanone pathway for converting pimeloyl-CoA to cyclohexanone can include a 2-ketocyclohexane-1-carboxylate decarboxylase. The 2-ketocyclohexane-1-carboxylate decarboxylase can be encoded by one or more genes selected from adc, cbei_{—}3835, CLL_A2135, and RBAM_{—}030030. Organisms having a cyclohexanone pathway for converting pimeloyl-CoA to cyclohexanone can also include a 2-ketocyclohexane-1-carboxyl-CoA hydrolase (acting on thioester). The 2-ketocyclohexane-1-carboxyl-CoA hydrolase (acting on thioester) can be encoded by one or more genes selected from acot12, gctA, gctB, and ACH1. Organisms having a cyclohexanone pathway for converting pimeloyl-CoA to cyclohexanone can also include a 2-ketocyclohexane-1-carboxyl-CoA transferase. The 2-ketocyclohexane-1-carboxyl-CoA transferase can be encoded by one or more genes selected from pcaI, pcaJ, catI, catJ, HPAG1_{—}0676, HPAG1_{—}0677, ScoA, ScoB, OXCT1, OXCT2, ctfA, ctfB, atoA, and atoD. Organisms having a cyclohexanone pathway for converting pimeloyl-CoA to cyclohexanone can also include a 2-ketocyclohexane-1-carboxyl-CoA synthetase. The 2-ketocyclohexane-1-carboxyl-CoA synthetase can be encoded by one or more genes selected from AF1211, AF1983, scs, PAE3250, sucC, sucD, aliA, phl, phlB, paaF, and bioW.

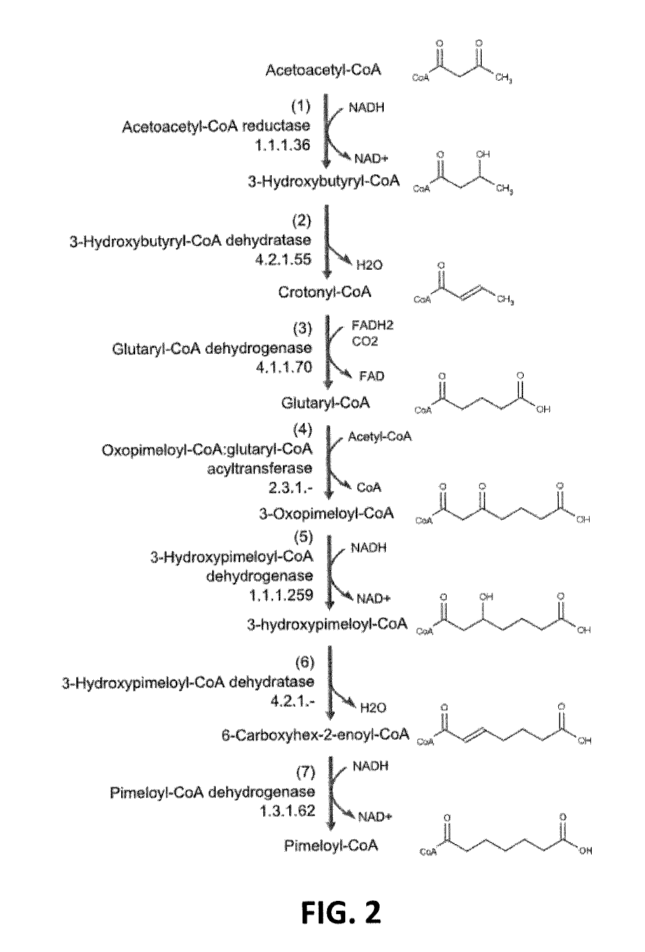

In some embodiments, the non-naturally occurring microbial organism has a native pimeloyl-CoA pathway, while in other embodiments a pimeloyl-CoA pathway can be provided by addition of further exogenous nucleic acids encoding a pimeloyl-CoA pathway enzyme for the production of pimeloyl-CoA from acetoacetyl-CoA, as shown in FIG. 2. Thus, a microbial organism can further include a pimeloyl-CoA pathway that includes at least one exogenous nucleic acid encoding a pimeloyl-CoA pathway enzyme expressed in a sufficient amount to produce pimeloyl-CoA. The pimeloyl-CoA pathway includes an acetoacetyl-CoA reductase, a 3-hydroxybutyryl-CoA dehydratase, a glutaryl-CoA dehydrogenase, a oxopimeloyl-CoA:glutaryl-CoA acyltransferase, a 3-hydroxypimeloyl-CoA dehydrogenase, a 3-hydroxypimeloyl-CoA dehydratase, and a pimeloyl-CoA dehydrogenase. Any number of enzymes can be provided exogenously to provide a non-naturally occurring microbial organism with a complete pimeloyl-CoA pathway for the production of pimeloyl-CoA. For example, the organism can include two, three, four, five, six, seven, that is up to all exogenous nucleic acids each encoding a pimeloyl-CoA pathway enzyme.

Organisms having a pimeloyl-CoA pathway for converting acetoacetyl-CoA to pimeloyl-CoA can include an acetoacetyl-CoA reductase. The acetoacetyl-CoA reductase can be encoded by one or more genes selected from Fox2, phaB, phbB, hbd, Msed_{—}1423, Msed_{—}0399, Msed_{—}0389, Msed_{—}1993, Hbd2, Hbd1, HSD17B10, pimF, fadB, syn_{—}01310, and syn_{—}01680.

Organisms having a pimeloyl-CoA pathway for converting acetoacetyl-CoA to pimeloyl-CoA can include a 3-hydroxybutyryl-CoA dehydratase. The 3-hydroxybutyryl-CoA dehydratase can be encoded by one or more genes selected from the group consisting of crt, crt1, pimF, syn_{—}01309, syn_{—}01653, syn_{—}01654, syn_{—}02400, syn_{—}03076, ech, paaA, paaB, phaA, phaB, maoC, paaF, paaG, fadA, fadB, fadI, fadJ, and fadR.

Organisms having a pimeloyl-CoA pathway for converting acetoacetyl-CoA to pimeloyl-CoA can include a glutaryl-CoA dehydrogenase. The glutaryl-CoA dehydrogenase can be encoded by one or more genes selected from gcdH, gcdR, PP_{—}0157, gcvA, gcd, gcdR, syn_{—}00480, syn_{—}01146, gcdA, gcdC, gcdD, gcdR, FN0200, FN0201, FN204, syn_{—}00479, syn_{—}00481, syn_{—}01431, and syn_{—}00480.

Organisms having a pimeloyl-CoA pathway for converting acetoacetyl-CoA to pimeloyl-CoA can include an oxopimeloyl-CoA:glutaryl-CoA acyltransferase. The oxopimeloyl-CoA:glutaryl-CoA acyltransferase can be encoded by one or more genes selected from bktB, pimB, syn_{—}02642, phaA, h16_A1713, pcaF, h16_B1369, h16_A0170, h16_A0462, h16_A1528, h16_B0381, h16_B0662, h16_B0759, h16_B0668, h16_A 1720, h16_A 1887, phbA, Rmet_{—}1362, Bphy_{—}0975, atoB, thlA, thlB, ERG10, and catF.

Organisms having a pimeloyl-CoA pathway for converting acetoacetyl-CoA to pimeloyl-CoA can include a 3-hydroxypimeloyl-CoA dehydrogenase. The 3-hydroxypimeloyl-CoA dehydrogenase can be encoded by one or more genes selected from Fox2, phaB, phbB, hbd, Msed_{—}1423, Msed_{—}0399, Msed_{—}0389, Msed_{—}1993, Hbd2, Hbd1, HSD17B10, pimF, fadB, syn_{—}01310, and syn_{—}01680.

Organisms having a pimeloyl-CoA pathway for converting acetoacetyl-CoA to pimeloyl-CoA can include a 3-hydroxypimeloyl-CoA dehydratase. The 3-hydroxypimeloyl-CoA dehydratase is encoded by one or more genes selected from the group consisting of crt, crt1, pimF, syn_{—}01309, syn_{—}01653, syn_{—}01654, syn_{—}02400, syn_{—}03076, ech, paaA, paaB, phaA, phaB, maoC, paaF, paaG, fadA, fadB, fadI, fadJ, and fadR.

Organisms having a pimeloyl-CoA pathway for converting acetoacetyl-CoA to pimeloyl-CoA can include a pimeloyl-CoA dehydrogenase. The pimeloyl-CoA dehydrogenase can be encoded by one or more genes selected from bcd, etfA, etfB, TER, TDE0597, syn_{—}02587, syn_{—}02586, syn_{—}01146, syn_{—}00480, syn_{—}02128, syn_{—}01699, syn_{—}02637, syn_{—}02636, pimC, pimD, acad1, and acad.

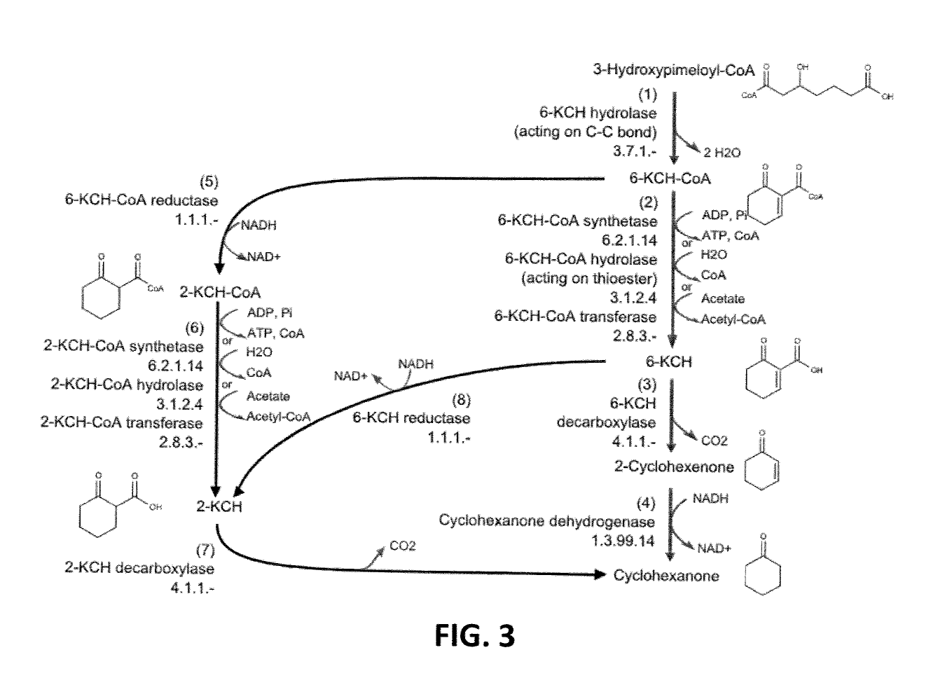

In some embodiments, the present invention provides a non-naturally occurring microbial organism that includes a microbial organism having a cyclohexanone pathway having at least one exogenous nucleic acid encoding a cyclohexanone pathway enzyme expressed in a sufficient amount to produce cyclohexanone. The cyclohexanone pathway includes an enzyme selected from a PEP carboxykinase, a 6-ketocyclohex-1-ene-1-carboxyl-CoA hydrolase (acting on C—C bond), a 6-ketocyclohex-1-ene-1-carboxyl-CoA synthetase, a 6-ketocyclohex-1-ene-1-carboxyl-CoA hydrolase (acting on thioester), a 6-ketocyclohex-1-ene-1-carboxyl-CoA transferase, a 6-ketocyclohex-1-ene-1-carboxyl-CoA reductase, a 6-ketocyclohex-1-ene-1-carboxylate decarboxylase, a 6-ketocyclohex-1-ene-1-carboxylate reductase, a 2-ketocyclohexane-1-carboxyl-CoA synthetase, a 2-ketocyclohexane-1-carboxyl-CoA transferase, a 2-ketocyclohexane-1-carboxyl-CoA hydrolase (acting on thioester), a 2-ketocyclohexane-1-carboxylate decarboxylase, and a cyclohexanone dehydrogenase. Combinations of the foregoing enzymes are capable of converting 3-hydroxypimeloyl-CoA to cyclohexanone, as exemplified in FIG. 3.

The non-naturally occurring microbial organism that can convert 3-hydroxypimeloyl-CoA to cyclohexanone can include any number of exogenous enzymes to complete a cyclohexanone pathway, including two, three, four, five, up to all the enzymes in the pathway. Any number of such exogenous nucleic acids can be a heterologous nucleic acid. Such a non-naturally occurring microbial organism can be provided in (and cultured in) a substantially anaerobic culture medium.

Exemplary sets of enzymes constituting a complete set of cyclohexanone pathway enzymes for converting 3-hydroxypimeloyl-Coa to cyclohexanone include, without limitation, (a) PEP carboxykinase, 6-ketocyclohex-1-ene-1-carboxyl-CoA hydrolase (acting on C—C bond), 6-ketocyclohex-1-ene-1-carboxylate decarboxylase, cyclohexanone dehydrogenase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; (b) PEP carboxykinase, 6-ketocyclohex-1-ene-1-carboxyl-CoA hydrolase (acting on C—C bond), 6-ketocyclohex-1-ene-1-carboxylate reductase, 2-ketocyclohexane-1-carboxylate decarboxylase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; and (c) PEP carboxykinase, 6-ketocyclohex-1-ene-1-carboxyl-CoA hydrolase (acting on C—C bond), 6-ketocyclohex-1-ene-1-carboxyl-CoA reductase, 2-ketocyclohexane-1-carboxylate decarboxylase, and an enzyme selected from the group consisting of 2-ketocyclohexane-1-carboxyl-CoA synthetase, 2-ketocyclohexane-1-carboxyl-CoA transferase, and 2-ketocyclohexane-1-carboxyl-CoA hydrolase (acting on thioester).

Organisms having a cyclohexanone pathway for converting 3-hydroxypimeloyl-CoA to cyclohexanone can include a PEP carboxykinase. The PEP carboxykinase can be encoded by one or more genes selected from the group consisting of PCK1, pck, and pckA.

Organisms having a cyclohexanone pathway for converting 3-hydroxypimeloyl-CoA to cyclohexanone can include a 6-ketocyclohex-1-ene-1-carboxyl-CoA hydrolase (acting on C—C bond). The 6-ketocyclohex-1-ene-1-carboxyl-CoA hydrolase (acting on C—C bond) can be encoded by one or more genes selected from bzdY, oah, bamA, syn_{—}01653, syn_{—}02400, syn_{—}03076, and syn_{—}01309.

Organisms having a cyclohexanone pathway for converting 3-hydroxypimeloyl-CoA to cyclohexanone can include a 6-ketocyclohex-1-ene-1-carboxyl-CoA synthetase. The 6-ketocyclohex-1-ene-1-carboxyl-CoA synthetase can be encoded by one or more genes selected from AF1211, AF1983, scs, PAE3250, sucC, sucD, aliA, phl, phlB, paaF, and bioW.

Organisms having a cyclohexanone pathway for converting 3-hydroxypimeloyl-CoA to cyclohexanone can include a 6-ketocyclohex-1-ene-1-carboxyl-CoA hydrolase (acting on thioester). The 6-ketocyclohex-1-ene-1-carboxyl-CoA hydrolase (acting on thioester) can be encoded by one or more genes selected from the group consisting of acot12, gctA, gctB, and ACH1.

Organisms having a cyclohexanone pathway for converting 3-hydroxypimeloyl-CoA to cyclohexanone can include a 6-ketocyclohex-1-ene-1-carboxyl-CoA transferase. The 6-ketocyclohex-1-ene-1-carboxyl-CoA transferase can be encoded by one or more genes selected from pcaI, pcaJ, catI, catJ, HPAG1_{—}0676, HPAG1_{—}0677, ScoA, ScoB, OXCT1, OXCT2, ctfA, ctfB, atoA, and atoD.

Organisms having a cyclohexanone pathway for converting 3-hydroxypimeloyl-CoA to cyclohexanone can include a 6-ketocyclohex-1-ene-1-carboxyl-CoA reductase. The 6-ketocyclohex-1-ene-1-carboxyl-CoA reductase can be encoded by one or more genes selected from bcd, etfA, etfB, TER, TDE0597, syn_{—}02587, syn_{—}02586, syn_{—}01146, syn_{—}00480, syn_{—}02128, syn_{—}01699, syn_{—}02637, syn_{—}02636, pimC, pimD, acad1, and acad.

Organisms having a cyclohexanone pathway for converting 3-hydroxypimeloyl-CoA to cyclohexanone can include a 6-ketocyclohex-1-ene-1-carboxylate decarboxylase. The 6-ketocyclohex-1-ene-1-carboxylate decarboxylase can be encoded by one or more genes selected from adc, cbei_{—}3835, CLL_A2135, and RBAM_{—}030030.

Organisms having a cyclohexanone pathway for converting 3-hydroxypimeloyl-CoA to cyclohexanone can include a 6-ketocyclohex-1-ene-1-carboxylate reductase. The 6-ketocyclohex-1-ene-1-carboxylate reductase can be encoded by one or more genes selected from NtRed1, AtDBR1, P2, PulR, PtPPDBR, YML131W, ispR, AT3G61220, cbr, CBR1, CHO-CR, YIR036c, enr and fadH.

Organisms having a cyclohexanone pathway for converting 3-hydroxypimeloyl-CoA to cyclohexanone can include a 2-ketocyclohexane-1-carboxyl-CoA synthetase. The 2-ketocyclohexane-1-carboxyl-CoA synthetase can be encoded by one or more genes selected from AF1211, AF1983, scs, PAE3250, sucC, sucD, aliA, phl, phlB, paaF, and bioW.

Organisms having a cyclohexanone pathway for converting 3-hydroxypimeloyl-CoA to cyclohexanone can include a 2-ketocyclohexane-1-carboxyl-CoA transferase. The 2-ketocyclohexane-1-carboxyl-CoA transferase can be encoded by one or more genes selected from pcaI, pcaJ, catI, catJ, HPAG1_{—}0676, HPAG1_{—}0677, ScoA, ScoB, OXCT1, OXCT2, ctfA, ctfB, atoA, and atoD.

Organisms having a cyclohexanone pathway for converting 3-hydroxypimeloyl-CoA to cyclohexanone can include a 2-ketocyclohexane-1-carboxyl-CoA hydrolase (acting on thioester). The 2-ketocyclohexane-1-carboxyl-CoA hydrolase (acting on thioester) can be encoded by one or more genes selected from acot12, gctA, gctB, and ACH1.

Organisms having a cyclohexanone pathway for converting 3-hydroxypimeloyl-CoA to cyclohexanone can include a 2-ketocyclohexane-1-carboxylate decarboxylase. The 2-ketocyclohexane-1-carboxylate decarboxylase can be encoded by one or more genes selected from adc, cbei_{—}3835, CLL_A2135, and RBAM_{—}030030.

Organisms having a cyclohexanone pathway for converting 3-hydroxypimeloyl-CoA to cyclohexanone can include a cyclohexanone dehydrogenase. The cyclohexanone dehydrogenase can be encoded by one or more genes selected from NtRed1, AtDBR1, P2, PulR, PtPPDBR, YML131W, ispR, AT3G61220, cbr, CBR1, CHO-CR, YIR036C, enr and fadH.

Organisms having a cyclohexanone pathway for converting 3-hydroxypimeloyl-CoA to cyclohexanone can include a 3-hydroxypimeloyl-CoA pathway that includes at least one exogenous nucleic acid encoding a 3-hydroxypimeloyl-CoA pathway enzyme expressed in a sufficient amount to produce 3-hydroxypimeloyl-CoA. The 3-hydroxypimeloyl-CoA pathway includes a acetoacetyl-CoA, a 3-hydroxybutyryl-CoA dehydratase, a glutaryl-CoA dehydrogenase, a oxopimeloyl-CoA:glutaryl-CoA acyltransferase, and a 3-hydroxypimeloyl-CoA dehydrogenase, as previously discussed with respect to FIG. 2. Any number of exogenous nucleic acids encoding a 3-hydroxypimeloyl-CoA enzyme can be provided in a non-naturally occurring microbial organism, including two, three, four, five, that is, up to all the enzymes to convert acetoacetyl-CoA to 3-hydroxypimeloyl-CoA as shown in FIG. 2. The same sets of genes used in the pathway for the production of pimeloyl-CoA can be used in a 3-hydroxypimeloyl-CoA pathway, leaving out the final dehydration and reduction steps used to produce pimeloyl-CoA.

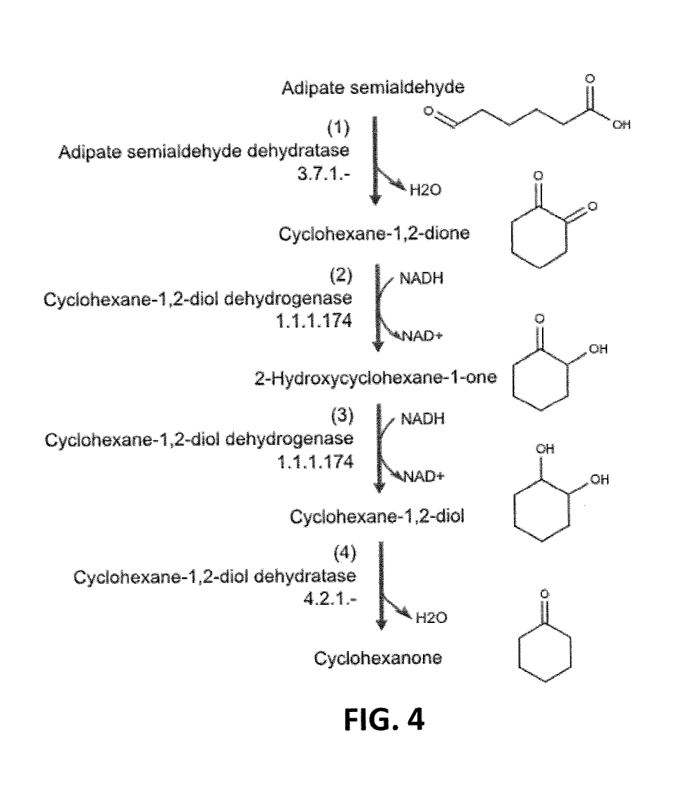

In yet further embodiments, the present invention provides a non-naturally occurring microbial organism that includes a microbial organism having a cyclohexanone pathway having at least one exogenous nucleic acid encoding a cyclohexanone pathway enzyme expressed in a sufficient amount to produce cyclohexanone, as shown in FIG. 4. The cyclohexanone pathway includes a PEP carboxykinase, an adipate semialdehyde dehydratase, a cyclohexane-1,2-diol dehydrogenase, and a cyclohexane-1,2-diol dehydratase. Any number of these enzymes in the cyclohexanone pathway can be included by providing an appropriate exogenous nucleic acid, including up to all the nucleic acids encoding each of the enzymes in the complete pathway. The non-naturally occurring microbial organism can include for example, two exogenous nucleic acids each encoding a cyclohexanone pathway enzyme. In other embodiments, the organism can include three exogenous nucleic acids each encoding a cyclohexanone pathway enzyme. In still further embodiments, the non-naturally occurring microbial organism can include four exogenous nucleic acids each encoding a cyclohexanone pathway enzyme. Any of the nucleic acids added exogenously can be provided a heterologous nucleic acid. Such non-naturally occurring microbial organism can be provided in (and cultured in) a substantially anaerobic culture medium.

Organisms having a cyclohexanone pathway for converting adipate semialdehyde to cyclohexanone can include a PEP carboxykinase. The PEP carboxykinase can be encoded by one or more genes selected from PCK1, pck, and pckA.

Organisms having a cyclohexanone pathway for converting adipate semialdehyde to cyclohexanone can include a cyclohexane-1,2-diol dehydrogenase. The cyclohexane-1,2-diol dehydrogenase can be encoded by one or more genes selected from chnA, Rmet_{—}1335, PP_{—}1946, ARA1, BDH1, GCY1, YPR1, GRE3, and YIR036c.

Organisms having a cyclohexanone pathway for converting adipate semialdehyde to cyclohexanone can include a cyclohexane-1,2-diol dehydratase. The cyclohexane-1,2-diol dehydratase can be encoded by one or more genes selected from pddC, pddB, pddA, pduC, pduD, pduE, dhaB, dhaC, dhaE, dhaB1, dhaB2, rdhtA, rdhtB, ilvD, iolE, ddrA, ddrB, pduG, and pduH.

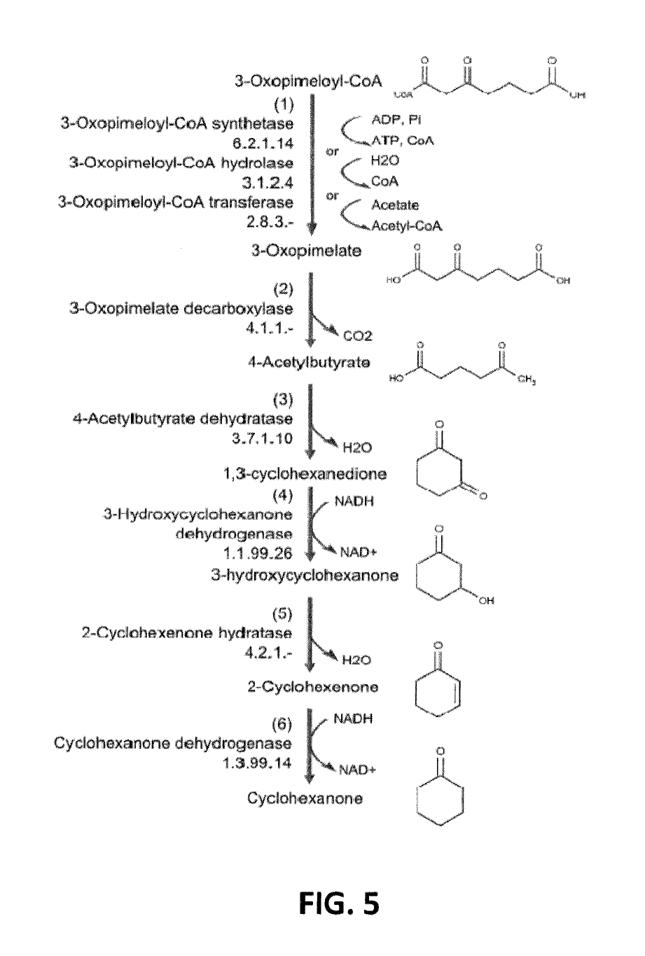

In still further embodiments, the invention provides a non-naturally occurring microbial organism that includes a microbial organism having a cyclohexanone pathway having at least one exogenous nucleic acid encoding a cyclohexanone pathway enzyme expressed in a sufficient amount to produce cyclohexanone. The cyclohexanone pathway includes a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from a 3-oxopimeloyl-CoA synthetase, a 3-oxopimeloyl-CoA hydrolase (acting on thioester), and a 3-oxopimeloyl-coA transferase. Such an organism converts 3-oxopimeloyl-CoA to cyclohexanone as shown in FIG. 5. The microbial organism can include two, three, four, five, six, seven, that is up to all the enzymes in a cyclohexanone pathway by providing exogenous nucleic acids each encoding a cyclohexanone pathway enzyme. The non-naturally occurring microbial organism can provide any number of these nucleic as a heterologous nucleic acid. Additionally, such organisms can be provided in (or cultured in) a substantially anaerobic culture medium.

Organisms having a cyclohexanone pathway for converting 3-oxopimeloyl-CoA to cyclohexanone can include a PEP carboxykinase. The PEP carboxykinase can be encoded by one or more genes selected from PCK1, pck, and pckA.

Organisms having a cyclohexanone pathway for converting 3-oxopimeloyl-CoA to cyclohexanone can include a 3-oxopimelate decarboxylase. The 3-oxopimelate decarboxylase can be encoded by one or more genes selected from adc, cbei_{—}3835, CLL_A2135, and RBAM_{—}030030.

Organisms having a cyclohexanone pathway for converting 3-oxopimeloyl-CoA to cyclohexanone can include a 3-hydroxycyclohexanone dehydrogenase. The 3-hydroxycyclohexanone dehydrogenase can be encoded by one or more genes selected from YMR226c, YDR368w, YOR120w, YGL157w, YGL039w, chnA, Rmet_{—}1335, PP_{—}1946, ARA1, BDH1, GCY1, YPR1, GRE3 and Y1R036c.

Organisms having a cyclohexanone pathway for converting 3-oxopimeloyl-CoA to cyclohexanone can include a 2-cyclohexenone hydratase. The 2-cyclohexenone hydratase can be encoded by one or more genes selected from aroD, aroQ, HIDH, and HIDM.

Organisms having a cyclohexanone pathway for converting 3-oxopimeloyl-CoA to cyclohexanone can include a cyclohexanone dehydrogenase. The cyclohexanone dehydrogenase can be encoded by one or more genes selected from NtRed1, AtDBR1, P2, PulR, PtPPDBR, YML131W, ispR, AT3G61220, cbr, CBR1, CHO-CR, YIR036c, enr and fadH.

Organisms having a cyclohexanone pathway for converting 3-oxopimeloyl-CoA to cyclohexanone can include a 3-oxopimeloyl-CoA synthetase. The 3-oxopimeloyl-CoA synthetase can be encoded by one or more genes selected from AF1211, AF1983, scs, PAE3250, sucC, sucD, aliA, phl, phlB, paaF, and bioW.

Organisms having a cyclohexanone pathway for converting 3-oxopimeloyl-CoA to cyclohexanone can include a 3-oxopimeloyl-CoA hydrolase. The 3-oxopimeloyl-CoA hydrolase can be encoded by one or more genes selected from the group consisting of acot12, gctA, gctB, and ACH1.

Organisms having a cyclohexanone pathway for converting 3-oxopimeloyl-CoA to cyclohexanone can include a 3-oxopimeloyl-CoA transferase. The 3-oxopimeloyl-CoA transferase can be encoded by one or more genes selected from pcaI, pcaJ, catI, catJ, HPAG1_{—}0676, HPAG1_{—}0677, ScoA, ScoB, OXCT1, OXCT2, ctfA, ctfB, atoA, and atoD.

Organisms having a cyclohexanone pathway for converting 3-oxopimeloyl-CoA to cyclohexanone can include a 3-oxopimeloyl-CoA pathway that includes at least one exogenous nucleic acid encoding a 3-oxopimeloyl-CoA pathway enzyme expressed in a sufficient amount to produce 3-oxopimeloyl-CoA. The 3-oxopimeloyl-CoA pathway includes an acetoacetyl-CoA, a 3-hydroxybutyryl-CoA dehydratase, a glutaryl-CoA dehydrogenase, and a oxopimeloyl-CoA:glutaryl-CoA acyltransferase, as previously discussed with respect to FIG. 2. Any number of exogenous nucleic acids encoding a 3-oxopimeloyl-CoA enzyme can be provided in a non-naturally occurring microbial organism, including two, three, four, that is, up to all the enzymes to convert acetoacetyl-CoA to 3-oxopimeloyl-CoA as shown in FIG. 2. The same sets of genes used in the pathway for the production of pimeloyl-CoA can be used in a 3-oxopimeloyl-CoA pathway, leaving out the final ketone reduction, dehydration and olefin reduction steps used to produce pimeloyl-CoA.

In an additional embodiment, the invention provides a non-naturally occurring microbial organism having a cyclohexanone pathway, wherein the non-naturally occurring microbial organism comprises at least one exogenous nucleic acid encoding an enzyme or protein that converts a substrate to a product selected from the group consisting of pimeloyl-CoA to 2-ketocyclohexane-1-carboxyl-CoA, 2-ketocyclohexane-1-carboxyl-CoA to 2-ketocyclohexane-1-carboxylate, and 2-ketocyclohexane-1-carboxylate to cyclohexanone. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 1.

In an additional embodiment, the invention provides a non-naturally occurring microbial organism having a pimeloyl-CoA pathway, wherein the non-naturally occurring microbial organism comprises at least one exogenous nucleic acid encoding an enzyme or protein that converts a substrate to a product selected from the group consisting of acetoacetyl-CoA to 3-hydroxybutyryl-CoA, 3-hydroxybutyryl-CoA to crotonyl-CoA, crotonyl-CoA to glutaryl-CoA, glutaryl-CoA to 3-oxopimeloyl-CoA, 3-oxopimeloyl-CoA to 3-hydroxypimeloyl-CoA, 3-hydroxypimeloyl-CoA to 6-carboxyhex-2-enoyl-CoA, and 6-carboxyhex-2-enoyl-CoA to pimeloyl-CoA. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 2.

In an additional embodiment, the invention provides a non-naturally occurring microbial organism having a cyclohexanone pathway, wherein the non-naturally occurring microbial organism comprises at least one exogenous nucleic acid encoding an enzyme or protein that converts a substrate to a product selected from the group consisting of 3-hydroxypimeloyl-CoA to 6-ketocyclohex-1-ene-1-carboxyl-CoA, 6-ketocyclohex-1-ene-1-carboxyl-CoA to 6-ketocyclohex-1-ene-1-carboxylate, 6-ketocyclohex-1-ene-1-carboxylate to 2-cyclohexenone, and 2-cyclohexenone to cyclohexanone. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 3.

In an additional embodiment, the invention provides a non-naturally occurring microbial organism having a cyclohexanone pathway, wherein the non-naturally occurring microbial organism comprises at least one exogenous nucleic acid encoding an enzyme or protein that converts a substrate to a product selected from the group consisting of 3-hydroxypimeloyl-CoA to 6-ketocyclohex-1-ene-1-carboxyl-CoA, 6-ketocyclohex-1-ene-1-carboxyl-CoA to 6-ketocyclohex-1-ene-1-carboxylate, 6-ketocyclohex-1-ene-1-carboxylate to 2-ketocyclohexane-1-carboxylate, and 2-ketocyclohexane-1-carboxylate to cyclohexanone. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 3.

In an additional embodiment, the invention provides a non-naturally occurring microbial organism having a cyclohexanone pathway, wherein the non-naturally occurring microbial organism comprises at least one exogenous nucleic acid encoding an enzyme or protein that converts a substrate to a product selected from the group consisting of 3-hydroxypimeloyl-CoA to 6-ketocyclohex-1-ene-1-carboxyl-CoA, 6-ketocyclohex-1-ene-1-carboxyl-CoA to 2-ketocyclohexane-1-carboxyl-CoA, 2-ketocyclohexane-1-carboxyl-CoA to 2-ketocyclohexane-1-carboxylate, and 2-ketocyclohexane-1-carboxylate to cyclohexanone. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 3.

In an additional embodiment, the invention provides a non-naturally occurring microbial organism having a cyclohexanone pathway, wherein the non-naturally occurring microbial organism comprises at least one exogenous nucleic acid encoding an enzyme or protein that converts a substrate to a product selected from the group consisting of adipate semialdehyde to cyclohexane-1,2-dione, cyclohexane-1,2-dione to 2-hydroxycyclohexan-1-one, 2-hydroxycyclohexan-1-one to cyclohexane-1,2-diol, and cyclohexane-1,2-diol to cyclohexanone. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 4.

In an additional embodiment, the invention provides a non-naturally occurring microbial organism having a cyclohexanone pathway, wherein the non-naturally occurring microbial organism comprises at least one exogenous nucleic acid encoding an enzyme or protein that converts a substrate to a product selected from the group consisting of 3-oxopimeloyl-CoA to 3-oxopimelate, 3-oxopimelate to 4-acetylbutyrate, 4-acetylbutyrate to 1,3-cyclohexanedione, 1,3-cyclohexanedione to 3-hydroxycyclohexanone, 3-hydroxycyclohexanone to 2-cyclohexenone, and 2-cyclohexenone to cyclohexanone. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 5.

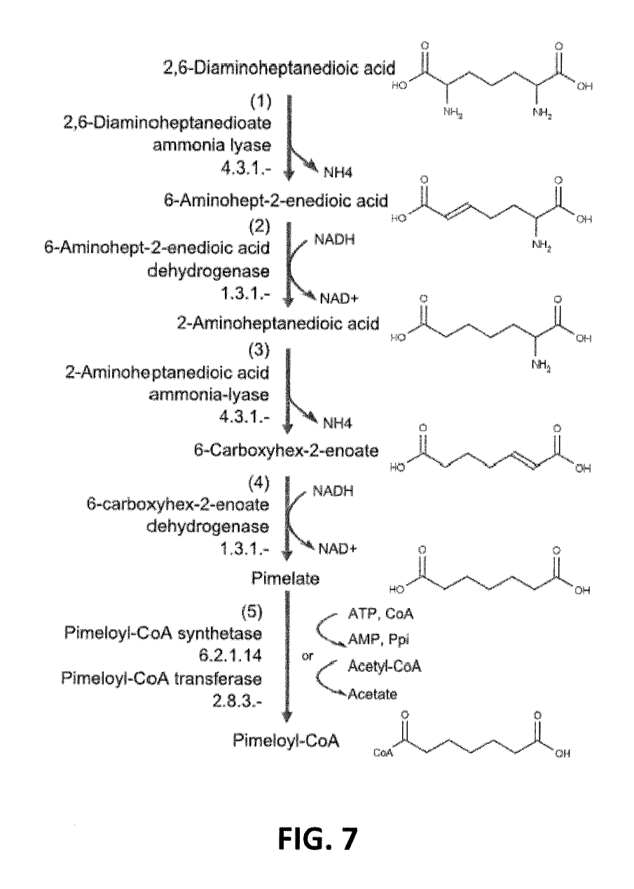

In an additional embodiment, the invention provides a non-naturally occurring microbial organism having a pimeloyl-CoA pathway, wherein the non-naturally occurring microbial organism comprises at least one exogenous nucleic acid encoding an enzyme or protein that converts a substrate to a product selected from the group consisting of 2,6-diaminoheptanedioc acid to 6-aminohept-2-enedioic acid, 6-aminohept-2-enedioic acid to 2-aminoheptanedioic acid, 2-aminoheptanedioic acid to 6-carboxyhex-2-eneoate, 6-carboxyhex-2-eneoate to pimelate, and pimelate to pimeloyl-CoA. Thus, the invention provides a non-naturally occurring microbial organism containing at least one exogenous nucleic acid encoding an enzyme or protein, where the enzyme or protein converts the substrates and products of a cyclohexanone pathway, such as that shown in FIG. 7.

While generally described herein as a microbial organism that contains a cyclohexanone pathway, it is understood that the invention additionally provides a non-naturally occurring microbial organism comprising at least one exogenous nucleic acid encoding a cyclohexanone pathway enzyme expressed in a sufficient amount to produce an intermediate of a cyclohexanone pathway. For example, as disclosed herein, a cyclohexanone pathway is exemplified in FIGS. 1-5 and 7. Therefore, in addition to a microbial organism containing a cyclohexanone pathway that produces cyclohexanone, the invention additionally provides a non-naturally occurring microbial organism comprising at least one exogenous nucleic acid encoding a cyclohexanone pathway enzyme, where the microbial organism produces a cyclohexanone pathway intermediate, for example, 2-KCH-CoA or 2-KCH as shown in FIG. 1, 3-hydroxybutyryl-CoA, crontonyl-CoA, glutaryl-CoA, 3-oxopimeloyl-CoA, 3-hydroxypimeloyl-CoA, or pimeloyl-CoA as shown in FIG. 2, 2-KCH, 2-KCH-CoA, 6-KCH-CoA, 6-KCH, or 2-cyclohexenone, as shown in FIG. 3, cyclohexane-1,2-dione, 2-hydroxycyclohexane-1-one, or cyclohexan-1,2-diol, as shown in FIG. 4, 3-oxopimelate, 4-acetylbutyrate, 1,3-cyclohexanedione, 3-hydroxycyclohexanone, or 2-cyclohexenone, as shown in FIG. 5, and 6-aminohept-2-enedioc acid, 2-aminoheptanedioic acid, 6-carboxyhex-2-enoate, or pimelate, as shown in FIG. 7.

It is understood that any of the pathways disclosed herein, as described in the Examples and exemplified in the Figures, including the pathways of FIGS. 1-5 and 7, can be utilized to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces a cyclohexanone pathway intermediate can be utilized to produce the intermediate as a desired product.

This invention is also directed, in part to engineered biosynthetic pathways to improve carbon flux through a central metabolism intermediate en route to cyclohexanone. The present invention provides non-naturally occurring microbial organisms having one or more exogenous genes encoding enzymes that can catalyze various enzymatic transformations en route to cyclohexanone. In some embodiments, these enzymatic transformations are part of the reductive tricarboxylic acid (RTCA) cycle and are used to improve product yields, including but not limited to, from carbohydrate-based carbon feedstock.

In numerous engineered pathways, realization of maximum product yields based on carbohydrate feedstock is hampered by insufficient reducing equivalents or by loss of reducing equivalents and/or carbon to byproducts. In accordance with some embodiments, the present invention increases the yields of cyclohexanone by (i) enhancing carbon fixation via the reductive TCA cycle, and/or (ii) accessing additional reducing equivalents from gaseous carbon sources and/or syngas components such as CO, CO₂, and/or H₂. In addition to syngas, other sources of such gases include, but are not limited to, the atmosphere, either as found in nature or generated.

The CO₂-fixing reductive tricarboxylic acid (RTCA) cycle is an endergenic anabolic pathway of CO₂ assimilation which uses reducing equivalents and ATP (FIG. 2a). One turn of the RTCA cycle assimilates two moles of CO₂ into one mole of acetyl-CoA, or four moles of CO₂ into one mole of oxaloacetate. This additional availability of acetyl-CoA improves the maximum theoretical yield of product molecules derived from carbohydrate-based carbon feedstock. Exemplary carbohydrates include but are not limited to glucose, sucrose, xylose, arabinose and glycerol.

In some embodiments, the reductive TCA cycle, coupled with carbon monoxide dehydrogenase and/or hydrogenase enzymes, can be employed to allow syngas, CO₂, CO, H₂, and/or other gaseous carbon source utilization by microorganisms. Synthesis gas (syngas), in particular is a mixture of primarily H₂ and CO, sometimes including some amounts of CO₂, that can be obtained via gasification of any organic feedstock, such as coal, coal oil, natural gas, biomass, or waste organic matter. Numerous gasification processes have been developed, and most designs are based on partial oxidation, where limiting oxygen avoids full combustion, of organic materials at high temperatures (500-1500° C.) to provide syngas as a 0.5:1-3:1 H₂/CO mixture. In addition to coal, biomass of many types has been used for syngas production and represents an inexpensive and flexible feedstock for the biological production of renewable chemicals and fuels. Carbon dioxide can be provided from the atmosphere or in condensed from, for example, from a tank cylinder, or via sublimation of solid CO₂. Similarly, CO and hydrogen gas can be provided in reagent form and/or mixed in any desired ratio. Other gaseous carbon forms can include, for example, methanol or similar volatile organic solvents.

The components of synthesis gas and/or other carbon sources can provide sufficient CO₂, reducing equivalents, and ATP for the reductive TCA cycle to operate. One turn of the RTCA cycle assimilates two moles of CO₂ into one mole of acetyl-CoA and requires 2 ATP and 4 reducing equivalents. CO and/or H₂ can provide reducing equivalents by means of carbon monoxide dehydrogenase and hydrogenase enzymes, respectively. Reducing equivalents can come in the form of NADH, NADPH, FADH, reduced quinones, reduced ferredoxins, and reduced flavodoxins. The reducing equivalents, particularly NADH, NADPH, and reduced ferredoxin, can serve as cofactors for the RTCA cycle enzymes, for example, malate dehydrogenase, fumarate reductase, alpha-ketoglutarate:ferredoxin oxidoreductase (alternatively known as 2-oxoglutarate:ferredoxin oxidoreductase, alpha-ketoglutarate synthase, or 2-oxoglutarate synthase), pyruvate:ferredoxin oxidoreductase and isocitrate dehydrogenase. The electrons from these reducing equivalents can alternatively pass through an ion-gradient producing electron transport chain where they are passed to an acceptor such as oxygen, nitrate, oxidized metal ions, protons, or an electrode. The ion-gradient can then be used for ATP generation via an ATP synthase or similar enzyme.

The reductive TCA cycle was first reported in the green sulfur photosynthetic bacterium Chlorobium limicola (Evans et al., Proc. Natl. Acad. Sci. USA. 55:928-934 (1966)). Similar pathways have been characterized in some prokaryotes (proteobacteria, green sulfur bacteria and thermophillic Knallgas bacteria) and sulfur-dependent archaea (Hugler et al., J. Bacteriol. 187:3020-3027 (2005; Hugler et al., Environ. Microbiol. 9:81-92 (2007). In some cases, reductive and oxidative (Krebs) TCA cycles are present in the same organism (Hugler et al., supra (2007); Siebers et al., J. Bacteriol. 186:2179-2194 (2004)). Some methanogens and obligate anaerobes possess incomplete oxidative or reductive TCA cycles that may function to synthesize biosynthetic intermediates (Ekiel et al., J. Bacteriol. 162:905-908 (1985); Wood et al., FEMS Microbiol. Rev. 28:335-352 (2004)).

The key carbon-fixing enzymes of the reductive TCA cycle are alpha-ketoglutarate:ferredoxin oxidoreductase, pyruvate:ferredoxin oxidoreductase and isocitrate dehydrogenase. Additional carbon may be fixed during the conversion of phosphoenolpyruvate to oxaloacetate by phosphoenolpyruvate carboxylase or phosphoenolpyruvate carboxykinase.

Many of the enzymes in the TCA cycle are reversible and can catalyze reactions in the reductive and oxidative directions. However, some TCA cycle reactions are irreversible in vivo and thus different enzymes are used to catalyze these reactions in the directions required for the reverse TCA cycle. These reactions are: (1) conversion of citrate to oxaloacetate and acetyl-CoA, (2) conversion of fumarate to succinate, and (3) conversion of succinyl-CoA to alpha-ketoglutarate. In the TCA cycle, citrate is formed from the condensation of oxaloacetate and acetyl-CoA. The reverse reaction, cleavage of citrate to oxaloacetate and acetyl-CoA, is ATP-dependent and catalyzed by ATP-citrate lyase, or citryl-CoA synthetase and citryl-CoA lyase. Alternatively, citrate lyase can be coupled to acetyl-CoA synthetase, an acetyl-CoA transferase, or phosphotransacetylase and acetate kinase to form acetyl-CoA and oxaloacetate from citrate. The conversion of succinate to fumarate is catalyzed by succinate dehydrogenase while the reverse reaction is catalyzed by fumarate reductase. In the TCA cycle succinyl-CoA is formed from the NAD(P)⁺ dependent decarboxylation of oxaloacetate by the alpha-ketoglutarate dehydrogenase complex. The reverse reaction is catalyzed by alpha-ketoglutarate:ferredoxin oxidoreductase.

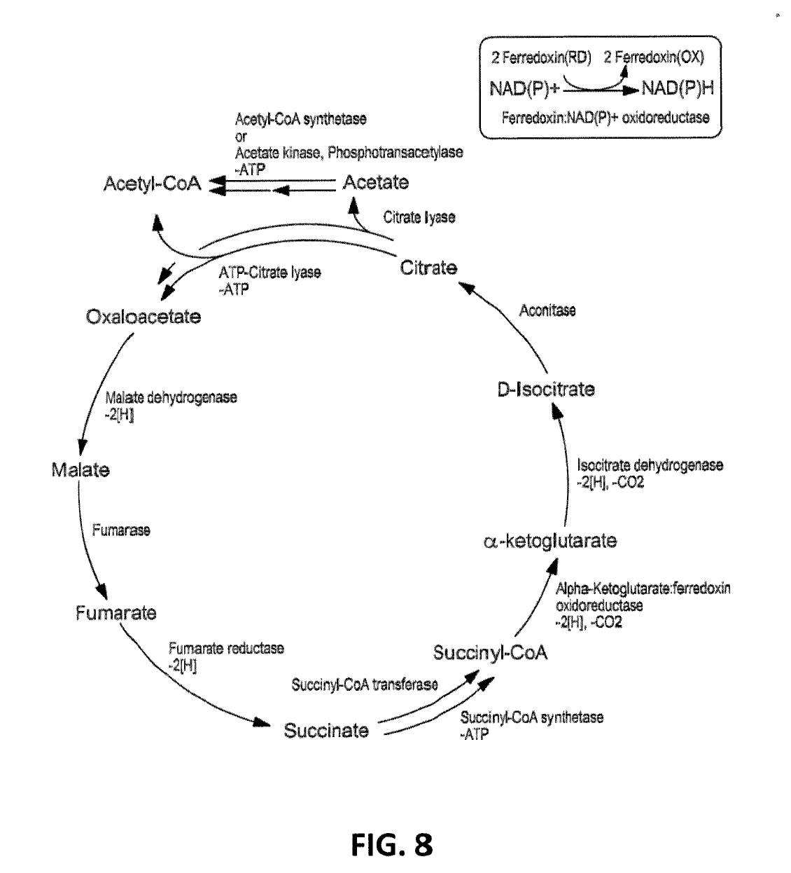

An organism capable of utilizing the reverse tricarboxylic acid cycle to enable production of acetyl-CoA-derived products on 1) CO, 2) CO₂ and H₂, 3) CO and CO₂, 4) synthesis gas comprising CO and H₂, and 5) synthesis gas or other gaseous carbon sources comprising CO, CO₂, and H₂ can include any of the following enzyme activities: ATP-citrate lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, acetate kinase, phosphotransacetylase, acetyl-CoA synthetase, pyruvate:ferredoxin oxidoreductase, NAD(P)H:ferredoxin oxidoreductase, carbon monoxide dehydrogenase, hydrogenase, and ferredoxin (see FIG. 8). Enzyme enzymes and the corresponding genes required for these activities are described herein above.

Carbon from syngas or other gaseous carbon sources can be fixed via the reverse TCA cycle and components thereof. Specifically, the combination of certain carbon gas-utilization pathway components with the pathways for formation of cyclohexanone from acetyl-CoA results in high yields of these products by providing an efficient mechanism for fixing the carbon present in carbon dioxide, fed exogenously or produced endogenously from CO, into acetyl-CoA.

In some embodiments, a cyclohexanone pathway in a non-naturally occurring microbial organism of the invention can utilize any combination of (1) CO, (2) CO₂, (3) H₂, or mixtures thereof to enhance the yields of biosynthetic steps involving reduction, including addition to driving the reductive TCA cycle.

In some embodiments a non-naturally occurring microbial organism having a cyclohexanone pathway includes at least one exogenous nucleic acid encoding a reductive TCA pathway enzyme. The at least one exogenous nucleic acid is selected from an ATP-citrate lyase, citrate lyase, a fumarate reductase, and an alpha-ketoglutarate:ferredoxin oxidoreductase; and at least one exogenous enzyme selected from a carbon monoxide dehydrogenase, a hydrogenase, a NAD(P)H:ferredoxin oxidoreductase, and a ferredoxin, expressed in a sufficient amount to allow the utilization of (1) CO, (2) CO₂, (3) H₂, (4) CO₂ and H₂, (5) CO and CO₂, (6) CO and H₂, or (7) CO, CO₂, and H₂.

In some embodiments a method includes culturing a non-naturally occurring microbial organism having a cyclohexanone pathway also comprising at least one exogenous nucleic acid encoding a reductive TCA pathway enzyme. The at least one exogenous nucleic acid is selected from an ATP-citrate lyase, citrate lyase, a fumarate reductase, and an alpha-ketoglutarate:ferredoxin oxidoreductase. Additionally, such an organism can also include at least one exogenous enzyme selected from a carbon monoxide dehydrogenase, a hydrogenase, a NAD(P)H:ferredoxin oxidoreductase, and a ferredoxin, expressed in a sufficient amount to allow the utilization of (1) CO, (2) CO₂, (3) H₂, (4) CO₂ and H₂, (5) CO and CO₂, (6) CO and H₂, or (7) CO, CO₂, and H₂ to produce a product.

In some embodiments a non-naturally occurring microbial organism having a cyclohexanone pathway further includes at least one exogenous nucleic acid encoding a reductive TCA pathway enzyme expressed in a sufficient amount to enhance carbon flux through acetyl-CoA. The at least one exogenous nucleic acid is selected from an ATP-citrate lyase, citrate lyase, a fumarate reductase, a pyruvate:ferredoxin oxidoreductase and an alpha-ketoglutarate:ferredoxin oxidoreductase.

In some embodiments a non-naturally occurring microbial organism having a cyclohexanone pathway includes at least one exogenous nucleic acid encoding an enzyme expressed in a sufficient amount to enhance the availability of reducing equivalents in the presence of carbon monoxide and/or hydrogen, thereby increasing the yield of redox-limited products via carbohydrate-based carbon feedstock. The at least one exogenous nucleic acid is selected from a carbon monoxide dehydrogenase, a hydrogenase, an NAD(P)H:ferredoxin oxidoreductase, and a ferredoxin. In some embodiments, the present invention provides a method for enhancing the availability of reducing equivalents in the presence of carbon monoxide or hydrogen thereby increasing the yield of redox-limited products via carbohydrate-based carbon feedstock, such as sugars or gaseous carbon sources, the method includes culturing this non-naturally occurring microbial organism under conditions and for a sufficient period of time to produce cyclohexanone.

In some embodiments, the non-naturally occurring microbial organism having a cyclohexanone pathway includes two exogenous nucleic acids each encoding a reductive TCA pathway enzyme. In some embodiments, the non-naturally occurring microbial organism having a cyclohexanone pathway includes three exogenous nucleic acids each encoding a reductive TCA pathway enzyme. In some embodiments, the non-naturally occurring microbial organism includes three exogenous nucleic acids encoding an ATP-citrate lyase, a fumarate reductase, and an alpha-ketoglutarate:ferredoxin oxidoreductase. In some embodiments, the non-naturally occurring microbial organism includes three exogenous nucleic acids encoding a citrate lyase, a fumarate reductase, and an alpha-ketoglutarate:ferredoxin oxidoreductase.

In some embodiments, the non-naturally occurring microbial organisms having a cyclohexanone pathway further include an exogenous nucleic acid encoding an enzyme selected from a pyruvate:ferredoxin oxidoreductase, an aconitase, an isocitrate dehydrogenase, a succinyl-CoA synthetase, a succinyl-CoA transferase, a fumarase, a malate dehydrogenase, an acetate kinase, a phosphotransacetylase, an acetyl-CoA synthetase, an NAD(P)H:ferredoxin oxidoreductase, and combinations thereof.

In some embodiments, the non-naturally occurring microbial organism having a cyclohexanone pathway further includes an exogenous nucleic acid encoding an enzyme selected from carbon monoxide dehydrogenase, acetyl-CoA synthase, ferredoxin, NAD(P)H:ferredoxin oxidoreductase and combinations thereof.

In some embodiments, the non-naturally occurring microbial organism having a cyclohexanone pathway utilizes a carbon feedstock selected from (1) CO, (2) CO₂, (3) CO₂ and H₂, (4) CO and H₂, or (5) CO, CO₂, and H₂. In some embodiments, the non-naturally occurring microbial organism having a cyclohexanone pathway utilizes hydrogen for reducing equivalents. In some embodiments, the non-naturally occurring microbial organism having a cyclohexanone pathway utilizes CO for reducing equivalents. In some embodiments, the non-naturally occurring microbial organism having a cyclohexanone pathway utilizes combinations of CO and hydrogen for reducing equivalents.

In some embodiments, the non-naturally occurring microbial organism having a cyclohexanone pathway further includes one or more nucleic acids encoding an enzyme selected from a phosphoenolpyruvate carboxylase, a phosphoenolpyruvate carboxykinase, a pyruvate carboxylase, and a malic enzyme.

In some embodiments, the non-naturally occurring microbial organism having a cyclohexanone pathway further includes one or more nucleic acids encoding an enzyme selected from a malate dehydrogenase, a fumarase, a fumarate reductase, a succinyl-CoA synthetase, and a succinyl-CoA transferase.

It is understood by those skilled in the art that the above-described pathways for increasing product yield can be combined with any of the pathways disclosed herein, including those pathways depicted in the figures. One skilled in the art will understand that, depending on the pathway to a desired product and the precursors and intermediates of that pathway, a particular pathway for improving product yield, as discussed herein above and in the examples, or combination of such pathways, can be used in combination with a pathway to a desired product to increase the yield of that product or a pathway intermediate.

In some embodiments, the non-naturally occurring microbial organism having a cyclohexanone pathway further includes at least one exogenous nucleic acid encoding a citrate lyase, an ATP-citrate lyase, a citryl-CoA synthetase, a citryl-CoA lyase an aconitase, an isocitrate dehydrogenase, a succinyl-CoA synthetase, a succinyl-CoA transferase, a fumarase, a malate dehydrogenase, an acetate kinase, a phosphotransacetylase, an acetyl-CoA synthetase, and a ferredoxin.

In some embodiments a non-naturally occurring microbial organism includes a microbial organism having a cyclohexanone pathway that includes at least one exogenous nucleic acid encoding a cyclohexanone pathway enzyme expressed in a sufficient amount to produce cyclohexanone; the non-naturally occurring microbial organism further includes:

(i) a reductive TCA pathway comprising at least one exogenous nucleic acid encoding a reductive TCA pathway enzyme, wherein said at least one exogenous nucleic acid is selected from an ATP-citrate lyase, citrate lyase, a fumarate reductase, and an alpha-ketoglutarate:ferredoxin oxidoreductase;

(ii) a reductive TCA pathway comprising at least one exogenous nucleic acid encoding a reductive TCA pathway enzyme, wherein said at least one exogenous nucleic acid is selected from a pyruvate:ferredoxin oxidoreductase, a phosphoenolpyruvate carboxylase, a phosphoenolpyruvate carboxykinase, a CO dehydrogenase, and an H₂ hydrogenase; or

(iii) at least one exogenous nucleic acid encodes an enzyme selected from a CO dehydrogenase, an H₂ hydrogenase, and combinations thereof;

wherein the cyclohexanone pathway includes a pathway selected from:

(a) a PEP carboxykinase, a 2-ketocyclohexane-1-carboxyl-CoA hydrolase (acting on C—C bond), a 2-ketocyclohexane-1-carboxylate decarboxylase and an enzyme selected from the group consisting of a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase;

(b) a PEP carboxykinase, a 6-ketocyclohex-1-ene-1-carboxyl-CoA hydrolase (acting on C—C bond), a 6-ketocyclohex-1-ene-1-carboxyl-CoA synthetase, a 6-ketocyclohex-1-ene-1-carboxyl-CoA hydrolase (acting on thioester), a 6-ketocyclohex-1-ene-1-carboxyl-CoA transferase, a 6-ketocyclohex-1-ene-1-carboxyl-CoA reductase, a 6-ketocyclohex-1-ene-1-carboxylate decarboxylase, a 6-ketocyclohex-1-ene-1-carboxylate reductase, a 2-ketocyclohexane-1-carboxyl-CoA synthetase, a 2-ketocyclohexane-1-carboxyl-CoA transferase, a 2-ketocyclohexane-1-carboxyl-CoA hydrolase (acting on thioester), a 2-ketocyclohexane-1-carboxylate decarboxylase, and a cyclohexanone dehydrogenase;

(c) a PEP carboxykinase, 6-ketocyclohex-1-ene-1-carboxyl-CoA hydrolase (acting on C—C bond), 6-ketocyclohex-1-ene-1-carboxylate decarboxylase, cyclohexanone dehydrogenase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase;

(d) PEP carboxykinase, 6-ketocyclohex-1-ene-1-carboxyl-CoA hydrolase (acting on C—C bond), 6-ketocyclohex-1-ene-1-carboxylate reductase, 2-ketocyclohexane-1-carboxylate decarboxylase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase;

(e) PEP carboxykinase, 6-ketocyclohex-1-ene-1-carboxyl-CoA hydrolase (acting on C—C bond), 6-ketocyclohex-1-ene-1-carboxyl-CoA reductase, 2-ketocyclohexane-1-carboxylate decarboxylase, and an enzyme selected from the group consisting of 2-ketocyclohexane-1-carboxyl-CoA synthetase, 2-ketocyclohexane-1-carboxyl-CoA transferase, and 2-ketocyclohexane-1-carboxyl-CoA hydrolase (acting on thioester);

(f) a PEP carboxykinase, an adipate semialdehyde dehydratase, a cyclohexane-1,2-diol dehydrogenase, and a cyclohexane-1,2-diol dehydratase; and

(g) a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from the group consisting of a 3-oxopimeloyl-CoA synthetase, a 3-oxopimeloyl-CoA hydrolase (acting on thioester), and a 3-oxopimeloyl-coA transferase.

In some embodiments, the non-naturally occurring microbial organism has a cyclohexanone pathway that includes at least one exogenous nucleic acid encoding a cyclohexanone pathway enzyme from (a) and wherein the microbial organism further includes a pimeloyl-CoA pathway that includes at least one exogenous nucleic acid encoding a pimeloyl-CoA pathway enzyme expressed in a sufficient amount to produce pimeloyl-CoA, the pimeloyl-CoA pathway includes an acetoacetyl-CoA reductase, a 3-hydroxybutyryl-CoA dehydratase, a glutaryl-CoA dehydrogenase, a oxopimeloyl-CoA:glutaryl-CoA acyltransferase, a 3-hydroxypimeloyl-CoA dehydrogenase, a 3-hydroxypimeloyl-CoA dehydratase, and a pimeloyl-CoA dehydrogenase.

In some embodiments, the non-naturally occurring microbial organism has a cyclohexanone pathway that includes at least one exogenous nucleic acid encoding a cyclohexanone pathway enzyme from (b), and wherein the microbial organism has a native 3-hydroxypimeloyl-CoA pathway.

In some embodiments, the non-naturally occurring microbial organism has a cyclohexanone pathway that includes at least one exogenous nucleic acid encoding a cyclohexanone pathway enzyme from (b) and wherein the microbial organism further includes a 3-hydroxypimeloyl-CoA pathway that includes at least one exogenous nucleic acid encoding a 3-hydroxypimeloyl-CoA pathway enzyme expressed in a sufficient amount to produce 3-hydroxypimeloyl-CoA, the 3-hydroxypimeloyl-CoA pathway includes a acetoacetyl-CoA reductase, a 3-hydroxybutyryl-CoA dehydratase, a glutaryl-CoA dehydrogenase, a oxopimeloyl-CoA:glutaryl-CoA acyltransferase, and a 3-hydroxypimeloyl-CoA dehydrogenase.

In some embodiments, the non-naturally occurring microbial organism (e.g., having pathway (i)) further includes an exogenous nucleic acid encoding an enzyme selected from a pyruvate:ferredoxin oxidoreductase, an aconitase, an isocitrate dehydrogenase, a succinyl-CoA synthetase, a succinyl-CoA transferase, a fumarase, a malate dehydrogenase, an acetate kinase, a phosphotransacetylase, an acetyl-CoA synthetase, an NAD(P)H:ferredoxin oxidoreductase, ferredoxin, and combinations thereof.

In some embodiments, the non-naturally occurring microbial organism (e.g., having pathway (ii)) further includes an exogenous nucleic acid encoding an enzyme selected from an aconitase, an isocitrate dehydrogenase, a succinyl-CoA synthetase, a succinyl-CoA transferase, a fumarase, a malate dehydrogenase, and combinations thereof.

In some embodiments, the non-naturally occurring microbial organism includes two, three, four, five, six or seven exogenous nucleic acids each encoding a cyclohexanone pathway enzyme.

In some embodiments, the non-naturally occurring microbial organism includes exogenous nucleic acids encoding each of the enzymes selected from:

(a) a PEP carboxykinase, a 2-ketocyclohexane-1-carboxyl-CoA hydrolase (acting on C—C bond), a 2-ketocyclohexane-1-carboxylate decarboxylase and an enzyme selected from the group consisting of a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase;

(b) a PEP carboxykinase, a 6-ketocyclohex-1-ene-1-carboxyl-CoA hydrolase (acting on C—C bond), a 6-ketocyclohex-1-ene-1-carboxyl-CoA synthetase, a 6-ketocyclohex-1-ene-1-carboxyl-CoA hydrolase (acting on thioester), a 6-ketocyclohex-1-ene-1-carboxyl-CoA transferase, a 6-ketocyclohex-1-ene-1-carboxyl-CoA reductase, a 6-ketocyclohex-1-ene-1-carboxylate decarboxylase, a 6-ketocyclohex-1-ene-1-carboxylate reductase, a 2-ketocyclohexane-1-carboxyl-CoA synthetase, a 2-ketocyclohexane-1-carboxyl-CoA transferase, a 2-ketocyclohexane-1-carboxyl-CoA hydrolase (acting on thioester), a 2-ketocyclohexane-1-carboxylate decarboxylase, and a cyclohexanone dehydrogenase;

(c) a PEP carboxykinase, 6-ketocyclohex-1-ene-1-carboxyl-CoA hydrolase (acting on C—C bond), 6-ketocyclohex-1-ene-1-carboxylate decarboxylase, cyclohexanone dehydrogenase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase;

(d) PEP carboxykinase, 6-ketocyclohex-1-ene-1-carboxyl-CoA hydrolase (acting on C—C bond), 6-ketocyclohex-1-ene-1-carboxylate reductase, 2-ketocyclohexane-1-carboxylate decarboxylase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase;

(e) PEP carboxykinase, 6-ketocyclohex-1-ene-1-carboxyl-CoA hydrolase (acting on C—C bond), 6-ketocyclohex-1-ene-1-carboxyl-CoA reductase, 2-ketocyclohexane-1-carboxylate decarboxylase, and an enzyme selected from the group consisting of 2-ketocyclohexane-1-carboxyl-CoA synthetase, 2-ketocyclohexane-1-carboxyl-CoA transferase, and 2-ketocyclohexane-1-carboxyl-CoA hydrolase (acting on thioester);

(f) a PEP carboxykinase, an adipate semialdehyde dehydratase, a cyclohexane-1,2-diol dehydrogenase, and a cyclohexane-1,2-diol dehydratase; and

(g) a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from the group consisting of a 3-oxopimeloyl-CoA synthetase, a 3-oxopimeloyl-CoA hydrolase (acting on thioester), and a 3-oxopimeloyl-coA transferase

In some embodiments, the non-naturally occurring microbial organism includes two, three, four or five exogenous nucleic acids each encoding enzymes of (i), (ii) or (iii).

In some embodiments, the non-naturally occurring microbial organism having pathway (i) includes four exogenous nucleic acids encoding ATP-citrate lyase, citrate lyase, a fumarate reductase, and an alpha-ketoglutarate:ferredoxin oxidoreductase;

The microbial organism having pathway (ii) includes five exogenous nucleic acids encoding pyruvate:ferredoxin oxidoreductase, a phosphoenolpyruvate carboxylase, a phosphoenolpyruvate carboxykinase, a CO dehydrogenase, and an H₂ hydrogenase; or

The microbial organism having pathway (iii) includes two exogenous nucleic acids encoding CO dehydrogenase and H₂ hydrogenase.

In some embodiments, the non-naturally occurring microbial organism has at least one exogenous nucleic acid that is a heterologous nucleic acid.

In some embodiments, the non-naturally occurring microbial organism is in a substantially anaerobic culture medium.

In some embodiments, a method for producing cyclohexanone includes culturing any of the aforementioned non-naturally occurring microbial organisms under conditions and for a sufficient period of time to produce cyclohexanone.

In certain embodiments, the microbial organism comprises a nucleic acid encoding each of the enzymes in the recited pathway.

Also provided herein is a non-naturally occurring microbial organism having a cyclohexanone pathway, wherein said microbial organism comprises at least one exogenous nucleic acid encoding a cyclohexanone pathway enzyme expressed in a sufficient amount to produce cyclohexanone; said non-naturally occurring microbial organism further comprising:

(i) a reductive TCA pathway, wherein said microbial organism comprises at least one exogenous nucleic acid encoding a reductive TCA pathway enzyme selected from the group consisting of an ATP-citrate lyase, citrate lyase, a fumarate reductase, and an alpha-ketoglutarate:ferredoxin oxidoreductase;

(ii) a reductive TCA pathway, wherein said microbial organism comprises at least one exogenous nucleic acid encoding a reductive TCA pathway enzyme selected from the group consisting of a pyruvate:ferredoxin oxidoreductase, a phosphoenolpyruvate carboxylase, a phosphoenolpyruvate carboxykinase, a CO dehydrogenase, and an H₂ hydrogenase; or

(iii) at least one exogenous nucleic acid encodes an enzyme selected from a CO dehydrogenase, an H₂ hydrogenase, and combinations thereof;

wherein said cyclohexanone pathway comprises a pathway selected from the group consisting of:

(a) a PEP carboxykinase; a 2-ketocyclohexane-1-carboxyl-CoA hydrolase (acting on C—C bond); a 2-ketocyclohexane-1-carboxylate decarboxylase; and a 2-ketocyclohexane-1-carboxyl-CoA hydrolase (acting on thioester), 2-ketocyclohexane-1-carboxyl-CoA transferase, or 2-ketocyclohexane-1-carboxyl-CoA synthetase;

(b) a PEP carboxykinase; a 6-ketocyclohex-1-ene-1-carboxyl-CoA hydrolase (acting on C—C bond); a 6-ketocyclohex-1-ene-1-carboxyl-CoA synthetase; a 6-ketocyclohex-1-ene-1-carboxyl-CoA hydrolase (acting on thioester); a 6-ketocyclohex-1-ene-1-carboxyl-CoA transferase; a 6-ketocyclohex-1-ene-1-carboxyl-CoA reductase; a 6-ketocyclohex-1-ene-1-carboxylate decarboxylase; a 6-ketocyclohex-1-ene-1-carboxylate reductase; a 2-ketocyclohexane-1-carboxyl-CoA synthetase; a 2-ketocyclohexane-1-carboxyl-CoA transferase; a 2-ketocyclohexane-1-carboxyl-CoA hydrolase (acting on thioester); a 2-ketocyclohexane-1-carboxylate decarboxylase; and a cyclohexanone dehydrogenase;

(c) a PEP carboxykinase; a 6-ketocyclohex-1-ene-1-carboxyl-CoA hydrolase (acting on C—C bond); a 6-ketocyclohex-1-ene-1-carboxylate decarboxylase; a cyclohexanone dehydrogenase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;

(d) a PEP carboxykinase; a 6-ketocyclohex-1-ene-1-carboxyl-CoA hydrolase (acting on C—C bond); a 6-ketocyclohex-1-ene-1-carboxylate reductase; a 2-ketocyclohexane-1-carboxylate decarboxylase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;

(e) a PEP carboxykinase; a 6-ketocyclohex-1-ene-1-carboxyl-CoA hydrolase (acting on C—C bond); a 6-ketocyclohex-1-ene-1-carboxyl-CoA reductase; a 2-ketocyclohexane-1-carboxylate decarboxylase, and a 2-ketocyclohexane-1-carboxyl-CoA synthetase, 2-ketocyclohexane-1-carboxyl-CoA transferase, or 2-ketocyclohexane-1-carboxyl-CoA hydrolase (acting on thioester);

(f) a PEP carboxykinase; an adipate semialdehyde dehydratase; a cyclohexane-1,2-diol dehydrogenase; and a cyclohexane-1,2-diol dehydratase; and

(g) a PEP carboxykinase; a 3-oxopimelate decarboxylase; a 4-acetylbutyrate dehydratase; a 3-hydroxycyclohexanone dehydrogenase; a 2-cyclohexenone hydratase; a cyclohexanone dehydrogenase; and a 3-oxopimeloyl-CoA synthetase, 3-oxopimeloyl-CoA hydrolase (acting on thioester), or a 3-oxopimeloyl-coA transferase.

In certain embodiments, the microbial organism has a cyclohexanone pathway comprising at least one exogenous nucleic acid encoding a cyclohexanone pathway enzyme from (a); and wherein the microbial organism further comprises a pimeloyl-CoA pathway comprising at least one exogenous nucleic acid encoding a pimeloyl-CoA pathway enzyme expressed in a sufficient amount to produce pimeloyl-CoA, said pimeloyl-CoA pathway comprising an acetoacetyl-CoA reductase, a 3-hydroxybutyryl-CoA dehydratase, a glutaryl-CoA dehydrogenase, a oxopimeloyl-CoA:glutaryl-CoA acyltransferase, a 3-hydroxypimeloyl-CoA dehydrogenase, a 3-hydroxypimeloyl-CoA dehydratase, and a pimeloyl-CoA dehydrogenase.

In some embodiments, the microbial organism has a cyclohexanone pathway comprising at least one exogenous nucleic acid encoding a cyclohexanone pathway enzyme from (b), and wherein said microbial organism has a native 3-hydroxypimeloyl-CoA pathway.

In some embodiments, the microbial organism has a cyclohexanone pathway comprising at least one exogenous nucleic acid encoding a cyclohexanone pathway enzyme from (b), and wherein the microbial organism further comprises a 3-hydroxypimeloyl-CoA pathway comprising at least one exogenous nucleic acid encoding a 3-hydroxypimeloyl-CoA pathway enzyme expressed in a sufficient amount to produce 3-hydroxypimeloyl-CoA, said 3-hydroxypimeloyl-CoA pathway comprising a acetoacetyl-CoA reductase, a 3-hydroxybutyryl-CoA dehydratase, a glutaryl-CoA dehydrogenase, a oxopimeloyl-CoA:glutaryl-CoA acyltransferase, and a 3-hydroxypimeloyl-CoA dehydrogenase.

In some embodiments, the microbial organism comprising (i) further comprises an exogenous nucleic acid encoding an enzyme selected from the group consisting of a pyruvate:ferredoxin oxidoreductase, an aconitase, an isocitrate dehydrogenase, a succinyl-CoA synthetase, a succinyl-CoA transferase, a fumarase, a malate dehydrogenase, an acetate kinase, a phosphotransacetylase, an acetyl-CoA synthetase, an NAD(P)H:ferredoxin oxidoreductase, ferredoxin, and combinations thereof.

In some embodiments, the microbial organism comprising (ii) further comprises an exogenous nucleic acid encoding an enzyme selected from the group consisting of an aconitase, an isocitrate dehydrogenase, a succinyl-CoA synthetase, a succinyl-CoA transferase, a fumarase, a malate dehydrogenase, and combinations thereof.

In some embodiments, the microbial organism comprises two, three, four, five, six or seven exogenous nucleic acids, each encoding a cyclohexanone pathway enzyme.

In some embodiments, the microbial organism comprises exogenous nucleic acids encoding each of the enzymes of a cyclohexanone pathway selected from the group consisting of:

(a) a PEP carboxykinase; a 2-ketocyclohexane-1-carboxyl-CoA hydrolase (acting on C—C bond); a 2-ketocyclohexane-1-carboxylate decarboxylase; and a 2-ketocyclohexane-1-carboxyl-CoA hydrolase (acting on thioester), 2-ketocyclohexane-1-carboxyl-CoA transferase, or 2-ketocyclohexane-1-carboxyl-CoA synthetase;

(b) a PEP carboxykinase; a 6-ketocyclohex-1-ene-1-carboxyl-CoA hydrolase (acting on C—C bond); a 6-ketocyclohex-1-ene-1-carboxyl-CoA synthetase; a 6-ketocyclohex-1-ene-1-carboxyl-CoA hydrolase (acting on thioester); a 6-ketocyclohex-1-ene-1-carboxyl-CoA transferase; a 6-ketocyclohex-1-ene-1-carboxyl-CoA reductase; a 6-ketocyclohex-1-ene-1-carboxylate decarboxylase; a 6-ketocyclohex-1-ene-1-carboxylate reductase; a 2-ketocyclohexane-1-carboxyl-CoA synthetase; a 2-ketocyclohexane-1-carboxyl-CoA transferase; a 2-ketocyclohexane-1-carboxyl-CoA hydrolase (acting on thioester); a 2-ketocyclohexane-1-carboxylate decarboxylase; and a cyclohexanone dehydrogenase;

(c) a PEP carboxykinase; a 6-ketocyclohex-1-ene-1-carboxyl-CoA hydrolase (acting on C—C bond); a 6-ketocyclohex-1-ene-1-carboxylate decarboxylase; a cyclohexanone dehydrogenase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;

(d) a PEP carboxykinase; a 6-ketocyclohex-1-ene-1-carboxyl-CoA hydrolase (acting on C—C bond); a 6-ketocyclohex-1-ene-1-carboxylate reductase; a 2-ketocyclohexane-1-carboxylate decarboxylase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;

(e) a PEP carboxykinase; a 6-ketocyclohex-1-ene-1-carboxyl-CoA hydrolase (acting on C—C bond); a 6-ketocyclohex-1-ene-1-carboxyl-CoA reductase; a 2-ketocyclohexane-1-carboxylate decarboxylase, and a 2-ketocyclohexane-1-carboxyl-CoA synthetase, 2-ketocyclohexane-1-carboxyl-CoA transferase, or 2-ketocyclohexane-1-carboxyl-CoA hydrolase (acting on thioester);

(f) a PEP carboxykinase; an adipate semialdehyde dehydratase; a cyclohexane-1,2-diol dehydrogenase; and a cyclohexane-1,2-diol dehydratase; and

(g) a PEP carboxykinase; a 3-oxopimelate decarboxylase; a 4-acetylbutyrate dehydratase; a 3-hydroxycyclohexanone dehydrogenase; a 2-cyclohexenone hydratase; a cyclohexanone dehydrogenase; and a 3-oxopimeloyl-CoA synthetase, 3-oxopimeloyl-CoA hydrolase (acting on thioester), or a 3-oxopimeloyl-coA transferase.

In some embodiments, the microbial organism comprises two, three, four or five exogenous nucleic acids each encoding enzymes of (i), (ii) or (iii).

In some embodiments, wherein the microbial organism comprising (i) comprises four exogenous nucleic acids encoding ATP-citrate lyase, citrate lyase, a fumarate reductase, and an alpha-ketoglutarate:ferredoxin oxidoreductase; wherein said microbial organism comprising (ii) comprises five exogenous nucleic acids encoding pyruvate:ferredoxin oxidoreductase, a phosphoenolpyruvate carboxylase, a phosphoenolpyruvate carboxykinase, a CO dehydrogenase, and an H₂ hydrogenase; or wherein said microbial organism comprising (iii) comprises two exogenous nucleic acids encoding CO dehydrogenase and H₂ hydrogenase.

In some embodiments, the at least one exogenous nucleic acid is a heterologous nucleic acid.

In some embodiments, the non-naturally occurring microbial organism is in a substantially anaerobic culture medium.

Also provided herein is a method for producing cyclohexanone, comprising culturing a non-naturally occurring microbial organism provided herein under conditions and for a sufficient period of time to produce cyclohexanone.

In some embodiments, the carbon feedstock and other cellular uptake sources such as phosphate, ammonia, sulfate, chloride and other halogens can be chosen to alter the isotopic distribution of the atoms present in cyclohexanone or any cyclohexanone pathway intermediate. The various carbon feedstock and other uptake sources enumerated above will be referred to herein, collectively, as “uptake sources.” Uptake sources can provide isotopic enrichment for any atom present in the product cyclohexanone or cyclohexanone pathway intermediate including any cyclohexanone impurities generated in diverging away from the pathway at any point. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, a target isotopic ratio of an uptake source can be obtained via synthetic chemical enrichment of the uptake source. Such isotopically enriched uptake sources can be purchased commercially or prepared in the laboratory. In some embodiments, a target isotopic ratio of an uptake source can be obtained by choice of origin of the uptake source in nature. In some such embodiments, a source of carbon, for example, can be selected from a fossil fuel-derived carbon source, which can be relatively depleted of carbon-14, or an environmental carbon source, such as CO₂, which can possess a larger amount of carbon-14 than its petroleum-derived counterpart.

Isotopic enrichment is readily assessed by mass spectrometry using techniques known in the art such as Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC) and/or high performance liquid chromatography (HPLC).

In some embodiments, the present invention provides cyclohexanone or a cyclohexanone intermediate that has a carbon-12, carbon-13, and carbon-14 ratio that reflects an atmospheric carbon uptake source. In some such embodiments, the uptake source is CO₂. In some embodiments, In some embodiments, the present invention provides cyclohexanone or a cyclohexanone intermediate that has a carbon-12, carbon-13, and carbon-14 ratio that reflects petroleum-based carbon uptake source. In some embodiments, the present invention provides cyclohexanone or a cyclohexanone intermediate that has a carbon-12, carbon-13, and carbon-14 ratio that is obtained by a combination of an atmospheric carbon uptake source with a petroleum-based uptake source. Such combination of uptake sources is one means by which the carbon-12, carbon-13, and carbon-14 ratio can be varied.

The invention is described herein with general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

In some embodiments, a cyclohexanone pathway includes enzymes that convert pimeloyl-CoA to cyclohexanone in three enzymatic steps as shown in FIG. 1. In this route, pimeloyl-CoA is cyclized to 2-ketocyclohexane-1-carboxyl-CoA (2KCH-CoA) by 2-ketocyclohexane-1-carboxyl-CoA hydrolase (acting on C—C bond). The 2KCH-CoA hydrolase is run in the reverse, i.e. ring-closing direction as shown in FIG. 1. The CoA ester is then converted to 2-ketocyclohexane-1-carboxylate by reaction of 2-ketocyclohexane-1-carboxyl-CoA with a CoA synthetase, hydrolase or transferase. Finally decarboxylation of 2-ketocyclohexane-1-carboxylate yields cyclohexanone.

The energetics and theoretical cyclohexanone yield of this pathway, shown in Table 1, are dependent on: 1) the type of enzyme utilized for removing the CoA moiety in step 2, 2) the biosynthetic pathway for producing pimeloyl-CoA, and 3) the ability of PEP carboxykinase to operate in the ATP-generating direction.

TABLE 1

| | Cyclohexanone | ATP @ max yield |
|---|---|---|
| | (mol/mol glucose) | (mol/mol glucose) |
| Hydrolase | 0.738 | 0 |
| Hydrolase, PPCKr | .075 | 0.31 |
| Transferase | 0.75 | 0.56 |
| Transferase, PPCKr | 0.75 | 1.06 |

A strain that produces pimeloyl-CoA as described herein, with a transferase or synthetase in step (2), and a reversible PEP carboxykinase has a theoretical yield of 0.75 moles of cyclohexanone per mole glucose utilized (0.41 g/g). This strain has an energetic yield of 1.06 moles ATP per mole glucose utilized.

Enzymes for each step of a cyclohexanone pathway are described below. In some embodiments, native pathways for producing pimeloyl-CoA can be utilized, while in other embodiments novel pathways for synthesizing pimeloyl-CoA from central metabolic precursors are used.

The first step of the pathway involves formation of 2-ketocyclohexane-1-carboxyl-CoA from pimeloyl-CoA as shown in step 1 of FIG. 1. This transformation has been indicated to occur in the ring-closing direction in Syntrophus aciditrophicus during growth on crotonate (Mouttaki et al., Appl. Environ. Microbiol. 73:930-938 (2007)). This enzyme activity was also demonstrated in cell-free extracts of S. aciditrophicus in co-culture with another microbe during growth on benzoate (Elshahed et al., Appl. Environ. Microbiol. 67:1728-1738 (2001)). An enzyme catalyzing this activity in the ring-opening direction has been characterized in Rhodopseudomonas palustris, where it is encoded by badI (Pelletier et al., J. Bacteriol. 180:2330-2336 (1998)). The R. palustris enzyme has been expressed in E. coli where it was assayed for enzymatic activity in the ring-opening direction; however, such activity was not observed (Egland et al., Proc. Natl. Acad. Sci U.S.A. 94:6484-6489 (1997)). Several genes in the S. aciditrophicus genome bear sequence homology to the badI gene of R. palustris (McInerney et al., Proc. Natl. Acad. Sci. U.S.A. 104:7600-7605 (2007)), including syn_{—}01653 (38%), syn_{—}03076 (33%), syn_{—}02400 (33%), syn_{—}03076 (30%) and syn_{—}01309 (31%). The protein sequences for exemplary gene products can be found using the following GenBank accession numbers shown below in Table 2.

TABLE 2

| Protein | GenBank ID | GI Number | Organism |
|---|---|---|---|
| badI | NP_946006.1 | 39933730 | Rhodopseudomonas |
| | | | palustris |
| syn_01653 | YP_463074.1 | 85860872 | Syntrophus aciditrophicus |
| syn_01654 | YP_463073.1 | 85860871 | Syntrophus aciditrophicus |
| syn_02400 | YP_462924.1 | 85860722 | Syntrophus aciditrophicus |
| syn_03076 | YP_463118.1 | 85860916 | Syntrophus aciditrophicus |
| syn_01309 | YP_461962.1 | 85859760 | Syntrophus aciditrophicus |

Napthoyl-CoA synthetase (EC 4.1.3.36), an enzyme participating in menaquinone biosynthesis, catalyzes the ring-closing conversion of succinyl-benzoyl-CoA to 1,4-dihydroxy-2-napthoyl-CoA. The badI gene product of R. palustris shares as much as 53% sequence identity with 1,4-dihydroxynapthoyl-CoA synthetase homologs in other organisms (Eberhard et al., J. Am. Chem. Soc. 126:7188-7189 (2004)), and enzymes catalyzing this transformation can demonstrate 2-ketocyclohexane-1-carboxyl-CoA hydrolase (acting on C—C bond) activity in the ring-closing direction. Such enzymes are found in Escherichia coli (Sharma et al., J. Bacteriol. 174:5057-5062 (1992)), Bacillus subtilis (Driscoll et al., J. Bacteriol. 174:5063-5071 (1992)), Staphylococcus aureus (Ulaganathan et al., Acta Crstyallogr. Sect. F. Struct. Biol. Cyst. Commun. 63:908-913 (2007)) and Geobacillus kaustophilus (Kanajunia et al., Acta Crstyallogr. Sect. F. Struct. Biol. Cyst. Commun. 63:103-105 (2007)). Additionally, structural data is available for the enzymes from Mycobacterium tuberculosis (Johnston et al., Acta Crstyallogr. D. Biol. Crystallogr. 61:1199-1206 (2005)), S. aureus (Ulaganathan et al., supra) and Geobacillus kaustophilus (Kanaujia et al., supra). The protein sequences for exemplary gene products can be found using the following GenBank accession numbers shown below in Table 3.

TABLE 3

| Protein | GenBank ID | GI Number | Organism |
|---|---|---|---|
| menB | AAC75322 | 1788597 | Escherichia coli K12 sp. MG1655 |
| menB | AAC37016 | 143186 | Bacillus subtilis |
| menB | NP_215062 | 15607688 | Mycobacterium tuberculosis |
| menB | BAB57207 | 14246815 | Staphylococcus aureus |
| menB | BAD77158 | 56381250 | Geobacillus kaustophilus |

The reaction of 2-ketocyclohexane-1-carboxyl-CoA to 2-ketocyclohexane-1-carboxylate, shown in FIG. 1, step 2, can be accomplished by a CoA hydrolase, transferase or synthetase. 3-oxoacid CoA transferases include 3-oxoadipate CoA-transferase (EC 2.8.3.6), 3-oxoacid CoA transferase (2.8.3.5) and acetate-acetoacetate CoA-transferase (2.8.3.-). 3-Oxoadipate CoA transferase (EC 2.8.3.6) catalyzes the transfer of the CoA moiety from succinyl-CoA to 3-oxoadipate, a molecule close in structure to 3-oxopimelate. Participating in beta-ketoadipate pathways for aromatic compound degradation (Harwood et al., Annu. Rev. Microbiol. 50:553-590 (1996)), this enzyme has been characterized in Pseudomonas putida (Parales et al., J. Bacteriol. 174:4657-4666 (1992)), Acinetobacter calcoaceticus (sp. ADP1) (Dal et al., Appl. Environ. Microbiol. 71:1025-1034 (2005); Yeh et al., J. Biol. Chem. 256:1565-1569 (1981) and Pseudomonas knackmussii (formerly sp. B13) (Gobel et al., J. Bacteriol. 184:216-223 (2002); Kaschabek et al., J. Bacteriol. 184:207-215 (2002). The protein sequences for exemplary gene products can be found using the following GenBank accession numbers shown below in Table 4.

TABLE 4

| Protein | GenBank ID | GI Number | Organism |
|---|---|---|---|
| pcaI | Q01103.2 | 24985644 | Pseudomonas putida |
| pcaJ | P0A102.2 | 26990657 | Pseudomonas putida |
| pcaI (catI) | AAC37146.1 | 684991 | Acinetobacter calcoaceticus |
| | | | (sp. ADP1) |
| pcaJ (catJ) | AAC37147.1 | 141776 | Acinetobacter calcoaceticus |
| | | | (sp. ADP1) |
| catI | Q8VPF3.1 | 75404583 | Pseudomonas knackmussii |
| catJ | Q8VPF2.1 | 75404582 | Pseudomonas knackmussii |

Another CoA transferase for this reaction step is succinyl-CoA:3-ketoacid-CoA transferase. This enzyme converts succinate to succinyl-CoA while converting a 3-ketoacyl-CoA to a 3-ketoacid. Exemplary succinyl-CoA:3:ketoacid-CoA transferases are present in Helicobacter pylori (Corthesy-Theulaz et al., J. Biol. Chem. 272:25659-25667 (1997)), Bacillus subtilis (Stols et al., Protein. Expr. Purif. 53:396-403 (2007), and Homo sapiens (Fukao et al., Genomics 68:144-151 (2000); Tanaka et al., Mol. Hum. Reprod. 8:16-23 (2002)). The protein sequences for exemplary gene products can be found using the following GenBank accession numbers shown below in Table 5.

TABLE 5

| Protein | GenBank ID | GI Number | Organism |
|---|---|---|---|
| HPAG1_0676 | YP_627417 | 108563101 | Helicobacter pylori |
| HPAG1_0677 | YP_627418 | 108563102 | Helicobacter pylori |
| ScoA | NP_391778 | 16080950 | Bacillus subtilis |
| ScoB | NP_391777 | 16080949 | Bacillus subtilis |
| OXCT1 | NP_000427 | 4557817 | Homo sapiens |
| OXCT2 | NP_071403 | 11545841 | Homo sapiens |

Acetate-acetoacetate CoA transferase naturally transfers the CoA moiety from acetoacetyl-CoA to acetate, forming acetyl-CoA and acetoacetate. Exemplary enzymes include the gene products of ctfAB in Clostridium acetobutylicum (Weisenborn et al., App. Environ. Microbiol 55:323-329 (1989)), atoAD from Escherichia coli K12 (Sramek et al., Arch. Biochem. Biophys. 171:14-26 (1975)), and ctfAB from Clostridium saccharoperbutylacetonicum (Kosaka et al., Bopsco. Biotechnol. Biochem. 71:58-68 (2007)). The Clostridium acetobutylicum enzyme has been functionally expressed in E. coli (Cary et al., Appl. Environ. Microbiol. 56:1576-1583 (1990)). The CoA transferase in E. coli K12, encoded by atoA and atoD, has a fairly broad substrate specificity and has been shown to react with alternate 3-oxoacyl-CoA substrates (Sramek et al., supra). This enzyme is induced at the transcriptional level by acetoacetate, so modification of regulatory control can be performed to utilize this enzyme in a pathway (Pauli et al., Euro. J. Biochem. 29:553-562 (1972)). The protein sequences for exemplary gene products can be found using the following GenBank accession numbers shown below in Table 6.

TABLE 6

| Protein | GenBank ID | GI Number | Organism |
|---|---|---|---|
| ctfA | NP_149326.1 | 15004866 | Clostridium acetobutylicum |
| ctfB | NP_149327.1 | 15004867 | Clostridium acetobutylicum |
| atoA | NP_416726 | 2492994 | Escherichia coli K12 substr |
| | | | MG1655 |
| atoD | NP_416725 | 2492990 | Escherichia coli K12 substr |
| | | | MG1655 |
| ctfA | AAP42564.1 | 31075384 | Clostridium |
| | | | saccharoperbutylacetonicum |
| ctfB | AAP42565.1 | 31075385 | Clostridium |
| | | | saccharoperbutylacetonicum |

One ATP synthetase is ADP-forming acetyl-CoA synthetase (ACD, EC 6.2.1.13), an enzyme that couples the conversion of acyl-CoA esters to their corresponding acids with the concomitant synthesis of ATP. Although this enzyme has not been shown to react with 2-ketocyclohexane-1-carboxyl-CoA as a substrate, several enzymes with broad substrate specificities have been described in the literature. ACD I from Archaeoglobus fulgidus, encoded by AF1211, was shown to operate on a variety of linear and branched-chain substrates including isobutyrate, isopentanoate, and fumarate (Musfeldt et al., J. Bacteriol. 184:636-644 (2002)). A second reversible ACD in Archaeoglobus fulgidus, encoded by AF1983, was also shown to have a broad substrate range with high activity on cyclic compounds phenylacetate and indoleacetate (Musfeldt et al., supra). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al., supra). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen et al., supra; Musfeldt et al, supra). An additional enzyme is encoded by sucCD in E. coli, which naturally catalyzes the formation of succinyl-CoA from succinate with the concomitant consumption of one ATP, a reaction which is reversible in vivo (Buck et al., Biochemistry 24:6245-6252 (1985)). The protein sequences for exemplary gene products can be found using the following GenBank accession numbers shown below in Table 7.

TABLE 7

| Protein | GenBank ID | GI Number | Organism |
|---|---|---|---|
| AF1211 | NP_070039.1 | 11498810 | Archaeoglobus fulgidus |
| | | | DSM 4304 |
| AF1983 | NP_070807.1 | 11499565 | Archaeoglobus fulgidus |
| | | | DSM 4304 |
| scs | YP_135572.1 | 55377722 | Haloarcula marismortui |
| | | | ATCC 43049 |
| PAE3250 | NP_560604.1 | 18313937 | Pyrobaculum aerophilum |
| | | | str. IM2 |
| sucC | NP_415256.1 | 16128703 | Escherichia coli |
| sucD | AAC73823.1 | 1786949 | Escherichia coli |

Another possibility is mutating an AMP-forming CoA ligase to function in the reverse direction. The AMP-forming cyclohexanecarboxylate CoA-ligase from Rhodopseudomonas palustris, encoded by aliA, is active on a substrate similar to 2-ketocyclohexane-1-carboxyl-CoA, and alteration of the active site has been shown to impact the substrate specificity of the enzyme (Samanta et al., Mol. Microbiol. 55:1151-1159 (2005)). This enzyme also functions as a cyclohex-1-ene-1-carboxylate CoA-ligase during anaerobic benzene ring degradation (Egland et al., supra). It is unlikely, however, that the native form of this enzyme can function in the ATP-generating direction, as is required for formation of cyclohexane-1-carboxylate. Protein engineering or directed evolution can be used achieve this functionality. Additional exemplary CoA ligases include two characterized phenylacetate-CoA ligases from P. chrysogenum (Lamas-Maceiras et al., Biochem. J. 395:147-155 (2006); Wang et al., Biochem. Biophys. Res. Commun. 360:453-458 (2007)), the phenylacetate-CoA ligase from Pseudomonas putida (Martinez-Blanco et al., J. Biol. Chem. 265:7085-7090 (1990), and the 6-carboxyhexanoate-CoA ligase from Bacillus subtilis (Bower et al., J. Bacteriol 178:4122-4130 (1996)). The protein sequences for exemplary gene products can be found using the following GenBank accession numbers shown below in Table 8.

TABLE 8

| Protein | GenBank ID | GI Number | Organism |
|---|---|---|---|
| aliA | AAC23919 | 2190573 | Rhodopseudomonas palustris |
| phl | CAJ15517.1 | 77019264 | Penicillium chrysogenum |
| phlB | ABS19624.1 | 152002983 | Penicillium chrysogenum |
| paaF | AAC24333.2 | 22711873 | Pseudomonas putida |
| bioW | NP_390902.2 | 50812281 | Bacillus subtilis |

2-Ketocyclohexane-1-carboxyl-CoA can also be hydrolyzed to 2-ketocyclohexane-1-carboxylate by a CoA hydrolase. Several eukaryotic acetyl-CoA hydrolases (EC 3.1.2.1) have broad substrate specificity. The enzyme from Rattus norvegicus brain (131) can react with butyryl-CoA, hexanoyl-CoA and malonyl-CoA. The enzyme from the mitochondrion of the pea leaf is active on diverse substrates including acetyl-CoA, propionyl-CoA, butyryl-CoA, palmitoyl-CoA, oleoyl-CoA, succinyl-CoA, and crotonyl-CoA (Zeiher et al., Plant. Physiol. 94:20-27 (1990)). Additionally, a glutaconate CoA-transferase from Acidaminococcus fermentans was transformed by site-directed mutagenesis into an acyl-CoA hydrolase with activity on glutaryl-CoA, acetyl-CoA and 3-butenoyl-CoA (Mack et al., FEBS Lett. 405:209-212 (1997)). This indicates that the enzymes encoding succinyl-CoA:3-ketoacid-CoA transferases and acetoacetyl-CoA:acetyl-CoA transferases can also serve as CoA hydrolase enzymes but would require certain mutations to change their function. The acetyl-CoA hydrolase, ACH1, from S. cerevisiae represents another hydrolase (Buu et al., J. Biol. Chem. 278:17203-17209 (2003)). The protein sequences for exemplary gene products can be found using the following GenBank accession numbers shown below in Table 9.

TABLE 9

| Protein | GenBank ID | GI Number | Organism |
|---|---|---|---|
| acot12 | _570103.1 | 18543355 | Rattus norvegicus |
| gctA | CAA57199 | 559392 | Acidaminococcus fermentans |
| gctB | CAA57200 | 559393 | Acidaminococcus fermentans |
| ACH1 | NP_009538 | 6319456 | Saccharomyces cerevisiae |

In the final step of the pathway cyclohexanone is formed by the decarboxylation of 2-ketocyclohexane carboxylate (FIG. 2, step 3). This reaction is catalyzed by a 3-oxoacid decarboxylase such as acetoacetate decarboxylase (EC 4.1.1.4). The acetoacetate decarboxylase from Clostridium acetobutylicum, encoded by adc, has a broad substrate range and has been shown to decarboxylate 2-ketocyclohexane carboxylate to yield cyclohexanone (Benner et al., J. Am. Chem. Soc. 103:993-994 (1981); Rozzel et al., J. Am. Chem. Soc. 106:4937-4941 (1984)). The acetoacetate decarboxylase from Bacillus polymyxa, characterized in cell-free extracts, also has a broad substrate specificity for 3-keto acids and has been shown to decarboxylate the alternative substrate 3-oxopentanoate (Matiasek et al., Curr. Microbiol. 42:276-281 (2001)). Additional acetoacetate decarboxylase enzymes are found in Clostridium beijerinckii (Ravagnani et al., Mol. Microbiol. 37:1172-1185 (2000)) and Clostridium saccharoperbutylacetonicum (Kosaka et al., Biosci. Biotechnol. Biochem. 71:58-68 (2007)). Genes in other organisms, including Clostridium botulinum and Bacillus amyloliquefaciens FZB42, can be inferred by sequence homology. Decarboxylation of 3-oxoacids can also occur spontaneously in the absence of enzymes (Matiasek et al., supra)). The protein sequences for exemplary gene products can be found using the following GenBank accession numbers shown below in Table 10.

TABLE 10

| Protein | GenBank ID | GI Number | Organism |
|---|---|---|---|
| adc | NP_149328.1 | 15004868 | Clostridium acetobutylicum ATCC 824 |
| cbei_3835 | YP_001310906.1 | 150018652 | lostridium beijerinckii NCIMB 8052 |
| adc | AAP42566.1 | 31075386 | Clostridium saccharoperbutylacetonicum |
| CLL_A2135 | YP_001886324.1 | 187933144 | Clostridium botulinum |
| RBAM_030030 | YP_001422565.1 | 154687404 | Bacillus amyloliquefaciens FZB42 |

Although the net conversion of phosphoenolpyruvate to oxaloacetate is redox-neutral, the mechanism of this conversion is important to the overall energetics of the cyclohexanone production pathway. One enzyme for the conversion PEP to oxaloacetate is PEP carboxykinase which simultaneously forms an ATP while carboxylating PEP. In most organisms, however, PEP carboxykinase serves a gluconeogenic function and converts oxaloacetate to PEP at the expense of one ATP. S. cerevisiae is one such organism whose native PEP carboxykinase, PCK1, serves a gluconeogenic role (Valdes-Hevia et al., FEBS Lett. 258:313-316 (1989)). E. coli is another such organism, as the role of PEP carboxykinase in producing oxaloacetate is believed to be minor when compared to PEP carboxylase, which does not form ATP, possibly due to the higher Kₘ for bicarbonate of PEP carboxykinase (Kim et al., Appl. Environ Microbiol. 70:1238-1241 (2004)). Nevertheless, activity of the native E. coli PEP carboxykinase from PEP towards oxaloacetate has been recently demonstrated in ppc mutants of E. coli K-12 (Kwon et al., J. Microbiol. Biotechnol. 16:1448-1452 (2006)). These strains exhibited no growth defects and had increased succinate production at high NaHCO₃ concentrations. In some organisms, particularly rumen bacteria, PEP carboxykinase is quite efficient in producing oxaloacetate from PEP and generating ATP. Examples of PEP carboxykinase genes that have been cloned into E. coli include those from Mannheimia succiniciproducens (Lee et al., Biotechnol. Bioprocess. Eng 7:95-99 (2002)), Anaerobiospirillum succiniciproducens (Laivenieks et al., Appl. Environ. Microbiol. 63:2273-2280 (1997)), and Actinobacillus succinogenes (Kim et al., supra)). Internal experiments have also found that the PEP carboxykinase enzyme encoded by Haemophilus influenza is highly efficient at forming oxaloacetate from PEP. The protein sequences for exemplary gene products can be found using the following GenBank accession numbers shown below in Table 11.

TABLE 11

| Protein | GenBank ID | GI Number | Organism |
|---|---|---|---|
| PCK1 | NP_013023 | 6322950 | Saccharomyces cerevisiae |
| pck | NP_417862.1 | 16131280 | Escherichia coli |
| pckA | YP_089485.1 | 52426348 | Mannheimia succiniciproducens |
| pckA | O09460.1 | 3122621 | Anaerobiospirillum |
| | | | succiniciproducens |
| pckA | Q6W6X5 | 75440571 | Actinobacillus succinogenes |
| pckA | P43923.1 | 1172573 | Haemophilus influenza |

Pimeloyl-CoA is an intermediate of biotin biosynthesis. The enzymatic steps catalyzing biotin formation from pimeloyl-CoA are well-known and have been studied in several organisms, including Escherichia coli, Bacillus subtilis and Bacillus sphaericus, but pathways for synthesizing pimeloyl-CoA are not fully elucidated. In gram-negative bacteria such as E. coli the gene products of bioC and bioH are required for pimeloyl-CoA synthesis and strains deficient in these genes require addition of exogenous biotin to support growth (Del Campillo-Campbell et al., J. Bacteriol. 94:2065-2066 (1967)). The bioC gene product is thought to serve as a specific acyl-carrier protein catalyzing the stepwise condensation of malonyl-CoA units (Lemoine et al., Mol. Microbiol. 19:645-647 (1996)). The BioH protein contains a CoA binding site and is thought to function as an acyltransferase, shifting pimeloyl from BioC to CoA (Akatsuka et al., Gene 302:185-192 (2003); Lemoine et al., supra)). A novel feature of BioC would then be to restrict the acyl-transfer to a starter malonyl-CoA unit, and to limit chain extension to two extender units (Lemoine et al., supra)). A ¹³C labeling study in E. coli demonstrated that pimeloyl-CoA is derived from three acetate units and one unit of bicarbonate, implying that the synthetic mechanism is analogous to that of fatty acid and polyketide synthesis (Sanyai et al., J. Am. Chem. Soc. 116:2637-2638 (1994)). Gram-positive bacteria, such as B. subtilis and B. sphaericus, utilize a different pathway for synthesizing pimeloyl-CoA from pimelate, but this pathway is also poorly understood. In all biotin-producing organisms, open questions remain about the exact metabolic transformations involved, the function of gene products in the biotin operon, the role of classical fatty acid biosynthetic complex(es), the nature of the carrier protein, and pathway regulation.

Fatty acid and polyketide synthesis pathways are well-understood. In the first step of fatty acid synthesis, acetyl-CoA carboxylase consumes one ATP equivalent to form malonyl-CoA from acetyl-CoA and bicarbonate (Barber et al., Biochim. Biophys. Acta 1733:1-28 (2005)). If the pimeloyl-CoA carbon skeleton is composed of 3 extender units of malonyl-CoA, as proposed by Lemoine (Lemoin et al., supra)), three ATP equivalents are required. If the other required enzymatic activities (malonyl-CoA acyltransferase, beta-ketoacyl synthase, beta-ketoacyl reductase, beta-hydroxyacyl dehydratase, and enoyl-CoA reductase) are catalyzed by enzymes analogous to the common fatty acid complex, the net reaction for synthesizing one mole of pimeloyl-CoA from 3 acetyl-CoA building blocks becomes:

3Acetyl-CoA+3ATP+4NADH+Bicarbonate→Pimeloyl-CoA+4NAD⁺+3ADP+3Pi+2CoA+H⁺

Such a pathway is costly from an energetic standpoint, and moreover is not able to achieve the maximum theoretical yield of cyclohexanone, in a strain containing the enzymatic activities to convert pimeloyl-CoA to cyclohexanone. Under anaerobic conditions this pathway is predicted to achieve a maximum yield of 0.7 moles of cyclohexanone per mole glucose utilized. As the pathway is energetically limited, no ATP is available to support cell growth and maintenance at the maximum product yield. These facts indicate that aerobic conditions are required to achieve high cyclohexanone yields via a pathway similar to fatty acid biosynthesis. Another potential challenge is that this pathway will face competition from the well-known fatty acid ACP for malonyl-CoA extender units.

Attempts to engineer biotin-overproducing strains have had moderate success, although the development of cost-effective strains remains a technical challenge (Streit et al., Appl. Microbiol. Biotechnol. 61:21-31 (2003)). Strategies applied to improve biotin production, such as mutagenesis, cloning and/or overexpression of genes involved in the early stages of pimeloyl-CoA synthesis, could also be applied to improve cyclohexanone production.

In accordance with some embodiments of the present invention, pimeloyl-CoA is synthesized from acetoacetyl-CoA in seven enzymatic steps as shown in FIG. 2. This pathway occurs naturally in some organisms that degrade benzoyl-CoA. Although this pathway normally operates in the degradative direction, there is evidence that the bacterium Syntrophus aciditrophicus is able to grow on crotonate as a carbon source and form pimeloyl-CoA, providing evidence that the enzymes in this pathway can operate in the synthetic direction (Mouttaki et al., supra).

In the pathway shown in FIG. 2, the 3-keto group of acetoacetyl-CoA is reduced and dehydrated to form crotonyl-CoA. Glutaryl-CoA is formed from the reductive carboxylation of crotonyl-CoA. A beta-ketothiolase then combines glutaryl-CoA with acetyl-CoA to form 3-oxopimeloyl-CoA. Reduction and dehydration yield the 2-enoyl-CoA, which is then reduced to pimeloyl-CoA.

The reduction of acetoacetyl-CoA to 3-hydroxybutyryl-CoA is catalyzed by 3-hydroxyacyl-CoA dehydrogenase, also called acetoacetyl-CoA reductase (EC 1.1.1.36). This enzyme participates in polyhydroxybutyrate biosynthesis in many organisms, and has also been used in metabolic engineering strategies for overproducing PHB and 3-hydroxyisobutyrate (Liu et al., Appl. Microbiol. Biotechnol. 76:811-818 (2007); Qui et al., Appl. Microbiol. Biotechnol. 69:537-542 (2006)). The enzyme from Candida tropicalis is a component of the peroxisomal fatty acid beta-oxidation multifunctional enzyme type 2 (MFE-2). The dehydrogenase B domain of this protein is catalytically active on acetoacetyl-CoA. The domain has been functionally expressed in E. coli, a crystal structure is available, and the catalytic mechanism is well-understood (Yliantilla et al., J. Mol. Biol. 358 1286-1295 (2006), Ylianttila et al., Biochem. Biophys. Res. Commun. 324:25-30 (2004)). Acetoacetyl-CoA reductase has also been studied for its role in acetate assimilation in Rhodobacter sphaeroides (Alber et al., Mol. Microbiol. 61:297-309 (2006)). The enzyme from Zoogloea ramigera has a very low Km for acetoacetyl-CoA and has been cloned and overproduced in E. coli (Ploux et al., Eur J. Biochem. 174:177-182 (1988)). The enzyme from Paracoccus denitrificans has been functionally expressed and characterized in E. coli (Yabutani et al., FEMS Microbiol. Lett. 133:85-90 (1995)). The protein sequences for exemplary gene products can be found using the following GenBank accession numbers shown below in Table 11.

TABLE 11

| Protein | GenBank ID | GI Number | Organism |
|---|---|---|---|
| Fox2 | Q02207 | 399508 | Candida tropicalis |
| phaB | YP_353825 | 77464321 | Rhodobacter sphaeroides |
| phbB | P23238 | 130017 | Zoogloea ramigera |
| phaB | BAA08358 | 675524 | Paracoccus denitrificans |

The conversion of acetoacetyl-CoA to 3-hydroxybutyryl-CoA can also be catalyzed by acetoacetyl-CoA reductase, also known as 3-hydroxyacyl dehydrogenase (EC 1.1.1.35). Exemplary enzymes include hbd from C. acetobutylicum (Boynton et al., J. Bacteriol. 178:3015-3024 (1996)), hbd from C. beijerinckii (Colby et al., Appl. Environ Microbiol. 58:3297-3302 (1992)) and a number of similar enzymes from Metallosphaera sedula (Berg et al., Science 318:1782-1786 (2007)). Additional genes include Hbd1 (C-terminal domain) and Hbd2 (N-terminal domain) in Clostridium kluyveri (Hillmer et al., FEBS Lett. 21:351-354 (1972)) and HSD17B10 in Bos taurus (Wakil et al., J. Biol. Chem. 207:631-638 (1954)). The protein sequences for exemplary gene products can be found using the following GenBank accession numbers shown below in Table 12.

TABLE 12

| Protein | GenBank ID | GI Number | Organism |
|---|---|---|---|
| hbd | NP_349314.1 | 15895965 | Clostridium |
| | | | acetobutylicum |
| hbd | AAM14586.1 | 20162442 | Clostridium beijerinckii |
| Msed_1423 | YP_001191505 | 146304189 | Metallosphaera sedula |
| Msed_0399 | YP_001190500 | 146303184 | Metallosphaera sedula |
| Msed_0389 | YP_001190490 | 146303174 | Metallosphaera sedula |
| Msed_1993 | YP_001192057 | 146304741 | Metallosphaera sedula |
| Hbd2 | EDK34807.1 | 146348271 | Clostridium kluyveri |
| Hbd1 | EDK32512.1 | 146345976 | Clostridium kluyveri |
| HSD17B10 | O02691.3 | 3183024 | Bos taurus |

The gene product of pimF in Rhodopseudomonas palustris, predicted to encode a 3-hydroxy-acyl-CoA dehydratase, can also function as a 3-hydroxyacyl-CoA dehydrogenase during pimeloyl-CoA degradation (Harrison et al., Microbiology 151:727-736 (2005)). The gene product of fadB catalyzes these two functions during fatty acid beta-oxidation in E. coli (Yang et al., Biochem. 30:6788-6795 (1991)). 3-Hydroxyacyl-CoA dehydrogenase genes in S. aciditrophicus, inferred by sequence homology and genomic context, include syn_{—}01310 and syn_{—}01680 (McInerney et al., Proc. Natl. Acad. Sci. U.S.A. 104:7600-7605 (2007)). The protein sequences for exemplary gene products can be found using the following GenBank accession numbers shown below in Table 13.

TABLE 13

| Protein | GenBank ID | GI Number | Organism |
|---|---|---|---|
| pimF | CAE29158 | 39650635 | Rhodopseudomonas palustris |
| fadB | P21177 | 119811 | Escherichia coli |
| syn_01310 | YP_461961 | 85859759 | Syntrophus aciditrophicus |
| syn_01680 | ABC78882 | 85723939 | Syntrophus aciditrophicus |

3-Hydroxybutyryl-CoA dehydratase (EC 4.2.1.55), also called crotonase, dehydrates 3-hydroxyisobutyryl-CoA to form crotonoyl-CoA (FIG. 3, step 2). Crotonase enzymes are required for n-butanol formation in some organisms, particularly Clostridial species, and also comprise one step of the 3-hydroxypropionate/4-hydroxybutyrate cycle in thermoacidophilic Archaea of the genera Sulfolobus, Acidianus, and Metallosphaera. Exemplary genes encoding crotonase enzymes can be found in C. acetobutylicum (Atsumi et al., Metab. Eng. 10:305-311 (2007); Boynton et al., supra), C. kluyveri (Hillmer et al., supra), and Metallosphaera sedula (Berg et al., supra). The gene product of pimF in Rhodopseudomonas palustris is predicted to encode a 3-hydroxy-acyl-CoA dehydratase that participates in pimeloyl-CoA degradation (Harrison et al., Microbiol. 151:727-736 (2005)). A number of genes in S. aciditrophicus were identified by sequence similarity to the 3-hydroxybutyryl-CoA dehydratases of C. acetobutylicum and C. kluyveri. The protein sequences for exemplary gene products can be found using the following GenBank accession numbers shown below in Table 14.

TABLE 14

| Protein | GenBank ID | GI Number | Organism |
|---|---|---|---|
| crt | NP_349318.1 | 15895969 | Clostridium |
| | | | acetobutylicum |
| crt1 | YP_001393856.1 | 153953091 | Clostridium kluyveri |
| pimF | CAE29158 | 39650635 | Rhodopseudomonas |
| | | | palustris |
| syn_01309 | YP_461962 | 85859760 | Syntrophus |
| | | | aciditrophicus |
| syn_01653 | YP_463074 | 85860872 | Syntrophus |
| | | | aciditrophicus |
| syn_01654 | YP_463073.1 | 85860871 | Syntrophus |
| | | | aciditrophicus |
| syn_02400 | YP_462924.1 | 85860722 | Syntrophus |
| | | | aciditrophicus |
| syn_03076 | YP_463074.1 | 85860872 | Syntrophus |
| | | | aciditrophicus |

Enoyl-CoA hydratases (EC 4.2.1.17) also catalyze the dehydration of 3-hydroxyacyl-CoA substrates (Agnihotri et al., Bioorg. Med. Chem. 11:9-20 (2003); Conrad et al., J. Bacteriol. 118:103-111 (1974); Roberts et al., Arch. Microbiol. 117:99-108 (1978)). The enoyl-CoA hydratase of Pseudomonas putida, encoded by ech, catalyzes the conversion of 3-hydroxybutyryl-CoA to crotonyl-CoA (Roberts et al., supra). Additional enoyl-CoA hydratases are phaA and phaB, of P. putida, and paaA and paaB from P. fluorescens (Olivera et al., Proc. Natl. Acad. Sci. U.S.A. 95:6419-6424 (1998)). Lastly, a number of Escherichia coli genes have been shown to demonstrate enoyl-CoA hydratase functionality including maoC (Park et al., J. Bacteriol. 185:5391-5397 (2003)), paaF (Ismail et al., Eur. J. Biochem. 270:3047-3054 (2003); Park et al., Appl. Biochem. Biotechnol. 113:335-346 (2004); Park et al., Biotechnol. Bioeng. 86:681-686 (2004)) and paaG (Ismail et al, supra; Park et al., (2003) supra; Park et al., (2004) supra)). The protein sequences for exemplary gene products can be found using the following GenBank accession numbers shown below in Table 15.

TABLE 15

| Protein | GenBank ID | GI Number | Organism |
|---|---|---|---|
| ech | NP_745498.1 | 26990073 | Pseudomonas putida |
| phaA | NP_745427.1 | 26990002 | Pseudomonas putida |
| phaB | NP_745426.1 | 26990001 | Pseudomonas putida |
| paaA | ABF82233.1 | 106636093 | Pseudomonas fluorescens |
| paaB | ABF82234.1 | 106636094 | Pseudomonas fluorescens |
| maoC | NP_415905.1 | 16129348 | Escherichia coli |
| paaF | NP_415911.1 | 16129354 | Escherichia coli |
| paaG | NP_415912.1 | 16129355 | Escherichia coli |

Alternatively, the E. coli gene products of fadA and fadB encode a multienzyme complex involved in fatty acid oxidation that exhibits enoyl-CoA hydratase activity (Nakahigashi et al., Nucleic Acids Res. 18:4937 (1990); Yang, s. Y. J. Bacteriol. 173:7405-7406 (1991); Yang et al., Biochemistry 30:6788-6795 (1991)). Knocking out a negative regulator encoded by fadR can be utilized to activate the fadB gene product (Sato et al., J. Biosci. Bioeng. 103:38-44 (2007)). The fadI and fadJ genes encode similar functions and are naturally expressed under anaerobic conditions (Campbell et al., Mol. Microbiol. 47:793-805 (2003)). The protein sequences for exemplary gene products can be found using the following GenBank accession numbers shown below in Table 16.

TABLE 16

| Protein | GenBank ID | GI Number | Organism |
|---|---|---|---|
| fadA | YP_026272.1 | 49176430 | Escherichia coli |
| fadB | NP_418288.1 | 16131692 | Escherichia coli |
| fadI | NP_416844.1 | 16130275 | Escherichia coli |
| fadJ | NP_416843.1 | 16130274 | Escherichia coli |
| fadR | NP_415705.1 | 16129150 | Escherichia coli |

Glutaryl-CoA dehydrogenase (GCD, EC 1.3.99.7 and EC 4.1.1.70) is a bifunctional enzyme that catalyzes the oxidative decarboxylation of glutaryl-CoA to crotonyl-CoA (FIG. 3, step 3). Bifunctional GCD enzymes are homotetramers that utilize electron transfer flavoprotein as an electron acceptor (Hartel et al., Arch. Microbiol. 159:174-181 (1993)). Such enzymes were first characterized in cell extracts of Pseudomonas strains KB740 and K172 during growth on aromatic compounds (Hartel et al., supra), but the associated genes in these organisms is unknown. Genes encoding glutaryl-CoA dehydrogenase (gcdH) and its cognate transcriptional regulator (gcdR) were identified in Azoarcus sp. CIB (Blazquez et al., Environ. Microbiol. 10:474-482 (2008)). An Azoarcus strain deficient in gcdH activity was used to identify the a heterologous gene gcdH from Pseudomonas putida (Blazquez et al, supra). The cognate transcriptional regulator in Pseudomonas putida has not been identified but the locus PP_{—}0157 has a high sequence homology (>69% identity) to the Azoarcus enzyme. Additional GCD enzymes are found in Pseudomonas fluorescens and Paracoccus denitrificans (Husain et al., J. Bacteriol. 163:709-715 (1985)). The human GCD has been extensively studied, overexpressed in E. coli (Dwyer et al., Biochemistry 39:11488-11499 (2000)), crystallized, and the catalytic mechanism involving a conserved glutamate residue in the active site has been described (Fu et al., Biochemistry 43:9674-9684 (2004)). A GCD in Syntrophus aciditrophicus operates in the CO₂-assimilating direction during growth on crotonate (Mouttaki et al., supra)). Two GCD genes in S. aciditrophicus were identified by protein sequence homology to the Azoarcus GcdH: syn_{—}00480 (31%) and syn_{—}01146 (31%). No significant homology was found to the Azoarcus GcdR regulatory protein. The protein sequences for exemplary gene products can be found using the following GenBank accession numbers shown below in Table 17.

TABLE 17

| Protein | GenBank ID | GI Number | Organism |
|---|---|---|---|
| gcdH | ABM69268.1 | 123187384 | Azoarcus sp. CIB |
| gcdR | ABM69269.1 | 123187385 | Azoarcus sp. CIB |
| gcdH | AAN65791.1 | 24981507 | Pseudomonas putida |
| | | | KT2440 |
| PP_0157 (gcdR) | AAN65790.1 | 24981506 | Pseudomonas putida |
| | | | KT2440 |
| gcdH | YP_257269.1 | 70733629 | Pseudomonas |
| | | | fluorescens Pf-5 |
| gcvA (gcdR) | YP_257268.1 | 70733628 | Pseudomonas |
| | | | fluorescens Pf-5 |
| gcd | YP_918172.1 | 119387117 | Paracoccus |
| | | | denitrificans |
| gcdR | YP_918173.1 | 119387118 | Paracoccus |
| | | | denitrificans |
| gcd | AAH02579.1 | 12803505 | Homo sapiens |
| syn_00480 | ABC77899 | 85722956 | Syntrophus |
| | | | aciditrophicus |
| syn_01146 | ABC76260 | 85721317 | Syntrophus |
| | | | aciditrophicus |

Alternatively, the carboxylation of crotonyl-CoA to glutaconyl-CoA and subsequent reduction to glutaryl-CoA can be catalyzed by separate enzymes: glutaconyl-CoA decarboxylase and glutaconyl-CoA reductase. Glutaconyl-CoA decarboxylase enzymes, characterized in glutamate-fermenting anaerobic bacteria, are sodium-ion translocating decarboxylases that utilize biotin as a cofactor and are composed of four subunits (alpha, beta, gamma, and delta) (Boiangiu et al., J. Mol. Microbiol. Biotechnol. 10:105-119 (2005); Buckel et al., Biochim. Biophys. Acta 1505:15-27 (2001)). Such enzymes have been characterized in Fusobacterium nucleatum (Beatriz et al., Arch. Microbiol. 154:362-369 (1990)) and Acidaminococcus fermentans (Braune et al., Mol. Microbiol. 31:473-487 (1999)). Analogs to the F. nucleatum glutaconyl-CoA decarboxylase alpha, beta and delta subunits are found in S. aciditrophicus. A gene annotated as an enoyl-CoA dehydrogenase, syn_{—}00480, another GCD, is located in a predicted operon between a biotin-carboxyl carrier (syn_{—}00479) and a glutaconyl-CoA decarboxylase alpha subunit (syn_{—}00481). The protein sequences for exemplary gene products can be found using the following GenBank accession numbers shown below in Table 18.

TABLE 18

| Protein | GenBank ID | GI Number | Organism |
|---|---|---|---|
| gcdA | CAA49210 | 49182 | Acidaminococcus fermentans |
| gcdC | AAC69172 | 3777506 | Acidaminococcus fermentans |
| gcdD | AAC69171 | 3777505 | Acidaminococcus fermentans |
| gcdB | AAC69173 | 3777507 | Acidaminococcus fermentans |
| FN0200 | AAL94406 | 19713641 | Fusobacterium nucleatum |
| FN0201 | AAL94407 | 19713642 | Fusobacterium nucleatum |
| FN0204 | AAL94410 | 19713645 | Fusobacterium nucleatum |
| syn_00479 | YP_462066 | 85859864 | Syntrophus aciditrophicus |
| syn_00481 | YP_462068 | 85859866 | Syntrophus aciditrophicus |
| syn_01431 | YP_460282 | 85858080 | Syntrophus aciditrophicus |
| syn_00480 | ABC77899 | 85722956 | Syntrophus aciditrophicus |

If glutaconyl-CoA is formed by an enzyme with crotonyl-CoA carboxylase activity, reduction of glutaconyl-CoA to glutaryl-CoA can be accomplished by an enzyme with glutaconyl-CoA reductase activity. Enoyl-CoA reductase enzymes for catalyzing the reduction of 6-carboxyhex-2-enoyl-CoA to pimeloyl-CoA, described below, are also applicable here. One enzyme for this step is syn_{—}00480 of S. aciditrophicus, due to its genomic context adjacent to genes predicted to catalyze related functions.

Glutaryl-CoA and acetyl-CoA are condensed to form 3-oxopimeloyl-CoA by oxopimeloyl-CoA:glutaryl-CoA acyltransferase, a beta-ketothiolase (EC 2.3.1.16). An enzyme catalyzing this transformation is found in Ralstonia eutropha (formerly known as Alcaligenes eutrophus), encoded by genes bktB and bktC (Haywood et al., FEMS Microbiol. Lett. 52:91-96 (1988); Slater et al., J. Bacteriol. 180:1979-1987 (1998)). The sequence of the BktB protein is known; however, the sequence of the BktC protein has not been reported. The pim operon of Rhodopseudomonas palustris also encodes a beta-ketothiolase, encoded by pimB, predicted to catalyze this transformation in the degradative direction during benzoyl-CoA degradation (Harrison et al., supra). A beta-ketothiolase enzyme in S. aciditrophicus was identified by sequence homology to bktB (43% identity, evalue=1e−93). The protein sequences for exemplary gene products can be found using the following GenBank accession numbers shown below in Table 19.

TABLE 19

| Protein | GenBank ID | GI Number | Organism |
|---|---|---|---|
| bktB | YP_725948 | 11386745 | Ralstonia eutropha |
| pimB | CAE29156 | 39650633 | Rhodopseudomonas |
| | | | palustris |
| syn_02642 | YP_462685.1 | 85860483 | Syntrophus aciditrophicus |

Beta-ketothiolase enzymes catalyzing the formation of beta-ketovalerate from acetyl-CoA and propionyl-CoA can also catalyze the formation of 3-oxopimeloyl-CoA. Zoogloea ramigera possesses two ketothiolases that can form beta-ketovaleryl-CoA from propionyl-CoA and acetyl-CoA and R. eutropha has a beta-oxidation ketothiolase that is also capable of catalyzing this transformation (Gruys et al., U.S. Pat. No. 5,958,745). The sequences of these genes or their translated proteins have not been reported, but several genes in R. eutropha, Z. ramigera, or other organisms can be identified based on sequence homology to bktB from R. eutropha. The protein sequences for exemplary gene products can be found using the following GenBank accession numbers shown below in Table 20.

TABLE 20

| Protein | GenBank ID | GI Number | Organism |
|---|---|---|---|
| phaA | YP_725941.1 | 113867452 | Ralstonia eutropha |
| h16_A1713 | YP_726205.1 | 113867716 | Ralstonia eutropha |
| pcaF | YP_728366.1 | 116694155 | Ralstonia eutropha |
| h16_B1369 | YP_840888.1 | 116695312 | Ralstonia eutropha |
| h16_A0170 | YP_724690.1 | 113866201 | Ralstonia eutropha |
| h16_A0462 | YP_724980.1 | 113866491 | Ralstonia eutropha |
| h16_A1528 | YP_726028.1 | 113867539 | Ralstonia eutropha |
| h16_B0381 | YP_728545.1 | 116694334 | Ralstonia eutropha |
| h16_B0662 | YP_728824.1 | 116694613 | Ralstonia eutropha |
| h16_B0759 | YP_728921.1 | 116694710 | Ralstonia eutropha |
| h16_B0668 | YP_728830.1 | 116694619 | Ralstonia eutropha |
| h16_A1720 | YP_726212.1 | 113867723 | Ralstonia eutropha |
| h16_A1887 | YP_726356.1 | 113867867 | Ralstonia eutropha |
| phbA | P07097.4 | 135759 | Zoogloea ramigera |
| bktB | YP_002005382.1 | 194289475 | Cupriavidus taiwanensis |
| Rmet_1362 | YP_583514.1 | 94310304 | Ralstonia metallidurans |
| Bphy_0975 | YP_001857210.1 | 186475740 | Burkholderia phymatum |

Additional enzymes include beta-ketothiolases that are known to convert two molecules of acetyl-CoA into acetoacetyl-CoA (EC 2.1.3.9). Exemplary acetoacetyl-CoA thiolase enzymes include the gene products of atoB from E. coli (Martin et al., Nat. Biotechnol. 21:796-802 (2003)), thlA and thlB from C. acetobutylicum (Hanai et al., Appl. Environ. Microbiol. 73:7814-7818 (2007); Winzer et al., J. Mol. Microbiol. Biotechnol. 2:531-541 (2000)), and ERG10 from S. cerevisiae (Hiser et al., J. Biol. Chem. 269:31383-31389 (1994)). The protein sequences for exemplary gene products can be found using the following GenBank accession numbers shown below in Table 21.

TABLE 21

| Protein | GenBank ID | GI Number | Organism |
|---|---|---|---|
| atoB | NP_416728 | 16130161 | Escherichia coli |
| thlA | NP_349476.1 | 15896127 | Clostridium acetobutylicum |
| thlB | NP_149242.1 | 15004782 | Clostridium acetobutylicum |
| ERG10 | NP_015297 | 6325229 | Saccharomyces cerevisiae |

Beta-ketoadipyl-CoA thiolase (EC 2.3.1.174), also called 3-oxoadipyl-CoA thiolase, converts beta-ketoadipyl-CoA to succinyl-CoA and acetyl-CoA, and is a key enzyme of the beta-ketoadipate pathway for aromatic compound degradation. The enzyme is widespread in soil bacteria and fungi including Pseudomonas putida (Harwood et al., J. Bacteriol. 176-6479-6488 (1994)) and Acinetobacter calcoaceticus (Doten et al., J. Bacteriol. 169:3168-3174 (1987)). The P. putida enzyme is a homotetramer bearing 45% sequence homology to beta-ketothiolases involved in PHB synthesis in Ralstonia eutropha, fatty acid degradation by human mitochondria and butyrate production by Clostridium acetobutylicum (Harwood et al., supra). A beta-ketoadipyl-CoA thiolase in Pseudomonas knackmussii (formerly sp. B13) has also been characterized (Gobel et al., J. Bacteriol. 184:216-223 (2002); Kaschabek et al., supra). The protein sequences for exemplary gene products can be found using the following GenBank accession numbers shown below in Table 22.

TABLE 22

| Protein | GenBank ID | GI Number | Organism |
|---|---|---|---|
| pcaF | NP_743536.1 | 506695 | Pseudomonas putida |
| pcaF | AAC37148.1 | 141777 | Acinetobacter calcoaceticus |
| catF | Q8VPF1.1 | 75404581 | Pseudomonas knackmussii |

Reduction of 3-oxopimeloyl-CoA to 3-hydroxypimeloyl-CoA is catalyzed by 3-hydroxypimeloyl-CoA dehydrogenase (EC 1.1.1.259). This activity has been demonstrated in cell extracts of Rhodopseudomonas palustris and Pseudomonas sp (Koch et al., Eur. J. Biochem. 211:649-661 (1993); Koch et al., Eur. J. Biochem. 205:195-202 (1992)) but genes have not been reported. This transformation is also predicted to occur in Syntrophus aciditrophicus during growth on crotonate (Mouttaki et al., supra). Enzymes with 3-hydroxyacyl-CoA dehydrogenase and/or acetoacetyl-CoA reductase activities can also catalyze this reaction.

Dehydration of 3-hydroxypimeloyl-CoA to 6-carboxyhex-2-enoyl-CoA is predicted to occur in S. aciditrophicus during crotonate utilization to cyclohexane carboxylate (Mouttaki et al., supra). This reaction can be catalyzed by an enoyl-CoA hydratase (4.2.1.17) or a 3-hydroxybutyryl-CoA dehydratase (EC 4.2.1.55).

The reduction of 6-carboxyhex-2-enoyl-CoA to pimeloyl-CoA by pimeloyl-CoA dehydrogenase (EC 1.3.1.62) has been characterized in Syntrophus aciditrophicus cell extracts (Elshahed et al., supra). Enoyl-CoA reductase enzymes are suitable enzymes for catalyzing this transformation. One exemplary enoyl-CoA reductase is the gene product of bcd from C. acetobutylicum (Atsumi et al., supra; Boynton et al., supra), which naturally catalyzes the reduction of crotonyl-CoA to butyryl-CoA. Activity of this enzyme can be enhanced by expressing bcd in conjunction with expression of the C. acetobutylicum etfAB genes, which encode an electron transfer flavoprotein. An additional enzyme for the enoyl-CoA reductase step is the mitochondrial enoyl-CoA reductase from E. gracilis (Hoffmeister et al., J. Biol. Chem. 280:4329-4338 (2005)). A construct derived from this sequence following the removal of its mitochondrial targeting leader sequence was cloned in E. coli resulting in an active enzyme (Hoffmeister et al., supra). This approach is well known to those skilled in the art of expressing eukaryotic genes, particularly those with leader sequences that can target the gene product to a specific intracellular compartment, in prokaryotic organisms. A close homolog of this gene, TDE0597, from the prokaryote Treponema denticola represents a third enoyl-CoA reductase which has been cloned and expressed in E. coli (Tucci et al., FEBS Lett. 581:1561-1566 (2007)). Six genes in S. aciditrophicus were identified by sequence homology to the C. acetobutylicum bcd gene product. The S. aciditrophicus genes syn_{—}02637 and syn_{—}02636 bear high sequence homology to the etfAB genes of C. acetobutylicum, and are predicted to encode the alpha and beta subunits of an electron transfer flavoprotein. The protein sequences for exemplary gene products can be found using the following GenBank accession numbers shown below in Table 23.

TABLE 23

| Protein | GenBank ID | GI Number | Organism |
|---|---|---|---|
| bcd | NP_349317.1 | 15895968 | Clostridium acetobutylicum |
| etfA | NP_349315.1 | 15895966 | Clostridium acetobutylicum |
| etfB | NP_349316.1 | 15895967 | Clostridium acetobutylicum |
| TER | Q5EU90.1 | 62287512 | Euglena gracilis |
| TDE0597 | NP_971211.1 | 42526113 | Treponema denticola |
| syn_02587 | ABC76101 | 85721158 | Syntrophus aciditrophicus |
| syn_02586 | ABC76100 | 85721157 | Syntrophus aciditrophicus |
| syn_01146 | ABC76260 | 85721317 | Syntrophus aciditrophicus |
| syn_00480 | ABC77899 | 85722956 | Syntrophus aciditrophicus |
| syn_02128 | ABC76949 | 85722006 | Syntrophus aciditrophicus |
| syn_01699 | ABC78863 | 85723920 | Syntrophus aciditrophicus |
| syn_02637 | ABC78522.1 | 85723579 | Syntrophus aciditrophicus |
| syn_02636 | ABC78523.1 | 85723580 | Syntrophus aciditrophicus |

Additional enoyl-CoA reductase enzymes are found in organisms that degrade aromatic compounds. Rhodopseudomonas palustris, a model organism for benzoate degradation, has the enzymatic capability to degrade pimelate via beta-oxidation of pimeloyl-CoA. Adjacent genes in the pim operon, pimC and pimD, bear sequence homology to C. acetobutylicum bcd and are predicted to encode a flavin-containing pimeloyl-CoA dehydrogenase (Harrison et al., supra). The genome of nitrogen-fixing soybean symbiont Bradyrhizobium japonicum also contains a pim operon composed of genes with high sequence similarity to pimC and pimD of R. palustris (Harrison et al., supra). The protein sequences for exemplary gene products can be found using the following GenBank accession numbers shown below in Table 24.

TABLE 24

| Protein | GenBank ID | GI Number | Organism |
|---|---|---|---|
| pimC | CAE29155 | 39650632 | Rhodopseudomonas palustris |
| pimD | CAE29154 | 39650631 | Rhodopseudomonas palustris |
| pimC | BAC53083 | 27356102 | Bradyrhizobium japonicum |
| pimD | BAC53082 | 27356101 | Bradyrhizobium japonicum |

An additional enzyme is 2-methyl-branched chain enoyl-CoA reductase (EC 1.3.1.52), an enzyme catalyzing the reduction of sterically hindered trans-enoyl-CoA substrates. This enzyme participates in branched-chain fatty acid synthesis in the nematode Ascarius suum and is capable of reducing a variety of linear and branched chain substrates including 2-methylbutanoyl-CoA, 2-methylpentanoyl-CoA, octanoyl-CoA and pentanoyl-CoA (Duran et al., J. Biol. Chem. 268:22391-22396 (1993)). Two isoforms of the enzyme, encoded by genes acad1 and acad, have been characterized. The protein sequences for exemplary gene products can be found using the following GenBank accession numbers shown below in Table 25.

TABLE 25

| Protein | GenBank ID | GI Number | Organism |
|---|---|---|---|
| acad1 | AAC48316.1 | 2407655 | Ascarius suum |
| acad | AAA16096.1 | 347404 | Ascarius suum |

Alternative routes for producing a cyclic compound from 3-hydroxypimeloyl-CoA that do not proceed through pimeloyl-CoA are shown in FIG. 3. This route is found in Geobacter metallireducens and Thauera aromatica, among others, in the direction of beta-oxidation. In the route, the biosynthesis of 3-hydroxypimelyl-CoA proceeds from acetoacetyl-CoA, as described above. 3-Hydroxypimeloyl-CoA is dehydrated to form a cyclic product, 6-oxocylohex-1-ene-1-carboxyl-CoA (6-KCH-CoA). 6-KCH-CoA is then converted to cyclohexanone in three enzymatic steps: removal of the CoA moiety, decarboxylation and reduction. With a reversible PEP carboxykinase, this pathway is predicted to achieve a theoretical yield of cyclohexanone (0.75 mol/mol) and is able to achieve an ATP yield of 0.56 mol/mol if a transferase or ATP synthase is utilized in step 2.

6-KCH-CoA hydrolase (EC 3.7.1.-) converts 6-ketocyclohex-1-ene-1-carboxyl-CoA (6-KCH-CoA) to 3-hydroxypimeloyl-CoA. This enzyme belongs to the crotonase superfamily and is unusual in that it incorporates two water molecules in the ring-opening direction (Eberhard et al., J. Am. Chem. Soc. 126:7188-7189 (2004)). This enzyme has been studied in the context of anaerobic benzoyl-CoA degradation in the obligate anaerobes Thauera aromatica (Breese et al., Eur. J. Biochem. 256:148-154 (1998), Laempe et al., Eur. J. Biochem. 263:420-429 (1999)), Geobacter metallireducens (Kuntze et al., Environ Microbiol. 10:1547-1556 (2008)), S. aciditrophicus (Kuntze et al., supra), Azoarcus evansii (Harwood et al., FEBS Microbiol. Rev. 22:439-458 (1999)) and Azoarcus sp. Strain CIB (Lopez-Barragan et al., J. Bacteriol. 186:5762-5774 (2004)). The 6-KCH-CoA hydrolase genes gmet_{—}2088 from G. metallireducens and syn_{—}01654 from S. aciditrophicus were heterologously expressed and characterized in E. coli (Kuntze et al., supra). The S. aciditrophicus 6-KCH-CoA hydrolase (syn_{—}01654) was assayed for activity in the ring-closing direction but this activity was not observed (Kuntze et al., supra). Additional genes encoding 6-KCH-CoA hydrolases were identified in Desulfococcus multivorans and an m-xylene degrading enrichment culture (Kuntze et al., supra). Additional hydrolases in S. aciditrophicus are syn_{—}01653, syn_{—}02400, syn_{—}03076 and syn_{—}01309. Syn_{—}01653 is adjacent to syn_{—}01654 and predicted to be in the same operon. The protein sequences for exemplary gene products can be found using the following GenBank accession numbers shown below in Table 26.

TABLE 26

| Protein | GenBank ID | GI Number | Organism |
|---|---|---|---|
| bzdY | AAQ08817.1 | 33326786 | Azoarcus sp. CIB |
| bzdY | CAD21638.1 | 18369665 | Azoarcus evansii |
| oah | CAA12245.1 | 3724166 | Thauera aromatica |
| bamA | YP_385042.1 | 78223295 | Geobacter |
| (gmet_2088) | | | metallireducens |
| bamA | YP_463073.1 | 85860871 | Syntrophus |
| (syn_01654) | | | aciditrophicus |
| N/A | ABY89672.2 | 262284543 | Desulfococcus |
| | | | multivorans |
| N/A | ABY89673.1 | 166798254 | [bacterium enrichment |
| | | | culture clone ZzG1mX] |
| syn_01653 | YP_463074.1 | 85860872 | Syntrophus |
| | | | aciditrophicus |
| syn_02400 | YP_462924.1 | 85860722 | Syntrophus |
| | | | aciditrophicus |
| syn_03076 | YP_463118.1 | 85860916 | Syntrophus |
| | | | aciditrophicus |
| syn_01309 | YP_461962.1 | 85859760 | Syntrophus |
| | | | aciditrophicus |

The de-acylation of 6-KCH-CoA is similar to the de-acylation of 2-ketocyclohexane-1-carboxyl-CoA (2-KCH-CoA) to 2-ketocyclohexane-1-carboxylate (2-KCH) by a CoA-transferase, synthetase or hydrolase. Exemplary enzymes include those discussed above. The decarboxylation of 6-KCH to 2-cyclohexenone (step 3) is similar to the decarboxylation of 2-KCH (FIG. 1, step 3 and FIG. 3, step 7). Exemplary enzymes for that transformation are also applicable here.

In the final step of the pathway, 2-cyclohexen-1-one is reduced to form cyclohexanone by cyclohexanone dehydrogenase (EC 1.3.99.14), an NAD(P)H-dependent enone reductase. This reaction occurs in cell extracts of the denitrifying bacteria Alicycliphilus denitrificans sp. K601 (formerly known as Pseudomonas sp. K601) during anaerobic growth on cyclohexanol (Dangel et al., Arch. Microbiol. 152:271-279; Dangel et al., Arch. Microbiol. 150:358-362 (1988); Mechichi et al., In. J. Syst. Evol. Microbiol. 53:147-152 (2003)). Purified cyclohexanone dehydrogenase was characterized in cell extracts.

Enzymes with enone reductase activity that naturally react with cyclic compounds have been identified in prokaryotes, eukaryotes and plants (Shimoda et al., Bulletin of the Chemical Society of Japan 77:2269-2 (2004); Wanner et al., Eur. J. Biochem. 255:271-278 (1998)). Two enone reductases from the cytosolic fraction of Saccharomyces cerevisiae were purified and characterized, and found to accept 2-cyclohexen-1-one as a substrate (Wanner et al., supra). Cell extracts of cyanobacterium Synechococcus sp. PCC7942 reduced a variety of cyclic and acyclic substrates, including 2-methyl-2-cyclohexen-1-one and 2-ethyl-2-cyclohexen-1-one, to their corresponding alkyl ketones (Shimoda et al., supra). Genes have not been associated with these activities. A recombinant NADPH-dependent enone reductase from Nicotiana tabacum, encoded by NtRed1, was functionally expressed and characterized in E. coli (Matsushima et al., Bioorganic Chemistry 36:23-28 (2008)). This reductase was functional on exocyclic enoyl ketones but did not react with carvone, a sterically hindered endocyclic enoyl ketone (Matsushima et al., supra). This enzyme was not tested on 2-cyclohexen-1-one as a substrate. An enzyme in S. cerevisiae at the locus YML131W, bears 30% identity to NtRed1(evalue=1e−26). Endocyclic enoate reductase activity has also been detected in N. tabacum (Hirata et al., Phytochemistry 28:3331-3333 (1989)). The amino acid sequence of NtRed1 shares significant homology with 2-alkenal reductase from Arabidopsis thaliana, zeta-crystallin homolog from A. thaliana, pulegone reductase from Menthe piperita and phenylpropenal double bond reductase from Pinus taeda. These enzymes are known to catalyze the reduction of alkenes of α,β-unsaturated ketones or aldehydes. The protein sequences for exemplary gene products can be found using the following GenBank accession numbers shown below in Table 27.

TABLE 27

| Protein | GenBank ID | GI Number | Organism |
|---|---|---|---|
| NtRed1 | BAA89423 | 6692816 | Nicotiana tabacum |
| AtDBR1 | NP_197199 | 15237888 | Arabidopsis thaliana |
| P2 | CAA89262 | 886430 | Arabidopsis thaliana |
| PulR | AAQ75423 | 34559418 | Menthe piperita |
| PtPPDBR | ABG91753 | 110816011 | Pinus taeda |
| YML131W | AAS56318.1 | 45269874 | Saccharomyces cerevisiae |

Another endocyclic enone reductase is (−)-isopiperitenone reductase (IspR), an enzyme participating in monoterpene biosynthesis in Menthe piperita (Ringer et al., Arch. Biochem. Biophys 418:80-92 (2003)). The protein sequence of this enzyme shows significant homology to putative short-chain reductases in human, pig, CHO-K1/hamster cells and Arabidopsis thaliana (Ringer et al., supra). The M. piperita IspR protein sequence was compared to the S. cerevisiae and Synechococcus sp. PCC 7942 genomes, but no high-confidence hits were identified. The closest was a putative benzil reductase in S. cerevisiae at the locus YIR036C bearing 26% identity to IspR. The protein sequences for exemplary gene products can be found using the following GenBank accession numbers shown below in Table 28.

TABLE 28

| Protein | GenBank ID | GI Number | Organism |
|---|---|---|---|
| ispR | AAQ75422.1 | 34559416 | Menthe piperita |
| AT3G61220 | NP_191681.1 | 15233062 | Arabidopsis thaliana |
| cbr | NP_001748.1 | 4502599 | Homo sapiens |
| CBR1 | NP_999238.1 | 47522960 | Sus scrofa |
| CHO-CR | BAB07797.1 | 9711233 | Cricetulus griseus |
| YIR036C | NP_012302.1 | 6322227 | Saccharomyces cerevisiae |

Enzymes with 2-enoate reductase activity (EC 1.3.1.31) can also catalyze this conversion. 2-Enoate reductase enzymes are known to catalyze the NADH-dependent reduction of a wide variety of α,β-unsaturated carboxylic acids and aldehydes (Rohdich et al., J. Biol. Chem. 276:5779-5787 (2001)). 2-Enoate reductases is encoded by enr in several species of Clostridia including C. tyrobutyricum, and C. thermoaceticum (now called Moorella thermoaceticum) (Geisel et al., Arch. Microbiol. 135:51-57 (1983); Rohdich et al., supra). In the recently published genome sequence of C. kluyveri, 9 coding sequences for enoate reductases were reported, out of which one has been characterized (Seedorf et al., Proc. Natl. Acad. Sci. USA. 105:2128-2133 (2008)). The enr genes from both C. tyrobutyricum and M. thermoaceticum have been cloned and sequenced and show 59% identity to each other. The former gene is also found to have approximately 75% similarity to the characterized gene in C. kluyveri (Geisel et al., supra). It has been reported based on these sequence results that enr is very similar to the dienoyl CoA reductase in E. coli (fades) (Rohdich et al., supra). The C. thermoaceticum enr gene has also been expressed in a catalytically active form in E. coli (Rohdich et al., supra). The protein sequences for exemplary gene products can be found using the following GenBank accession numbers shown below in Table 29.

TABLE 29

| Protein | GenBank ID | GI Number | Organism |
|---|---|---|---|
| enr | ACA54153.1 | 169405742 | Clostridium botulinum A3 str |
| enr | CAA71086.1 | 2765041 | Clostridium tyrobutyricum |
| enr | CAA76083.1 | 3402834 | Clostridium kluyveri |
| enr | YP_430895.1 | 83590886 | Moorella thermoacetica |
| fadH | NP_417552.1 | 16130976 | Escherichia coli |

An alternate route for synthesizing cyclohexanone from 6-ketocyclohex-1-ene-1-carboxyl-CoA (6-KCH-CoA) employs similar enzymes applied in a different order. In this route, 6-KCH-CoA is first reduced to 2-ketocyclohexane-1-carboxyl-CoA (2-KCH-CoA) by an enoyl-CoA reductase (EC 1.3.1.-) (FIG. 3, step 5). Exemplary enoyl-CoA reductase enzymes are described above for the reduction of 6-carboxyhex-2-enoyl-CoA to pimeloyl-CoA.

In step 8 of FIG. 3, 6-KCH is reduced to 2-KCH by an enoate reductase (EC 1.3.1.-). Enzymes for enoate reductases are described above for the reduction of 2-cyclohexene-1-one to cyclohexanone. 2-KCH is subsequently decarboxylated to cyclohexanone via 2-KCH decarboxylase using the decarboxylase enzymes described above.

In some embodiments cyclohexanone is produced via a pathway for converting adipate semialdehyde to cyclohexanone. Adipate semialdehyde is not a naturally occurring metabolite in commonly used production organisms such as Escherichia coli and Saccharomyces cerevisiae. However, a number of biosynthetic routes for adipate biosynthesis have recently been disclosed [U.S. patent application Ser. No. 12/413,355]. In this report, we assume that adipate semialdehyde is produced from molar equivalents of acetyl-CoA and succinyl-CoA, joined by a beta-ketothiolase to form oxoadipyl-CoA. Oxoadipyl-CoA is then converted to adipyl-CoA in three enzymatic steps: reduction of the ketone, dehydration, and reduction of the enoyl-CoA. Once formed, adipyl-CoA is converted to adipate semialdehyde by a CoA-dependent aldehyde dehydrogenase.

The pathway to cyclohexanone from adipate semialdehyde entails four enzymatic steps as shown in FIG. 4. In the first step, adipate semialdehyde is dehydrated and cyclized, forming cyclohexane-1,2-dione (12-CHDO). 12-CHDO is then reduced to the diol by cyclohexane-1,2-diol dehydrogenase. Finally, a diol dehydratase converts cyclohexane-1,2-diol to cyclohexanone.

This pathway is capable of achieving high product and energetic yields. The maximum theoretical cyclohexanone yield is 0.75 mol/mol from glucose. With a wild-type PPCK activity, the pathway achieves an ATP yield of 1.362 mole ATP per mole glucose utilized at the maximum cyclohexanone yield. With PEP carboxykinase able to function in the ATP-generating direction, the ATP yield is further increased to 2.11 mol/mol.

In organisms that degrade caprolactam such as Pseudomonas aeruginosa (Kulkarni et al., Curr. Microbiol. 37:191-194 (1998); Steffensen et al., Appl. Environ Microbiol 61:2859-2862 (1995)), adipate is readily converted to cyclohexa-1,2-dione by a dehydratase in the EC 3.7.1 family. This transformation was also identified in cell extracts of Azoarcus species, as part of an anaerobic cyclohexan-1,2-diol degradation pathway (Harder, J., Arch. Microbiol. 168:199-203 (1997)). A similar transformation is catalyzed in the myo-inositol degradation pathway, in which the cyclic dione 2,3-diketo-4-deoxy-epi-inositol is hydrolyzed to a linear product, 5-dehydro-2-deoxy-D-gluconate, by a diketodeoxyinositol hydrolase (EC 3.7.1.-). A partially purified protein catalyzing this reaction has been studied in Klebsiella aerogenes (Berman et al., J. Biol. Chem. 241:800-806 (1966)).

The conversion of cyclohexane-1,2-dione to a diol can be accomplished by cyclohexane-1,2-diol dehydrogenase (EC 1.1.1.174). This enzymatic activity has been demonstrated in Acinetobacter TD63 (Davey et al., Eur. J. Biochem. 74:115-127 (1977)). It has been indicated that cyclohexanol dehydrogenase (EC 1.1.1.245), an enzyme with a broad substrate range, can catalyze these conversions. Cyclohexanol dehydrogenase enzymes from Rhodococcus sp TK6 (Tae-Kang et al., J. Microbiol. Biotechnol. 12:39-45 (2002)), a denitrifying Pseudomonas sp. (Dangel et al., supra), Nocardia sp (Stirling et al., Curr. Microbiol. 4:37-40 (1980)) and Xanthobacter sp. (Trower et al., App. Environ. Microbiol. 49″1282-1289 (1985)) have all been shown to convert cyclohexan-1,2-diol to cyclohexan-1,2-dione. The gene associated with a cyclohexanol dehydrogenase in Acinetobacter sp NCIMB9871 was identified in 2000 (Cheng et al., J. Bacteriol. 182:4744-4751). This enzyme, encoded by chnA, has not been tested for activity on cyclohexan-1,2-dione or cyclohexan-1,2-diol. A BLAST comparison of the Acinetobacter ChnA protein sequence identifies genes from other organisms including Ralstonia metallireducens (57% identity), and Pseudomonas putida (47% identity). A cyclohexanol dehydrogenase gene from Comamonas testosteroni has also been expressed and characterized in E. coli (Van Beilen et al., Environ. Microbiol. 5:174-182 (2003)); a similar gene was also identified in Xanthobacter flavus (Van Beilen et al., supra). The protein sequences for exemplary gene products can be found using the following GenBank accession numbers shown below in Table 30.

TABLE 30

| Protein | GenBank ID | GI Number | Organism |
|---|---|---|---|
| chnA | BAC80215.1 | 33284995 | Acinetobacter sp |
| | | | NCIMB9871 |
| chnA | CAD10799.1 | 16943680 | Comamonas testosteroni |
| chnA | CAD10802.1 | 18495819 | Xanthobacter flavus |
| Rmet_1335 | YP_583487.1 | 94310277 | Ralstonia metallireducens |
| PP_1946 | NP_744098.1 | 26988673 | Pseudomonas putida |

Another enzyme which can accomplish this conversion is diacetyl reductase (EC 1.1.1.5). Naturally catalyzing the conversion of diacetyl (2,3-butanedione) to acetoin and subsequent reduction to 2,3-butanediol, two NADPH-dependent diacetyl reductase enzymes from S. cerevisiae have been shown to also accept cyclohexan-1,2-dione as a substrate (Heidlas et al., Eur. J. Biochem. 188:165-174 (1990)). The (S)-specific NADPH-dependent diacetyl reductase from this study was later identified as D-arabinose dehydrogenase, the gene product of ARA1 (Katz et al., Enzyme Microb. Technol. 33:163-172 (2003)). The NADH-dependent gene product of BDH1 of S. cerevisiae also has diacetyl reductase functionality (Gonzalez et al., J. Biol. Chem. 275:33876-35885 (2000)). Several other enzymes with diketone reductase functionality have been identified in yeast, encoded by genes GCY1, YPR1, GRE3, Y1R036c (Johanson et al., FEMS Yeast Res. 5:513-525 (2005)). The protein sequences for exemplary gene products can be found using the following GenBank accession numbers shown below in Table 31.

TABLE 31

| Protein | GenBank ID | GI Number | Organism |
|---|---|---|---|
| ARA1 | NP_009707.1 | 6319625 | Saccharomyces cerevisiae |
| BDH1 | NP_009341.1 | 6319258 | Saccharomyces cerevisiae |
| GCY1 | NP_014763.1 | 6324694 | Saccharomyces cerevisiae |
| YPR1 | NP_010656.1 | 6320576 | Saccharomyces cerevisiae |
| GRE3 | NP_011972.1 | 6321896 | Saccharomyces cerevisiae |
| YIR036c | AAS56566.1 | 45270370 | Saccharomyces cerevisiae |

Conversion of the cyclohexan-1,2-diol to cyclohexanone has not been demonstrated enzymatically. A similar transformation is catalyzed by the diol dehydratase myo-inosose-2-dehydratase (EC 4.2.1.44). Myo-inosose is a six-membered ring containing adjacent alcohol groups, similar to cyclohexan-1,2-diol. A purified enzyme encoding myo-inosose-2-dehydratase functionality has been studied in Klebsiella aerogenes in the context of myo-inositol degradation (Berman et al., supra), but has not been associated with a gene to date.

Diol dehydratase or propanediol dehydratase enzymes (EC 4.2.1.28) capable of converting the secondary diol 2,3-butanediol to methyl ethyl ketone would be appropriate for this transformation. Adenosylcobalamin-dependent diol dehydratases contain alpha, beta and gamma subunits, which are all required for enzyme function. Exemplary genes are found in Klebsiella pneumoniae (Tobimatsu et al., Biosci Biotechnol. Biochem. 62:1774-1777 (1998); Toraya et al., Biochem. Biophys. Res. Commun. 69:475-480 (1976)), Salmonella typhimurium (Bobik et al., J. Bacteriol. 179:6633-6639 (1997)), Klebsiella oxytoca (Tobimatsu et al., J. Biol. Chem. 270:7142-7148 (1995)) and Lactobacillus collinoides (Sauvageot et al., FEMS Microbiol. Lett. 209:69-74 (2002)). Methods for isolating diol dehydratase genes in other organisms are well known in the art (e.g. U.S. Pat. No. 5,686,276). The protein sequences for exemplary gene products can be found using the following GenBank accession numbers shown below in Table 32.

TABLE 32

| Protein | GenBank ID | GI Number | Organism |
|---|---|---|---|
| pddC | AAC98386.1 | 4063704 | Klebsiella pneumoniae |
| pddB | AAC98385.1 | 4063703 | Klebsiella pneumoniae |
| pddA | AAC98384.1 | 4063702 | Klebsiella pneumoniae |
| pduC | AAB84102.1 | 2587029 | Salmonella typhimurium |
| pduD | AAB84103.1 | 2587030 | Salmonella typhimurium |
| pduE | AAB84104.1 | 2587031 | Salmonella typhimurium |
| pddA | BAA08099.1 | 868006 | Klebsiella oxytoca |
| pddB | BAA08100.1 | 868007 | Klebsiella oxytoca |
| pddC | BAA08101.1 | 868008 | Klebsiella oxytoca |
| pduC | CAC82541.1 | 18857678 | Lactobacillus collinoides |
| pduD | CAC82542.1 | 18857679 | Lactobacillus collinoides |
| pduE | CAD01091.1 | 18857680 | Lactobacillus collinoides |

Enzymes in the glycerol dehydratase family (EC 4.2.1.30) can also be used to convert cyclohexan-1,2-diol to cyclohexanone. Exemplary genes can be found in Klebsiella pneumoniae (WO 2008/137403), Clostridium pasteuranum (Macis et al., FEMS Microbiol. Lett. 164:21-28 (1998)) and Citrobacter freundii (Seyfried et al., J. Bacteriol 178:5793-5796 (1996)). The protein sequences for exemplary gene products can be found using the following GenBank accession numbers shown below in Table 33.

TABLE 33

| Protein | GenBank ID | GI Number | Organism |
|---|---|---|---|
| dhaB | AAC27922.1 | 3360389 | Clostridium pasteuranum |
| dhaC | AAC27923.1 | 3360390 | Clostridium pasteuranum |
| dhaE | AAC27924.1 | 3360391 | Clostridium pasteuranum |
| dhaB | P45514.1 | 1169287 | Citrobacter freundii |
| dhaC | AAB48851.1 | 1229154 | Citrobacter freundii |
| dhaE | AAB48852.1 | 1229155 | Citrobacter freundii |

When a B12-dependent diol dehydratase is utilized, heterologous expression of the corresponding reactivating factor can be used. These factors are two-subunit proteins. Exemplary genes are found in Klebsiella oxytoca (Mori et al., J. Biol. Chem. 272:32034-32041 (1997)), Salmonella typhimurium (Bobik et al., supra; Chen et al., J. Bacteriol. 176:5474-5482 (1994)), Lactobacillus collinoides (Sauvageot et al., supra), Klebsiella pneumonia (WO 2008/137403). The protein sequences for exemplary gene products can be found using the following GenBank accession numbers shown below in Table 34.

TABLE 34

| Protein | GenBank ID | GI Number | Organism |
|---|---|---|---|
| ddrA | AAC15871 | 3115376 | Klebsiella oxytoca |
| ddrB | AAC15872 | 3115377 | Klebsiella oxytoca |
| pduG | AAB84105 | 16420573 | Salmonella typhimurium |
| pduH | AAD39008 | 16420574 | Salmonella typhimurium |
| pduG | YP_002236779 | 206579698 | Klebsiella pneumonia |
| pduH | YP_002236778 | 206579863 | Klebsiella pneumonia |
| pduG | CAD01092 | 29335724 | Lactobacillus collinoides |
| pduH | AJ297723 | 29335725 | Lactobacillus collinoides |

Exemplary B12-independent diol dehyratase enzymes include glycerol dehydrogenase and dihydroxyacid dehydratase (EC 4.2.1.9). Cyclohexan-1,2-diol is not a known substrate of either enzyme. B12-independent diol dehydratase enzymes utilize S-adenosylmethionine (SAM) as a cofactor and function under strictly anaerobic conditions. The glycerol dehydrogenase and corresponding activating factor of Clostridium butyricum, encoded by dhaB1 and dhaB2, have been well-characterized (O'Brien et al., Biochemistry 43:4635-4645 (2004); Raynaud et al., Proc. Natl. Acad. Sci U.S.A 100:5010-5015 (2003)). This enzyme was recently employed in a 1,3-propanediol overproducing strain of E. coli and was able to achieve very high titers of product (Tang et al., Appl. Environ. Microbiol. 75:1628-1634 (2009)). An additional B12-independent diol dehydratase enzyme and activating factor from Roseburia inulinivorans was shown to catalyze the conversion of 2,3-butanediol to 2-butanone (US 2009/09155870). Dihydroxy-acid dehydratase (DHAD, EC 4.2.1.9) is a B12-independent enzyme participating in branched-chain amino acid biosynthesis. In its native role, it converts 2,3-dihydroxy-3-methylvalerate to 2-keto-3-methyl-valerate, a precursor of isoleucine. In valine biosynthesis the enzyme catalyzes the dehydration of 2,3-dihydroxy-isovalerate to 2-oxoisovalerate. The DHAD from Sulfolobus solfataricus has a broad substrate range and activity of a recombinant enzyme expressed in E. coli was demonstrated on a variety of aldonic acids (KIM et al., J. Biochem. 139:591-596 (2006)). The S. solfataricus enzyme is tolerant of oxygen unlike many diol dehydratase enzymes. The E. coli enzyme, encoded by ilvD, is sensitive to oxygen, which inactivates its iron-sulfur cluster (Flint et al., J. Biol. Chem. 268:14732-14742 (1993)). Similar enzymes have been characterized in Neurospora crassa (Altmiller et al., Arch. Biochem. Biophys. 138:160-170 (1970)) and Salmonella typhimurium (Armstrong et al., Biochim. Biophys. Acta 498:282-293 (1977)). The protein sequences for exemplary gene products can be found using the following GenBank accession numbers shown below in Table 35.

TABLE 35

| Protein | GenBank ID | GI Number | Organism |
|---|---|---|---|
| dhaB1 | AAM54728.1 | 27461255 | Clostridium butryicum |
| dhaB2 | AAM54729.1 | 27461256 | Clostridium butryicum |
| rdhtA | ABC25539.1 | 83596382 | Roseburia inulinivorans |
| rdhtB | ABC25540.1 | 83596383 | Roseburia inulinivorans |
| ilvD | NP_344419.1 | 15899814 | Sulfolobus solfataricus |
| ilvD | AAT48208.1 | 48994964 | Escherichia coli |
| ilvD | NP_462795.1 | 16767180 | Salmonella typhimurium |
| ilvD | XP_958280.1 | 85090149 | Neurospora crassa |

The diol dehydratase myo-inosose-2-dehydratase (EC 4.2.1.44) is another exemplary candidate. Myo-inosose is a six-membered ring containing adjacent alcohol groups. A purified enzyme encoding myo-inosose-2-dehydratase functionality has been studied in Klebsiella aerogenes in the context of myo-inositol degradation (Berman et al., J Biol. Chem. 241:800-806 (1966)), but has not been associated with a gene to date. The myo-inosose-2-dehydratase of Sinorhizobium fredii was cloned and functionally expressed in E. coli (Yoshida et al., Biosci. Biotechnol. Biochem. 70:2957-2964 (2006)). A similar enzyme from B. subtilis, encoded by iolE, has also been studied (Yoshida et al., Microbiology 150:571-580 (2004)). The protein sequences for exemplary gene products can be found using the following GenBank accession numbers shown below in Table 36.

TABLE 36

| Protein | GenBank ID | GI Number | Organism |
|---|---|---|---|
| iolE | P42416.1 | 1176989 | Bacillus subtilis |
| iolE | AAX24114.1 | 60549621 | Sinorhizobium fredii |

In some embodiments, the present invention provides a route for producing cyclohexanone from 4-acetylbutyrate (also known as 5-oxohexanoate and 5-oxocaproic acid). In this pathway, 4-acetylbutyrate is cyclized to form 1,3-cyclohexanedione. Reduction of one of the keto groups and subsequent dehydration yields 2-cyclohexenone. 2-Cyclohexenone is then reduced to cyclohexanone. The enzyme activities of this pathway are naturally present in the denitrifying bacteria Alicycliphilus denitrificans sp. K601 (formerly known as Pseudomonas sp. K601) that metabolize cyclohexanol to support growth under anaerobic conditions (Dangel et al., (1989) supra; Dangel et al., (1988) supra; Mechichi et al., supra). Pathway intermediates 1,3-cyclohexanedione and 4-acetylbutyrate can also support growth of cells containing this pathway (Dangel et al., (1988) supra)).

Although 4-acetylbutyrate has been detected in cell extracts of Escherichia coli, the biosynthetic pathway to cyclohexanone includes two enzymatic steps for synthesizing 4-acetylbutyrate from 3-oxopimeloyl-CoA. 3-oxopimeloyl-CoA is an intermediate in the pathway for producing pimeloyl-CoA as described above. Enzymes for producing 3-oxopimeloyl-CoA from acetoacetyl-CoA are described in that section. Enzymes for transforming 3-oxopimeloyl-CoA to cyclohexanone (FIG. 5) are described herein.

The first step of this pathway entails removal of the CoA moiety of 3-oxopimeloyl-CoA, which can be accomplished by a CoA-transferase, synthetase or hydrolase. Several known enzymes that act on 3-oxoacids can likely act on 3-oxopimelyl-CoA as an alternate substrate. The various CoA-synthetase, CoA-hydrolase (acting on thioester) and CoA-transferase enzymes are detailed above.

The second step of the pathway entails decarboxylation of 3-oxopimelate to 4-acetylbutyrate by a 3-oxoacid decarboxylase such as acetoacetate decarboxylase (EC 4.1.1.4). Exemplary genes for 3-oxoacid decarboxylases are enumerated above. This decarboxylation reaction can also occur spontaneously, rather than enzyme-catalyzed. In E. coli, several 3-oxoacids produced during amino acid biosynthesis have been shown to undergo spontaneous decarboxylation (Boylan et al., Biochem Biophysc. Res. Commun. 85:190-197 (1978)).

Activity of 1,3-cyclohexanedione hydrolase (4-acetylbutyrate dehydratase) has been demonstrated in the hydrolytic ring-cleavage direction in Alicycliphilus denitrificans (Dangel (1989) supra). The enzyme catalyzing this step has been characterized in cell extracts.

3-Hydroxycyclohexanone dehydrogenase (EC 1.1.99.26) reduces one of the ketones of cyclohexane-1,3-dione to 3-hydroxycyclohexanone. This enzyme has been characterized in cell extracts of Alicycliphilus denitrificans (Dangel et al., (1989) supra). Cyclohexanol dehydrogenase enzymes (EC 1.1.1.245) from Rhodococcus sp TK6 (Tae-Kang et al., supra), Nocardia sp (Stirling et al., supra), Xanthobacter sp. (Trower et al., supra) have been shown to oxidize cyclohexan-1,3-diol to cyclohexan-1,3-dione. Diacetyl reductase and additional cyclohexanol dehydrogenase genes discussed above are also applicable here.

Five recently identified beta-diketone reductases in Saccharomyces cerevisiae are able to reduce the bicyclic diketone bicyclo[2.2.2]octane-2,6-dione (BCO2,6D) to the corresponding ketoalcohol (Katz et al., Biotechnol. Bioeng. 84:573-582 (2003)). This transformation is similar to the reduction of cyclohexane-1,3-dione (step 4, FIG. 5). The enzymes are encoded by at the loci YMR226c, YDR368w, YOR120w, YGL157w and YGL039w. The protein sequences for exemplary gene products can be found using the following GenBank accession numbers shown below in Table 37.

TABLE 37

| Protein | GenBank ID | GI Number | Organism |
|---|---|---|---|
| YMR226c | NP_013953.1 | 6323882 | Saccharomyces cerevisiae |
| YDR368w | NP_010656.1 | 6320576 | Saccharomyces cerevisiae |
| YOR120w | NP_014763.1 | 6324694 | Saccharomyces cerevisiae |
| YGL157w | NP_011358.1 | 6321281 | Saccharomyces cerevisiae |
| YGL039w | NP_011476.1 | 6321399 | Saccharomyces cerevisiae |

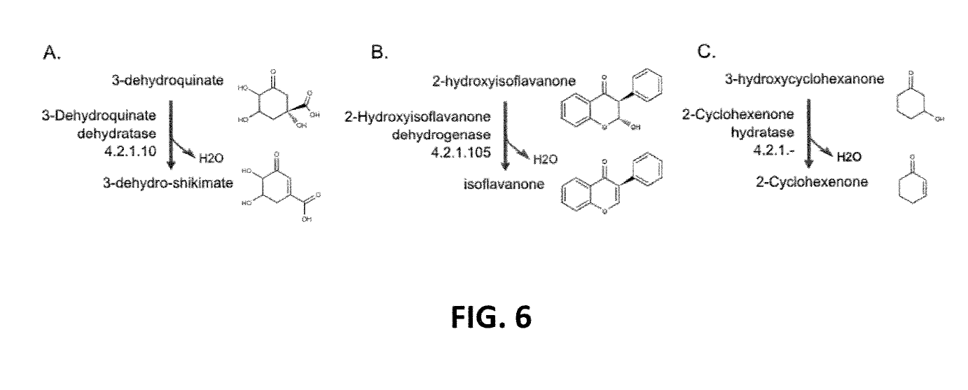

In the fifth step of the pathway, 3-hydroxycyclohexanone is dehydrated to form 2-cyclohexenone. This transformation is catalyzed by 2-cyclohexenone hydratase, characterized in cell extracts of Alicycliphilus denitrificans K601 (Dangel et al., (1989) supra). Another enzyme capable of dehydrating a cyclic beta-hydroxy ketone is 3-dehydroquinate dehydratase (EC 4.2.1.10), also known as dehydroquinase. This enzyme reversibly dehydrates 3-dehydroquinate to form 3-dehydro-shikimate (FIG. 6) and has been extensively studied as an antibiotic target. Activity on 3-hydroxycyclohexanone as a substrate has not been demonstrated. Two distinct types of dehydroquinase, type I and type II, catalyze identical reactions but differ in amino acid composition, structure and catalytic mechanism (Gourley et al., Nat. Struct. Biol. 6:521-525 (1999); Kleanthous et al., Biochem. J. 282 (Pt 3): 687-695 (1992)). High resolution structural data is available for the type I enzyme from Salmonella typhi (Gourley et al., supra) and for the type II enzymes from Mycobacterium tuberculosis (Gobel et al., J. Bacteriol. 184:216-223 (2002)) and Streptomyces coelicolor (Roszak et al., Structure 10:493-503 (2002)). Dehydroquinases have also been cloned, purified and characterized in Heliobacter pylori (Bottomley et al., Biochem. J. 319 (Pt 2):559-565 (1996)), Salmonella typhi and Escherichia coli (Chaudhuri et al., Biochem. J. 275 (pt 1):1-6 (1991)). The protein sequences for exemplary gene products can be found using the following GenBank accession numbers shown below in Table 38.

TABLE 38

| Protein | GenBank ID | GI Number | Organism |
|---|---|---|---|
| aroD | NP_416028.1 | 16129649 | Escherichia coli K12 |
| | | | sp. MG1655 |
| aroD | CAA38418.1 | 47642 | Salmonella enterica |
| | | | (Salmonella typhi) |
| aroQ | NP_626225.1 | 21220446 | Streptomyces coelicolor |
| aroD | NP_223105.1 | 15611454 | Heliobacter pylori |
| aroQ | P0A4Z6.2 | 61219243 | Mycobacterium |
| | | | tuberculosis |

The enzyme 2-hydroxyisoflavanone dehydrogenase dehydrates the cyclic beta-hydroxyl group of 2-hydroxyisoflavanone to form isoflavanone (FIG. 6B). Enzymes with this activity have been characterized in soybean (Glycine max) and Glycyrrhiza echinata (Akashi et al., Plant Physiol. 137:882-891 (2005)). The soybean enzyme HIDH was found to accept alternate substrates, whereas the G. echinata enzyme, HIDM, exhibited strict substrate specificity (Akashi et al., supra). The protein sequences for exemplary gene products can be found using the following GenBank accession numbers shown below in Table 39.

TABLE 39

| Protein | GenBank ID | GI Number | Organism |
|---|---|---|---|
| HIDM | BAD80840.1 | 56692180 | Glycine max |
| HIDM | BAD80839.1 | 56692178 | Glycyrrhiza echinata |

The final pathway step, reduction of 2-cyclohexenone to cyclohexanone, is catalyzed by cyclohexanone dehydrogenase (EC 1.3.99.14). This reaction is identical to the final step of the pathway described above

In some embodiments, the present invention provides an alternate pathway to pimeloyl-CoA starting from 2,6-diaminopentanoate. 2,6-diaminopimelate (26-DAP) is an intermediate in lysine biosynthesis and is also a constituent of bacterial cell wall peptidoglycan. Pathways I-IV of lysine biosynthesis generate 2,6-diaminopimelate from L-aspartate, wherein aspartate is converted to aspartate-semialdehyde, which is then hydrolyzed with pyruvate to form 2,3-dihydropicolinate. The conversion of 2,3-dihydropicolinate to 2,6-diaminopimelate can be accomplished by different enzymes, and involve different metabolic intermediates. In E. coli, the lysine biosynthesis pathway I accomplishes this conversion in four enzymatic steps.

Five enzymatic transformations convert 2,6-diaminopimelate to pimeloyl-CoA: deamination of the secondary amines at the 2- and 6-positions, reduction of the resulting alkenes, and formation of a thioester bond with Coenzyme A (FIG. 7). Thioester bond formation can be performed by a CoA transferase or ligase. In conjunction with the pimeloyl-CoA to cyclohexanone pathway in Section 2, the pathway is able to achieve a maximum theoretical yield of 0.75 moles of cyclohexanone per mole of glucose utilized. Even with a reversible PEP carboxykinase, the pathway is energetically limited with an ATP yield of 0.125 mol/mol. Yields were calculated under the assumption that enzymes with CoA transferase functionality are utilized (FIG. 7, step 5, FIG. 1, step 2). Aeration is not predicted to improve energetic yield.

Enzymes encoding the deamination of 2,6-dimaniopimelate and 2-aminoheptanedioate (FIG. 7, steps 1 and 3) can be provide by an aspartase (EC 4.3.1.1) which catalyzes a similar transformation, deamination of aspartate to fumarate (Viola, R. E., Adv. Enzymol. Relat. Areas. Mol. Biol. 74:295-341 (2000)). The crystal structure of the E. coli aspartase, encoded by aspA, provides insights into the catalytic mechanism and substrate specificity (Shi et al., Biochemistry 36:9136-9144 (1997)). The E. coli enzyme has been shown to react with alternate substrates aspartatephenylmethylester, asparagine, benzyl-aspartate and malate (Ma et al., Ann. N.Y. Acad. Sci. 672:60-65 (1992)). In a separate study, directed evolution was been employed on this enzyme to alter substrate specificity (Asano et al., Biomol. Eng 22:95-101 (2005)). Enzymes with aspartase functionality have also been characterized in Haemophilus influenzae (Sjostrom et al., Biochim. Biophys. Acta 1324:182-190 (1997)), Pseudomonas fluorescens (Takagi et al., J. Biochem. 96:545-552 (1984)), Bacillus subtilis (Sjostrom et al., supra) and Serratia marcescens (Takagi et al., J. Bacteriol. 161:1-6 (1985)). The protein sequences for exemplary gene products can be found using the following GenBank accession numbers shown below in Table 40.

TABLE 40

| Protein | GenBank ID | GI Number | Organism |
|---|---|---|---|
| aspA | NP_418562 | 90111690 | Escherichia coli K12 subsp. MG1655 |
| aspA | P44324.1 | 1168534 | Haemophilus influenzae |
| aspA | P07346.1 | 114273 | Pseudomonas fluorescens |
| ansB | P26899.1 | 251757243 | Bacillus subtilis |
| aspA | P33109.1 | 416661 | Serratia marcescens |

Reduction of the pathway intermediates, 6-aminohept-2-enedioate and 6-carboxyhex-2-enoate, can be performed by a 2-enoate reductase (EC 1.3.1.31) as described above.

The acylation of pimelate to pimeloyl-CoA is catalyzed by pimeloyl-CoA synthetase, also called 6-carboxyhexanoate-CoA ligase (EC 6.2.1.14). This enzyme concomitantly forms AMP and pyrophosphate and consumes 2 ATP equivalents if pyrophosphate is hydrolyzed. The enzymes from Bacillus subtilis (Bower et al., supra), Bacillus sphaericus (Ploux et al., Biochem. J. 287 (pt 3):685-690 (1992)) and Pseudomonas mendocina (Binieda et al., Biochem. J. 340 (pt 3):793-801 (1999)) have been cloned, sequenced and characterized. The protein sequences for exemplary gene products can be found using the following GenBank accession numbers shown below in Table 41.

TABLE 41

| Protein | GenBank ID | GI Number | Organism |
|---|---|---|---|
| bioW | NP_390902.2 | 50812281 | Bacillus substilis |
| bioW | CAA10043.1 | 3850837 | Pseudomonas mendocina |
| bioW | P22822.1 | 115012 | Bacillus sphaericus |

An enzyme capable of transferring the CoA moiety from acetyl-CoA or succinyl-CoA to pimelate is the E. coli acyl-CoA:acetate-CoA transferase, also known as acetate-CoA transferase (EC 2.8.3.8). This enzyme has been shown to transfer the CoA moiety to acetate from a variety of branched and linear acyl-CoA substrates, including isobutyrate (Matthies et al., Appl. Environ. Microbiol. 58:1435-1439 (1992)), valerate (Vanderwinkel et al., Biochem. Biophys. Res. Commun. 33:902-908 (1968)) and butanoate (Vanderwinkel et al., supra). This enzyme is encoded by atoA (alpha subunit) and atoD (beta subunit) in E. coli sp. K12 (Korolev et al., Acta Crystallogr. D Biol Crystallogr. 58:2116-2121 (2002); Vanderwinkel et al., supra) and actA and cg0592 in Corynebacterium glutamicum ATCC 13032 (Duncan et al., Appl. Environ. Microbiol. 68:5186-5190 (2002)). Similar enzymes exist in Clostridium acetobutylicum (Cary et al., Appl Environ Microbiol 56:1576-1583 (1990)) and Clostridium saccharoperbutylacetonicum (Kosaka et al., Biosci. Biotechnol Biochem. 71:58-68 (2007)). The protein sequences for exemplary gene products can be found using the following GenBank accession numbers shown below in Table 42.

TABLE 42

| Protein | GenBank ID | GI Number | Organism |
|---|---|---|---|
| atoA | P76459.1 | 2492994 | Escherichia coli K12 |
| atoD | P76458.1 | 2492990 | Escherichia coli K12 |
| actA | YP_226809.1 | 62391407 | Corynebacterium glutamicum ATCC |
| | | | 13032 |
| cg0592 | YP_224801.1 | 62389399 | Corynebacterium glutamicum ATCC |
| | | | 13032 |
| ctfA | NP_149326.1 | 15004866 | Clostridium acetobutylicum |
| ctfB | NP_149327.1 | 15004867 | Clostridium acetobutylicum |
| ctfA | AAP42564.1 | 31075384 | Clostridium saccharoperbutyl- |
| | | | acetonicum |
| ctfB | AAP42565.1 | 31075385 | Clostridium saccharoperbutyl- |
| | | | acetonicum |

The gene products of cat1, cat2, and cat3 of Clostridium kluyveri catalyze analogous transformations, forming succinyl-CoA, 4-hydroxybutyryl-CoA, and butyryl-CoA from their corresponding acids (Seedorf et al., supra; Gourley et al., supra). Succinyl-CoA transferase activity is also present in Trichomonas vaginalis (van Grinsven et al., J. Biol. Chem. 283:1311-1418 (2008)) and Trypanosoma brucei (Riviere et al., J. Biol. Chem. 279:45337-45346 (2004)). The protein sequences for exemplary gene products can be found using the following GenBank accession numbers shown below in Table 43.

TABLE 43

| Protein | GenBank ID | GI Number | Organism |
|---|---|---|---|
| cat1 | P38946.1 | 729048 | Clostridium kluyveri |
| cat2 | P38942.2 | 172046066 | Clostridium kluyveri |
| cat3 | EDK35586.1 | 146349050 | Clostridium kluyveri |
| TVAG_395550 | XP_001330176 | 123975034 | Trichomonas vaginalis G3 |
| Tb11.02.0290 | XP_828352 | 71754875 | Trypanosoma brucei |

An alternate route for producing pimeloyl-CoA from 2,6-diaminopentanoate involves forming a thioester bond from one of the enoic acid pathway intermediates, 6-aminohept-2-enedioic acid or 6-carboxyhex-2-enoate. An enoyl-CoA transferase such as glutaconate CoA-transferase (EC 2.8.3.12) would be a good enzyme for this transformation. This enzyme from Acidaminococcus fermentans, which has been cloned and functionally expressed in E. coli (Mack et al., Eur. J. Biochem. 226:41-51 (1994)), reacts with multiple enoyl-CoA substrates including 3-butenoyl-CoA, acrylyl-CoA, and 2-hydroxyglutaryl-CoA (Buckel et al., Eur. J. Biochem. 118:315-321 (1981)). Glutaconate CoA-transferase activity has also been detected in Clostridium sporosphaeroides and Clostridium symbiosum. Additional glutaconate CoA-transferase enzymes can be inferred by homology to the Acidaminococcus fermentans protein sequence. The protein sequences for exemplary gene products can be found using the following GenBank accession numbers shown below in Table 44.

TABLE 44

| Protein | GenBank ID | GI Number | Organism |
|---|---|---|---|
| gctA | CAA57199.1 | 559392 | Acidaminococcus fermentans |
| gctB | CAA57200.1 | 559393 | Acidaminococcus fermentans |
| gctA | ACJ24333.1 | 212292816 | Clostridium symbiosum |
| gctB | ACJ24326.1 | 212292808 | Clostridium symbiosum |
| gctA | NP_603109.1 | 19703547 | Fusobacterium nucleatum |
| gctB | NP_603110.1 | 19703548 | Fusobacterium nucleatum |

When an enoyl-CoA intermediate is formed from 6-aminohept-2-enedioate or 6-carboxyhex-2-enoate, reduction of the alkene can be performed by an enoyl-CoA reductase. Exemplary enoyl-CoA reductase genes are described above.

The non-naturally occurring microbial organisms of the invention can be produced by introducing expressible nucleic acids encoding one or more of the enzymes or proteins participating in one or more cyclohexanone biosynthetic pathways. Depending on the host microbial organism chosen for biosynthesis, nucleic acids for some or all of a particular cyclohexanone biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve cyclohexanone biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as cyclohexanone.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizobus oryzae, Yarrowia lipolytica, and the like. E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Depending on the cyclohexanone biosynthetic pathway constituents of a selected host microbial organism, the non-naturally occurring microbial organisms of the invention will include at least one exogenously expressed cyclohexanone pathway-encoding nucleic acid and up to all encoding nucleic acids for one or more cyclohexanone biosynthetic pathways. For example, cyclohexanone biosynthesis can be established in a host deficient in a pathway enzyme or protein through exogenous expression of the corresponding encoding nucleic acid. In a host deficient in all enzymes or proteins of a cyclohexanone pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of cyclohexanone can be included, such as a PEP carboxykinase, a 2-ketocyclohexane-1-carboxyl-CoA hydrolase (acting on C—C bond), a 2-ketocyclohexane-1-carboxylate decarboxylase and an enzyme selected from a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase. Such a pathway can also include a complete set of exogenous enzymes for the production of pimeloyl-CoA, which includes a 3-hydroxybutyryl-CoA dehydratase, a glutaryl-CoA dehydrogenase, a oxopimeloyl-CoA:glutaryl-CoA acyltransferase, a 3-hydroxypimeloyl-CoA dehydrogenase, a 3-hydroxypimeloyl-CoA dehydratase, and a pimeloyl-CoA dehydrogenase.

Other examples of complete enzyme sets for the production of cyclohexanone include for example (a) PEP carboxykinase, 6-ketocyclohex-1-ene-1-carboxyl-CoA hydrolase (acting on C—C bond), 6-ketocyclohex-1-ene-1-carboxylate decarboxylase, cyclohexanone dehydrogenase, and an enzyme selected from 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; (b) PEP carboxykinase, 6-ketocyclohex-1-ene-1-carboxyl-CoA hydrolase (acting on C—C bond), 6-ketocyclohex-1-ene-1-carboxylate reductase, 2-ketocyclohexane-1-carboxylate decarboxylase, and an enzyme selected from 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; and (c) PEP carboxykinase, 6-ketocyclohex-1-ene-1-carboxyl-CoA hydrolase (acting on C—C bond), 6-ketocyclohex-1-ene-1-carboxyl-CoA reductase, 2-ketocyclohexane-1-carboxylate decarboxylase, and an enzyme selected from the group consisting of 2-ketocyclohexane-1-carboxyl-CoA synthetase, 2-ketocyclohexane-1-carboxyl-CoA transferase, and 2-ketocyclohexane-1-carboxyl-CoA hydrolase (acting on thioester). Any of pathways (a)-(c) can also have a complete set of nucleic acids encoding a 3-hydroxypimeloyl-CoA pathway which includes an acetoacetyl-CoA reductase, a 3-hydroxybutyryl-CoA dehydratase, a glutaryl-CoA dehydrogenase, a oxopimeloyl-CoA:glutaryl-CoA acyltransferase, and a 3-hydroxypimeloyl-CoA dehydrogenase.

In still further exemplary embodiments a set of nucleic acids encoding a complete cyclohexanone pathway can include a PEP carboxykinase, an adipate semialdehyde dehydratase, a cyclohexane-1,2-diol dehydrogenase, and a cyclohexane-1,2-diol dehydratase. In yet still further embodiments a complete cyclohexanone pathway can include nucleic acids encoding a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from a 3-oxopimeloyl-CoA synthetase, a 3-oxopimeloyl-CoA hydrolase (acting on thioester), and a 3-oxopimeloyl-coA transferase. In some embodiments, this latter pathway can also include a 3-oxopimeloyl-CoA pathway which includes a 3-hydroxyacyl-CoA dehydrogenase, a 3-hydroxybutyryl-CoA dehydratase, a glutaryl-CoA dehydrogenase, and a oxopimeloyl-CoA:glutaryl-CoA acyltransferase.

Given the teachings and guidance provided herein, those skilled in the art will understand that the number of encoding nucleic acids to introduce in an expressible form will, at least, parallel the cyclohexanone pathway deficiencies of the selected host microbial organism. Therefore, a non-naturally occurring microbial organism of the invention can have one, two, three, four, five, six, seven, eight, nine, ten, eleven, twelve, thirteen, fourteen, fifteen or up to all nucleic acids encoding the enzymes or proteins constituting a cyclohexanone biosynthetic pathway disclosed herein. In some embodiments, the non-naturally occurring microbial organisms also can include other genetic modifications that facilitate or optimize cyclohexanone biosynthesis or that confer other useful functions onto the host microbial organism. One such other functionality can include, for example, augmentation of the synthesis of one or more of the cyclohexanone pathway precursors such as 2-ketocyclohexane-1-carboxylate, 2-ketocyclohexane-1-carboxyl-CoA, pimeloyl-CoA, 6-carboxyhex-2-enoyl-CoA, 3-hydroxypimeloyl-CoA, glutaryl-CoA, crotonyl-CoA, 3-hydroxybutyryl-CoA, acetoacetyl-CoA, 6-ketocyclohex-1-ene-1-carboxyl-CoA, 6-ketocyclohex-1-ene-1-carboxylate, 2-cyclohexenone, cyclohexane-1,2-diol, 2-hydroxycyclohexanone, cyclohexane-1,2-dione, adipate semialdehyde, 3-hydroxycyclohexanone, 1,3-cyclohexanedione, 4-acetylbutyrate, or 3-oxopimelate.

Generally, a host microbial organism is selected such that it produces the precursor of a cyclohexanone pathway, either as a naturally produced molecule or as an engineered product that either provides de novo production of a desired precursor or increased production of a precursor naturally produced by the host microbial organism. For example, acetoacetyl-CoA is produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of a cyclohexanone pathway.

In some embodiments, a non-naturally occurring microbial organism of the invention is generated from a host that contains the enzymatic capability to synthesize cyclohexanone. In this specific embodiment it can be useful to increase the synthesis or accumulation of a cyclohexanone pathway product to, for example, drive cyclohexanone pathway reactions toward cyclohexanone production. Increased synthesis or accumulation can be accomplished by, for example, overexpression of nucleic acids encoding one or more of the above-described cyclohexanone pathway enzymes or proteins. Over expression the enzyme or enzymes and/or protein or proteins of the cyclohexanone pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing cyclohexanone, through overexpression of one, two, three, four, five, six, seven, eight, nine, ten, eleven, twelve, thirteen, fourteen, fifteen, that is, up to all nucleic acids encoding cyclohexanone biosynthetic pathway enzymes or proteins. In addition, a non-naturally occurring organism can be generated by mutagenesis of an endogenous gene that results in an increase in activity of an enzyme in the cyclohexanone biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention. The nucleic acids can be introduced so as to confer, for example, a cyclohexanone biosynthetic pathway onto the microbial organism. Alternatively, encoding nucleic acids can be introduced to produce an intermediate microbial organism having the biosynthetic capability to catalyze some of the required reactions to confer cyclohexanone biosynthetic capability. For example, a non-naturally occurring microbial organism having a cyclohexanone biosynthetic pathway can comprise at least two exogenous nucleic acids encoding desired enzymes or proteins, such as the combination of a 2-ketocyclohexane-1-carboxylate decarboxylase and a 2-ketocyclohexanecarboxyl-CoA hydrolase (acting on C—C bond; reaction run in reverse), or a 2-ketocyclohexane-1-carboxylate decarboxylase and a CoA synthetase, hydrolase or transferase, or a 2-ketocyclohexanecarboxyl-CoA hydrolase (acting on C—C bond; reaction run in reverse) and a CoA synthetase, hydrolase, or transferase, and the like. These are merely exemplary, and one skilled in the art will appreciate that any combination of two enzymes from any of the disclosed pathways can be provided by introduction of exogenous nucleic acids. Thus, it is understood that any combination of two or more enzymes or proteins of a biosynthetic pathway can be included in a non-naturally occurring microbial organism of the invention. Similarly, it is understood that any combination of three or more enzymes or proteins of a biosynthetic pathway can be included in a non-naturally occurring microbial organism of the invention, for example, a 2-ketocyclohexane-1-carboxylate decarboxylase, a 2-ketocyclohexanecarboxyl-CoA hydrolase (acting on C—C bond; reaction run in reverse), and a CoA synthetase, or a 2-ketocyclohexane-1-carboxylate decarboxylase, a 2-ketocyclohexanecarboxyl-CoA hydrolase (acting on C—C bond; reaction run in reverse), and a CoA hydrolase or a 2-ketocyclohexane-1-carboxylate decarboxylase, a 2-ketocyclohexanecarboxyl-CoA hydrolase (acting on C—C bond; reaction run in reverse), and a CoA transferase, and so forth, as desired, so long as the combination of enzymes and/or proteins of the desired biosynthetic pathway results in production of the corresponding desired product. Similarly, any combination of four, five, six, seven, eight, nine, ten, eleven, twelve, thirteen, fourteen, fifteen or more enzymes or proteins of a biosynthetic pathway as disclosed herein can be included in a non-naturally occurring microbial organism of the invention, as desired, so long as the combination of enzymes and/or proteins of the desired biosynthetic pathway results in production of the corresponding desired product.

In addition to the biosynthesis of cyclohexanone as described herein, the non-naturally occurring microbial organisms and methods of the invention also can be utilized in various combinations with each other and with other microbial organisms and methods well known in the art to achieve product biosynthesis by other routes. For example, one alternative to produce cyclohexanone other than use of the cyclohexanone producers is through addition of another microbial organism capable of converting a cyclohexanone pathway intermediate to cyclohexanone. One such procedure includes, for example, the fermentation of a microbial organism that produces a cyclohexanone pathway intermediate. The cyclohexanone pathway intermediate can then be used as a substrate for a second microbial organism that converts the cyclohexanone pathway intermediate to cyclohexanone. The cyclohexanone pathway intermediate can be added directly to another culture of the second organism or the original culture of the cyclohexanone pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, cyclohexanone. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of cyclohexanone can be accomplished by constructing a microbial organism that contains biosynthetic pathways for conversion of one pathway intermediate to another pathway intermediate or the product. Alternatively, cyclohexanone also can be biosynthetically produced from microbial organisms through co-culture or co-fermentation using two organisms in the same vessel, where the first microbial organism produces a cyclohexanone intermediate, such as pimeloyl-CoA, and the second microbial organism converts the intermediate to cyclohexanone.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce cyclohexanone.

Sources of encoding nucleic acids for a cyclohexanone pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Saccharomyces cerevisae, Clostridium acetobutylicum, Zoogloea ramigera, Pseudomonas putida, Syntrophus aciditrophicus, Haemophilus influenza, Azoarcus sp. CIB, Thauera aromatica, Glycine max, and Ascarius suum, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite cyclohexanone biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of cyclohexanone described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative cyclohexanone biosynthetic pathway exists in an unrelated species, cyclohexanone biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms can differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize cyclohexanone.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris. E. coli is a particularly useful host organism since it is a well characterized microbial organism suitable for genetic engineering. Other particularly useful host organisms include yeast such as Saccharomyces cerevisiae.

Methods for constructing and testing the expression levels of a non-naturally occurring cyclohexanone-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of cyclohexanone can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more cyclohexanone biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments, the present invention provides a method for producing cyclohexanone, that includes culturing a non-naturally occurring microbial organism having a cyclohexanone pathway. The pathway includes at least one exogenous nucleic acid encoding a cyclohexanone pathway enzyme expressed in a sufficient amount to produce cyclohexanone, under conditions and for a sufficient period of time to produce cyclohexanone. The cyclohexanone pathway comprising a PEP carboxykinase, a 2-ketocyclohexane-1-carboxyl-CoA hydrolase (acting on C—C bond), a 2-ketocyclohexane-1-carboxylate decarboxylase and an enzyme selected from the group consisting of a 2-ketocyclohexane-1-carboxyl-CoA hydrolase (acting on thioester), a 2-ketocyclohexane-1-carboxyl-CoA transferase, and a 2-ketocyclohexane-1-carboxyl-CoA synthetase.

The present invention also provides a method for producing cyclohexanone that includes culturing a non-naturally occurring microbial organism having a cyclohexanone pathway that includes at least one exogenous nucleic acid encoding a cyclohexanone pathway enzyme expressed in a sufficient amount to produce cyclohexanone, under conditions and for a sufficient period of time to produce cyclohexanone, wherein the cyclohexanone pathway includes a set of cyclohexanone pathway enzymes. The set of cyclohexanone pathway enzymes selected from (a) PEP carboxykinase, 6-ketocyclohex-1-ene-1-carboxyl-CoA hydrolase (acting on C—C bond), 6-ketocyclohex-1-ene-1-carboxylate decarboxylase, cyclohexanone dehydrogenase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; (b) PEP carboxykinase, 6-ketocyclohex-1-ene-1-carboxyl-CoA hydrolase (acting on C—C bond), 6-ketocyclohex-1-ene-1-carboxylate reductase, 2-ketocyclohexane-1-carboxylate decarboxylase, and an enzyme selected from the group consisting of 6-ketocyclohex-1-ene-1-carboxyl-CoA synthetase, 6-ketocyclohex-1-ene-1-carboxyl-CoA hydrolase (acting on thioester), and 6-ketocyclohex-1-ene-1-carboxyl-CoA transferase; and (c) PEP carboxykinase, 6-ketocyclohex-1-ene-1-carboxyl-CoA hydrolase (acting on C—C), 6-ketocyclohex-1-ene-1-carboxyl-CoA reductase, 2-ketocyclohexane-1-carboxylate decarboxylase, and an enzyme selected from the group consisting of 2-ketocyclohexane-1-carboxyl-CoA synthetase, 2-ketocyclohexane-1-carboxyl-CoA transferase, 2-ketocyclohexane-1-carboxyl-CoA hydrolase (acting on thioester).

The present invention also provides a method for producing cyclohexanone that includes culturing a non-naturally occurring microbial organism having a cyclohexanone pathway which includes at least one exogenous nucleic acid encoding a cyclohexanone pathway enzyme expressed in a sufficient amount to produce cyclohexanone, under conditions and for a sufficient period of time to produce cyclohexanone. Such a cyclohexanone pathway includes a PEP carboxykinase, an adipate semialdehyde dehydratase, a cyclohexane-1,2-diol dehydrogenase, and a cyclohexane-1,2-diol dehydratase.

In yet a further embodiment, the present invention provides a method for producing cyclohexanone that includes culturing a non-naturally occurring microbial organism having a cyclohexanone pathway having at least one exogenous nucleic acid encoding a cyclohexanone pathway enzyme expressed in a sufficient amount to produce cyclohexanone, under conditions and for a sufficient period of time to produce cyclohexanone. In such embodiments, the cyclohexanone pathway includes a PEP carboxykinase, a 3-oxopimelate decarboxylase, a 4-acetylbutyrate dehydratase, a 3-hydroxycyclohexanone dehydrogenase, a 2-cyclohexenone hydratase, a cyclohexanone dehydrogenase and an enzyme selected from the group consisting of a 3-oxopimeloyl-CoA synthetase, a 3-oxopimeloyl-CoA hydrolase (acting on thioester), and a 3-oxopimeloyl-coA transferase.

Suitable purification and/or assays to test for the production of cyclohexanone can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art. For example, the specific activity of cyclohexanone dehydrogenase can be assayed in the reductive direction using a colorimetric assay adapted from the literature (Dune et al., FEMS Microbial. Rev. 17:251-262 (1995); Palosaari et al., J. Bacteriol. 170:2971-2976 (1988); Welch et al., Arch. Biochem. Biophys. 273:309-318 (1989)). In this assay, the substrates 2-cyclohexenone and NADH are added to cell extracts in a buffered solution, and the oxidation of NADH is followed by reading absorbance at 340 nM at regular intervals. The resulting slope of the reduction in absorbance at 340 nM per minute, along with the molar extinction coefficient of NADH at 340 nM (6000) and the protein concentration of the extract, can be used to determine the specific activity of cyclohexanone dehydrogenase.

The cyclohexanone can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products of the invention. For example, the cyclohexanone producers can be cultured for the biosynthetic production of cyclohexanone.

For the production of cyclohexanone, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic conditions can be applied by perforating the septum with a small hole for limited aeration. Exemplary anaerobic conditions have been described previously and are well-known in the art. Exemplary aerobic and anaerobic conditions are described, for example, in U.S. patent application Ser. No. 11/891,602, filed Aug. 10, 2007. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

In addition to renewable feedstocks such as those exemplified above, the microbial organisms of the invention also can be modified for growth on syngas as its source of carbon. In this specific embodiment, one or more proteins or enzymes are expressed in the non-naturally occurring microbial organisms to provide a metabolic pathway for utilization of syngas or other gaseous carbon source.

Organisms of the present invention can utilize, and the growth medium can include, for example, carbohydrate source which can supply a source of carbon to the non-naturally occurring microorganism. Such sources include, for example, sugars such as glucose, xylose, arabinose, galactose, mannose, fructose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of cyclohexanone.

In addition to renewable feedstocks such as those exemplified above, the cyclohexanone microbial organisms of the invention also can be modified for growth on syngas as its source of carbon. In this specific embodiment, one or more proteins or enzymes are expressed in the cyclohexanone producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+nADP+nPi→CH₃COOH+2H₂O+nATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a cyclohexanone pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to the cyclohexanone precursors, glyceraldehyde-3-phosphate, phosphoenolpyruvate, and pyruvate, by pyruvate:ferredoxin oxidoreductase and the enzymes of gluconeogenesis. Following the teachings and guidance provided herein for introducing a sufficient number of encoding nucleic acids to generate a cyclohexanone pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms of the invention such that the modified organism contains a reductive TCA pathway can confer syngas utilization ability.

Accordingly, given the teachings and guidance provided herein, those skilled in the art will understand that a non-naturally occurring microbial organism can be produced that secretes the biosynthesized compounds of the invention when grown on a carbon source such as a carbohydrate, syngas, CO and/or CO2. Such compounds include, for example, cyclohexanone and any of the intermediate metabolites in the cyclohexanone pathway. All that is required is to engineer in one or more of the required enzyme or protein activities to achieve biosynthesis of the desired compound or intermediate including, for example, inclusion of some or all of the cyclohexanone biosynthetic pathways. Accordingly, the invention provides a non-naturally occurring microbial organism that produces and/or secretes cyclohexanone when grown on a carbohydrate or other carbon source and produces and/or secretes any of the intermediate metabolites shown in the cyclohexanone pathway when grown on a carbohydrate or other carbon source. The cyclohexanone producing microbial organisms of the invention can initiate synthesis from an intermediate, for example, acetyl-CoA.

The non-naturally occurring microbial organisms of the invention are constructed using methods well known in the art as exemplified herein to exogenously express at least one nucleic acid encoding a cyclohexanone pathway enzyme or protein in sufficient amounts to produce cyclohexanone. It is understood that the microbial organisms of the invention are cultured under conditions sufficient to produce cyclohexanone. Following the teachings and guidance provided herein, the non-naturally occurring microbial organisms of the invention can achieve biosynthesis of cyclohexanone resulting in intracellular concentrations between about 0.1-200 mM or more. Generally, the intracellular concentration of cyclohexanone is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the cyclohexanone producers can synthesize cyclohexanone at intracellular concentrations of 5-10 mM or more as well as all other concentrations exemplified herein. It is understood that, even though the above description refers to intracellular concentrations, cyclohexanone producing microbial organisms can produce cyclohexanone intracellularly and/or secrete the product into the culture medium.

In addition to the culturing and fermentation conditions disclosed herein, growth condition for achieving biosynthesis of cyclohexanone can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

In some embodiments, the carbon feedstock and other cellular uptake sources such as phosphate, ammonia, sulfate, chloride and other halogens can be chosen to alter the isotopic distribution of the atoms present in cyclohexanone or any cyclohexanone pathway intermediate. The various carbon feedstock and other uptake sources enumerated above will be referred to herein, collectively, as “uptake sources.” Uptake sources can provide isotopic enrichment for any atom present in the product cyclohexanone or cyclohexanone pathway intermediate including any cyclohexanone impurities, or for side products generated in reactions diverging away from a cyclohexanone pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, a target isotopic ratio of an uptake source can be obtained via synthetic chemical enrichment of the uptake source. Such isotopically enriched uptake sources can be purchased commercially or prepared in the laboratory. In some embodiments, a target isotopic ratio of an uptake source can be obtained by choice of origin of the uptake source in nature. In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO2, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S-B)/(M-B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil. (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mille. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content and/or prepared downstream products that utilize of the invention having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, the present invention provides cyclohexanone or a cyclohexanone intermediate that has a carbon-12, carbon-13, and carbon-14 ratio that reflects an atmospheric carbon, also referred to as environmental carbon, uptake source. For example, in some aspects the cyclohexanone or a cyclohexanone intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, the present invention provides cyclohexanone or a cyclohexanone intermediate that has a carbon-12, carbon-13, and carbon-14 ratio that reflects petroleum-based carbon uptake source. In this aspect, the cyclohexanone or a cyclohexanone intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, the present invention provides cyclohexanone or a cyclohexanone intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present invention relates to the biologically produced cyclohexanone or cyclohexanone intermediate as disclosed herein, and to the products derived therefrom, wherein the cyclohexanone or a cyclohexanone intermediate has a carbon-12, carbon-13, and carbon-14 isotope ratio of about the same value as the CO₂ that occurs in the environment. For example, in some aspects the invention provides: bioderived cyclohexanone or a bioderived cyclohexanone intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived cyclohexanone or a bioderived cyclohexanone intermediate as disclosed herein, wherein the bioderived product is chemically modified to generate a final product. Methods of chemically modifying a bioderived product of cyclohexanone, or an intermediate thereof, to generate a desired product are well known to those skilled in the art, as described herein. The invention further provides organic solvents, polyurethane resins, polyester resins, hypoglycaemic agents, butadiene and/or butadiene-based products having a carbon-12 versus carbon-13 versus carbon-14 isotope ratio of about the same value as the CO₂ that occurs in the environment, wherein the organic solvents, polyurethane resins, polyester resins, hypoglycaemic agents, butadiene and/or butadiene-based products are generated directly from or in combination with bioderived cyclohexanone or a bioderived cyclohexanone intermediate as disclosed herein.

Cyclohexanone is a chemical used in commercial and industrial applications and is also used as a raw material in the production of a wide range of products. Non-limiting examples of such applications and products include Nylon 6 and Nylon 66.

Accordingly, in some embodiments, the invention provides biobased used as a raw material in the production of a wide range of products comprising one or more bioderived cyclohexanone or bioderived cyclohexanone intermediate produced by a non-naturally occurring microorganism of the invention or produced using a method disclosed herein.

As used herein, the term “bioderived” means derived from or synthesized by a biological organism and can be considered a renewable resource since it can be generated by a biological organism. Such a biological organism, in particular the microbial organisms of the invention disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the invention. A biobased or bioderived product is in contrast to a petroleum derived product, wherein such a product is derived from or synthesized from petroleum or a petrochemical feedstock.

In some embodiments, the invention provides products, such as Nylon 6 and Nylon 66, comprising bioderived cyclohexanone or bioderived cyclohexanone intermediate, wherein the bioderived cyclohexanone or bioderived cyclohexanone intermediate includes all or part of the cyclohexanone or cyclohexanone intermediate used in the production of Nylon 6 and Nylon 66 comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived cyclohexanone or bioderived cyclohexanone intermediate as disclosed herein. Additionally, in some aspects, the invention provides biobased Nylon 6 and Nylon 66, wherein the cyclohexanone or cyclohexanone intermediate used in its production is a combination of bioderived and petroleum derived cyclohexanone or cyclohexanone intermediate. For example, biobased Nylon 6 and Nylon 66 and other cyclohexanone-based products can be produced using 50% bioderived cyclohexanone and 50% petroleum derived cyclohexanone or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing Nylon 6 and Nylon 66 and other cyclohexanone-based products using the bioderived cyclohexanone or bioderived cyclohexanone intermediate of the invention are well known in the art.

The non-naturally occurring microbial organisms of the invention are constructed using methods well known in the art as exemplified herein to exogenously express at least one nucleic acid encoding a cyclohexanone pathway enzyme or protein in sufficient amounts to produce cyclohexanone. It is understood that the microbial organisms of the invention are cultured under conditions sufficient to produce cyclohexanone. Following the teachings and guidance provided herein, the non-naturally occurring microbial organisms of the invention can achieve biosynthesis of cyclohexanone resulting in intracellular concentrations between about 0.1-200 mM or more. Generally, the intracellular concentration of cyclohexanone is between about 3-200 mM, particularly between about 10-175 mM and more particularly between about 50-150 mM, including about 50 mM, 75 mM, 100 mM, 125 mM, or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. patent application Ser. No. 11/891,602, filed Aug. 10, 2007. Any of these conditions can be employed with the non-naturally occurring microbial organisms as well as other anaerobic conditions well known in the art. Under such anaerobic conditions, the cyclohexanone producers can synthesize cyclohexanone at intracellular concentrations of 5-10 mM or more as well as all other concentrations exemplified herein. It is understood that, even though the above description refers to intracellular concentrations, cyclohexanone producing microbial organisms can produce cyclohexanone intracellularly and/or secrete the product into the culture medium.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of cyclohexanone includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of cyclohexanone. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cyclohexanone. Generally, and as with non-continuous culture procedures, the continuous and/or near-continuous production of cyclohexanone can include culturing a non-naturally occurring cyclohexanone producing organism of the invention in sufficient nutrients and medium to sustain and/or nearly sustain growth in an exponential phase. Continuous culture under such conditions can include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of cyclohexanone can be utilized in, for example, fed-batch fermentation and batch separation; fed-batch fermentation and continuous separation, or continuous fermentation and continuous separation. Examples of batch and continuous fermentation procedures are well known in the art.

In addition to the culturing and fermentation conditions disclosed herein, growth condition for achieving biosynthesis of cyclohexanone can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

In addition to the above fermentation procedures using the cyclohexanone producers of the invention for continuous production of substantial quantities of cyclohexanone, the cyclohexanone producers also can be, for example, simultaneously subjected to chemical synthesis procedures to convert the product to other compounds or the product can be separated from the fermentation culture and sequentially subjected to chemical or enzymatic conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of cyclohexanone.

Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (e.g., >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened.

Numerous directed evolution technologies have been developed (for reviews, see Hibbert et al., Biomol. Eng 22:11-19 (2005); Huisman et al., Biocatalysis in the pharmaceutical and biotechnology industries, pp. 717-742 (2007) CRC Press, R. N. Patel, Ed.); Otten et al., Biomol. Eng 22:1-9 (2005); and Sen et al., Appl Biochem. Biotechnol 143:212-223 (2007).) to be effective at creating diverse variant libraries and these methods have been successfully applied to the improvement of a wide range of properties across many enzyme classes.

Enzyme characteristics that have been improved and/or altered by directed evolution technologies include, for example, selectivity/specificity—for conversion of non-natural substrates; temperature stability—for robust high temperature processing; pH stability—for bioprocessing under lower or higher pH conditions; substrate or product tolerance—so that high product titers can be achieved; binding (Kₘ)—broadens substrate binding to include non-natural substrates; inhibition (Kᵢ)—to remove inhibition by products, substrates, or key intermediates; activity (kcat)—increases enzymatic reaction rates to achieve desired flux; expression levels—increases protein yields and overall pathway flux; oxygen stability—for operation of air sensitive enzymes under aerobic conditions; and anaerobic activity—for operation of an aerobic enzyme in the absence of oxygen.

The following exemplary methods have been developed for the mutagenesis and diversification of genes to target desired properties of specific enzymes. Any of these can be used to alter/optimize activity of a decarboxylase enzyme.

EpPCR (Pritchard et al., J Theor. Biol. 234:497-509 (2005).) introduces random point mutations by reducing the fidelity of DNA polymerase in PCR reactions by the addition of Mn²⁺ ions, by biasing dNTP concentrations, or by other conditional variations. The five step cloning process to confine the mutagenesis to the target gene of interest involves: 1) error-prone PCR amplification of the gene of interest; 2) restriction enzyme digestion; 3) gel purification of the desired DNA fragment; 4) ligation into a vector; 5) transformation of the gene variants into a suitable host and screening of the library for improved performance. This method can generate multiple mutations in a single gene simultaneously, which can be useful. A high number of mutants can be generated by EpPCR, so a high-throughput screening assay or a selection method (especially using robotics) is useful to identify those with desirable characteristics.

Error-prone Rolling Circle Amplification (epRCA) (Fujii et al., Nucl. Acids Res 32:e145 (2004); and Fujii et al., Nat. Protoc. 1:2493-2497 (2006).) has many of the same elements as epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats. Adjusting the Mn²⁺ concentration can vary the mutation rate somewhat. This technique uses a simple error-prone, single-step method to create a full copy of the plasmid with 3-4 mutations/kbp. No restriction enzyme digestion or specific primers are required. Additionally, this method is typically available as a kit.

DNA or Family Shuffling (Stemmer, W. P., Proc Natl Acad Sci U S.A. 91:10747-10751 (1994); and Stemmer, W. P., Nature 370:389-391 (1994).) typically involves digestion of 2 or more variant genes with nucleases such as Dnase I or EndoV to generate a pool of random fragments that are reassembled by cycles of annealing and extension in the presence of DNA polymerase to create a library of chimeric genes. Fragments prime each other and recombination occurs when one copy primes another copy (template switch). This method can be used with >1 kbp DNA sequences. In addition to mutational recombinants created by fragment reassembly, this method introduces point mutations in the extension steps at a rate similar to error-prone PCR. The method can be used to remove deleterious random neutral mutations that might confer antigenicity.

Staggered Extension (StEP) (Zhao et al., Nat. Biotechnol 16:258-261 (1998).) entails template priming followed by repeated cycles of 2 step PCR with denaturation and very short duration of annealing/extension (as short as 5 sec). Growing fragments anneal to different templates and extend further, which is repeated until full-length sequences are made. Template switching means most resulting fragments have multiple parents. Combinations of low-fidelity polymerases (Taq and Mutazyme) reduce error-prone biases because of opposite mutational spectra.

In Random Priming Recombination (RPR) random sequence primers are used to generate many short DNA fragments complementary to different segments of the template. (Shao et al., Nucleic Acids Res 26:681-683 (1998).) Base misincorporation and mispriming via epPCR give point mutations. Short DNA fragments prime one another based on homology and are recombined and reassembled into full-length by repeated thermocycling. Removal of templates prior to this step assures low parental recombinants. This method, like most others, can be performed over multiple iterations to evolve distinct properties. This technology avoids sequence bias, is independent of gene length, and requires very little parent DNA for the application.

In Heteroduplex Recombination linearized plasmid DNA is used to form heteroduplexes that are repaired by mismatch repair. (Volkov et al., Nucleic Acids Res 27:e18 (1999); and Volkov et al., Methods Enzymol. 328:456-463 (2000).) The mismatch repair step is at least somewhat mutagenic. Heteroduplexes transform more efficiently than linear homoduplexes. This method is suitable for large genes and whole operons.

Random Chimeragenesis on Transient Templates (RACHITT) (Coco et al., Nat. Biotechnol 19:354-359 (2001).) employs Dnase I fragmentation and size fractionation of ssDNA. Homologous fragments are hybridized in the absence of polymerase to a complementary ssDNA scaffold. Any overlapping unhybridized fragment ends are trimmed down by an exonuclease. Gaps between fragments are filled in, and then ligated to give a pool of full-length diverse strands hybridized to the scaffold (that contains U to preclude amplification). The scaffold then is destroyed and is replaced by a new strand complementary to the diverse strand by PCR amplification. The method involves one strand (scaffold) that is from only one parent while the priming fragments derive from other genes; the parent scaffold is selected against. Thus, no reannealing with parental fragments occurs. Overlapping fragments are trimmed with an exonuclease. Otherwise, this is conceptually similar to DNA shuffling and StEP. Therefore, there should be no siblings, few inactives, and no unshuffled parentals. This technique has advantages in that few or no parental genes are created and many more crossovers can result relative to standard DNA shuffling.

Recombined Extension on Truncated templates (RETT) entails template switching of unidirectionally growing strands from primers in the presence of unidirectional ssDNA fragments used as a pool of templates. (Lee et al., J. Molec. Catalysis 26:119-129 (2003).) No DNA endonucleases are used. Unidirectional ssDNA is made by DNA polymerase with random primers or serial deletion with exonuclease. Unidirectional ssDNA are only templates and not primers. Random priming and exonucleases don't introduce sequence bias as true of enzymatic cleavage of DNA shuffling/RACHITT. RETT can be easier to optimize than StEP because it uses normal PCR conditions instead of very short extensions. Recombination occurs as a component of the PCR steps—no direct shuffling. This method can also be more random than StEP due to the absence of pauses.

In Degenerate Oligonucleotide Gene Shuffling (DOGS) degenerate primers are used to control recombination between molecules; (Bergquist et al., Methods Mol. Biol. 352:191-204 (2007); Bergquist et al., Biomol. Eng 22:63-72 (2005); Gibbs et al., Gene 271:13-20 (2001).) This can be used to control the tendency of other methods such as DNA shuffling to regenerate parental genes. This method can be combined with random mutagenesis (epPCR) of selected gene segments. This can be a good method to block the reformation of parental sequences. No endonucleases are needed. By adjusting input concentrations of segments made, one can bias towards a desired backbone. This method allows DNA shuffling from unrelated parents without restriction enzyme digests and allows a choice of random mutagenesis methods.

Incremental Truncation for the Creation of Hybrid Enzymes (ITCHY) creates a combinatorial library with 1 base pair deletions of a gene or gene fragment of interest. (Ostermeier et al., Proc Natl Acad Sci U.S.A 96:3562-3567 (1999); Ostermeier et la., Nat. Biotechnol 17:1205-1209 (1999).) Truncations are introduced in opposite direction on pieces of 2 different genes. These are ligated together and the fusions are cloned. This technique does not require homology between the 2 parental genes. When ITCHY is combined with DNA shuffling, the system is called SCRATCHY (see below). A major advantage of both is no need for homology between parental genes; for example, functional fusions between an E. coli and a human gene were created via ITCHY. When ITCHY libraries are made, all possible crossovers are captured.

Thio-Incremental Truncation for the Creation of Hybrid Enzymes (THIO-ITCHY) is almost the same as ITCHY except that phosphothioate dNTPs are used to generate truncations. (Lutz et al., Nucleic Acids Res 29:E16 (2001).) Relative to ITCHY, THIO-ITCHY can be easier to optimize, provide more reproducibility, and adjustability.

SCRATCHY-ITCHY combined with DNA shuffling is a combination of DNA shuffling and ITCHY; therefore, allowing multiple crossovers. (Lutz et al. 2001, Proc Natl Acad Sci U.S.A. 98:11248-11253 (2001).) SCRATCHY combines the best features of ITCHY and DNA shuffling. Computational predictions can be used in optimization. SCRATCHY is more effective than DNA shuffling when sequence identity is below 80%.

In Random Drift Mutagenesis (RNDM) mutations made via epPCR followed by screening/selection for those retaining usable activity. (Bergquist et al., Biomol. Eng 22:63-72 (2005).) Then, these are used in DOGS to generate recombinants with fusions between multiple active mutants or between active mutants and some other desirable parent. Designed to promote isolation of neutral mutations; its purpose is to screen for retained catalytic activity whether or not this activity is higher or lower than in the original gene. RNDM is usable in high throughput assays when screening is capable of detecting activity above background. RNDM has been used as a front end to DOGS in generating diversity. The technique imposes a requirement for activity prior to shuffling or other subsequent steps; neutral drift libraries are indicated to result in higher/quicker improvements in activity from smaller libraries. Though published using epPCR, this could be applied to other large-scale mutagenesis methods.

Sequence Saturation Mutagenesis (SeSaM) is a random mutagenesis method that: 1) generates pool of random length fragments using random incorporation of a phosphothioate nucleotide and cleavage; this pool is used as a template to 2) extend in the presence of “universal” bases such as inosine; 3) replication of a inosine-containing complement gives random base incorporation and, consequently, mutagenesis. (Wong et al., Biotechnol J 3:74-82 (2008); Wong et al., Nucleic Acids Res 32:e26 (2004); and Wong et al., Anal. Biochem. 341:187-189 (2005).) Using this technique it can be possible to generate a large library of mutants within 2-3 days using simple methods. This is very non-directed compared to mutational bias of DNA polymerases. Differences in this approach makes this technique complementary (or alternative) to epPCR.

In Synthetic Shuffling, overlapping oligonucleotides are designed to encode “all genetic diversity in targets” and allow a very high diversity for the shuffled progeny. (Ness et al., Nat. Biotechnol 20:1251-1255 (2002).) In this technique, one can design the fragments to be shuffled. This aids in increasing the resulting diversity of the progeny. One can design sequence/codon biases to make more distantly related sequences recombine at rates approaching more closely related sequences and it doesn't require possessing the template genes physically.

Nucleotide Exchange and Excision Technology NexT exploits a combination of dUTP incorporation followed by treatment with uracil DNA glycosylase and then piperidine to perform endpoint DNA fragmentation. (Muller et al., Nucleic Acids Res 33:e117 (2005).) The gene is reassembled using internal PCR primer extension with proofreading polymerase. The sizes for shuffling are directly controllable using varying dUPT::dTTP ratios. This is an end point reaction using simple methods for uracil incorporation and cleavage. One can use other nucleotide analogs such as 8-oxo-guanine with this method. Additionally, the technique works well with very short fragments (86 bp) and has a low error rate. Chemical cleavage of DNA means very few unshuffled clones.

In Sequence Homology-Independent Protein Recombination (SHIPREC) a linker is used to facilitate fusion between 2 distantly/unrelated genes; nuclease treatment is used to generate a range of chimeras between the two. Result is a single crossover library of these fusions. (Sieber et al., Nat Biotechnol 19:456-460 (2001).) This produces a limited type of shuffling; mutagenesis is a separate process. This technique can create a library of chimeras with varying fractions of each of 2 unrelated parent genes. No homology is needed. SHIPREC was tested with a heme-binding domain of a bacterial CP450 fused to N-terminal regions of a mammalian CP450; this produced mammalian activity in a more soluble enzyme.

In Gene Site Saturation Mutagenesis (GSSM) the starting materials are a supercoiled dsDNA plasmid with insert and 2 primers degenerate at the desired site for mutations. (Kretz et al., Methods Enzymol. 388:3-11 (2004).) Primers carry the mutation of interest and anneal to the same sequence on opposite strands of DNA; mutation in the middle of the primer and ˜20 nucleotides of correct sequence flanking on each side. The sequence in the primer is NNN or NNK (coding) and MNN (noncoding) (N=all 4, K=G, T, M=A, C). After extension, DpnI is used to digest dam-methylated DNA to eliminate the wild-type template. This technique explores all possible amino acid substitutions at a given locus (i.e., one codon). The technique facilitates the generation of all possible replacements at one site with no nonsense codons and equal or near-equal representation of most possible alleles. It does not require prior knowledge of structure, mechanism, or domains of the target enzyme. If followed by shuffling or Gene Reassembly, this technology creates a diverse library of recombinants containing all possible combinations of single-site up-mutations. The utility of this technology combination has been demonstrated for the successful evolution of over 50 different enzymes, and also for more than one property in a given enzyme.

Combinatorial Cassette Mutagenesis (CCM) involves the use of short oligonucleotide cassettes to replace limited regions with a large number of possible amino acid sequence alterations. (Reidhaar-Olson et al., Methods Enzymol. 208:564-586 (1991); and Reidhaar-Olson et al., Science 241:53-57 (1988).) Simultaneous substitutions at 2 or 3 sites are possible using this technique. Additionally, the method tests a large multiplicity of possible sequence changes at a limited range of sites. It has been used to explore the information content of lambda repressor DNA-binding domain.

Combinatorial Multiple Cassette Mutagenesis (CMCM) is essentially similar to CCM except it is employed as part of a larger program: 1) Use of epPCR at high mutation rate to 2) ID hot spots and hot regions and then 3) extension by CMCM to cover a defined region of protein sequence space. (Reetz et al., Angew. Chem. Int. Ed Engl. 40:3589-3591 (2001).) As with CCM, this method can test virtually all possible alterations over a target region. If used along with methods to create random mutations and shuffled genes, it provides an excellent means of generating diverse, shuffled proteins. This approach was successful in increasing, by 51-fold, the enantioselectivity of an enzyme.

In the Mutator Strains technique conditional ts mutator plasmids allow increases of 20- to 4000-X in random and natural mutation frequency during selection and to block accumulation of deleterious mutations when selection is not required. (Selifonova et al., Appl Environ Microbiol 67:3645-3649 (2001).) This technology is based on a plasmid-derived mutD5 gene, which encodes a mutant subunit of DNA polymerase III. This subunit binds to endogenous DNA polymerase III and compromises the proofreading ability of polymerase III in any of the strain that harbors the plasmid. A broad-spectrum of base substitutions and frameshift mutations occur. In order for effective use, the mutator plasmid should be removed once the desired phenotype is achieved; this is accomplished through a temperature sensitive origin of replication, which allows plasmid curing at 41° C. It should be noted that mutator strains have been explored for quite some time (e.g., see Winter and coworkers, J. Mol. Biol. 260:359-3680 (1996). In this technique very high spontaneous mutation rates are observed. The conditional property minimizes non-desired background mutations. This technology could be combined with adaptive evolution to enhance mutagenesis rates and more rapidly achieve desired phenotypes.

“Look-Through Mutagenesis (LTM) is a multidimensional mutagenesis method that assesses and optimizes combinatorial mutations of selected amino acids.” (Rajpal et al., Proc Natl Acad Sci U S.A 102:8466-8471 (2005.) Rather than saturating each site with all possible amino acid changes, a set of 9 is chosen to cover the range of amino acid R-group chemistry. Fewer changes per site allows multiple sites to be subjected to this type of mutagenesis. A>800-fold increase in binding affinity for an antibody from low nanomolar to picomolar has been achieved through this method. This is a rational approach to minimize the number of random combinations and should increase the ability to find improved traits by greatly decreasing the numbers of clones to be screened. This has been applied to antibody engineering, specifically to increase the binding affinity and/or reduce dissociation. The technique can be combined with either screens or selections.

Gene Reassembly is a DNA shuffling method that can be applied to multiple genes at one time or to creating a large library of chimeras (multiple mutations) of a single gene. (on the world-wide web at verenium.com/Pages/Technology/EnzymeTech/TechEnzyTGR.html) Typically this technology is used in combination with ultra-high-throughput screening to query the represented sequence space for desired improvements. This technique allows multiple gene recombination independent of homology. The exact number and position of cross-over events can be pre-determined using fragments designed via bioinformatic analysis. This technology leads to a very high level of diversity with virtually no parental gene reformation and a low level of inactive genes. Combined with GSSM, a large range of mutations can be tested for improved activity. The method allows “blending” and “fine tuning” of DNA shuffling, e.g. codon usage can be optimized.

In Silico Protein Design Automation PDA is an optimization algorithm that anchors the structurally defined protein backbone possessing a particular fold, and searches sequence space for amino acid substitutions that can stabilize the fold and overall protein energetics. (Hayes et al., Proc Natl Acad Sci U.S.A. 99:15926-15931 (2002).) This technology allows in silico structure-based entropy predictions in order to search for structural tolerance toward protein amino acid variations. Statistical mechanics is applied to calculate coupling interactions at each position—structural tolerance toward amino acid substitution is a measure of coupling. Ultimately, this technology is designed to yield desired modifications of protein properties while maintaining the integrity of structural characteristics. The method computationally assesses and allows filtering of a very large number of possible sequence variants (10⁵⁰. Choice of sequence variants to test is related to predictions based on most favorable thermodynamics and ostensibly only stability or properties that are linked to stability can be effectively addressed with this technology. The method has been successfully used in some therapeutic proteins, especially in engineering immunoglobulins. In silico predictions avoid testing extraordinarily large numbers of potential variants. Predictions based on existing three-dimensional structures are more likely to succeed than predictions based on hypothetical structures. This technology can readily predict and allow targeted screening of multiple simultaneous mutations, something not possible with purely experimental technologies due to exponential increases in numbers.

Iterative Saturation Mutagenesis (ISM) involves 1) Use knowledge of structure/function to choose a likely site for enzyme improvement. 2) Saturation mutagenesis at chosen site using Stratagene QuikChange (or other suitable means). 3) Screen/select for desired properties. 4) With improved clone(s), start over at another site and continue repeating. (Reetz et al., Nat. Protoc. 2:891-903 (2007); and Reetz et al., Angew. Chem. Int. Ed Engl. 45:7745-7751 (2006).) This is a proven methodology assures all possible replacements at a given position are made for screening/selection.

Any of the aforementioned methods for mutagenesis can be used alone or in any combination. Additionally, any one or combination of the directed evolution methods can be used in conjunction with adaptive evolution techniques.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of cyclohexanone.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that enable an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, teemed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

As disclosed herein, a nucleic acid encoding a desired activity of a cyclohexanone pathway can be introduced into a host organism. In some cases, it can be desirable to modify an activity of a cyclohexanone pathway enzyme or protein to increase production of cyclohexanone. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of a cyclohexanone pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol. 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Osteimeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional ts mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

It is understood that modifications which do not substantially affect the activity of the various embodiments of this invention are also included within the definition of the invention provided herein. Accordingly, the following examples are intended to illustrate but not limit the present invention.

### Example I

### Preparation of a Cyclohexanone Producing Microbial Organism Having a Pimeloyl-CoA Pathway

This example describes the generation of a microbial organism capable of producing cyclohexanone from pimeloyl-CoA, as demonstrated in FIG. 1.

Escherichia coli is used as a target organism to engineer a cyclohexanone-producing pathway from pimeloyl-CoA as shown in FIG. 1. E. coli provides a good host for generating a non-naturally occurring microorganism capable of producing cyclohexanone. E. coli is amenable to genetic manipulation and is known to be capable of producing various products, like ethanol, acetic acid, formic acid, lactic acid, and succinic acid, effectively under anaerobic or microaerobic conditions. Moreover, pimeloyl-CoA is naturally produced in E. coli as an intermediate in biotin biosynthesis.

To generate an E. coli strain engineered to produce cyclohexanone from pimeloyl-CoA, nucleic acids encoding the enzymes utilized in the pathway of FIG. 1, described previously, are expressed in E. coli using well known molecular biology techniques (see, for example, Sambrook, supra, 2001; Ausubel supra, 1999; Roberts et al., supra, 1989). In particular, the syn_{—}01653 (YP_{—}463074.1), adc (NP_{—}149328.1), pcaIJ (Q01103.2 and POA102.2), and pckA (P43923.1) genes encoding the 2-ketocyclohexane-1-carboxyl-CoA hydrolase (acting on C—C bond), 2-ketocyclohexane-1-carboxylate decarboxylase, 2-ketocyclohexane-1-carboxyl-CoA transferase and phosphoenolpyruvate carboxykinase, respectively, are cloned into the pZE13 vector (Expressys, Ruelzheim, Germany), under the control of the PA1/lacO promoter. This plasmid is then transformed into a host strain containing lacI^{Q}, which allows inducible expression by addition of isopropyl-beta-D-1-thiogalactopyranoside (IPTG).

The resulting genetically engineered organism is cultured in glucose containing medium following procedures well known in the art (see, for example, Sambrook et al., supra, 2001). The expression of cyclohexanone pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce cyclohexanone is confirmed using HPLC, gas chromatography-mass spectrometry (GCMS) or liquid chromatography-mass spectrometry (LCMS).

Microbial strains engineered to have a functional cyclohexanone synthesis pathway are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers. Strategies are also applied to improve production of cyclohexanone precursor pimeloyl-CoA, such as mutagenesis, cloning and/or overexpression of native genes involved in the early stages of pimeloyl-CoA synthesis.

To generate better producers, metabolic modeling is utilized to optimize growth conditions. Modeling is also used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379). Modeling analysis allows reliable predictions of the effects on cell growth of shifting the metabolism towards more efficient production of cyclohexanone. One modeling method is the bilevel optimization approach, OptKnock (Burgard et al., Biotechnol. Bioengineer. 84:647-657 (2003)), which is applied to select gene knockouts that collectively result in better production of cyclohexanone. Adaptive evolution also can be used to generate better producers of, for example, the pimeloyl-CoA intermediate or the cyclohexanone product. Adaptive evolution is performed to improve both growth and production characteristics (Fong and Palsson, Nat. Genet. 36:1056-1058 (2004); Alper et al., Science 314:1565-1568 (2006)). Based on the results, subsequent rounds of modeling, genetic engineering and adaptive evolution can be applied to the cyclohexanone producer to further increase production.

For large-scale production of cyclohexanone, the above cyclohexanone pathway-containing organism is cultured in a fermenter using a medium known in the art to support growth of the organism under anaerobic conditions. Fermentations are performed in either a batch, fed-batch or continuous manner. Anaerobic conditions are maintained by first sparging the medium with nitrogen and then sealing culture vessel (e.g., flasks can be sealed with a septum and crimp-cap). Microaerobic conditions also can be utilized by providing a small hole for limited aeration. The pH of the medium is maintained at a pH of 7 by addition of an acid, such as H2SO4. The growth rate is determined by measuring optical density using a spectrophotometer (600 nm), and the glucose uptake rate by monitoring carbon source depletion over time. Byproducts such as undesirable alcohols, organic acids, and residual glucose can be quantified by HPLC (Shimadzu) with an HPX-087 column (BioRad), using a refractive index detector for glucose and alcohols, and a UV detector for organic acids, Lin et al., Biotechnol. Bioeng., 775-779 (2005).

### Example II

### Preparation of a Cyclohexanone Producing Microbial Organism, in which the Cyclohexanone is Derived from Acetoacetyl-CoA Via Pimeloyl-CoA

This example describes the generation of a microbial organism that has been engineered to produce enhanced levels of the cyclohexanone precursor pimeloyl-CoA from acetoacetyl-CoA, shown in FIG. 2. This engineered strain is then used as a host organism and further engineered to express enzymes or proteins for producing cyclohexanone from pimeloyl-CoA, via the pathway of FIG. 1.

Escherichia coli is used as a target organism to engineer a cyclohexanone-producing pathway as shown in FIG. 1. E. coli provides a good host for generating a non-naturally occurring microorganism capable of producing cyclohexanone. E. coli is amenable to genetic manipulation and is known to be capable of producing various products, like ethanol, acetic acid, formic acid, lactic acid, and succinic acid, effectively under anaerobic or microaerobic conditions.

To generate an E. coli strain engineered to produce cyclohexanone, nucleic acids encoding the enzymes utilized in the pathways of FIG. 1 and FIG. 2, described previously, are expressed in E. coli using well known molecular biology techniques (see, for example, Sambrook, supra, 2001; Ausubel supra, 1999; Roberts et al., supra, 1989).

In particular, an E. coli strain is engineered to produce pimeloyl-CoA from acetoacetyl-CoA via the route outlined in FIG. 2. For the first stage of pathway construction, genes encoding enzymes to transform acetoacetyl-CoA to pimeloyl-CoA (FIG. 2) are assembled onto vectors. In particular, the genes pckA (P43923.1), phbB (P23238), crt (NP_{—}349318.1), gcdH (ABM69268.1) and gcdR (ABM69269.1) encoding phosphoenolpyruvate carboxykinase, acetoacetyl-CoA reductase, 3-hydroxybutyryl-CoA dehydratase, a glutaryl-CoA dehydrogenase, and the cognate transcriptional regulator of the glutaryl-CoA dehydrogenase, respectively, are cloned into the pZE13 vector (Expressys, Ruelzheim, Germany), under the control of the PA1/lacO promoter. The genes syn_{—}02642 (YP_{—}462685.1), hbd (NP_{—}349314.1), syn_{—}01309 (YP_{—}461962) and syn_{—}23587 (ABC76101) encoding oxopimeloyl-CoA:glutaryl-CoA acyltransferase, 3-hydroxypimeloyl-CoA dehydrogenase, 3-hydroxypimeloyl-CoA dehydratase, and a pimeloyl-CoA dehydrogenase, respectively, are cloned into the pZA33 vector (Expressys, Ruelzheim, Germany) under the PA1/lacO promoter. Further, the syn_{—}02637 (ABC78522.1) and syn_{—}02636 (ABC78523.1) genes encoding alpha and beta subunits of an electron transfer flavoprotein are cloned into a third compatible plasmid, pZS23, under the PA1/lacO promoter. pZS23 is obtained by replacing the ampicillin resistance module of the pZS13 vector (Expressys, Ruelzheim, Germany) with a kanamycin resistance module by well-known molecular biology techniques. The three sets of plasmids are transformed into E. coli strain MG1655 to express the proteins and enzymes required for pimeloyl-CoA synthesis from acetoacetyl-CoA.

The resulting genetically engineered organism is cultured in glucose containing medium following procedures well known in the art (see, for example, Sambrook et al., supra, 2001). The expression of pimeloyl-CoA pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce pimeloyl-CoA through this pathway is confirmed using HPLC, gas chromatography-mass spectrometry (GCMS) or liquid chromatography-mass spectrometry (LCMS).

Microbial strains engineered to have a functional pimeloyl-CoA synthesis pathway from acetoacetyl-CoA are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers.

After successful demonstration of enhanced pimeloyl-CoA production via the activities of the exogenous enzymes, the genes encoding these enzymes are inserted into the chromosome of a wild type E. coli host using methods known in the art. Such methods include, for example, sequential single crossover (Gay et al., J. Bacteriol. 153:1424-1431 (1983)) and Red/ET methods from GeneBridges (Zhang et al., Improved RecT or RecET cloning and subcloning method (2001)). Chromosomal insertion provides several advantages over a plasmid-based system, including greater stability and the ability to co-localize expression of pathway genes.

The pimeloyl-CoA-overproducing host strain is further engineered to produce cyclohexanone. To generate a cyclohexanone-producing strain, nucleic acids encoding the enzymes utilized in the pathway of FIG. 1, described previously, are expressed in the host using well known molecular biology techniques (see, for example, Sambrook, supra, 2001; Ausubel supra, 1999; Roberts et al., supra, 1989).

In particular, the syn_{—}01653 (YP_{—}463074.1), adc (NP_{—}149328.1), pcaIJ (Q01103.2 and P0A102.2) genes encoding the 2-ketocyclohexane-1-carboxyl-CoA hydrolase (acting on C—C bond), 2-ketocyclohexane-1-carboxylate decarboxylase, and 2-ketocyclohexane-1-carboxyl-CoA transferase, respectively, are cloned into the pZE13 vector (Expressys, Ruelzheim, Germany), under the control of the PA1/lacO promoter. This plasmid is then transformed into a host strain containing lacI^{Q}, which allows inducible expression by addition of isopropyl-beta-D-1-thiogalactopyranoside (IPTG).

The resulting genetically engineered organism is cultured in glucose containing medium following procedures well known in the art (see, for example, Sambrook et al., supra, 2001). The expression of cyclohexanone pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce cyclohexanone through this pathway is confirmed using HPLC, gas chromatography-mass spectrometry (GCMS) or liquid chromatography-mass spectrometry (LCMS).

Microbial strains engineered to have a functional cyclohexanone synthesis pathway are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers.

To generate better producers, metabolic modeling is utilized to optimize growth conditions. Modeling is also used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379). Modeling analysis allows reliable predictions of the effects on cell growth of shifting the metabolism towards more efficient production of cyclohexanone. One modeling method is the bilevel optimization approach, OptKnock (Burgard et al., Biotechnol. Bioengineer. 84:647-657 (2003)), which is applied to select gene knockouts that collectively result in better production of cyclohexanone. Adaptive evolution also can be used to generate better producers of, for example, the pimeloyl-CoA intermediate or the cyclohexanone product. Adaptive evolution is performed to improve both growth and production characteristics (Fong and Palsson, Nat. Genet. 36:1056-1058 (2004); Alper et al., Science 314:1565-1568 (2006)). Based on the results, subsequent rounds of modeling, genetic engineering and adaptive evolution can be applied to the cyclohexanone producer to further increase production.

For large-scale production of cyclohexanone, the above cyclohexanone pathway-containing organism is cultured in a fermenter using a medium known in the art to support growth of the organism under anaerobic conditions. Fermentations are performed in either a batch, fed-batch or continuous manner. Anaerobic conditions are maintained by first sparging the medium with nitrogen and then sealing culture vessel (e.g., flasks can be sealed with a septum and crimp-cap). Microaerobic conditions also can be utilized by providing a small hole for limited aeration. The pH of the medium is maintained at a pH of 7 by addition of an acid, such as H2SO4. The growth rate is determined by measuring optical density using a spectrophotometer (600 nm), and the glucose uptake rate by monitoring carbon source depletion over time. Byproducts such as undesirable alcohols, organic acids, and residual glucose can be quantified by HPLC (Shimadzu) with an HPX-087 column (BioRad), using a refractive index detector for glucose and alcohols, and a UV detector for organic acids, Lin et al., Biotechnol. Bioeng., 775-779 (2005).

### Example III

### Preparation of a Cyclohexanone Producing Microbial Organism, in which the Cyclohexanone is Derived from Acetoacetyl-CoA and 3-Hydroxypimeloyl-CoA is a Pathway Intermediate

This example describes the generation of a microbial organism that has been engineered to produce cyclohexanone from acetoacetyl-CoA via 3-hydroxypimelate as an intermediate. 3-Hydroxypimelate is produced from acetoacetyl-CoA in five enzymatic steps, as shown in FIG. 2 (Steps 1-5). Cyclohexanone is then produced from 3-hydroxypimelate as shown in the pathway of FIG. 3 (Steps 1, 5, 6 and 7).

Escherichia coli is used as a target organism to engineer a cyclohexanone-producing pathway as shown in FIGS. 2 and 3. E. coli provides a good host for generating a non-naturally occurring microorganism capable of producing cyclohexanone. E. coli is amenable to genetic manipulation and is known to be capable of producing various products, like ethanol, acetic acid, formic acid, lactic acid, and succinic acid, effectively under anaerobic or microaerobic conditions.

To generate an E. coli strain engineered to produce cyclohexanone, nucleic acids encoding the enzymes utilized in the pathways of FIG. 2 (Steps 1-5) and FIG. 3 (Steps 1, 5, 6 and 7), described previously, are expressed in E. coli using well known molecular biology techniques (see, for example, Sambrook, supra, 2001; Ausubel supra, 1999; Roberts et al., supra, 1989).

To generate an E. coli strain for producing cyclohexanone from acetoacetyl-CoA via 3-hydroxypimeloyl-CoA, genes encoding enzymes to transform acetoacetyl-CoA to 3-hydroxypimeloyl-CoA (FIG. 2) and 3-hydroxypimeloyl-CoA to cyclohexanone (FIG. 3) are assembled onto vectors. In particular, the genes pckA (P43923.1), phbB (P23238), crt (NP_{—}349318.1), gcdH (ABM69268.1) and gcdR (ABM69269.1) genes encoding phosphoenolpyruvate carboxykinase, acetoacetyl-CoA reductase, 3-hydroxybutyryl-CoA dehydratase, a glutaryl-CoA dehydrogenase, and the cognate transcriptional regulator of the glutaryl-CoA dehydrogenase, respectively, are cloned into the pZE13 vector (Expressys, Ruelzheim, Germany), under the control of the PA1/lacO promoter. The genes syn_{—}02642 (YP_{—}462685.1), hbd (NP_{—}349314.1), bamA (YP_{—}463073.1) and pcaIJ (Q01103.2 and P0A102.2) encoding oxopimeloyl-CoA:glutaryl-CoA acyltransferase, 3-hydroxypimeloyl-CoA dehydrogenase, 6-ketocyclohex-1-ene-1-carboxyl-CoA hydrolases (acting on CC bond) and 2-ketocyclohexane-1-catboxyl-CoA transferase, respectively, are cloned into the pZA33 vector (Expressys, Ruelzheim, Germany) under the PA1/lacO promoter. Further, the genes acad1 (AAC48316.1), acad (AAA16096.1) and adc (NP_{—}149328.1), encoding 6-ketocyclohex-1-ene-1-carboxyl-CoA reductase and 2-ketocyclohexane-1-carboxylate decarboxylase, respectively, are cloned into a third compatible plasmid, pZS23, under the PA1/lacO promoter. pZS23 is obtained by replacing the ampicillin resistance module of the pZS13 vector (Expressys, Ruelzheim, Germany) with a kanamycin resistance module by well-known molecular biology techniques. The three sets of plasmids are transformed into E. coli strain MG1655 to express the proteins and enzymes required for cyclohexanone synthesis from acetoacetyl-CoA.

The resulting genetically engineered organism is cultured in glucose containing medium following procedures well known in the art (see, for example, Sambrook et al., supra, 2001). The expression of cyclohexanone pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce cyclohexanone through this pathway is confirmed using HPLC, gas chromatography-mass spectrometry (GCMS) or liquid chromatography-mass spectrometry (LCMS).

Microbial strains engineered to have a functional cyclohexanone synthesis pathway are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers.

To generate better producers, metabolic modeling is utilized to optimize growth conditions. Modeling is also used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379). Modeling analysis allows reliable predictions of the effects on cell growth of shifting the metabolism towards more efficient production of cyclohexanone. One modeling method is the bilevel optimization approach, OptKnock (Burgard et al., Biotechnol. Bioengineer. 84:647-657 (2003)), which is applied to select gene knockouts that collectively result in better production of cyclohexanone. Adaptive evolution also can be used to generate better producers of, for example, the 3-hydroxypimeloyl-CoA intermediate or the cyclohexanone product. Adaptive evolution is performed to improve both growth and production characteristics (Fong and Palsson, Nat. Genet. 36:1056-1058 (2004); Alper et al., Science 314:1565-1568 (2006)). Based on the results, subsequent rounds of modeling, genetic engineering and adaptive evolution can be applied to the cyclohexanone producer to further increase production.

For large-scale production of cyclohexanone, the above cyclohexanone pathway-containing organism is cultured in a fermenter using a medium known in the art to support growth of the organism under anaerobic conditions. Fermentations are performed in either a batch, fed-batch or continuous manner. Anaerobic conditions are maintained by first sparging the medium with nitrogen and then sealing culture vessel (e.g., flasks can be sealed with a septum and crimp-cap). Microaerobic conditions also can be utilized by providing a small hole for limited aeration. The pH of the medium is maintained at a pH of 7 by addition of an acid, such as H2SO4. The growth rate is determined by measuring optical density using a spectrophotometer (600 nm), and the glucose uptake rate by monitoring carbon source depletion over time. Byproducts such as undesirable alcohols, organic acids, and residual glucose can be quantified by HPLC (Shimadzu) with an HPX-087 column (BioRad), using a refractive index detector for glucose and alcohols, and a UV detector for organic acids, Lin et al., Biotechnol. Bioeng., 775-779 (2005).

### Example IV

### Preparation of a Cyclohexanone Producing Microbial Organism, in which the Cyclohexanone is Derived from Adipate Semialdehyde

This example describes the generation of a microbial organism that has been engineered to produce cyclohexanone from adipate semialdehyde, as shown in FIG. 4. First, an E. coli host strain is engineered to overproduce the cyclohexanone precursor adipate semialdehyde, according to the teachings of U.S. patent application Ser. No. 12/413,35. The adipate semialdehyde-overproducing host is further engineered to overproduce cyclohexanone.

Escherichia coli is used as a target organism to engineer a cyclohexanone-producing pathway as shown in FIG. 4. E. coli provides a good host for generating a non-naturally occurring microorganism capable of producing cyclohexanone. E. coli is amenable to genetic manipulation and is known to be capable of producing various products, like ethanol, acetic acid, formic acid, lactic acid, and succinic acid, effectively under anaerobic or microaerobic conditions.

Adipate semialdehyde is not a naturally occurring metabolite in Escherichia coli. However, a number of biosynthetic routes for adipate biosynthesis have recently been disclosed [U.S. patent application Ser. No. 12/413,355]. In one route, termed the “reverse degradation pathway”, adipate semialdehyde is produced from molar equivalents of acetyl-CoA and succinyl-CoA, joined by a beta-ketothiolase to form oxoadipyl-CoA. Oxoadipyl-CoA is then converted to adipyl-CoA in three enzymatic steps: reduction of the ketone, dehydration, and reduction of the enoyl-CoA. Once formed, adipyl-CoA is converted to adipate semialdehyde by a CoA-dependent aldehyde dehydrogenase.

To generate an E. coli strain engineered to produce adipate semialdehyde, nucleic acids encoding the enzymes of the reverse degradation pathway are expressed in E. coli using well known molecular biology techniques (see, for example, Sambrook, supra, 2001; Ausubel supra, 1999). In particular, the paaJ (NP_{—}415915.1), paaH (NP_{—}415913.1), maoC (NP_{—}415905.1) and pckA (P43923.1) genes encoding a succinyl-CoA:acetyl-CoA acyl transferase, 3-hydroxyacyl-CoA dehydrogenase, 3-hydroxyadipyl-CoA dehydratase and phosphoenolpyruvate carboxykinase activities, respectively, are cloned into the pZE13 vector (Expressys, Ruelzheim, Germany) under the PA1/lacO promoter. In addition, the bcd (NP_{—}349317.1), etfAB (349315.1 and 349316.1), and such (P38947.1) genes encoding 5-carboxy-2-pentenoyl-CoA reductase and adipyl-CoA aldehyde dehydrogenase activities, respectively, are cloned into the pZA33 vector (Expressys, Ruelzheim, Germany) under the PA1/lacO promoter. The two sets of plasmids are transformed into E. coli strain MG1655 to express the proteins and enzymes required for adipate synthesis via the reverse degradation pathway.

The resulting genetically engineered organism is cultured in glucose containing medium following procedures well known in the art (see, for example, Sambrook et al., supra, 2001). The expression of adipate semialdehyde pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce adipate semialdehyde through this pathway is confirmed using HPLC, gas chromatography-mass spectrometry (GCMS) or liquid chromatography-mass spectrometry (LCMS).

Microbial strains engineered to have a functional adipate semialdehyde synthesis pathway are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers.

After successful demonstration of enhanced adipate semialdehyde production via the activities of the exogenous enzymes, the genes encoding these enzymes are inserted into the chromosome of a wild type E. coli host using methods known in the art. Such methods include, for example, sequential single crossover (Gay et al., supra) and Red/ET methods from GeneBridges (Zhang et al., supra). The resulting strain is then utilized in subsequent efforts to engineer a cyclohexanone-overproducing pathway.

A requirement for engineering a cyclohexanone producing organism that utilizes the adipate semialdehyde pathway is identification of a gene with adipate semialdehyde dehydratase activity, that is, catalyzing the dehydration and concurrent cyclization of adipate semialdehyde to cyclohexane-1,2-dione. This activity has been demonstrated in the ring-opening direction in cell extracts of Azoarcus 22Lin (Harder, J., supra), but the gene associated with this activity has not been identified to date. To identify an enzyme with adipate semialdehyde dehydratase activity, a plasmid-based library composed of fragments of the Azoarcus 22Lin genome is constructed. Plasmids are transformed into E. coli and resulting colonies are isolated, supplied with cyclohexan-1,2-dione and screened for adipate semialdehyde dehydratase activity. Strains bearing plasmids with enzyme activity are isolated and the plasmids are sequenced. The sequences are examined to identify likely protein-encoding open reading frames (ORFs). Gene candidates are BLASTed against non-redundant protein sequences to determine potential function. Promising gene candidates encoded by the plasmid(s) are isolated by PCR, cloned into new vectors, transformed into E. coli and tested for adipate semialdehyde dehydratase activity.

Nucleic acids encoding the enzymes utilized in the pathway of FIG. 4, described previously, are expressed in E. coli using well known molecular biology techniques (see, for example, Sambrook, supra, 2001; Ausubel supra, 1999; Roberts et al., supra, 1989). In particular, the ARA1 (NP_{—}009707.1) and pddCBA (AAC98386.1, AAC98385.1 and AAC98384.1) genes encoding the cyclohexane-1,2-diol dehydrogenase and cyclohexane-1,2-diol dehydratase, respectively, are cloned into the pZE13 vector (Expressys, Ruelzheim, Germany), under the control of the PA1/lacO promoter. Further, the newly identified adipate semialdehyde dehydrogenase gene is cloned into the pZA33 vector (Expressys, Ruelzheim, Germany) under the PA1/lacO promoter. The two sets of plasmids are transformed into the adipate semialdehyde-overproducing E. coli host to express the proteins and enzymes required for adipate synthesis via the reverse degradation pathway.

The resulting genetically engineered organism is cultured in glucose containing medium following procedures well known in the art (see, for example, Sambrook et al., supra, 2001). The expression of cyclohexanone pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce cyclohexanone through this pathway is confirmed using HPLC, gas chromatography-mass spectrometry (GCMS) or liquid chromatography-mass spectrometry (LCMS).

Microbial strains engineered to have a functional cyclohexanone synthesis pathway are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers.

To generate better producers, metabolic modeling is utilized to optimize growth conditions. Modeling is also used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379). Modeling analysis allows reliable predictions of the effects on cell growth of shifting the metabolism towards more efficient production of cyclohexanone. One modeling method is the bilevel optimization approach, OptKnock (Burgard et al., Biotechnol. Bioengineer. 84:647-657 (2003)), which is applied to select gene knockouts that collectively result in better production of cyclohexanone. Adaptive evolution also can be used to generate better producers of, for example, the cyclohexane-1,2-dione intermediate or the cyclohexanone product. Adaptive evolution is performed to improve both growth and production characteristics (Fong and Palsson, Nat. Genet. 36:1056-1058 (2004); Alper et al., Science 314:1565-1568 (2006)). Based on the results, subsequent rounds of modeling, genetic engineering and adaptive evolution can be applied to the cyclohexanone producer to further increase production.

For large-scale production of cyclohexanone, the above cyclohexanone pathway-containing organism is cultured in a fermenter using a medium known in the art to support growth of the organism under anaerobic conditions. Fermentations are performed in either a batch, fed-batch or continuous manner. Anaerobic conditions are maintained by first sparging the medium with nitrogen and then sealing culture vessel (e.g., flasks can be sealed with a septum and crimp-cap). Microaerobic conditions also can be utilized by providing a small hole for limited aeration. The pH of the medium is maintained at a pH of 7 by addition of an acid, such as H2SO4. The growth rate is determined by measuring optical density using a spectrophotometer (600 nm), and the glucose uptake rate by monitoring carbon source depletion over time. Byproducts such as undesirable alcohols, organic acids, and residual glucose can be quantified by HPLC (Shimadzu) with an HPX-087 column (BioRad), using a refractive index detector for glucose and alcohols, and a UV detector for organic acids, Lin et al., Biotechnol. Bioeng., 775-779 (2005).

### Example V

### Preparation of a Cyclohexanone Producing Microbial Organism, in which the Cyclohexanone is Derived from 4-Acetylbutyrate

This example describes the generation of a microbial organism that has been engineered to produce cyclohexanone from 4-acetylbutyrate via 3-oxopimeloyl-CoA, as shown in FIG. 5. This example also teaches a method for engineering a strain that overproduces the pathway precursor 3-oxopimeloyl-CoA.

Escherichia coli is used as a target organism to engineer a cyclohexanone-producing pathway as shown in FIG. 5. E. coli provides a good host for generating a non-naturally occurring microorganism capable of producing cyclohexanone. E. coli is amenable to genetic manipulation and is known to be capable of producing various products, like ethanol, acetic acid, formic acid, lactic acid, and succinic acid, effectively under anaerobic or microaerobic conditions.

First, an E. coli strain is engineered to produce 3-oxopimeloyl-CoA from acetoacetyl-CoA via the route outlined in FIG. 2. For the first stage of pathway construction, genes encoding enzymes to transform acetoacetyl-CoA to 3-oxopimeloyl-CoA (FIG. 2, Steps 1-4) is assembled onto vectors. In particular, the genes phbB (P23238), crt (NP_{—}349318.1), gcdH (ABM69268.1) and gcdR (ABM69269.1) genes encoding acetoacetyl-CoA reductase, 3-hydroxybutyryl-CoA dehydratase, a glutaryl-CoA dehydrogenase, and the cognate transcriptional regulator of the glutaryl-CoA dehydrogenase, respectively, are cloned into the pZE13 vector (Expressys, Ruelzheim, Germany), under the control of the PA1/lacO promoter. The genes pckA (P43923.1) and syn_{—}02642 (YP_{—}462685.1), encoding phosphoenolpyruvate carboxykinase and oxopimeloyl-CoA:glutaryl-CoA acyltransferase, respectively, are cloned into the pZA33 vector (Expressys, Ruelzheim, Germany) under the PA1/lacO promoter. The two sets of plasmids are transformed into E. coli strain MG1655 to express the proteins and enzymes required for 3-oxopimeloyl-CoA synthesis from acetoacetyl-CoA.

The resulting genetically engineered organism is cultured in glucose containing medium following procedures well known in the art (see, for example, Sambrook et al., supra, 2001). The expression of 3-oxopimeloyl-CoA pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce 3-oxopimeloyl-CoA through this pathway is confirmed using HPLC, gas chromatography-mass spectrometry (GCMS) or liquid chromatography-mass spectrometry (LCMS).

Microbial strains engineered to have a functional 3-oxopimeloyl-CoA synthesis pathway from acetoacetyl-CoA are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers.

After successful demonstration of enhanced 3-oxopimeloyl-CoA production via the activities of the exogenous enzymes, the genes encoding these enzymes are inserted into the chromosome of a wild type E. coli host using methods known in the art. Such methods include, for example, sequential single crossover (Gay et al., supra) and Red/ET methods from GeneBridges (Zhang et al, supra). Chromosomal insertion provides several advantages over a plasmid-based system, including greater stability and the ability to co-localize expression of pathway genes.

A requirement for engineering a cyclohexanone producing organism that utilizes the 4-acetylbutyrate pathway is identification of a gene with 4-acetylbutyrate dehydratase activity, that is, catalyzing the dehydration and concurrent cyclization of 4-acetylbutyrate to cyclohexane-1,3-dione. This activity has been demonstrated in the hydrolytic cleavage (ring-opening) direction in cell extracts of Alicycliphilus denitrificans (Dangel et al., (1989) supra), but the gene associated with this activity has not been identified to date. To identify an enzyme with 4-acetylbutyrate dehydratase activity, a plasmid-based library composed of fragments of the Alicycliphilus denitrificans genome is constructed. Plasmids are transformed into E. coli and resulting colonies are isolated, supplied with cyclohexan-1,3-dione and screened for 4-acetylbutyrate dehydratase activity. Strains bearing plasmids with enzyme activity are isolated and the plasmids are sequenced. The sequences are examined to identify likely protein-encoding open reading frames (ORFs). Gene candidates are BLASTed against non-redundant protein sequences to determine potential function. Promising gene candidates encoded by the plasmid(s) are isolated by PCR, cloned into new vectors, transformed into E. coli and tested for 4-acetylbutyrate dehydratase activity.

To generate an E. coli strain engineered to produce cyclohexanone from 3-oxopimeloyl-CoA, nucleic acids encoding the enzymes utilized in the pathway of FIG. 5, described previously, are expressed in E. coli using well known molecular biology techniques (see, for example, Sambrook, supra, 2001; Ausubel supra, 1999; Roberts et al., supra, 1989). In particular, the pcaIJ (Q01103.2 and P0A102.2), adc (NP_{—}149328.1) and YMR226c (NP_{—}013953.1) genes encoding the 3-oxopimeloyl-CoA transferase, 3-oxopimelate decarboxylase and 3-hydroxycyclohexanone dehydrogenase, respectively, are cloned into the pZE13 vector (Expressys, Ruelzheim, Germany), under the control of the PA1/lacO promoter. Additionally, the genes HIDH (BAD80840.1) and YML131W (AAS56318.1), encoding 2-cyclohexenone hydratase and cyclohexanone dehydrogenase, respectively, and also the newly identified 4-acetylbutyrate dehydratase gene, are cloned into the pZA33 vector (Expressys, Ruelzheim, Germany) under the PA1/lacO promoter. The two sets of plasmids are transformed into E. coli strain MG1655 to express the proteins and enzymes required for cyclohexanone synthesis from 3-oxopimeloyl-CoA.

The resulting genetically engineered organism is cultured in glucose containing medium following procedures well known in the art (see, for example, Sambrook et al., supra, 2001). The expression of cyclohexanone pathway genes is corroborated using methods well known in the art for determining polypeptide expression or enzymatic activity, including for example, Northern blots, PCR amplification of mRNA and immunoblotting. Enzymatic activities of the expressed enzymes are confirmed using assays specific for the individually activities. The ability of the engineered E. coli strain to produce cyclohexanone through this pathway is confirmed using HPLC, gas chromatography-mass spectrometry (GCMS) or liquid chromatography-mass spectrometry (LCMS).

Microbial strains engineered to have a functional cyclohexanone synthesis pathway are further augmented by optimization for efficient utilization of the pathway. Briefly, the engineered strain is assessed to determine whether any of the exogenous genes are expressed at a rate limiting level. Expression is increased for any enzymes expressed at low levels that can limit the flux through the pathway by, for example, introduction of additional gene copy numbers.

To generate better producers, metabolic modeling is utilized to optimize growth conditions. Modeling is also used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379). Modeling analysis allows reliable predictions of the effects on cell growth of shifting the metabolism towards more efficient production of cyclohexanone. One modeling method is the bilevel optimization approach, OptKnock (Burgard et al., Biotechnol. Bioengineer. 84:647-657 (2003)), which is applied to select gene knockouts that collectively result in better production of cyclohexanone. Adaptive evolution also can be used to generate better producers of, for example, the 4-acetylbutyrate intermediate or the cyclohexanone product. Adaptive evolution is performed to improve both growth and production characteristics (Fong and Palsson, Nat. Genet. 36:1056-1058 (2004); Alper et al., Science 314:1565-1568 (2006)). Based on the results, subsequent rounds of modeling, genetic engineering and adaptive evolution can be applied to the cyclohexanone producer to further increase production.

For large-scale production of cyclohexanone, the above cyclohexanone pathway-containing organism is cultured in a fermenter using a medium known in the art to support growth of the organism under anaerobic conditions. Fermentations are performed in either a batch, fed-batch or continuous manner. Anaerobic conditions are maintained by first sparging the medium with nitrogen and then sealing culture vessel (e.g., flasks can be sealed with a septum and crimp-cap). Microaerobic conditions also can be utilized by providing a small hole for limited aeration. The pH of the medium is maintained at a pH of 7 by addition of an acid, such as H2SO4. The growth rate is determined by measuring optical density using a spectrophotometer (600 nm), and the glucose uptake rate by monitoring carbon source depletion over time. Byproducts such as undesirable alcohols, organic acids, and residual glucose can be quantified by HPLC (Shimadzu) with an HPX-087 column (BioRad), using a refractive index detector for glucose and alcohols, and a UV detector for organic acids, Lin et al., Biotechnol. Bioeng., 775-779 (2005).

### Example VI

### Exemplary Hydrogenase and Co Dehydrogenase Enzymes for Extracting Reducing Equivalents from Syngas and Exemplary Reductive TCA Cycle Enzymes

Enzymes of the reductive TCA cycle useful in the non-naturally occurring microbial organisms of the present invention include one or more of ATP-citrate lyase and three CO₂-fixing enzymes: isocitrate dehydrogenase, alpha-ketoglutarate:ferredoxin oxidoreductase, pyruvate:ferredoxin oxidoreductase. The presence of ATP-citrate lyase or citrate lyase and alpha-ketoglutarate:ferredoxin oxidoreductase indicates the presence of an active reductive TCA cycle in an organism. Enzymes for each step of the reductive TCA cycle are shown below.

ATP-citrate lyase (ACL, EC 2.3.3.8), also called ATP citrate synthase, catalyzes the ATP-dependent cleavage of citrate to oxaloacetate and acetyl-CoA. ACL is an enzyme of the RTCA cycle that has been studied in green sulfur bacteria Chlorobium limicola and Chlorobium tepidum. The alpha(4)beta(4) heteromeric enzyme from Chlorobium limicola was cloned and characterized in E. coli (Kanao et al., Eur. J. Biochem. 269:3409-3416 (2002). The C. limicola enzyme, encoded by aclAB, is irreversible and activity of the enzyme is regulated by the ratio of ADP/ATP. A recombinant ACL from Chlorobium tepidum was also expressed in E. coli and the holoenzyme was reconstituted in vitro, in a study elucidating the role of the alpha and beta subunits in the catalytic mechanism (Kim and Tabita, J. Bacteriol. 188:6544-6552 (2006). ACL enzymes have also been identified in Balnearium lithotrophicum, Sulfurihydrogenibium subterraneum and other members of the bacterial phylum Aquificae (Hugler et al., Environ. Microbiol. 9:81-92 (2007)). This activity has been reported in some fungi as well. Exemplary organisms include Sordaria macrospora (Nowrousian et al., Curr. Genet. 37:189-93 (2000), Aspergillus nidulans, Yarrowia lipolytica (Hynes and Murray, Eukaryotic Cell, July: 1039-1048, (2010) and Aspergillus niger (Meijer et al. J. Ind. Microbiol. Biotechnol. 36:1275-1280 (2009). Other candidates can be found based on sequence homology. Information related to these enzymes is tabulated below:

| Protein | GenBank ID | GI Number | Organism |
|---|---|---|---|
| aclA | BAB21376.1 | 12407237 | Chlorobium limicola |
| aclB | BAB21375.1 | 12407235 | Chlorobium limicola |
| aclA | AAM72321.1 | 21647054 | Chlorobium tepidum |
| aclB | AAM72322.1 | 21647055 | Chlorobium tepidum |
| aclA | ABI50076.1 | 114054981 | Balnearium lithotrophicum |
| aclB | ABI50075.1 | 114054980 | Balnearium lithotrophicum |
| aclA | ABI50085.1 | 114055040 | Sulfurihydrogenibium subterraneum |
| aclB | ABI50084.1 | 114055039 | Sulfurihydrogenibium subterraneum |

| Protein | GenBank ID | GI Number | Organism |
|---|---|---|---|
| aclA | AAX76834.1 | 62199504 | Sulfurimonas denitrificans |
| aclB | AAX76835.1 | 62199506 | Sulfurimonas denitrificans |
| acl1 | XP_504787.1 | 50554757 | Yarrowia lipolytica |
| acl2 | XP_503231.1 | 50551515 | Yarrowia lipolytica |
| SPBC1703.07 | NP_596202.1 | 19112994 | Schizosaccharomyces pombe |
| SPAC22A12.16 | NP_593246.1 | 19114158 | Schizosaccharomyces pombe |
| acl1 | CAB76165.1 | 7160185 | Sordaria macrospora |
| acl2 | CAB76164.1 | 7160184 | Sordaria macrospora |
| aclA | CBF86850.1 | 25987849 | Aspergillus nidulans |
| aclB | CBF86848 | 25987848 | Aspergillus nidulans |

In some organisms the conversion of citrate to oxaloacetate and acetyl-CoA proceeds through a citryl-CoA intermediate and is catalyzed by two separate enzymes, citryl-CoA synthetase (EC 6.2.1.18) and citryl-CoA lyase (EC 4.1.3.34) (Aoshima, M., Appl. Microbiol. Biotechnol. 75:249-255 (2007). Citryl-CoA synthetase catalyzes the activation of citrate to citryl-CoA. The Hydrogenobacter thermophilus enzyme is composed of large and small subunits encoded by ccsA and ccsB, respectively (Aoshima et al., Mol. Microbiol. 52:751-761 (2004)). The citryl-CoA synthetase of Aquifex aeolicus is composed of alpha and beta subunits encoded by sucC1 and sucD1 (Hugler et al., Environ. Microbiol. 9:81-92 (2007)). Citryl-CoA lyase splits citryl-CoA into oxaloacetate and acetyl-CoA. This enzyme is a homotrimer encoded by ccl in Hydrogenobacter thermophilus (Aoshima et al., Mol. Microbiol. 52:763-770 (2004)) and aq_{—}150 in Aquijex aeolicus (Hugler et al., supra (2007)). The genes for this mechanism of converting citrate to oxaloacetate and citryl-CoA have also been reported recently in Chlorobium tepidum (Eisen et al., PNAS 99(14): 9509-14 (2002).

| Protein | GenBank ID | GI Number | Organism |
|---|---|---|---|
| ccsA | BAD17844.1 | 46849514 | Hydrogenobacter thermophilus |
| ccsB | BAD17846.1 | 46849517 | Hydrogenobacter thermophilus |
| sucC1 | AAC07285 | 2983723 | Aquifex aeolicus |
| sucD1 | AAC07686 | 2984152 | Aquifex aeolicus |

| Protein | GenBank ID | GI Number | Organism |
|---|---|---|---|
| ccl | BAD17841.1 | 46849510 | Hydrogenobacter thermophilus |
| aq_150 | AAC06486 | 2982866 | Aquifex aeolicus |
| CT0380 | NP_661284 | 21673219 | Chlorobium tepidum |
| CT0269 | NP_661173.1 | 21673108 | Chlorobium tepidum |
| CT1834 | AAM73055.1 | 21647851 | Chlorobium tepidum |

Oxaloacetate is converted into malate by malate dehydrogenase (EC 1.1.1.37), an enzyme which functions in both the forward and reverse direction. S. cerevisiae possesses three copies of malate dehydrogenase, MDH1 (McAlister-Henn and Thompson, J. Bacteriol. 169:5157-5166 (1987), MDH2 (Minard and McAlister-Henn, Mol. Cell. Biol. 11:370-380 (1991); Gibson and McAlister-Henn, J. Biol. Chem. 278:25628-25636 (2003)), and MDH3 (Steffan and McAlister-Henn, J. Biol. Chem. 267:24708-24715 (1992)), which localize to the mitochondrion, cytosol, and peroxisome, respectively. E. coli is known to have an active malate dehydrogenase encoded by mdh.

| Protein | GenBank ID | GI Number | Organism |
|---|---|---|---|
| MDH1 | NP_012838 | 6322765 | Saccharomyces cerevisiae |
| MDH2 | NP_014515 | 116006499 | Saccharomyces cerevisiae |
| MDH3 | NP_010205 | 6320125 | Saccharomyces cerevisiae |
| Mdh | NP_417703.1 | 16131126 | Escherichia coli |

Fumarate hydratase (EC 4.2.1.2) catalyzes the reversible hydration of fumarate to malate. The three fumarases of E. coli, encoded by fumA, fumB and fumC, are regulated under different conditions of oxygen availability. FumB is oxygen sensitive and is active under anaerobic conditions. FumA is active under microanaerobic conditions, and FumC is active under aerobic growth conditions (Tseng et al., J. Bacteriol. 183:461-467 (2001); Woods et al., Biochim. Biophys. Acta 954:14-26 (1988); Guest et al., J. Gen. Microbiol. 131:2971-2984 (1985)). S. cerevisiae contains one copy of a fumarase-encoding gene, FUM1, whose product localizes to both the cytosol and mitochondrion (Sass et al., J. Biol. Chem. 278:45109-45116 (2003)). Additional fumarase enzymes are found in Campylobacter jejuni (Smith et al., Int. J. Biochem. Cell. Biol. 31:961-975 (1999)), Thermus thermophilus (Mizobata et al., Arch. Biochem. Biophys. 355:49-55 (1998)) and Rattus norvegicus (Kobayashi et al., J. Biochem. 89:1923-1931 (1981)). Similar enzymes with high sequence homology include fum1 from Arabidopsis thaliana and fumC from Corynebacterium glutamicum. The MmcBC fumarase from Pelotomaculum thermopropionicum is another class of fumarase with two subunits (Shimoyama et al., FEMS Microbiol. Lett. 270:207-213 (2007)).

| Protein | GenBank ID | GI Number | Organism |
|---|---|---|---|
| fumA | NP_416129.1 | 16129570 | Escherichia coli |
| fumB | NP_418546.1 | 16131948 | Escherichia coli |
| fumC | NP_416128.1 | 16129569 | Escherichia coli |
| FUM1 | NP_015061 | 6324993 | Saccharomyces cerevisiae |
| fumC | Q8NRN8.1 | 39931596 | Corynebacterium glutamicum |
| fumC | O69294.1 | 9789756 | Campylobacter jejuni |
| fumC | P84127 | 75427690 | Thermus thermophilus |
| fumH | P14408.1 | 120605 | Rattus norvegicus |
| MmcB | YP_001211906 | 147677691 | Pelotomaculum |
| | | | thermopropionicum |
| MmcC | YP_001211907 | 147677692 | Pelotomaculum |
| | | | thermopropionicum |

Fumarate reductase catalyzes the reduction of fumarate to succinate. The fumarate reductase of E. coli, composed of four subunits encoded by frdABCD, is membrane-bound and active under anaerobic conditions. The electron donor for this reaction is menaquinone and the two protons produced in this reaction do not contribute to the proton gradient (Iverson et al., Science 284:1961-1966 (1999)). The yeast genome encodes two soluble fumarate reductase isozymes encoded by FRDS1 (Enomoto et al., DNA Res. 3:263-267 (1996)) and FRDS2 (Muratsubaki et al., Arch. Biochem. Biophys. 352:175-181 (1998)), which localize to the cytosol and promitochondrion, respectively, and are used during anaerobic growth on glucose (Arikawa et al., FEMS Microbiol. Lett. 165:111-116 (1998)).

| Protein | GenBank ID | GI Number | Organism |
|---|---|---|---|
| FRDS1 | P32614 | 418423 | Saccharomyces cerevisiae |
| FRDS2 | NP_012585 | 6322511 | Saccharomyces cerevisiae |

| Protein | GenBank ID | GI Number | Organism |
|---|---|---|---|
| frdA | NP_418578.1 | 16131979 | Escherichia coli |
| frdB | NP_418577.1 | 16131978 | Escherichia coli |
| frdC | NP_418576.1 | 16131977 | Escherichia coli |
| frdD | NP_418475.1 | 16131877 | Escherichia coli |

The ATP-dependent acylation of succinate to succinyl-CoA is catalyzed by succinyl-CoA synthetase (EC 6.2.1.5). The product of the LSC1 and LSC2 genes of S. cerevisiae and the sucC and sucD genes of E. coli naturally form a succinyl-CoA synthetase complex that catalyzes the formation of succinyl-CoA from succinate with the concomitant consumption of one ATP, a reaction which is reversible in vivo (Buck et al., Biochemistry 24:6245-6252 (1985)). These proteins are identified below:

| Protein | GenBank ID | GI Number | Organism |
|---|---|---|---|
| LSC1 | NP_014785 | 6324716 | Saccharomyces cerevisiae |
| LSC2 | NP_011760 | 6321683 | Saccharomyces cerevisiae |
| sucC | NP_415256.1 | 16128703 | Escherichia coli |
| sucD | AAC73823.1 | 1786949 | Escherichia coli |

Alpha-ketoglutarate:ferredoxin oxidoreductase (EC 1.2.7.3), also known as 2-oxoglutarate synthase or 2-oxoglutarate:ferredoxin oxidoreductase (OFOR), forms alpha-ketoglutarate from CO2 and succinyl-CoA with concurrent consumption of two reduced ferredoxin equivalents. OFOR and pyruvate:ferredoxin oxidoreductase (PFOR) are members of a diverse family of 2-oxoacid:ferredoxin (flavodoxin) oxidoreductases which utilize thiamine pyrophosphate, CoA and iron-sulfur clusters as cofactors and ferredoxin, flavodoxin and FAD as electron carriers (Adams et al., Archaea. Adv. Protein Chem. 48:101-180 (1996)). Enzymes in this class are reversible and function in the carboxylation direction in organisms that fix carbon by the RTCA cycle such as Hydrogenobacter thermophilus, Desulfobacter hydrogenophilus and Chlorobium species (Shiba et al. 1985; Evans et al., Proc. Natl. Acad. ScI. U.S.A. 55:92934 (1966); Buchanan, 1971). The two-subunit enzyme from H. thermophilus, encoded by korAB, has been cloned and expressed in E. coli (Yun et al., Biochem. Biophys. Res. Commun. 282:589-594 (2001)). A five subunit OFOR from the same organism with strict substrate specificity for succinyl-CoA, encoded by forDABGE, was recently identified and expressed in E. coli (Yun et al. 2002). The kinetics of CO2 fixation of both H. thermophilus OFOR enzymes have been characterized (Yamamoto et al., Extremophiles 14:79-85 (2010)). A CO2-fixing OFOR from Chlorobium thiosulfatophilum has been purified and characterized but the genes encoding this enzyme have not been identified to date. Enzyme candidates in Chlorobium species can be inferred by sequence similarity to the H. thermophilus genes. For example, the Chlorobium limicola genome encodes two similar proteins. Acetogenic bacteria such as Moorella thermoacetica are predicted to encode two OFOR enzymes. The enzyme encoded by Moth_{—}0034 is predicted to function in the CO2-assimilating direction. The genes associated with this enzyme, Moth_{—}0034 have not been experimentally validated to date but can be inferred by sequence similarity to known OFOR enzymes.

OFOR enzymes that function in the decarboxylation direction under physiological conditions can also catalyze the reverse reaction. The OFOR from the thermoacidophilic archaeon Sulfolobus sp. strain 7, encoded by ST2300, has been extensively studied (Zhang et al. 1996. A plasmid-based expression system has been developed for efficiently expressing this protein in E. coli (Fukuda et al., Eur. J. Biochem. 268:5639-5646 (2001)) and residues involved in substrate specificity were determined (Fukuda and Wakagi, Biochim. Biophys. Acta 1597:74-80 (2002)). The OFOR encoded by Ape1472/Ape1473 from Aeropyrum pernix str. K1 was recently cloned into E. coli, characterized, and found to react with 2-oxoglutarate and a broad range of 2-oxoacids (Nishizawa et al., FEBS Lett. 579:2319-2322 (2005)). Another exemplary OFOR is encoded by oorDABC in Helicobacter pylori (Hughes et al. 1998). An enzyme specific to alpha-ketoglutarate has been reported in Thauera aromatica (Dorner and Boll, J, Bacteriol. 184 (14), 3975-83 (2002). A similar enzyme can be found in Rhodospirillum rubrum by sequence homology. A two subunit enzyme has also been identified in Chlorobium tepidum (Eisen et al., PNAS 99(14): 9509-14 (2002)).

| Protein | GenBank ID | GI Number | Organism |
|---|---|---|---|
| korA | BAB21494 | 12583691 | Hydrogenobacter |
| | | | thermophilus |
| korB | BAB21495 | 12583692 | Hydrogenobacter |
| | | | thermophilus |
| forD | BAB62132.1 | 14970994 | Hydrogenobacter |
| | | | thermophilus |
| forA | BAB62133.1 | 14970995 | Hydrogenobacter |
| | | | thermophilus |
| forB | BAB62134.1 | 14970996 | Hydrogenobacter |
| | | | thermophilus |
| forG | BAB62135.1 | 14970997 | Hydrogenobacter |
| | | | thermophilus |
| forE | BAB62136.1 | 14970998 | Hydrogenobacter |
| | | | thermophilus |
| Clim_0204 | ACD89303.1 | 189339900 | Chlorobium limicola |
| Clim_0205 | ACD89302.1 | 189339899 | Chlorobium limicola |
| Clim_1123 | ACD90192.1 | 189340789 | Chlorobium limicola |
| Clim_1124 | ACD90193.1 | 189340790 | Chlorobium limicola |
| Moth_1984 | YP_430825.1 | 83590816 | Moorella thermoacetica |
| Moth_1985 | YP_430826.1 | 83590817 | Moorella thermoacetica |
| Moth_0034 | YP_428917.1 | 83588908 | Moorella thermoacetica |
| ST2300 | NP_378302.1 | 15922633 | Sulfolobus sp. strain 7 |
| Ape1472 | BAA80470.1 | 5105156 | Aeropyrum pernix |
| Ape1473 | BAA80471.2 | 116062794 | Aeropyrum pernix |
| oorD | AAC38210.1 | 2935178 | Helicobacter pylori |
| oorA | AAC38211.1 | 2935179 | Helicobacter pylori |
| oorB | AAC38212.1 | 2935180 | Helicobacter pylori |
| oorC | AAC38213.1 | 2935181 | Helicobacter pylori |
| CT0163 | NP_661069.1 | 21673004 | Chlorobium tepidum |
| CT0162 | NP_661068.1 | 21673003 | Chlorobium tepidum |
| korA | CAA12243.2 | 19571179 | Thauera aromatica |
| korB | CAD27440.1 | 19571178 | Thauera aromatica |
| Rru_A2721 | YP_427805.1 | 83594053 | Rhodospirillum rubrum |
| Rru_A2722 | YP_427806.1 | 83594054 | Rhodospirillum rubrum |

Isocitrate dehydrogenase catalyzes the reversible decarboxylation of isocitrate to 2-oxoglutarate coupled to the reduction of NAD(P)⁺. IDH enzymes in Saccharomyces cerevisiae and Escherichia coli are encoded by IDP1 and icd, respectively (Haselbeck and McAlister-Henn, J. Biol. Chem. 266:2339-2345 (1991); Nimmo, H. G., Biochem. J. 234:317-2332 (1986)). The reverse reaction in the reductive TCA cycle, the reductive carboxylation of 2-oxoglutarate to isocitrate, is favored by the NADPH-dependent CO₂-fixing IDH from Chlorobium limicola and was functionally expressed in E. coli (Kanao et al., Eur. J. Biochem. 269:1926-1931 (2002)). A similar enzyme with 95% sequence identity is found in the C. tepidum genome in addition to some other candidates listed below.

| Protein | GenBank ID | GI Number | Organism |
|---|---|---|---|
| Icd | ACI84720.1 | 209772816 | Escherichia coli |
| IDP1 | AAA34703.1 | 171749 | Saccharomyces cerevisiae |
| Idh | BAC00856.1 | 21396513 | Chlorobium limicola |
| Icd | AAM71597.1 | 21646271 | Chlorobium tepidum |
| icd | NP_952516.1 | 39996565 | Geobacter sulfurreducens |
| icd | YP_393560. | 78777245 | Sulfurimonas denitrificans |

In H. thermophilus the reductive carboxylation of 2-oxoglutarate to isocitrate is catalyzed by two enzymes: 2-oxoglutarate carboxylase and oxalosuccinate reductase. 2-Oxoglutarate carboxylase (EC 6.4.1.7) catalyzes the ATP-dependent carboxylation of alpha-ketoglutarate to oxalosuccinate (Aoshima and Igarashi, Mol. Microbiol. 62:748-759 (2006)). This enzyme is a large complex composed of two subunits. Biotinylation of the large (A) subunit is required for enzyme function (Aoshima et al., Mol. Microbiol. 51:791-798 (2004)). Oxalosuccinate reductase (EC 1.1.1.-) catalyzes the NAD-dependent conversion of oxalosuccinate to D-threo-isocitrate. The enzyme is a homodimer encoded by icd in H. thermophilus. The kinetic parameters of this enzyme indicate that the enzyme only operates in the reductive carboxylation direction in vivo, in contrast to isocitrate dehydrogenase enzymes in other organisms (Aoshima and Igarashi, J. Bacteriol. 190:2050-2055 (2008)). Based on sequence homology, gene candidates have also been found in Thiobacillus denitrificans and Thermocrinis albus.

| Protein | GenBank ID | GI Number | Organism |
|---|---|---|---|
| cfiA | BAF34932.1 | 116234991 | Hydrogenobacter thermophilus |
| cifB | BAF34931.1 | 116234990 | Hydrogenobacter thermophilus |
| Icd | BAD02487.1 | 38602676 | Hydrogenobacter thermophilus |
| Tbd_1556 | YP_315314 | 74317574 | Thiobacillus denitrificans |

| Protein | GenBank ID | GI Number | Organism |
|---|---|---|---|
| Tbd_1555 | YP_315313 | 74317573 | Thiobacillus denitrificans |
| Tbd_0854 | YP_314612 | 74316872 | Thiobacillus denitrificans |
| Thal_0268 | YP_003473030 | 289548042 | Thermocrinis albus |
| Thal_0267 | YP_003473029 | 289548041 | Thermocrinis albus |
| Thal_0646 | YP_003473406 | 289548418 | Thermocrinis albus |

Aconitase (EC 4.2.1.3) is an iron-sulfur-containing protein catalyzing the reversible isomerization of citrate and iso-citrate via the intermediate cis-aconitate. Two aconitase enzymes are encoded in the E. coli genome by acnA and acnB. AcnB is the main catabolic enzyme, while AcnA is more stable and appears to be active under conditions of oxidative or acid stress (Cunningham et al., Microbiology 143 (Pt 12):3795-3805 (1997)). Two isozymes of aconitase in Salmonella typhimurium are encoded by acnA and acnB (Horswill and Escalante-Semerena, Biochemistry 40:4703-4713 (2001)). The S. cerevisiae aconitase, encoded by ACO1, is localized to the mitochondria where it participates in the TCA cycle (Gangloff et al., Mol. Cell. Biol. 10:3551-3561 (1990)) and the cytosol where it participates in the glyoxylate shunt (Regev-Rudzki et al., Mol. Biol. Cell. 16:4163-4171 (2005)).

| Protein | GenBank ID | GI Number | Organism |
|---|---|---|---|
| acnA | AAC7438.1 | 1787531 | Escherichia coli |
| acnB | AAC73229.1 | 2367097 | Escherichia coli |
| acnA | NP_460671.1 | 16765056 | Salmonella typhimurium |
| acnB | NP_459163.1 | 16763548 | Salmonella typhimurium |
| ACO1 | AAA34389.1 | 170982 | Saccharomyces cerevisiae |

Pyruvate:ferredoxin oxidoreductase (PFOR) catalyzes the reversible oxidation of pyruvate to form acetyl-CoA. The PFOR from Desulfovibrio africanus has been cloned and expressed in E. coli resulting in an active recombinant enzyme that was stable for several days in the presence of oxygen (Pieulle et al., J. Bacteriol. 179:5684-5692 (1997)). Oxygen stability is relatively uncommon in PFORs and is believed to be conferred by a 60 residue extension in the polypeptide chain of the D. africanus enzyme. Two cysteine residues in this enzyme form a disulfide bond that protects it against inactivation in the form of oxygen. This disulfide bond and the stability in the presence of oxygen has been found in other Desulfovibrio species also (Vita et al., Biochemistry, 47: 957-64 (2008)). The M. thermoacetica PFOR is also well characterized (Menon and Ragsdale, Biochemistry 36:8484-8494 (1997)) and was shown to have high activity in the direction of pyruvate synthesis during autotrophic growth (Furdui and Ragsdale, J. Biol. Chem. 275:28494-28499 (2000)). Further, E. coli possesses an uncharacterized open reading frame, ydbK, encoding a protein that is 51% identical to the M. thermoacetica PFOR. Evidence for pyruvate oxidoreductase activity in E. coli has been described (Blaschkowski et al., Eur. J. Biochem. 123:563-569 (1982)). PFORs have also been described in other organisms, including Rhodobacter capsulatas (Yakunin and Hallenbeck, Biochimica et Biophysica Acta 1409 (1998) 39-49 (1998)) and Choloboum tepidum (Eisen et al., PNAS 99(14): 9509-14 (2002)). The five subunit PFOR from H. thermophilus, encoded by porEDABG, was cloned into E. coli and shown to function in both the decarboxylating and CO₂-assimilating directions (Ikeda et al. 2006; Yamamoto et al., Extremophiles 14:79-85 (2010)). Homologs also exist in C. carboxidivorans P7. Several additional PFOR enzymes are described in the following review (Ragsdale, S. W., Chem. Rev. 103:2333-2346 (2003)). Finally, flavodoxin reductases (e.g., fqrB from Helicobacter pylori or Campylobacter jejuni) (St Maurice et al., J. Bacteriol. 189:4764-4773 (2007)) or Rnf-type proteins (Seedorf et al., Proc. Natl. Acad. Sci. U.S.A. 105:2128-2133 (2008); and Herrmann, J. Bacteriol 190:784-791 (2008)) provide a means to generate NADH or NADPH from the reduced ferredoxin generated by PFOR. These proteins are identified below.

| Protein | GenBank ID | GI Number | Organism |
|---|---|---|---|
| Por | CAA70873.1 | 1770208 | Desulfovibrio africanus |
| por | YP_012236.1 | 46581428 | Desulfovibrio vulgaris str. Hildenborough |
| Dde_3237 | ABB40031.1 | 78220682 | DesulfoVibrio desulfuricans G20 |
| Ddes_0298 | YP_002478891.1 | 220903579 | Desulfovibrio desulfuricans subsp. |
| | | | desulfuricans str. ATCC 27774 |
| Por | YP_428946.1 | 83588937 | Moorella thermoacetica |
| YdbK | NP_415896.1 | 16129339 | Escherichia coli |
| nifJ (CT1628) | NP_662511.1 | 21674446 | Chlorobium tepidum |
| CJE1649 | YP_179630.1 | 57238499 | Campylobacter jejuni |
| nifJ | ADE85473.1 | 294476085 | Rhodobacter capsulatus |
| porE | BAA95603.1 | 7768912 | Hydrogenobacter thermophilus |
| porD | BAA95604.1 | 7768913 | Hydrogenobacter thermophilus |
| porA | BAA95605.1 | 7768914 | Hydrogenobacter thermophilus |
| porB | BAA95606.1 | 776891 | Hydrogenobacter thermophilus |
| porG | BAA95607.1 | 7768916 | Hydrogenobacter thermophilus |
| FqrB | YP_001482096.1 | 157414840 | Campylobacter jejuni |
| HP1164 | NP_207955.1 | 15645778 | Helicobacter pylori |
| RnfC | EDK33306.1 | 146346770 | Clostridium kluyveri |
| RnfD | EDK33307.1 | 146346771 | Clostridium kluyveri |
| RnfG | EDK33308.1 | 146346772 | Clostridium kluyveri |
| RnfE | EDK33309.1 | 146346773 | Clostridium kluyveri |
| RnfA | EDK33310.1 | 146346774 | Clostridium kluyveri |
| RnfB | EDK33311.1 | 146346775 | Clostridium kluyveri |

The conversion of pyruvate into acetyl-CoA can be catalyzed by several other enzymes or their combinations thereof. For example, pyruvate dehydrogenase can transform pyruvate into acetyl-CoA with the concomitant reduction of a molecule of NAD into NADH. It is a multi-enzyme complex that catalyzes a series of partial reactions which results in acylating oxidative decarboxylation of pyruvate. The enzyme comprises of three subunits: the pyruvate decarboxylase (E1), dihydrolipoamide acyltransferase (E2) and dihydrolipoamide dehydrogenase (E3). This enzyme is naturally present in several organisms, including E. coli and S. cerevisiae. In the E. coli enzyme, specific residues in the E1 component are responsible for substrate specificity (Bisswanger, H., J. Biol. Chem. 256:815-82 (1981); Bremer, J., Eur. J. Biochem. 8:535-540 (1969); Gong et al., J. Biol. Chem. 275:13645-13653 (2000)). Enzyme engineering efforts have improved the E. coli PDH enzyme activity under anaerobic conditions (Kim et al., J. Bacteriol. 190:3851-3858 (2008); Kim et al., Appl. Environ. Microbiol. 73:1766-1771 (2007); Zhou et al., Biotechnol. Lett. 30:335-342 (2008)). In contrast to the E. coli PDH, the B. subtilis complex is active and required for growth under anaerobic conditions (Nakano et al., J. Bacteriol. 179:6749-6755 (1997)). The Klebsiella pneumoniae PDH, characterized during growth on glycerol, is also active under anaerobic conditions (5). Crystal structures of the enzyme complex from bovine kidney (18) and the E2 catalytic domain from Azotobacter vinelandii are available (4). Yet another enzyme that can catalyze this conversion is pyruvate formate lyase. This enzyme catalyzes the conversion of pyruvate and CoA into acetyl-CoA and formate. Pyruvate formate lyase is a common enzyme in prokaryotic organisms that is used to help modulate anaerobic redox balance. Exemplary enzymes can be found in Escherichia coli encoded by pflB (Knappe and Sawers, FEMS. Microbiol Rev. 6:383-398 (1990)), Lactococcus lactis (Melchiorsen et al., Appl Microbiol Biotechnol 58:338-344 (2002)), and Streptococcus mutans (Takahashi-Abbe et al., Oral. Microbiol Immunol. 18:293-297 (2003)). E. coli possesses an additional pyruvate formate lyase, encoded by tdcE, that catalyzes the conversion of pyruvate or 2-oxobutanoate to acetyl-CoA or propionyl-CoA, respectively (Hesslinger et al., Mol. Microbiol 27:477-492 (1998)). Both pflB and tdcE from E. coli require the presence of pyruvate formate lyase activating enzyme, encoded by pflA. Further, a short protein encoded by yfiD in E. coli can associate with and restore activity to oxygen-cleaved pyruvate formate lyase (Vey et al., Proc. Natl. Acad. Sci. U.S.A. 105:16137-16141 (2008). Note that pflA and pflB from E. coli were expressed in S. cerevisiae as a means to increase cytosolic acetyl-CoA for butanol production as described in WO/2008/080124]. Additional pyruvate formate lyase and activating enzyme candidates, encoded by pfl and act, respectively, are found in Clostridium pasteurianum (Weidner et al., J. Bacteriol. 178:2440-2444 (1996)).

Further, different enzymes can be used in combination to convert pyruvate into acetyl-CoA. For example, in S. cerevisiae, acetyl-CoA is obtained in the cytosol by first decarboxylating pyruvate to form acetaldehyde; the latter is oxidized to acetate by acetaldehyde dehydrogenase and subsequently activated to form acetyl-CoA by acetyl-CoA synthetase. Acetyl-CoA synthetase is a native enzyme in several other organisms including E. coli (Kumari et al., J. Bacteriol. 177:2878-2886 (1995)), Salmonella enterica (Starai et al., Microbiology 151:3793-3801 (2005); Starai et al., J. Biol. Chem. 280:26200-26205 (2005)), and Moorella thermoacetica (described already). Alternatively, acetate can be activated to form acetyl-CoA by acetate kinase and phosphotransacetylase. Acetate kinase first converts acetate into acetyl-phosphate with the accompanying use of an ATP molecule. Acetyl-phosphate and CoA are next converted into acetyl-CoA with the release of one phosphate by phosphotransacetylase. Both acetate kinase and phosphotransacetylyase are well-studied enzymes in several Clostridia and Methanosarcina thermophila.

Yet another way of converting pyruvate to acetyl-CoA is via pyruvate oxidase. Pyruvate oxidase converts pyruvate into acetate, using ubiquione as the electron acceptor. In E. coli, this activity is encoded by poxB. PoxB has similarity to pyruvate decarboxylase of S. cerevisiae and Zymomonas mobilis. The enzyme has a thiamin pyrophosphate cofactor (Koland and Gennis, Biochemistry 21:4438-4442 (1982)); O'Brien et al., Biochemistry 16:3105-3109 (1977); O'Brien and Gennis, J. Biol. Chem. 255:3302-3307 (1980)) and a flavin adenine dinucleotide (FAD) cofactor. Acetate can then be converted into acetyl-CoA by either acetyl-CoA synthetase or by acetate kinase and phosphotransacetylase, as described earlier. Some of these enzymes can also catalyze the reverse reaction from acetyl-CoA to pyruvate.

For enzymes that use reducing equivalents in the form of NADH or NADPH, these reduced carriers can be generated by transferring electrons from reduced ferredoxin. Two enzymes catalyze the reversible transfer of electrons from reduced ferredoxins to NAD(P)⁺, ferredoxin:NAD⁺ oxidoreductase (EC 1.18.1.3) and ferredoxin:NADP⁺ oxidoreductase (FNR, EC 1.18.1.2). Ferredoxin:NADP⁺ oxidoreductase (FNR, EC 1.18.1.2) has a noncovalently bound FAD cofactor that facilitates the reversible transfer of electrons from NADPH to low-potential acceptors such as ferredoxins or flavodoxins (Blaschkowski et al., Eur. J. Biochem. 123:563-569 (1982); Fujii et al., 1977). The Helicobacter pylori FNR, encoded by HP1164 (fqrB), is coupled to the activity of pyruvate:ferredoxin oxidoreductase (PFOR) resulting in the pyruvate-dependent production of NADPH (St et al. 2007). An analogous enzyme is found in Campylobacter jejuni (St et al. 2007). A ferredoxin:NADP⁺ oxidoreductase enzyme is encoded in the E. coli genome by fpr (Bianchi et al. 1993). Ferredoxin:NAD⁺ oxidoreductase utilizes reduced ferredoxin to generate NADH from NAD⁺. In several organisms, including E. coli, this enzyme is a component of multifunctional dioxygenase enzyme complexes. The ferredoxin:NAD⁺ oxidoreductase of E. coli, encoded by hcaD, is a component of the 3-phenylproppionate dioxygenase system involved in involved in aromatic acid utilization (Diaz et al. 1998). NADH:ferredoxin reductase activity was detected in cell extracts of Hydrogenobacter thermophilus strain TK-6, although a gene with this activity has not yet been indicated (Yoon et al. 2006). Finally, the energy-conserving membrane-associated Rnf-type proteins (Seedorf et al., Proc. Natl. Acad. Sci. U.S.A. 105:2128-2133 (2008); Herrmann et al., J. Bacteriol. 190:784-791 (2008)) provide a means to generate NADH or NADPH from reduced ferredoxin. Additional ferredoxin:NAD(P)+ oxidoreductases have been annotated in Clostridium carboxydivorans P7.

| Protein | GenBank ID | GI Number | Organism |
|---|---|---|---|
| HP1164 | NP_207955.1 | 15645778 | Helicobacter pylori |
| CJE0663 | AAW35824.1 | 57167045 | Campylobacter jejuni |
| fpr | P28861.4 | 399486 | Escherichia coli |
| hcaD | AAC75595.1 | 1788892 | Escherichia coli |
| LOC100282643 | NP_001149023.1 | 226497434 | Zea mays |
| RnfC | EDK33306.1 | 146346770 | Clostridium kluyveri |
| RnfD | EDK33307.1 | 146346771 | Clostridium kluyveri |
| RnfG | EDK33308.1 | 146346772 | Clostridium kluyveri |
| RnfE | EDK33309.1 | 146346773 | Clostridium kluyveri |
| RnfA | EDK33310.1 | 146346774 | Clostridium kluyveri |
| RnfB | EDK33311.1 | 146346775 | Clostridium kluyveri |
| CcarbDRAFT_2639 | ZP_05392639.1 | 255525707 | Clostridium carboxidivorans |
| | | | P7 |
| CcarbDRAFT_2638 | ZP_05392638.1 | 255525706 | Clostridium carboxidivorans |
| | | | P7 |
| CcarbDRAFT_2636 | ZP_05392636.1 | 255525704 | Clostridium carboxidivorans |
| | | | P7 |
| CcarbDRAFT_5060 | ZP_05395060.1 | 255528241 | Clostridium carboxidivorans |
| | | | P7 |
| CcarbDRAFT_2450 | ZP_05392450.1 | 255525514 | Clostridium carboxidivorans |
| | | | P7 |
| CcarbDRAFT_1084 | ZP_05391084.1 | 255524124 | Clostridium carboxidivorans |
| | | | P7 |

Ferredoxins are small acidic proteins containing one or more iron-sulfur clusters that function as intracellular electron carriers with a low reduction potential. Reduced ferredoxins donate electrons to Fe-dependent enzymes such as ferredoxin-NADP⁺ oxidoreductase, pyruvate:ferredoxin oxidoreductase (PFOR) and 2-oxoglutarate:ferredoxin oxidoreductase (OFOR). The H. thermophilus gene fdx1 encodes a [4Fe-4S]-type ferredoxin that is required for the reversible carboxylation of 2-oxoglutarate and pyruvate by OFOR and PFOR, respectively (Yamamoto et al., Extremophiles 14:79-85 (2010)). The ferredoxin associated with the Sulfolobus solfataricus 2-oxoacid:ferredoxin reductase is a monomeric dicluster [3Fe-4S][4Fe-4S] type ferredoxin (Park et al. 2006). While the gene associated with this protein has not been fully sequenced, the N-terminal domain shares 93% homology with the zfx ferredoxin from S. acidocaldarius. The E. coli genome encodes a soluble ferredoxin of unknown physiological function, fdx. Some evidence indicates that this protein can function in iron-sulfur cluster assembly (Takahashi and Nakamura, 1999). Additional ferredoxin proteins have been characterized in Helicobacter pylori (Mukhopadhyay et al. 2003) and Campylobacter jejuni (van Vliet et al. 2001). A 2Fe-2S ferredoxin from Clostridium pasteurianum has been cloned and expressed in E. coli (Fujinaga and Meyer, Biochemical and Biophysical Research Communications, 192(3): (1993)). Acetogenic bacteria such as Moorella thermoacetica, Clostridium carboxidivorans P7 and Rhodospirillum rubrum are predicted to encode several ferredoxins, listed in the table below.

| Protein | GenBank ID | GI Number | Organism |
|---|---|---|---|
| fdx1 | BAE02673.1 | 68163284 | Hydrogenobacter thermophilus |
| M11214.1 | AAA83524.1 | 144806 | Clostridium pasteurianum |
| Zfx | AAY79867.1 | 68566938 | Sulfolobus acidocalarius |
| Fdx | AAC75578.1 | 1788874 | Escherichia coli |
| hp_0277 | AAD07340.1 | 2313367 | Helicobacter pylori |
| fdxA | CAL34484.1 | 112359698 | Campylobacter jejuni |
| Moth_0061 | ABC18400.1 | 83571848 | Moorella thermoacetica |
| Moth_1200 | ABC19514.1 | 83572962 | Moorella thermoacetica |
| Moth_1888 | ABC20188.1 | 83573636 | Moorella thermoacetica |
| Moth_2112 | ABC20404.1 | 83573852 | Moorella thermoacetica |
| CcarbDRAFT_4383 | ZP_05394383.1 | 255527515 | Clostridium carboxidivorans P7 |
| CcarbDRAFT_2958 | ZP_05392958.1 | 255526034 | Clostridium carboxidivorans P7 |
| CcarbDRAFT_2281 | ZP_05392281.1 | 255525342 | Clostridium carboxidivorans P7 |
| CcarbDRAFT_5296 | ZP_05395295.1 | 255528511 | Clostridium carboxidivorans P7 |
| CcarbDRAFT_1615 | ZP_05391615.1 | 255524662 | Clostridium carboxidivorans P7 |
| CcarbDRAFT_1304 | ZP_05391304.1 | 255524347 | Clostridium carboxidivorans P7 |
| Rru_A2264 | ABC23064.1 | 83576513 | Rhodospirillum rubrum |
| Rru_A1916 | ABC22716.1 | 83576165 | Rhodospirillum rubrum |
| Rru_A2026 | ABC22826.1 | 83576275 | Rhodospirillum rubrum |

Succinyl-CoA transferase catalyzes the conversion of succinyl-CoA to succinate while transferring the CoA moiety to a CoA acceptor molecule. Many transferases have broad specificity and can utilize CoA acceptors as diverse as acetate, succinate, propionate, butyrate, 2-methylacetoacetate, 3-ketohexanoate, 3-ketopentanoate, valerate, crotonate, 3-mercaptopropionate, propionate, vinylacetate, and butyrate, among others.

The conversion of succinate to succinyl-CoA can be carried by a transferase which does not require the direct consumption of an ATP or GTP. This type of reaction is common in a number of organisms. The conversion of succinate to succinyl-CoA can also be catalyzed by succinyl-CoA:Acetyl-CoA transferase. The gene product of cat1 of Clostridium kluyveri has been shown to exhibit succinyl-CoA: acetyl-CoA transferase activity (Sohling and Gottschalk, J. Bacteriol. 178:871-880 (1996)). In addition, the activity is present in Trichomonas vaginalis (van Grinsven et al. 2008) and Trypanosoma brucei (Riviere et al. 2004). The succinyl-CoA:acetate CoA-transferase from Acetobacter aceti, encoded by aarC, replaces succinyl-CoA synthetase in a variant TCA cycle (Mullins et al. 2008). Similar succinyl-CoA transferase activities are also present in Trichomonas vaginalis (van Grinsven et al. 2008), Trypanosoma brucei (Riviere et al. 2004) and Clostridium kluyveri (Sohling and Gottschalk, 1996c). The beta-ketoadipate:succinyl-CoA transferase encoded by pcaI and pcaJ in Pseudomonas putida is yet another candidate (Kaschabek et al. 2002). The aforementioned proteins are identified below.

| Protein | GenBank ID | GI Number | Organism |
|---|---|---|---|
| cat1 | P38946.1 | 729048 | Clostridium kluyveri |
| TVAG_395550 | XP_001330176 | 123975034 | Trichomonas |
| | | | vaginalis G3 |
| Tb11.02.0290 | XP_828352 | 71754875 | Trypanosoma brucei |
| pcaI | AAN69545.1 | 24985644 | Pseudomonas putida |
| pcaJ | NP_746082.1 | 26990657 | Pseudomonas putida |
| aarC | ACD85596.1 | 189233555 | Acetobacter aceti |

An additional exemplary transferase that converts succinate to succinyl-CoA while converting a 3-ketoacyl-CoA to a 3-ketoacid is succinyl-CoA:3:ketoacid-CoA transferase (EC 2.8.3.5). Exemplary succinyl-CoA:3:ketoacid-CoA transferases are present in Helicobacter pylori (Corthesy-Theulaz et al. 1997), Bacillus subtilis, and Homo sapiens (Fukao et al. 2000; Tanaka et al. 2002). The aforementioned proteins are identified below.

| Protein | GenBank ID | GI Number | Organism |
|---|---|---|---|
| HPAG1_0676 | YP_627417 | 108563101 | Helicobacter pylori |
| HPAG1_0677 | YP_627418 | 108563102 | Helicobacter pylori |
| ScoA | NP_391778 | 16080950 | Bacillus subtilis |
| ScoB | NP_391777 | 16080949 | Bacillus subtilis |
| OXCT1 | NP_000427 | 4557817 | Homo sapiens |
| OXCT2 | NP_071403 | 11545841 | Homo sapiens |

Converting succinate to succinyl-CoA by succinyl-CoA:3:ketoacid-CoA transferase requires the simultaneous conversion of a 3-ketoacyl-CoA such as acetoacetyl-CoA to a 3-ketoacid such as acetoacetate. Conversion of a 3-ketoacid back to a 3-ketoacyl-CoA can be catalyzed by an acetoacetyl-CoA:acetate:CoA transferase. Acetoacetyl-CoA:acetate:CoA transferase converts acetoacetyl-CoA and acetate to acetoacetate and acetyl-CoA, or vice versa. Exemplary enzymes include the gene products of atoAD from E. coli (Hanai et al., Appl Environ Microbiol 73:7814-7818 (2007), ctfAB from C. acetobutylicum (Jojima et al., Appl Microbiol Biotechnol 77:1219-1224 (2008), and ctfAB from Clostridium saccharoperbutylacetonicum (Kosaka et al., Biosci. Biotechnol Biochem. 71:58-68 (2007)) are shown below.

| Protein | GenBank ID | GI Number | Organism |
|---|---|---|---|
| AtoA | NP_416726.1 | 2492994 | Escherichia coli |
| AtoD | NP_416725.1 | 2492990 | Escherichia coli |
| CtfA | NP_149326.1 | 15004866 | Clostridium acetobutylicum |
| CtfB | NP_149327.1 | 15004867 | Clostridium acetobutylicum |
| CtfA | AAP42564.1 | 31075384 | Clostridium |
| | | | saccharoperbutylacetonicum |
| CtfB | AAP42565.1 | 31075385 | Clostridium |
| | | | saccharoperbutylacetonicum |

Yet another possible CoA acceptor is benzylsuccinate. Succinyl-CoA:(R)-Benzylsuccinate CoA-Transferase functions as part of an anaerobic degradation pathway for toluene in organisms such as Thauera aromatica (Leutwein and Heider, J. Bact. 183(14) 4288-4295 (2001)). Homologs can be found in Azoarcus sp. T, Aromatoleum aromaticum EbN1, and Geobacter metallireducens GS-15. The aforementioned proteins are identified below.

| Protein | GenBank ID | GI Number | Organism |
|---|---|---|---|
| bbsE | AAF89840 | 9622535 | Thauera aromatic |
| Bbsf | AAF89841 | 9622536 | Thauera aromatic |
| bbsE | AAU45405.1 | 52421824 | Azoarcus sp. T |
| bbsF | AAU45406.1 | 52421825 | Azoarcus sp. T |
| bbsE | YP_158075.1 | 56476486 | Aromatoleum aromaticum |
| | | | EbN1 |
| bbsF | YP_158074.1 | 56476485 | Aromatoleum aromaticum |
| | | | EbN1 |
| Gmet_1521 | YP_384480.1 | 78222733 | Geobacter metallireducens |
| | | | GS-15 |
| Gmet_1522 | YP_384481.1 | 78222734 | Geobacter metallireducens |
| | | | GS-15 |

Additionally, ygfH encodes a propionyl CoA:succinate CoA transferase in E. coli (Haller et al., Biochemistry, 39(16) 4622-4629). Close homologs can be found in, for example, Citrobacter youngae ATCC 29220, Salmonella enterica subsp. arizonae serovar, and Yersinia intermedia ATCC 29909. The aforementioned proteins are identified below.

| Protein | GenBank ID | GI Number | Organism |
|---|---|---|---|
| ygfH | NP_417395.1 | 16130821 | Escherichia coli str. K-12 substr. |
| | | | MG1655 |
| CIT292_04485 | ZP_03838384.1 | 227334728 | Citrobacter youngae ATCC 29220 |
| SARI_04582 | YP_001573497.1 | 161506385 | Salmonella enterica subsp. arizonae |
| | | | serovar |
| yinte0001_14430 | ZP_04635364.1 | 238791727 | Yersinia intermedia ATCC 29909 |

Citrate lyase (EC 4.1.3.6) catalyzes a series of reactions resulting in the cleavage of citrate to acetate and oxaloacetate. The enzyme is active under anaerobic conditions and is composed of three subunits: an acyl-carrier protein (ACP, gamma), an ACP transferase (alpha), and a acyl lyase (beta). Enzyme activation uses covalent binding and acetylation of an unusual prosthetic group, 2′-(5″-phosphoribosyl)-3-′-dephospho-CoA, which is similar in structure to acetyl-CoA. Acylation is catalyzed by CitC, a citrate lyase synthetase. Two additional proteins, CitG and CitX, are used to convert the apo enzyme into the active holo enzyme (Schneider et al., Biochemistry 39:9438-9450 (2000)). Wild type E. coli does not have citrate lyase activity; however, mutants deficient in molybdenum cofactor synthesis have an active citrate lyase (Clark, FEMS Microbiol. Lett. 55:245-249 (1990)). The E. coli enzyme is encoded by citEFD and the citrate lyase synthetase is encoded by citC (Nilekani and SivaRaman, Biochemistry 22:4657-4663 (1983)). The Leuconostoc mesenteroides citrate lyase has been cloned, characterized and expressed in E. coli (Bekal et al., J. Bacteriol. 180:647-654 (1998)). Citrate lyase enzymes have also been identified in enterobacteria that utilize citrate as a carbon and energy source, including Salmonella typhimurium and Klebsiella pneumoniae (Bott, Arch. Microbiol. 167: 78-88 (1997); Bott and Dimroth, Mol. Microbiol. 14:347-356 (1994)). The aforementioned proteins are tabulated below.

| Protein | GenBank ID | GI Number | Organism |
|---|---|---|---|
| citF | AAC73716.1 | 1786832 | Escherichia coli |
| Cite | AAC73717.2 | 87081764 | Escherichia coli |
| citD | AAC73718.1 | 1786834 | Escherichia coli |
| citC | AAC73719.2 | 87081765 | Escherichia coli |
| citG | AAC73714.1 | 1786830 | Escherichia coli |
| citX | AAC73715.1 | 1786831 | Escherichia coli |
| citF | CAA71633.1 | 2842397 | Leuconostoc mesenteroides |
| Cite | CAA71632.1 | 2842396 | Leuconostoc mesenteroides |
| citD | CAA71635.1 | 2842395 | Leuconostoc mesenteroides |
| citC | CAA71636.1 | 3413797 | Leuconostoc mesenteroides |
| citG | CAA71634.1 | 2842398 | Leuconostoc mesenteroides |
| citX | CAA71634.1 | 2842398 | Leuconostoc mesenteroides |
| citF | NP_459613.1 | 16763998 | Salmonella typhimurium |
| cite | AAL19573.1 | 16419133 | Salmonella typhimurium |
| citD | NP_459064.1 | 16763449 | Salmonella typhimurium |
| citC | NP_459616.1 | 16764001 | Salmonella typhimurium |
| citG | NP_459611.1 | 16763996 | Salmonella typhimurium |
| citX | NP_459612.1 | 16763997 | Salmonella typhimurium |
| citF | CAA56217.1 | 565619 | Klebsiella pneumoniae |
| cite | CAA56216.1 | 565618 | Klebsiella pneumoniae |
| citD | CAA56215.1 | 565617 | Klebsiella pneumoniae |
| citC | BAH66541.1 | 238774045 | Klebsiella pneumoniae |
| citG | CAA56218.1 | 565620 | Klebsiella pneumoniae |
| citX | AAL60463.1 | 18140907 | Klebsiella pneumoniae |

Acetate kinase (EC 2.7.2.1) catalyzes the reversible ATP-dependent phosphorylation of acetate to acetylphosphate. Exemplary acetate kinase enzymes have been characterized in many organisms including E. coli, Clostridium acetobutylicum and Methanosarcina thermophila (Ingram-Smith et al., J. Bacteriol. 187:2386-2394 (2005); Fox and Roseman, J. Biol. Chem. 261:13487-13497 (1986); Winzer et al., Microbioloy 143 (Pt 10):3279-3286 (1997)). Acetate kinase activity has also been demonstrated in the gene product of E. coli purT (Marolewski et al., Biochemistry 33:2531-2537 (1994). Some butyrate kinase enzymes (EC 2.7.2.7), for example buk1 and buk2 from Clostridium acetobutylicum, also accept acetate as a substrate (Hartmanis, M. G., J. Biol. Chem. 262:617-621 (1987)).

| Protein | GenBank ID | GI Number | Organism |
|---|---|---|---|
| ackA | NP_416799.1 | 16130231 | Escherichia coli |
| Ack | AAB18301.1 | 1491790 | Clostridium acetobutylicum |
| Ack | AAA72042.1 | 349834 | Methanosarcina thermophila |
| purT | AAC74919.1 | 1788155 | Escherichia coli |
| buk1 | NP_349675 | 15896326 | Clostridium acetobutylicum |
| buk2 | Q97II1 | 20137415 | Clostridium acetobutylicum |

The formation of acetyl-CoA from acetylphosphate is catalyzed by phosphotransacetylase (EC 2.3.1.8). The pta gene from E. coli encodes an enzyme that reversibly converts acetyl-CoA into acetyl-phosphate (Suzuki, T., Biochim. Biophys. Acta 191:559-569 (969)). Additional acetyltransferase enzymes have been characterized in Bacillus subtilis (Rado and Hoch, Biochim. Biophys. Acta 321:114-125 (1973), Clostridium kluyveri (Stadtman, E., Methods Enzymol. 1:5896-599 (1955), and Thermotoga maritima (Bock et al., J. Bacteriol. 181:1861-1867 (1999)). This reaction is also catalyzed by some phosphotranbutyrylase enzymes (EC 2.3.1.19) including the ptb gene products from Clostridium acetobutylicum (Wiesenbom et al., App. Environ. Microbiol. 55:317-322 (1989); Walter et al., Gene 134:107-111 (1993)). Additional ptb genes are found in butyrate-producing bacterium L2-50 (Louis et al., J. Bacteriol. 186:2099-2106 (2004) and Bacillus megaterium (Vazquez et al., Curr. Microbiol. 42:345-349 (2001).

| Protein | GenBank ID | GI Number | Organism |
|---|---|---|---|
| Pta | NP_416800.1 | 71152910 | Escherichia coli |
| Pta | P39646 | 730415 | Bacillus subtilis |
| Pta | A5N801 | 146346896 | Clostridium kluyveri |
| Pta | Q9X0L4 | 6685776 | Thermotoga maritima |
| Ptb | NP_349676 | 34540484 | Clostridium acetobutylicum |
| Ptb | AAR19757.1 | 38425288 | butyrate-producing bacterium |
| | | | L2-50 |
| Ptb | CAC07932.1 | 10046659 | Bacillus megaterium |

The acylation of acetate to acetyl-CoA is catalyzed by enzymes with acetyl-CoA synthetase activity. Two enzymes that catalyze this reaction are AMP-forming acetyl-CoA synthetase (EC 6.2.1.1) and ADP-forming acetyl-CoA synthetase (EC 6.2.1.13). AMP-forming acetyl-CoA synthetase (ACS) is the predominant enzyme for activation of acetate to acetyl-CoA. Exemplary ACS enzymes are found in E. coli (Brown et al., J. Gen. Microbiol. 102:327-336 (1977)), Ralstonia eutropha (Priefert and Steinbuchel, J. Bacteriol. 174:6590-6599 (1992)), Methanothermobacter thermautotrophicus (Ingram-Smith and Smith, Archaea 2:95-107 (2007)), Salmonella enterica (Gulick et al., Biochemistry 42:2866-2873 (2003)) and Saccharomyces cerevisiae (Jogl and Tong, Biochemistry 43:1425-1431 (2004)). ADP-forming acetyl-CoA synthetases are reversible enzymes with a generally broad substrate range (Musfeldt and Schonheit, J. Bacteriol. 184:636-644 (2002)). Two isozymes of ADP-forming acetyl-CoA synthetases are encoded in the Archaeoglobus fulgidus genome by are encoded by AF1211 and AF1983 (Musfeldt and Schonheit, supra (2002)). The enzyme from Haloarcula marismortui (annotated as a succinyl-CoA synthetase) also accepts acetate as a substrate and reversibility of the enzyme was demonstrated (Brasen and Schonheit, Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetate, isobutyryl-CoA (preferred substrate) and phenylacetyl-CoA (Brasen and Schonheit, supra (2004)). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen and Schonheit, supra (2004); Musfeldt and Schonheit, supra (2002)). Additional candidates include the succinyl-CoA synthetase encoded by sucCD in E. coli (Buck et al., Biochemistry 24:6245-6252 (1985)) and the acyl-CoA ligase from Pseudomonas putida (Fernandez-Valverde et al., Appl. Environ. Microbiol. 59:1149-1154 (1993)). The aforementioned proteins are tabulated below.

| Protein | GenBank ID | GI Number | Organism |
|---|---|---|---|
| acs | AAC77039.1 | 1790505 | Escherichia coli |
| acoE | AAA21945.1 | 141890 | Ralstonia eutropha |
| acs1 | ABC87079.1 | 86169671 | Methanothermobacter |
| | | | thermautotrophicus |
| acs1 | AAL23099.1 | 16422835 | Salmonella enterica |
| ACS1 | Q01574.2 | 257050994 | Saccharomyces cerevisiae |
| AF1211 | NP_070039.1 | 11498810 | Archaeoglobus fulgidus |
| AF1983 | NP_070807.1 | 11499565 | Archaeoglobus fulgidus |
| scs | YP_135572.1 | 55377722 | Haloarcula marismortui |
| PAE3250 | NP_560604.1 | 18313937 | Pyrobaculum aerophilum str. |
| | | | IM2 |
| sucC | NP_415256.1 | 16128703 | Escherichia coli |
| sucD | AAC73823.1 | 1786949 | Escherichia coli |
| paaF | AAC24333.2 | 22711873 | Pseudomonas putida |

The product yields per C-mol of substrate of microbial cells synthesizing reduced fermentation products such as cyclohexanone, are limited by insufficient reducing equivalents in the carbohydrate feedstock. Reducing equivalents, or electrons, can be extracted from synthesis gas components such as CO and H₂ using carbon monoxide dehydrogenase (CODH) and hydrogenase enzymes, respectively. The reducing equivalents are then passed to acceptors such as oxidized ferredoxins, oxidized quinones, oxidized cytochromes, NAD(P)+, water, or hydrogen peroxide to form reduced ferredoxin, reduced quinones, reduced cytochromes, NAD(P)H, H₂, or water, respectively. Reduced ferredoxin and NAD(P)H are particularly useful as they can serve as redox carriers for various Wood-Ljungdahl pathway and reductive TCA cycle enzymes.

Here, we show specific examples of how additional redox availability from CO and/or H₂ can improve the yields of reduced products such as cyclohexanone.

When both feedstocks of sugar and syngas are available, the syngas components CO and H₂ can be utilized to generate reducing equivalents by employing the hydrogenase and CO dehydrogenase. The reducing equivalents generated from syngas components will be utilized to power the glucose to cyclohexanone production pathways. Theoretically, all carbons in glucose will be conserved, thus resulting in a maximal theoretical yield to produce cyclohexanone from glucose.

As shown in above example, a combined feedstock strategy where syngas is combined with a sugar-based feedstock or other carbon substrate can greatly improve the theoretical yields. In this co-feeding appoach, syngas components H₂ and CO can be utilized by the hydrogenase and CO dehydrogenase to generate reducing equivalents, that can be used to power chemical production pathways in which the carbons from sugar or other carbon substrates will be maximally conserved and the theoretical yields improved. In case of cyclohexanone production from glucose or sugar, the theoretical yields improve from XX mol cyclohexanone per mol of glucose to YY mol cyclohexanone per mol of glucose. Such improvements provide environmental and economic benefits and greatly enhance sustainable chemical production.

Herein below the enzymes and the corresponding genes used for extracting redox from syngas components are described. CODH is a reversible enzyme that interconverts CO and CO₂ at the expense or gain of electrons. The natural physiological role of the CODH in ACS/CODH complexes is to convert CO₂ to CO for incorporation into acetyl-CoA by acetyl-CoA synthase. Nevertheless, such CODH enzymes are suitable for the extraction of reducing equivalents from CO due to the reversible nature of such enzymes. Expressing such CODH enzymes in the absence of ACS allows them to operate in the direction opposite to their natural physiological role (i.e., CO oxidation).

In M. thermoacetica, C. hydrogenoformans, C. carboxidivorans P7, and several other organisms, additional CODH encoding genes are located outside of the ACS/CODH operons. These enzymes provide a means for extracting electrons (or reducing equivalents) from the conversion of carbon monoxide to carbon dioxide. The M. thermoacetica gene (Genbank Accession Number: YP 430813) is expressed by itself in an operon and is believed to transfer electrons from CO to an external mediator like ferredoxin in a “Ping-pong” reaction. The reduced mediator then couples to other reduced nicolinamide adenine dinucleotide phosphate (NAD(P)H) carriers or ferredoxin-dependent cellular processes (Ragsdale, Annals of the New York Academy of Sciences 1125: 129-136 (2008)). The genes encoding the C. hydrogenoformans CODH-II and CooF, a neighboring protein, were cloned and sequenced (Gonzalez and Robb, FEMS Microbiol Lett. 191:243-247 (2000)). The resulting complex was membrane-bound, although cytoplasmic fractions of CODH-II were shown to catalyze the formation of NADPH suggesting an anabolic role (Svetlitchnyi et al., J Bacteriol. 183:5134-5144 (2001)). The crystal structure of the CODH-II is also available (Dobbek et al., Science 293:1281-1285 (2001)). Similar ACS-free CODH enzymes can be found in a diverse array of organisms including Geobacter metallireducens GS-15, Chlorobium phaeobacteroides DSM 266, Clostridium cellulolyticum H10, Desulfovibrio desulfuricans subsp. desulfuricans str. ATCC 27774, Pelobacter carbinolicus DSM 2380, and Campylobacter curvus 525.92.

| Protein | GenBank ID | GI Number | Organism |
|---|---|---|---|
| CODH (putative) | YP_430813 | 83590804 | Moorella thermoacetica |
| CODH-II (CooS- | YP_358957 | 78044574 | Carboxydothermus |
| II) | | | hydrogenoformans |
| CooF | YP_358958 | 78045112 | Carboxydothermus |
| | | | hydrogenoformans |
| CODH (putative) | ZP_05390164.1 | 255523193 | Clostridium carboxidivorans P7 |
| CcarbDRAFT_0341 | ZP_05390341.1 | 255523371 | Clostridium carboxidivorans P7 |
| CcarbDRAFT_1756 | ZP_05391756.1 | 255524806 | Clostridium carboxidivorans P7 |
| CcarbDRAFT_2944 | ZP_05392944.1 | 255526020 | Clostridium carboxidivorans P7 |
| CODH | YP_384856.1 | 78223109 | Geobacter metallireducens GS-15 |
| Cpha266_0148 | YP_910642.1 | 119355998 | Chlorobium |
| (cytochrome c) | | | phaeobacteroides DSM 266 |
| Cpha266_0149 | YP_910643.1 | 119355999 | Chlorobium |
| (CODH) | | | phaeobacteroides DSM 266 |
| Ccel_0438 | YP_002504800.1 | 220927891 | Clostridium cellulolyticum H10 |
| Ddes_0382 | YP_002478973.1 | 220903661 | Desulfovibrio desulfuricans subsp. |
| (CODH) | | | desulfuricans str. ATCC 27774 |
| Ddes_0381 | YP_002478972.1 | 220903660 | Desulfovibrio desulfuricans subsp. |
| (CooC) | | | desulfuricans str. ATCC 27774 |
| Pcar_0057 | YP_355490.1 | 7791767 | Pelobacter carbinolicus DSM |
| (CODH) | | | 2380 |
| Pcar_0058 | YP_355491.1 | 7791766 | Pelobacter carbinolicus DSM |
| (CooC) | | | 2380 |
| Pcar_0058 | YP_355492.1 | 7791765 | Pelobacter carbinolicus DSM |
| (HypA) | | | 2380 |
| CooS (CODH) | YP_001407343.1 | 154175407 | Campylobacter curvus 525.92 |

In some cases, hydrogenase encoding genes are located adjacent to a CODH. In Rhodospirillum rubrum, the encoded CODH/hydrogenase proteins form a membrane-bound enzyme complex that has been indicated to be a site where energy, in the form of a proton gradient, is generated from the conversion of CO and H₂O to CO₂ and H₂ (Fox et al., J Bacteriol. 178:6200-6208 (1996)). The CODH-I of C. hydrogenoformans and its adjacent genes have been proposed to catalyze a similar functional role based on their similarity to the R. rubrum CODH/hydrogenase gene cluster (Wu et al., PLoS Genet. 1:e65 (2005)). The C. hydrogenoformans CODH-I was also shown to exhibit intense CO oxidation and CO₂ reduction activities when linked to an electrode (Parkin et al., J Am. Chem. Soc. 129:10328-10329 (2007)). The protein sequences of exemplary CODH and hydrogenase genes can be identified by the following GenBank accession numbers.

| Protein | GenBank ID | GI Number | Organism |
|---|---|---|---|
| CODH-I | YP_360644 | 78043418 | Carboxydothermus |
| (CooS-I) | | | hydrogenoformans |
| CooF | YP_360645 | 78044791 | Carboxydothermus |
| | | | hydrogenoformans |
| HypA | YP_360646 | 78044340 | Carboxydothermus |
| | | | hydrogenoformans |
| CooH | YP_360647 | 78043871 | Carboxydothermus |
| | | | hydrogenoformans |
| CooU | YP_360648 | 78044023 | Carboxydothermus |
| | | | hydrogenoformans |
| CooX | YP_360649 | 78043124 | Carboxydothermus |
| | | | hydrogenoformans |
| CooL | YP_360650 | 78043938 | Carboxydothermus |
| | | | hydrogenoformans |
| CooK | YP_360651 | 78044700 | Carboxydothermus |
| | | | hydrogenoformans |
| CooM | YP_360652 | 78043942 | Carboxydothermus |
| | | | hydrogenoformans |
| CooC | YP_360654.1 | 78043296 | Carboxydothermus |
| | | | hydrogenoformans |
| CooA-1 | YP_360655.1 | 78044021 | Carboxydothermus |
| | | | hydrogenoformans |
| CooL | AAC45118 | 1515468 | Rhodospirillum rubrum |
| CooX | AAC45119 | 1515469 | Rhodospirillum rubrum |
| CooU | AAC45120 | 1515470 | Rhodospirillum rubrum |
| CooH | AAC45121 | 1498746 | Rhodospirillum rubrum |
| CooF | AAC45122 | 1498747 | Rhodospirillum rubrum |
| CODH (CooS) | AAC45123 | 1498748 | Rhodospirillum rubrum |
| CooC | AAC45124 | 1498749 | Rhodospirillum rubrum |
| CooT | AAC45125 | 1498750 | Rhodospirillum rubrum |
| CooJ | AAC45126 | 1498751 | Rhodospirillum rubrum |

Native to E. coli and other enteric bacteria are multiple genes encoding up to four hydrogenases (Sawers, G., Antonie Van Leeuwenhoek 66:57-88 (1994); Sawers et al., J Bacteriol. 164:1324-1331 (1985); Sawers and Boxer, Eur. J Biochem. 156:265-275 (1986); Sawers et al., J Bacteriol. 168:398-404 (1986)). Given the multiplicity of enzyme activities, E. coli or another host organism can provide sufficient hydrogenase activity to split incoming molecular hydrogen and reduce the corresponding acceptor. E. coli possesses two uptake hydrogenases, Hyd-1 and Hyd-2, encoded by the hyaABCDEF and hybOABCDEFG gene clusters, respectively (Lukey et al., How E. coli is equipped to oxidize hydrogen under different redox conditions, J Biol Chem published online Nov. 16, 2009). Hyd-1 is oxygen-tolerant, irreversible, and is coupled to quinone reduction via the hyaC cytochrome. Hyd-2 is sensitive to O₂, reversible, and transfers electrons to the periplasmic ferredoxin hybA which, in turn, reduces a quinone via the hybB integral membrane protein. Reduced quinones can serve as the source of electrons for fumarate reductase in the reductive branch of the TCA cycle. Reduced ferredoxins can be used by enzymes such as NAD(P)H:ferredoxin oxidoreductases to generate NADPH or NADH. They can alternatively be used as the electron donor for reactions such as pyruvate ferredoxin oxidoreductase, AKG ferredoxin oxidoreductase, and 5,10-methylene-H4folate reductase.

| Protein | GenBank ID | GI Number | Organism |
|---|---|---|---|
| HyaA | AAC74057.1 | 1787206 | Escherichia coli |
| HyaB | AAC74058.1 | 1787207 | Escherichia coli |
| HyaC | AAC74059.1 | 1787208 | Escherichia coli |
| HyaD | AAC74060.1 | 1787209 | Escherichia coli |
| HyaE | AAC74061.1 | 1787210 | Escherichia coli |
| HyaF | AAC74062.1 | 1787211 | Escherichia coli |

| Protein | GenBank ID | GI Number | Organism |
|---|---|---|---|
| HybO | AAC76033.1 | 1789371 | Escherichia coli |
| HybA | AAC76032.1 | 1789370 | Escherichia coli |
| HybB | AAC76031.1 | 2367183 | Escherichia coli |
| HybC | AAC76030.1 | 1789368 | Escherichia coli |
| HybD | AAC76029.1 | 1789367 | Escherichia coli |
| HybE | AAC76028.1 | 1789366 | Escherichia coli |
| HybF | AAC76027.1 | 1789365 | Escherichia coli |
| HybG | AAC76026.1 | 1789364 | Escherichia coli |

The hydrogen-lyase systems of E. coli include hydrogenase 3, a membrane-bound enzyme complex using ferredoxin as an acceptor, and hydrogenase 4 that also uses a ferredoxin acceptor. Hydrogenase 3 and 4 are encoded by the hyc and hyf gene clusters, respectively. Hydrogenase 3 has been shown to be a reversible enzyme (Maeda et al., Appl Microbiol Biotechnol 76(5):1035-42 (2007)). Hydrogenase activity in E. coli is also dependent upon the expression of the hyp genes whose corresponding proteins are involved in the assembly of the hydrogenase complexes (Jacobi et al., Arch. Microbiol 158:444-451 (1992); Rangarajan et al., J. Bacteriol. 190:1447-1458 (2008)).

| Protein | GenBank ID | GI Number | Organism |
|---|---|---|---|
| HycA | NP_417205 | 16130632 | Escherichia coli |
| HycB | NP_417204 | 16130631 | Escherichia coli |
| HycC | NP_417203 | 16130630 | Escherichia coli |
| HycD | NP_417202 | 16130629 | Escherichia coli |
| HycE | NP_417201 | 16130628 | Escherichia coli |
| HycF | NP_417200 | 16130627 | Escherichia coli |
| HycG | NP_417199 | 16130626 | Escherichia coli |
| HycH | NP_417198 | 16130625 | Escherichia coli |
| HycI | NP_417197 | 16130624 | Escherichia coli |

| Protein | GenBank ID | GI Number | Organism |
|---|---|---|---|
| HyfA | NP_416976 | 90111444 | Escherichia coli |
| HyfB | NP_416977 | 16130407 | Escherichia coli |
| HyfC | NP_416978 | 90111445 | Escherichia coli |
| HyfD | NP_416979 | 16130409 | Escherichia coli |
| HyfE | NP_416980 | 16130410 | Escherichia coli |
| HyfF | NP_416981 | 16130411 | Escherichia coli |
| HyfG | NP_416982 | 16130412 | Escherichia coli |
| HyfH | NP_416983 | 16130413 | Escherichia coli |
| HyfI | NP_416984 | 16130414 | Escherichia coli |
| HyfJ | NP_416985 | 90111446 | Escherichia coli |
| HyfR | NP_416986 | 90111447 | Escherichia coli |

| Protein | GenBank ID | GI Number | Organism |
|---|---|---|---|
| HypA | NP_417206 | 16130633 | Escherichia coli |
| HypB | NP_417207 | 16130634 | Escherichia coli |
| HypC | NP_417208 | 16130635 | Escherichia coli |
| HypD | NP_417209 | 16130636 | Escherichia coli |
| HypE | NP_417210 | 226524740 | Escherichia coli |
| HypF | NP_417192 | 16130619 | Escherichia coli |

The M. thermoacetica hydrogenases are suitable for a host that lacks sufficient endogenous hydrogenase activity. M. thermoacetica can grow with CO₂ as the exclusive carbon source indicating that reducing equivalents are extracted from H₂ to enable acetyl-CoA synthesis via the Wood-Ljungdahl pathway (Drake, H. L., J. Bacteriol. 150:702-709 (1982); Drake and Daniel, Res. Microbiol. 155:869-883 (2004); Kellum and Drake, J. Bacteriol. 160:466-469 (1984)) (see FIG. 2A). M. thermoacetica has homologs to several hyp, hyc, and hyf genes from E. coli. The protein sequences encoded for by these genes are identified by the following GenBank accession numbers.

Proteins in M. thermoacetica whose genes are homologous to the E. coli hyp genes are shown below.

| Protein | GenBank ID | GI Number | Organism |
|---|---|---|---|
| Moth_2175 | YP_431007 | 83590998 | Moorella thermoacetica |
| Moth_2176 | YP_431008 | 83590999 | Moorella thermoacetica |
| Moth_2177 | YP_431009 | 83591000 | Moorella thermoacetica |
| Moth_2178 | YP_431010 | 83591001 | Moorella thermoacetica |
| Moth_2179 | YP_431011 | 83591002 | Moorella thermoacetica |
| Moth_2180 | YP_431012 | 83591003 | Moorella thermoacetica |
| Moth_2181 | YP_431013 | 83591004 | Moorella thermoacetica |

Proteins in M. thermoacetica that are homologous to the E. coli Hydrogenase 3 and/or 4 proteins are listed in the following table.

| Protein | GenBank ID | GI Number | Organism |
|---|---|---|---|
| Moth_2182 | YP_431014 | 83591005 | Moorella thermoacetica |
| Moth_2183 | YP_431015 | 83591006 | Moorella thermoacetica |
| Moth_2184 | YP_431016 | 83591007 | Moorella thermoacetica |
| Moth_2185 | YP_431017 | 83591008 | Moorella thermoacetica |
| Moth_2186 | YP_431018 | 83591009 | Moorella thermoacetica |
| Moth_2187 | YP_431019 | 83591010 | Moorella thermoacetica |
| Moth_2188 | YP_431020 | 83591011 | Moorella thermoacetica |
| Moth_2189 | YP_431021 | 83591012 | Moorella thermoacetica |
| Moth_2190 | YP_431022 | 83591013 | Moorella thermoacetica |
| Moth_2191 | YP_431023 | 83591014 | Moorella thermoacetica |
| Moth_2192 | YP_431024 | 83591015 | Moorella thermoacetica |

In addition, several gene clusters encoding hydrogenase functionality are present in M. thermoacetica and their corresponding protein sequences are provided below.

| Protein | GenBank ID | GI Number | Organism |
|---|---|---|---|
| Moth_0439 | YP_429313 | 83589304 | Moorella thermoacetica |
| Moth_0440 | YP_429314 | 83589305 | Moorella thermoacetica |
| Moth_0441 | YP_429315 | 83589306 | Moorella thermoacetica |
| Moth_0442 | YP_429316 | 83589307 | Moorella thermoacetica |
| Moth_0809 | YP_429670 | 83589661 | Moorella thermoacetica |
| Moth_0810 | YP_429671 | 83589662 | Moorella thermoacetica |
| Moth_0811 | YP_429672 | 83589663 | Moorella thermoacetica |
| Moth_0812 | YP_429673 | 83589664 | Moorella thermoacetica |
| Moth_0814 | YP_429674 | 83589665 | Moorella thermoacetica |
| Moth_0815 | YP_429675 | 83589666 | Moorella thermoacetica |
| Moth_0816 | YP_429676 | 83589667 | Moorella thermoacetica |
| Moth_1193 | YP_430050 | 83590041 | Moorella thermoacetica |
| Moth_1194 | YP_430051 | 83590042 | Moorella thermoacetica |
| Moth_1195 | YP_430052 | 83590043 | Moorella thermoacetica |
| Moth_1196 | YP_430053 | 83590044 | Moorella thermoacetica |
| Moth_1717 | YP_430562 | 83590553 | Moorella thermoacetica |
| Moth_1718 | YP_430563 | 83590554 | Moorella thermoacetica |
| Moth_1719 | YP_430564 | 83590555 | Moorella thermoacetica |
| Moth_1883 | YP_430726 | 83590717 | Moorella thermoacetica |
| Moth_1884 | YP_430727 | 83590718 | Moorella thermoacetica |
| Moth_1885 | YP_430728 | 83590719 | Moorella thermoacetica |
| Moth_1886 | YP_430729 | 83590720 | Moorella thermoacetica |
| Moth_1887 | YP_430730 | 83590721 | Moorella thermoacetica |
| Moth_1888 | YP_430731 | 83590722 | Moorella thermoacetica |
| Moth_1452 | YP_430305 | 83590296 | Moorella thermoacetica |
| Moth_1453 | YP_430306 | 83590297 | Moorella thermoacetica |
| Moth_1454 | YP_430307 | 83590298 | Moorella thermoacetica |

Ralstonia eutropha H16 uses hydrogen as an energy source with oxygen as a terminal electron acceptor. Its membrane-bound uptake [NiFe]-hydrogenase is an “O2-tolerant” hydrogenase (Cracknell, et al. Proc Nat Acad Sci, 106(49) 20681-20686 (2009)) that is periplasmically-oriented and connected to the respiratory chain via a b-type cytochrome (Schink and Schlegel, Biochim. Biophys. Acta, 567, 315-324 (1979); Bernhard et al., Eur. J. Biochem. 248, 179-186 (1997)). R. eutropha also contains an O₂-tolerant soluble hydrogenase encoded by the Hox operon which is cytoplasmic and directly reduces NAD+ at the expense of hydrogen (Schneider and Schlegel, Biochim. Biophys. Acta 452, 66-80 (1976); Burgdorf, J. Bact. 187(9) 3122-3132 (2005)). Soluble hydrogenase enzymes are additionally present in several other organisms including Geobacter sulfurreducens (Coppi, Microbiology 151, 1239-1254 (2005)), Synechocystis str. PCC 6803 (Germer, J. Biol. Chem., 284(52), 36462-36472 (2009)), and Thiocapsa roseopersicina (Rakhely, Appl. Environ. Microbiol. 70(2) 722-728 (2004)). The Synechocystis enzyme is capable of generating NADPH from hydrogen. Overexpression of both the Hox operon from Synechocystis str. PCC 6803 and the accessory genes encoded by the Hyp operon from Nostoc sp. PCC 7120 led to increased hydrogenase activity compared to expression of the Hox genes alone (Germer, J. Biol. Chem. 284(52), 36462-36472 (2009)).

| Protein | GenBank ID | GI Number | Organism |
|---|---|---|---|
| HoxF | NP_942727.1 | 38637753 | Ralstonia eutropha H16 |
| HoxU | NP_942728.1 | 38637754 | Ralstonia eutropha H16 |
| HoxY | NP_942729.1 | 38637755 | Ralstonia eutropha H16 |
| HoxH | NP_942730.1 | 38637756 | Ralstonia eutropha H16 |
| HoxW | NP_942731.1 | 38637757 | Ralstonia eutropha H16 |
| HoxI | NP_942732.1 | 38637758 | Ralstonia eutropha H16 |
| HoxE | NP_953767.1 | 39997816 | Geobacter sulfurreducens |
| HoxF | NP_953766.1 | 39997815 | Geobacter sulfurreducens |
| HoxU | NP_953765.1 | 39997814 | Geobacter sulfurreducens |
| HoxY | NP_953764.1 | 39997813 | Geobacter sulfurreducens |
| HoxH | NP_953763.1 | 39997812 | Geobacter sulfurreducens |
| GSU2717 | NP_953762.1 | 39997811 | Geobacter sulfurreducens |
| HoxE | NP_441418.1 | 16330690 | Synechocystis str. PCC |
| | | | 6803 |
| HoxF | NP_441417.1 | 16330689 | Synechocystis str. PCC |
| | | | 6803 |
| Unknown | NP_441416.1 | 16330688 | Synechocystis str. PCC |
| function | | | 6803 |
| HoxU | NP_441415.1 | 16330687 | Synechocystis str. PCC |
| | | | 6803 |
| HoxY | NP_441414.1 | 16330686 | Synechocystis str. PCC |
| | | | 6803 |
| Unknown | NP_441413.1 | 16330685 | Synechocystis str. PCC |
| function | | | 6803 |
| Unknown | NP_441412.1 | 16330684 | Synechocystis str. PCC |
| function | | | 6803 |
| HoxH | NP_441411.1 | 16330683 | Synechocystis str. PCC |
| | | | 6803 |
| HypF | NP_484737.1 | 17228189 | Nostoc sp. PCC 7120 |
| HypC | NP_484738.1 | 17228190 | Nostoc sp. PCC 7120 |
| HypD | NP_484739.1 | 17228191 | Nostoc sp. PCC 7120 |
| Unknown | NP_484740.1 | 17228192 | Nostoc sp. PCC 7120 |
| function | | | |
| HypE | NP_484741.1 | 17228193 | Nostoc sp. PCC 7120 |
| HypA | NP_484742.1 | 17228194 | Nostoc sp. PCC 7120 |
| HypB | NP_484743.1 | 17228195 | Nostoc sp. PCC 7120 |
| Hox1E | AAP50519.1 | 37787351 | Thiocapsa roseopersicina |
| Hox1F | AAP50520.1 | 37787352 | Thiocapsa roseopersicina |
| Hox1U | AAP50521.1 | 37787353 | Thiocapsa roseopersicina |
| Hox1Y | AAP50522.1 | 37787354 | Thiocapsa roseopersicina |
| Hox1H | AAP50523.1 | 37787355 | Thiocapsa roseopersicina |

Several enzymes and the corresponding genes used for fixing carbon dioxide to either pyruvate or phosphoenolpyruvate to form the TCA cycle intermediates, oxaloacetate or malate are described below.

Carboxylation of phosphoenolpyruvate to oxaloacetate is catalyzed by phosphoenolpyruvate carboxylase. Exemplary PEP carboxylase enzymes are encoded by ppc in E. coli (Kai et al., Arch. Biochem. Biophys. 414:170-179 (2003), ppcA in Methylobacterium extorquens AM1 (Arps et al., J. Bacteriol. 175:3776-3783 (1993), and ppc in Corynebacterium glutamicum (Eikmanns et al., Mol. Gen. Genet. 218:330-339 (1989).

| Protein | GenBank ID | GI Number | Organism |
|---|---|---|---|
| Ppc | NP_418391 | 16131794 | Escherichia coli |
| ppcA | AAB58883 | 28572162 | Methylobacterium extorquens |
| Ppc | ABB53270 | 80973080 | Corynebacterium glutamicum |

An alternative enzyme for converting phosphoenolpyruvate to oxaloacetate is PEP carboxykinase, which simultaneously fauns an ATP while carboxylating PEP. In most organisms PEP carboxykinase serves a gluconeogenic function and converts oxaloacetate to PEP at the expense of one ATP. S. cerevisiae is one such organism whose native PEP carboxykinase, PCK1, serves a gluconeogenic role (Valdes-Hevia et al., FEBS Lett. 258:313-316 (1989). E. coli is another such organism, as the role of PEP carboxykinase in producing oxaloacetate is believed to be minor when compared to PEP carboxylase, which does not form ATP, possibly due to the higher Kₘ for bicarbonate of PEP carboxykinase (Kim et al., Appl. Environ. Microbiol. 70:1238-1241 (2004)). Nevertheless, activity of the native E. coli PEP carboxykinase from PEP towards oxaloacetate has been recently demonstrated in ppc mutants of E. coli K-12 (Kwon et al., J. Microbiol. Biotechnol. 16:1448-1452 (2006)). These strains exhibited no growth defects and had increased succinate production at high NaHCO₃ concentrations. Mutant strains of E. coli can adopt Pck as the dominant CO2-fixing enzyme following adaptive evolution (Zhang et al. 2009). In some organisms, particularly rumen bacteria, PEP carboxykinase is quite efficient in producing oxaloacetate from PEP and generating ATP. Examples of PEP carboxykinase genes that have been cloned into E. coli include those from Mannheimia succiniciproducens (Lee et al., Biotechnol. Bioprocess Eng. 7:95-99 (2002)), Anaerobiospirillum succiniciproducens (Laivenieks et al., Appl. Environ. Microbiol. 63:2273-2280 (1997), and Actinobacillus succinogenes (Kim et al. supra). The PEP carboxykinase enzyme encoded by Haemophilus influenza is effective at forming oxaloacetate from PEP.

| Protein | GenBank ID | GI Number | Organism |
|---|---|---|---|
| PCK1 | NP_013023 | 6322950 | Saccharomyces cerevisiae |
| pck | NP_417862.1 | 16131280 | Escherichia coli |
| pckA | YP_089485.1 | 52426348 | Mannheimia succiniciproducens |
| pckA | O09460.1 | 3122621 | Anaerobiospirillum |
| | | | succiniciproducens |
| pckA | Q6W6X5 | 75440571 | Actinobacillus succinogenes |
| pckA | P43923.1 | 1172573 | Haemophilus influenza |

Pyruvate carboxylase (EC 6.4.1.1) directly converts pyruvate to oxaloacetate at the cost of one ATP. Pyruvate carboxylase enzymes are encoded by PYC1 (Walker et al., Biochem. Biophys. Res. Commun. 176:1210-1217 (1991) and PYC2 (Walker et al., supra) in Saccharomyces cerevisiae, and pyc in Mycobacterium smegmatis (Mukhopadhyay and Purwantini, Biochim. Biophys. Acta 1475:191-206 (2000)).

| Protein | GenBank ID | GI Number | Organism |
|---|---|---|---|
| PYC1 | NP_011453 | 6321376 | Saccharomyces cerevisiae |
| PYC2 | NP_009777 | 6319695 | Saccharomyces cerevisiae |
| Pyc | YP_890857.1 | 118470447 | Mycobacterium smegmatis |

Malic enzyme can be applied to convert CO₂ and pyruvate to malate at the expense of one reducing equivalent. Malic enzymes for this purpose can include, without limitation, malic enzyme (NAD-dependent) and malic enzyme (NADP-dependent). For example, one of the E. coli malic enzymes (Takeo, J. Biochem. 66:379-387 (1969)) or a similar enzyme with higher activity can be expressed to enable the conversion of pyruvate and CO₂ to malate. By fixing carbon to pyruvate as opposed to PEP, malic enzyme allows the high-energy phosphate bond from PEP to be conserved by pyruvate kinase whereby ATP is generated in the formation of pyruvate or by the phosphotransferase system for glucose transport. Although malic enzyme is typically assumed to operate in the direction of pyruvate formation from malate, overexpression of the NAD-dependent enzyme, encoded by maeA, has been demonstrated to increase succinate production in E. coli while restoring the lethal Δpfl-ΔldhA phenotype under anaerobic conditions by operating in the carbon-fixing direction (Stols and Donnelly, Appl. Environ. Microbiol. 63(7) 2695-2701 (1997)). A similar observation was made upon overexpressing the malic enzyme from Ascaris suum in E. coli (Stols et al., Appl. Biochem. Biotechnol. 63-65(1), 153-158 (1997)). The second E. coli malic enzyme, encoded by maeB, is NADP-dependent and also decarboxylates oxaloacetate and other alpha-keto acids (Iwakura et al., J. Biochem. 85(5):1355-65 (1979)).

| Protein | GenBank ID | GI Number | Organism |
|---|---|---|---|
| maeA | NP_415996 | 90111281 | Escherichia coli |
| maeB | NP_416958 | 16130388 | Escherichia coli |
| NAD-ME | P27443 | 126732 | Ascaris suum |

The enzymes used for converting oxaloacetate (foamed from, for example, PEP carboxylase, PEP carboxykinase, or pyruvate carboxylase) or malate (formed from, for example, malic enzyme or malate dehydrogenase) to succinyl-CoA via the reductive branch of the TCA cycle are malate dehydrogenase, fumarate dehydratase (fumarase), fumarate reductase, and succinyl-CoA transferase. The genes for each of the enzymes are described herein above.

Enzymes, genes and methods for engineering pathways from succinyl-CoA to various products into a microorganism are now known in the art. The additional reducing equivalents obtained from CO and/or H₂, as disclosed herein, improve the yields of cyclohexanone when utilizing carbohydrate-based feedstock.

Enzymes, genes and methods for engineering pathways from glycolysis intermediates to various products into a microorganism are known in the art. The additional reducing equivalents obtained from CO and H₂, as described herein, improve the yields of all these products on carbohydrates. For example, cyclohexanone can be produced from the glycolysis intermediate, acetyl-CoA.

### Example VII

### Methods for Handling CO and Anaerobic Cultures

This example describes methods used in handling CO and anaerobic cultures.

A. Handling of CO in Small Quantities for Assays and Small Cultures.

CO is an odorless, colorless and tasteless gas that is a poison. Therefore, cultures and assays that utilized CO required special handling. Several assays, including CO oxidation, acetyl-CoA synthesis, CO concentration using myoglobin, and CO tolerance/utilization in small batch cultures, called for small quantities of the CO gas that were dispensed and handled within a fume hood. Biochemical assays called for saturating very small quantities (<2 mL) of the biochemical assay medium or buffer with CO and then performing the assay. All of the CO handling steps were performed in a fume hood with the sash set at the proper height and blower turned on; CO was dispensed from a compressed gas cylinder and the regulator connected to a Schlenk line. The latter ensures that equal concentrations of CO were dispensed to each of several possible cuvettes or vials. The Schlenk line was set up containing an oxygen scrubber on the input side and an oil pressure release bubbler and vent on the other side. Assay cuvettes were both anaerobic and CO-containing. Therefore, the assay cuvettes were tightly sealed with a rubber stopper and reagents were added or removed using gas-tight needles and syringes. Secondly, small (˜50 mL) cultures were grown with saturating CO in tightly stoppered serum bottles. As with the biochemical assays, the CO-saturated microbial cultures were equilibrated in the fume hood using the Schlenk line setup. Both the biochemical assays and microbial cultures were in portable, sealed containers and in small volumes making for safe handling outside of the fume hood. The compressed CO tank was adjacent to the fume hood.

Typically, a Schlenk line was used to dispense CO to cuvettes, each vented. Rubber stoppers on the cuvettes were pierced with 19 or 20 gage disposable syringe needles and were vented with the same. An oil bubbler was used with a CO tank and oxygen scrubber. The glass or quartz spectrophotometer cuvettes have a circular hole on top into which a Kontes stopper sleeve, Sz7 774250-0007 was fitted. The CO detector unit was positioned proximal to the fume hood.

B. Handling of CO in Larger Quantities Fed to Large-Scale Cultures.

Fermentation cultures are fed either CO or a mixture of CO and H₂ to simulate syngas as a feedstock in fermentative production. Therefore, quantities of cells ranging from 1 liter to several liters can include the addition of CO gas to increase the dissolved concentration of CO in the medium. In these circumstances, fairly large and continuously administered quantities of CO gas are added to the cultures. At different points, the cultures are harvested or samples removed. Alternatively, cells are harvested with an integrated continuous flow centrifuge that is part of the fermenter.

The fermentative processes are carried out under anaerobic conditions. In some cases, it is uneconomical to pump oxygen or air into fermenters to ensure adequate oxygen saturation to provide a respiratory environment. In addition, the reducing power generated during anaerobic fermentation may be needed in product formation rather than respiration. Furthermore, many of the enzymes for various pathways are oxygen-sensitive to varying degrees. Classic acetogens such as M. thermoacetica are obligate anaerobes and the enzymes in the Wood-Ljungdahl pathway are highly sensitive to irreversible inactivation by molecular oxygen. While there are oxygen-tolerant acetogens, the repertoire of enzymes in the Wood-Ljungdahl pathway might be incompatible in the presence of oxygen because most are metallo-enzymes, key components are ferredoxins, and regulation can divert metabolism away from the Wood-Ljungdahl pathway to maximize energy acquisition. At the same time, cells in culture act as oxygen scavengers that moderate the need for extreme measures in the presence of large cell growth.

C. Anaerobic Chamber and Conditions.

Exemplary anaerobic chambers are available commercially (see, for example, Vacuum Atmospheres Company, Hawthorne Calif.; MBraun, Newburyport Mass.). Conditions included an O₂ concentration of 1 ppm or less and 1 atm pure N₂. In one example, 3 oxygen scrubbers/catalyst regenerators were used, and the chamber included an O₂ electrode (such as Teledyne; City of Industry Calif.). Nearly all items and reagents were cycled four times in the airlock of the chamber prior to opening the inner chamber door. Reagents with a volume>5 mL were sparged with pure N₂ prior to introduction into the chamber. Gloves are changed twice/yr and the catalyst containers were regenerated periodically when the chamber displays increasingly sluggish response to changes in oxygen levels. The chamber's pressure was controlled through one-way valves activated by solenoids. This feature allowed setting the chamber pressure at a level higher than the surroundings to allow transfer of very small tubes through the purge valve.

The anaerobic chambers achieved levels of O₂ that were consistently very low and were needed for highly oxygen sensitive anaerobic conditions. However, growth and handling of cells does not usually require such precautions. In an alternative anaerobic chamber configuration, platinum or palladium can be used as a catalyst that requires some hydrogen gas in the mix. Instead of using solenoid valves, pressure release can be controlled by a bubbler. Instead of using instrument-based O₂ monitoring, test strips can be used instead.

D. Anaerobic Microbiology.

Small cultures were handled as described above for CO handling. In particular, serum or media bottles are fitted with thick rubber stoppers and aluminum crimps are employed to seal the bottle. Medium, such as Terrific Broth, is made in a conventional manner and dispensed to an appropriately sized serum bottle. The bottles are sparged with nitrogen for ˜30 min of moderate bubbling. This removes most of the oxygen from the medium and, after this step, each bottle is capped with a rubber stopper (such as Bellco 20 mm septum stoppers; Bellco, Vineland, N.J.) and crimp-sealed (Bellco 20 mm). Then the bottles of medium are autoclaved using a slow (liquid) exhaust cycle. At least sometimes a needle can be poked through the stopper to provide exhaust during autoclaving; the needle needs to be removed immediately upon removal from the autoclave. The sterile medium has the remaining medium components, for example buffer or antibiotics, added via syringe and needle. Prior to addition of reducing agents, the bottles are equilibrated for 30-60 minutes with nitrogen (or CO depending upon use). A reducing agent such as a 100×150 mM sodium sulfide, 200 mM cysteine-HCl is added. This is made by weighing the sodium sulfide into a dry beaker and the cysteine into a serum bottle, bringing both into the anaerobic chamber, dissolving the sodium sulfide into anaerobic water, then adding this to the cysteine in the serum bottle. The bottle is stoppered immediately as the sodium sulfide solution generates hydrogen sulfide gas upon contact with the cysteine. When injecting into the culture, a syringe filter is used to sterilize the solution. Other components are added through syringe needles, such as B12 (10 μM cyanocobalamin), nickel chloride (NiCl₂, 20 microM final concentration from a 40 mM stock made in anaerobic water in the chamber and sterilized by autoclaving or by using a syringe filter upon injection into the culture), and ferrous ammonium sulfate (final concentration needed is 100 μM—made as 100-1000× stock solution in anaerobic water in the chamber and sterilized by autoclaving or by using a syringe filter upon injection into the culture). To facilitate faster growth under anaerobic conditions, the 1 liter bottles were inoculated with 50 mL of a preculture grown anaerobically. Induction of the pA1-lacO1 promoter in the vectors was performed by addition of isopropyl β-D-1-thiogalactopyranoside (IPTG) to a final concentration of 0.2 mM and was carried out for about 3 hrs.

Large cultures can be grown in larger bottles using continuous gas addition while bubbling. A rubber stopper with a metal bubbler is placed in the bottle after medium addition and sparged with nitrogen for 30 minutes or more prior to setting up the rest of the bottle. Each bottle is put together such that a sterile filter will sterilize the gas bubbled in and the hoses on the bottles are compressible with small C clamps. Medium and cells are stirred with magnetic stir bars. Once all medium components and cells are added, the bottles are incubated in an incubator in room air but with continuous nitrogen sparging into the bottles.

### Example VIII

### CO Oxidation (CODH) Assay

This example describes assay methods for measuring CO oxidation (CO dehydrogenase; CODH).

The 7 gene CODH/ACS operon of Moorella thermoacetica was cloned into E. coli expression vectors. The intact ˜10 kbp DNA fragment was cloned, and it is likely that some of the genes in this region are expressed from their own endogenous promoters and all contain endogenous ribosomal binding sites. These clones were assayed for CO oxidation, using an assay that quantitatively measures CODH activity. Antisera to the M. thermoacetica gene products was used for Western blots to estimate specific activity. M. thermoacetica is Gram positive, and ribosome binding site elements are expected to work well in E. coli. This activity, described below in more detail, was estimated to be ˜ 1/50th of the M. thermoacetica specific activity. It is possible that CODH activity of recombinant E. coli cells could be limited by the fact that M. thermoacetica enzymes have temperature optima around 55° C. Therefore, a mesophilic CODH/ACS pathway could be advantageous such as the close relative of Moorella that is mesophilic and does have an apparently intact CODH/ACS operon and a Wood-Ljungdahl pathway, Desulfitobacterium hafniense. Acetogens as potential host organisms include, but are not limited to, Rhodospirillum rubrum, Moorella thermoacetica and Desulfitobacterium hafniense.

CO oxidation is both the most sensitive and most robust of the CODH/ACS assays. It is likely that an E. coli-based syngas using system will ultimately need to be about as anaerobic as Clostridial (i.e., Moorella) systems, especially for maximal activity. Improvement in CODH should be possible but will ultimately be limited by the solubility of CO gas in water.

Initially, each of the genes was cloned individually into expression vectors. Combined expression units for multiple subunits/1 complex were generated. Expression in E. coli at the protein level was determined. Both combined M. thermoacetica CODH/ACS operons and individual expression clones were made.

CO oxidation assay. This assay is one of the simpler, reliable, and more versatile assays of enzymatic activities within the Wood-Ljungdahl pathway and tests CODH (Seravalli et al., Biochemistry 43:3944-3955 (2004)). A typical activity of M. thermoacetica CODH specific activity is 500 U at 55° C. or ˜60 U at 25° C. This assay employs reduction of methyl viologen in the presence of CO. This is measured at 578 nm in stoppered, anaerobic, glass cuvettes.

In more detail, glass rubber stoppered cuvettes were prepared after first washing the cuvette four times in deionized water and one time with acetone. A small amount of vacuum grease was smeared on the top of the rubber gasket. The cuvette was gassed with CO, dried 10 min with a 22 Ga. needle plus an exhaust needle. A volume of 0.98 mL of reaction buffer (50 mM Hepes, pH 8.5, 2 mM dithiothreitol (DTT) was added using a 22 Ga. needle, with exhaust needled, and 100% CO. Methyl viologen (CH₃ viologen) stock was 1 M in water. Each assay used 20 microliters for 20 mM final concentration. When methyl viologen was added, an 18 Ga needle (partial) was used as a jacket to facilitate use of a Hamilton syringe to withdraw the CH₃ viologen. 4-5 aliquots were drawn up and discarded to wash and gas equilibrate the syringe. A small amount of sodium dithionite (0.1 M stock) was added when making up the CH₃ viologen stock to slightly reduce the CH₃ viologen. The temperature was equilibrated to 55° C. in a heated Olis spectrophotometer (Bogart Ga.). A blank reaction (CH₃ viologen+buffer) was run first to measure the base rate of CH₃ viologen reduction. Crude E. coli cell extracts of ACS90 and ACS91 (CODH-ACS operon of M. thermoacetica with and without, respectively, the first cooC). 10 microliters of extract were added at a time, mixed and assayed. Reduced CH₃ viologen turns purple. The results of an assay are shown in Table I.

TABLE I
Crude extract CO Oxidation Activities.

| | | | |
|---|---|---|---|
| ACS90 | 7.7 mg/ml | ACS91 | 11.8 mg/ml |
| Mta98 | 9.8 mg/ml | Mta99 | 11.2 mg/ml |

| Extract | Vol | OD/ | U/ml | U/mg |
|---|---|---|---|---|
| ACS90 | 10 microliters | 0.073 | 0.376 | 0.049 |
| ACS91 | 10 microliters | 0.096 | 0.494 | 0.042 |
| Mta99 | 10 microliters | 0.0031 | 0.016 | 0.0014 |
| ACS90 | 10 microliters | 0.099 | 0.51 | 0.066 |
| Mta99 | 25 microliters | 0.012 | 0.025 | 0.0022 |
| ACS91 | 25 microliters | 0.215 | 0.443 | 0.037 |
| Mta98 | 25 microliters | 0.019 | 0.039 | 0.004 |
| ACS91 | 10 microliters | 0.129 | 0.66 | 0.056 |

| Averages | |
|---|---|
| ACS90 | 0.057 U/mg |
| ACS91 | 0.045 U/mg |
| Mta99 | 0.0018 U/mg |

Mta98/Mta99 are E. coli MG1655 strains that express methanol methyltransferase genes from M. thermoacetia and, therefore, are negative controls for the ACS90 ACS91 E. coli strains that contain M. thermoacetica CODH operons.

If ˜1% of the cellular protein is CODH, then these figures would be approximately 100× less than the 500 U/mg activity of pure M. thermoacetica CODH. Actual estimates based on Western blots are 0.5% of the cellular protein, so the activity is about 50× less than for M. thermoacetica CODH. Nevertheless, this experiment demonstrates CO oxidation activity in recombinant E. coli with a much smaller amount in the negative controls. The small amount of CO oxidation (CH₃ viologen reduction) seen in the negative controls indicates that E. coli may have a limited ability to reduce CH₃ viologen.

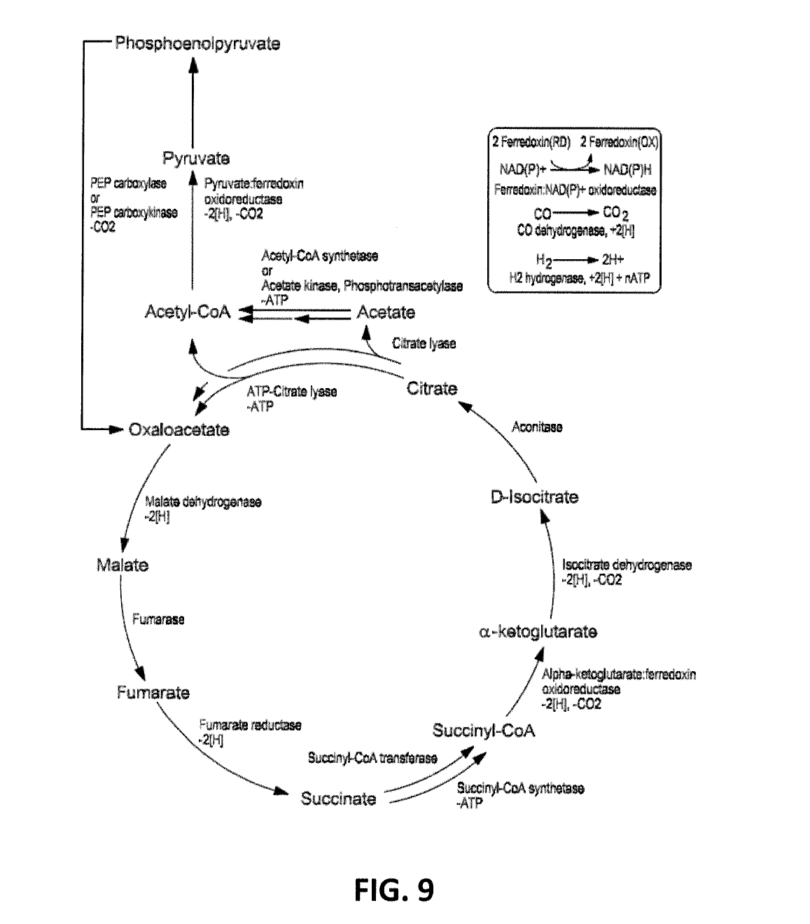

To estimate the final concentrations of CODH and Mtr proteins, SDS-PAGE followed by Western blot analyses were performed on the same cell extracts used in the CO oxidation, ACS, methyltransferase, and corrinoid Fe—S assays. The antisera used were polyclonal to purified M. thermoacetica CODH-ACS and Mtr proteins and were visualized using an alkaline phosphatase-linked goat-anti-rabbit secondary antibody. The Westerns were performed and results are shown in FIG. 9. The amounts of CODH in ACS90 and ACS91 were estimated at 50 ng by comparison to the control lanes. Expression of CODH-ACS operon genes including 2 CODH subunits and the methyltransferase were confirmed via Western blot analysis. Therefore, the recombinant E. coli cells express multiple components of a 7 gene operon. In addition, both the methyltransferase and corrinoid iron sulfur protein were active in the same recombinant E. coli cells. These proteins are part of the same operon cloned into the same cells.

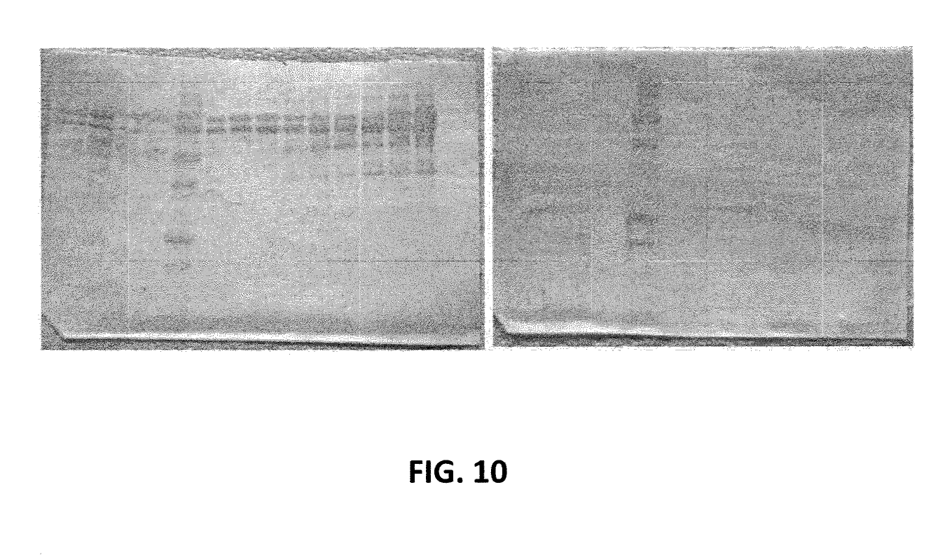

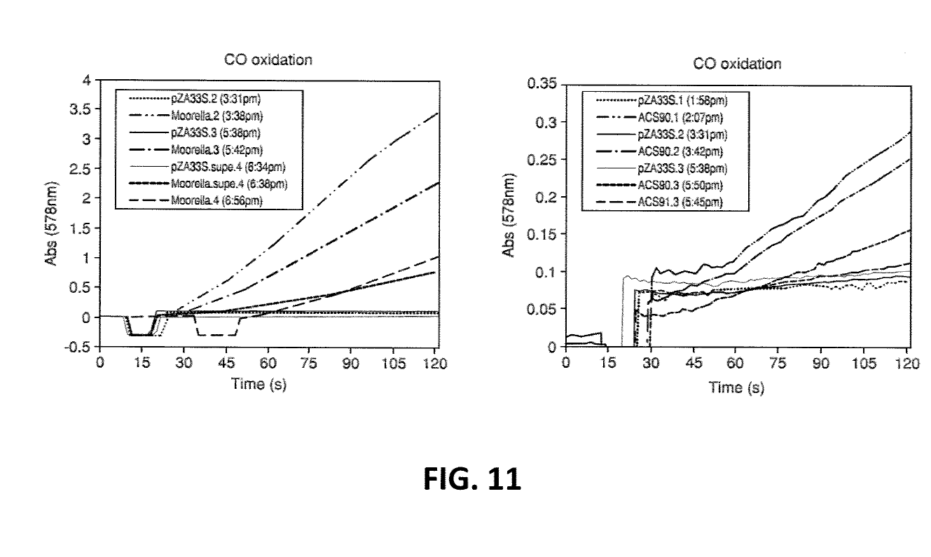

The CO oxidation assays were repeated using extracts of Moorella thermoacetica cells for the positive controls. Though CODH activity in E. coli ACS90 and ACS91 was measurable, it was at about 130-150× lower than the M. thermoacetica control. The results of the assay are shown in FIG. 10. Briefly, cells (M thermoacetica or E. coli with the CODH/ACS operon; ACS90 or ACS91 or empty vector: pZA33S) were grown and extracts prepared as described above. Assays were performed as described above at 55° C. at various times on the day the extracts were prepared. Reduction of methylviologen was followed at 578 nm over a 120 sec time course.

These results describe the CO oxidation (CODH) assay and results. Recombinant E. coli cells expressed CO oxidation activity as measured by the methyl viologen reduction assay.

### Example IX

### E. Coli CO Tolerance Experiment and CO Concentration Assay

### Myoglobin Assay

This example describes the tolerance of E. coli for high concentrations of CO.

To test whether or not E. coli can grow anaerobically in the presence of saturating amounts of CO, cultures were set up in 120 ml serum bottles with 50 ml of Terrific Broth medium (plus reducing solution, NiCl₂, Fe(II)NH₄SO₄, cyanocobalamin, IPTG, and chloramphenicol) as described above for anaerobic microbiology in small volumes. One half of these bottles were equilibrated with nitrogen gas for 30 min. and one half was equilibrated with CO gas for 30 min. An empty vector (pZA33) was used as a control, and cultures containing the pZA33 empty vector as well as both ACS90 and ACS91 were tested with both N₂ and CO. All were inoculated and grown for 36 hrs with shaking (250 rpm) at 37° C. At the end of the 36 hour period, examination of the flasks showed high amounts of growth in all. The bulk of the observed growth occurred overnight with a long lag.

Given that all cultures appeared to grow well in the presence of CO, the final CO concentrations were confirmed. This was performed using an assay of the spectral shift of myoglobin upon exposure to CO. Myoglobin reduced with sodium dithionite has an absorbance peak at 435 nm; this peak is shifted to 423 nm with CO. Due to the low wavelength and need to record a whole spectrum from 300 nm on upwards, quartz cuvettes must be used. CO concentration is measured against a standard curve and depends upon the Henry's Law constant for CO of maximum water solubility=970 micromolar at 20° C. and 1 atm.

For the myoglobin test of CO concentration, cuvettes were washed 10× with water, 1× with acetone, and then stoppered as with the CODH assay. N₂ was blown into the cuvettes for ˜10 min. A volume of 1 ml of anaerobic buffer (HEPES, pH 8.0, 2 mM DTT) was added to the blank (not equilibrated with CO) with a Hamilton syringe. A volume of 10 microliter myoglobin (˜1 mM—can be varied, just need a fairly large amount) and 1 microliter dithionite (20 mM stock) were added. A CO standard curve was made using CO saturated buffer added at 1 microliter increments. Peak height and shift was recorded for each increment. The cultures tested were pZA33/CO, ACS90/CO, and ACS91/CO. Each of these was added in 1 microliter increments to the same cuvette. Midway through the experiment a second cuvette was set up and used. The results are shown in Table II.

TABLE II
Carbon Monoxide Concentrations, 36 hrs.

| Strain and Growth Conditions | Final CO concentration (micromolar) |
|---|---|
| pZA33-CO | 930 |
| ACS90-CO | 638 |
| | 494 |
| | 734 |
| | 883 |
| ave | 687 |
| SD | 164 |
| ACS91-CO | 728 |
| | 812 |
| | 760 |
| | 611 |
| ave. | 728 |
| SD | 85 |

The results shown in Table II indicate that the cultures grew whether or not a strain was cultured in the presence of CO or not. These results indicate that E. coli can tolerate exposure to CO under anaerobic conditions and that E. coli cells expressing the CODH-ACS operon can metabolize some of the CO.

These results demonstrate that E. coli cells, whether expressing CODH/ACS or not, were able to grow in the presence of saturating amounts of CO. Furthermore, these grew equally well as the controls in nitrogen in place of CO. This experiment demonstrated that laboratory strains of E. coli are insensitive to CO at the levels achievable in a syngas project performed at normal atmospheric pressure. In addition, preliminary experiments indicated that the recombinant E. coli cells expressing CODH/ACS actually consumed some CO, probably by oxidation to carbon dioxide.

### Example X

### Exemplary Carboxylic Acid Reductases

This example describes the use of carboxylic acid reductases (CAR) to carry out the conversion of a carboxylic acid to an aldehyde.

Any intermediate carboxylic acid in a cyclohexanone pathway (or accessible carboxylic acid via its CoA derivative) can be converted to an aldehyde, if so desired. The conversion of unactivated acids to aldehydes can be carried out by an acid reductase. Examples of such conversions include, but are not limited, the conversion of 4-hydroxybutyrate, succinate, alpha-ketoglutarate, and 4-aminobutyrate to 4-hydroxybutanal, succinate semialdehyde, 2,5-dioxopentanoate, and 4-aminobutanal, respectively. One notable carboxylic acid reductase can be found in Nocardia iowensis which catalyzes the magnesium, ATP and NADPH-dependent reduction of carboxylic acids to their corresponding aldehydes (Venkitasubramanian et al., J. Biol. Chem. 282:478-485 (2007)). This enzyme is encoded by the car gene and was cloned and functionally expressed in E. coli (Venkitasubramanian et al., J. Biol. Chem. 282:478-485 (2007)). Expression of the npt gene product improved activity of the enzyme via post-transcriptional modification. The npt gene encodes a specific phosphopantetheine transferase (PPTase) that converts the inactive apo-enzyme to the active holo-enzyme. The natural substrate of this enzyme is vanillic acid, and the enzyme exhibits broad acceptance of aromatic and aliphatic substrates (Venkitasubramanian et al., in Biocatalysis in the Pharmaceutical and Biotechnology Industries, ed. R. N. Patel, Chapter 15, pp. 425-440, CRC Press LLC, Boca Raton, Fla. (2006)).

| Gene | Accession No. | GI No. | Organism |
|---|---|---|---|
| car | AAR91681.1 | 40796035 | Nocardia iowensis (sp. NRRL |
| | | | 5646) |
| npt | ABI83656.1 | 114848891 | Nocardia iowensis (sp. NRRL |
| | | | 5646) |

Additional car and npt genes can be identified based on sequence homology.

| Gene | Accession No. | GI No. | Organism |
|---|---|---|---|
| fadD9 | YP_978699.1 | 121638475 | Mycobacterium bovis |
| | | | BCG |
| BCG_2812c | YP_978898.1 | 121638674 | Mycobacterium bovis |
| | | | BCG |
| nfa20150 | YP_118225.1 | 54023983 | Nocardia farcinica |
| | | | IFM 10152 |
| nfa40540 | YP_120266.1 | 54026024 | Nocardia farcinica |
| | | | IFM 10152 |
| SGR_6790 | YP_001828302.1 | 182440583 | Streptomyces griseus |
| | | | subsp. griseus NBRC |
| | | | 13350 |
| SGR_665 | YP_001822177.1 | 182434458 | Streptomyces griseus |
| | | | subsp. griseus NBRC |
| | | | 13350 |

An additional enzyme candidate found in Streptomyces griseus is encoded by the griC and griD genes. This enzyme is believed to convert 3-amino-4-hydroxybenzoic acid to 3-amino-4-hydroxybenzaldehyde as deletion of either griC or griD led to accumulation of extracellular 3-acetylamino-4-hydroxybenzoic acid, a shunt product of 3-amino-4-hydroxybenzoic acid metabolism (Suzuki, et al., J. Antibiot. 60(6):380-387 (2007)). Co-expression of griC and griD with SGR_{—}665, an enzyme similar in sequence to the Nocardia iowensis npt, can be beneficial.

| Gene | Accession No. | GI No. | Organism |
|---|---|---|---|
| griC | 182438036 | YP_001825755.1 | Streptomyces griseus subsp. |
| | | | griseus NBRC 13350 |
| griD | 182438037 | YP_001825756.1 | Streptomyces griseus subsp. |
| | | | griseus NBRC 13350 |
| MSMEG_2956 | YP_887275.1 | YP_887275.1 | Mycobacterium smegmatis |
| | | | MC2 155 |
| MSMEG_5739 | YP_889972.1 | 118469671 | Mycobacterium smegmatis |
| | | | MC2 155 |
| MSMEG_2648 | YP_886985.1 | 118471293 | Mycobacterium smegmatis |
| | | | MC2 155 |
| MAP1040c | NP_959974.1 | 41407138 | Mycobacterium avium |
| | | | subsp. paratuberculosis K- |
| | | | 10 |
| MAP2899c | NP_961833.1 | 41408997 | Mycobacterium avium |
| | | | subsp. paratuberculosis K- |
| | | | 10 |
| MMAR_2117 | YP_001850422.1 | 183982131 | Mycobacterium marinum M |
| MMAR_2936 | YP_001851230.1 | 183982939 | Mycobacterium marinum M |
| MMAR_1916 | YP_001850220.1 | 183981929 | Mycobacterium marinum M |
| TpauDRAFT_33060 | ZP_04027864.1 | 227980601 | Tsukamurella |
| | | | paurometabola DSM |
| | | | 20162 |
| TpauDRAFT_20920 | ZP_04026660.1 | 227979396 | Tsukamurella |
| | | | paurometabola DSM |
| | | | 20162 |
| CPCC7001_1320 | ZP_05045132.1 | 254431429 | Cyanobium PCC7001 |
| DDBDRAFT_0187729 | XP_636931.1 | 66806417 | Dictyostelium discoideum |
| | | | AX4 |

An enzyme with similar characteristics, alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)). The AAR from Penicillium chrysogenum accepts S-carboxymethyl-L-cysteine as an alternate substrate, but did not react with adipate, L-glutamate or diaminopimelate (Hijarrubia et al., J. Biol. Chem. 278:8250-8256 (2003)). The gene encoding the P. chrysogenum PPTase has not been identified to date.

| Gene | Accession No. | GI No. | Organism |
|---|---|---|---|
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

Cloning and Expression of Carboxylic Acid Reductase.

Escherichia coli is used as a target organism to engineer the pathway for cyclohexanone. E. coli provides a good host for generating a non-naturally occurring microorganism capable of producing cyclohexanone. E. coli is amenable to genetic manipulation and is known to be capable of producing various intermediates and products effectively under various oxygenation conditions.

To generate a microbial organism strain such as an E. coli strain engineered to produce cyclohexanone, nucleic acids encoding a carboxylic acid reductase and phosphopantetheine transferase are expressed in E. coli using well known molecular biology techniques (see, for example, Sambrook, supra, 2001; Ausubel supra, 1999). In particular, car genes from Nocardia iowensis (designated 720), Mycobacterium smegmatis mc(2)155 (designated 890), Mycobacterium avium subspecies paratuberculosis K-10 (designated 891) and Mycobacterium marinum M (designated 892) were cloned into pZS*13 vectors (Expressys, Ruelzheim, Germany) under control of PA1/lacO promoters. The npt (ABI83656.1) gene (i.e., 721) was cloned into the pKJL33S vector, a derivative of the original mini-F plasmid vector PML31 under control of promoters and ribosomal binding sites similar to those used in pZS*13.

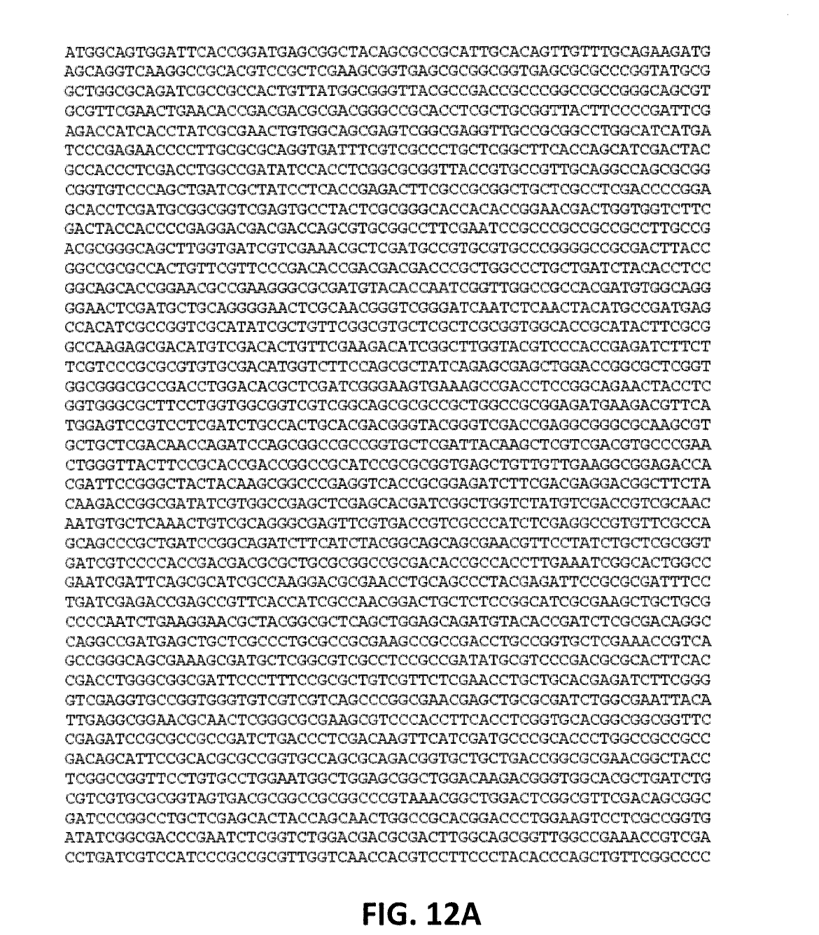

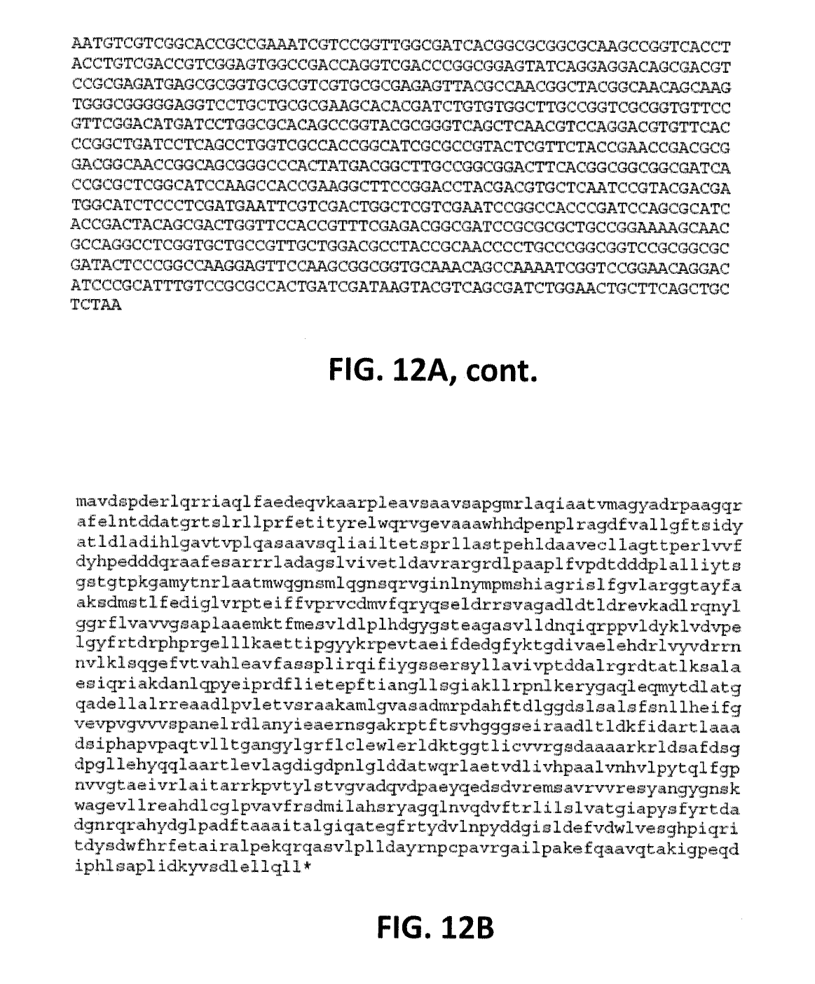

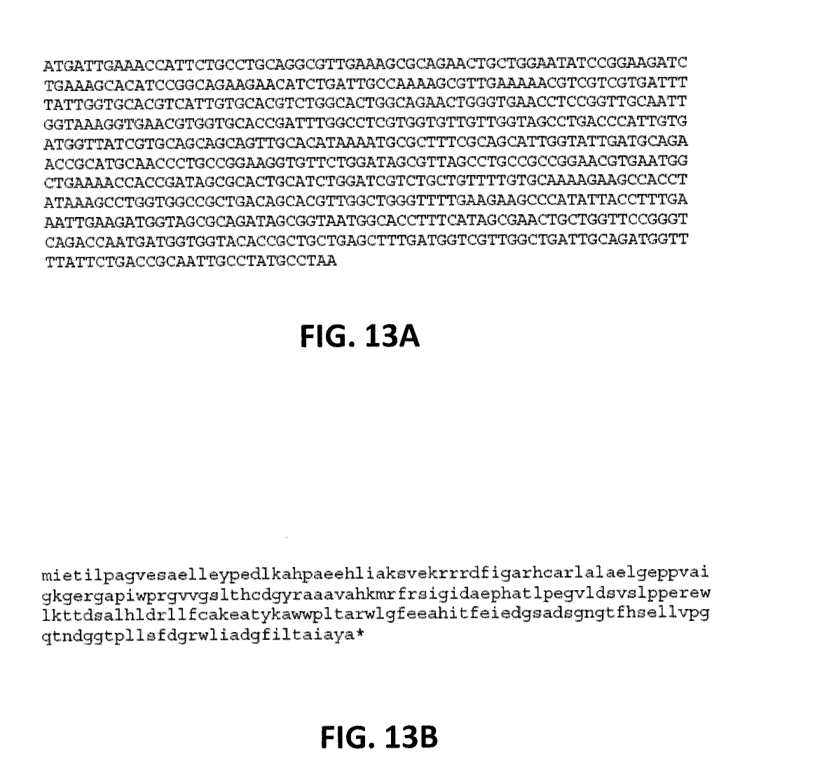

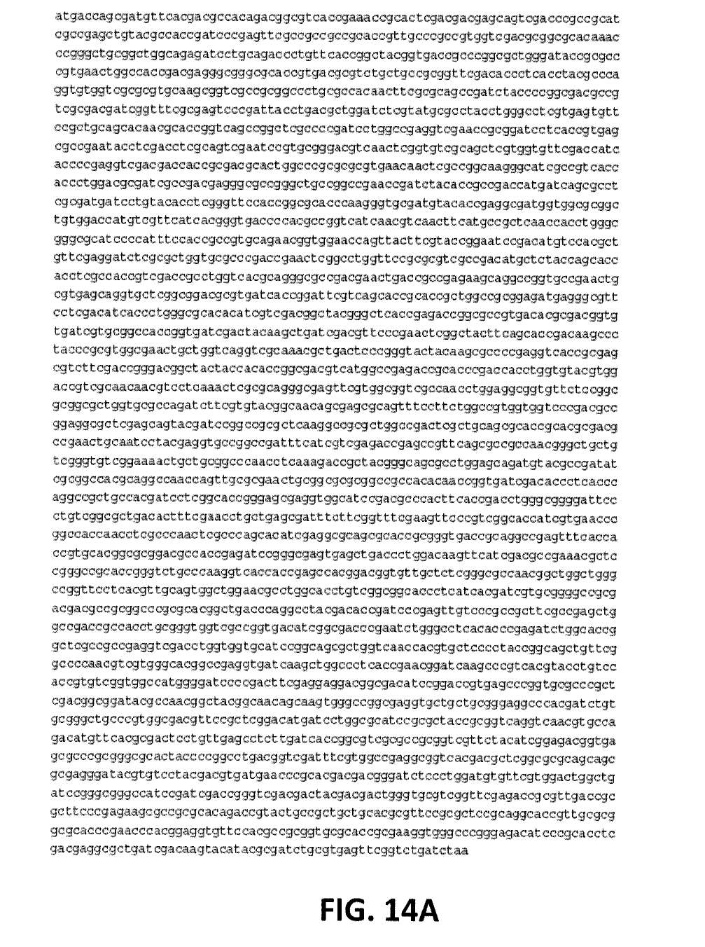

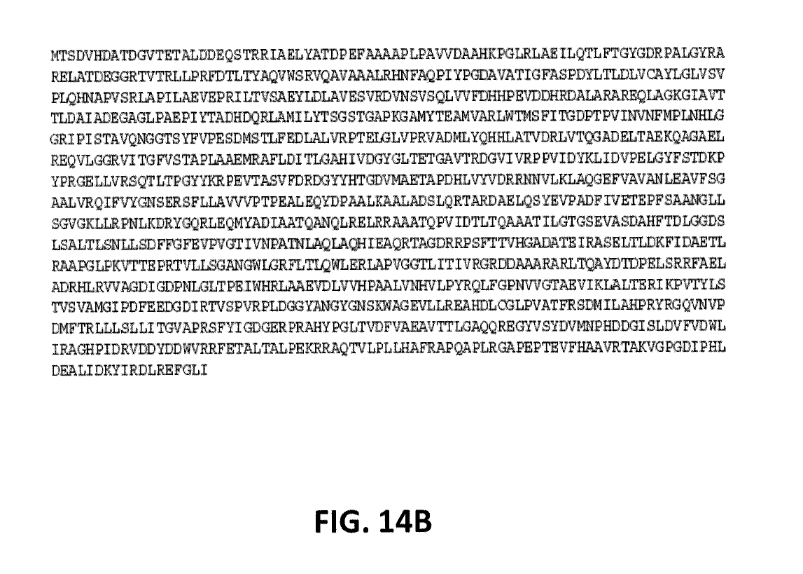

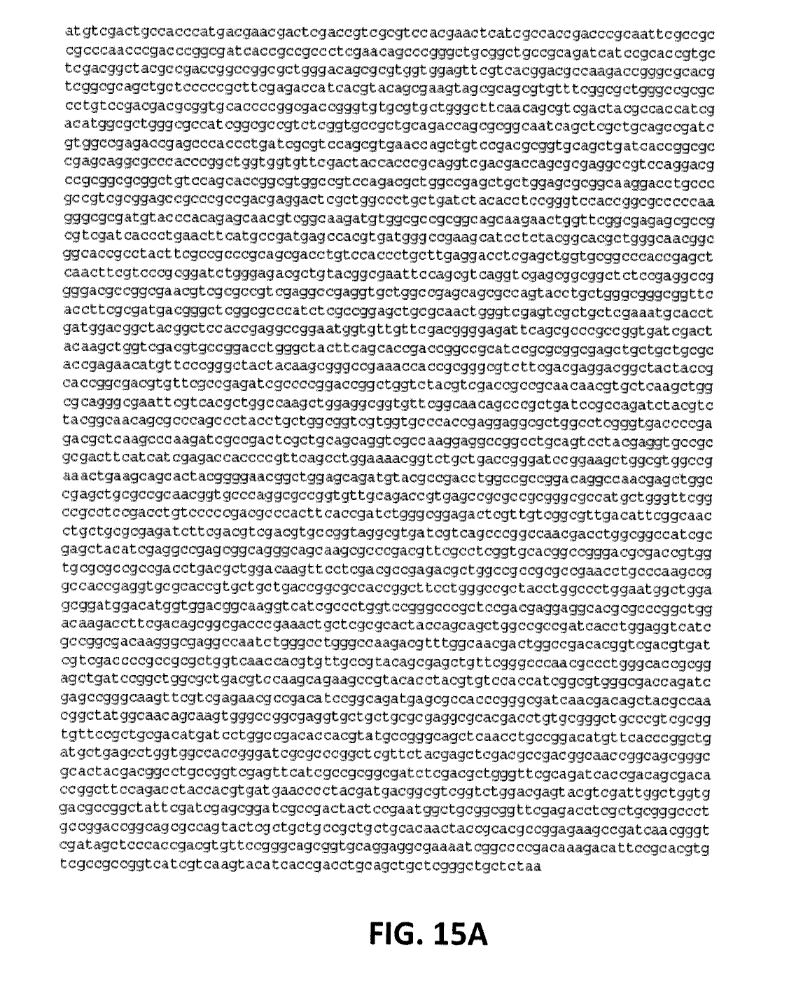

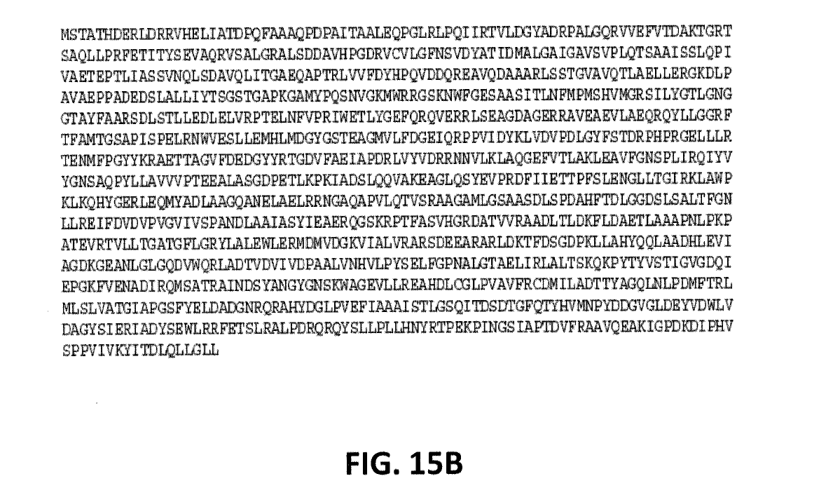

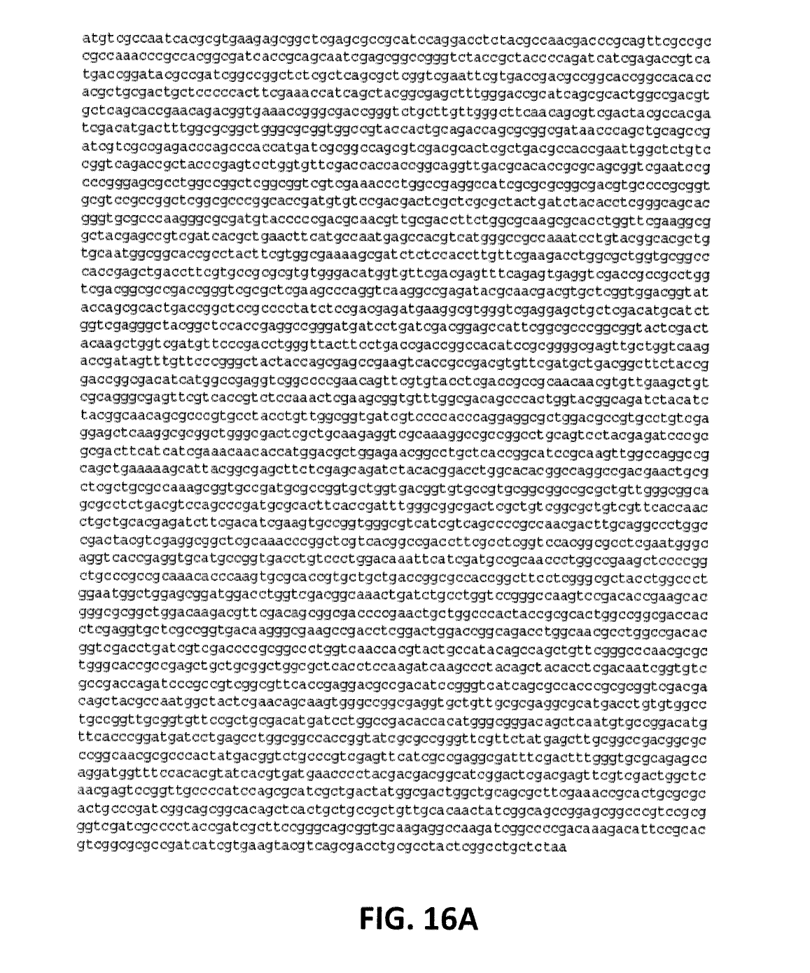

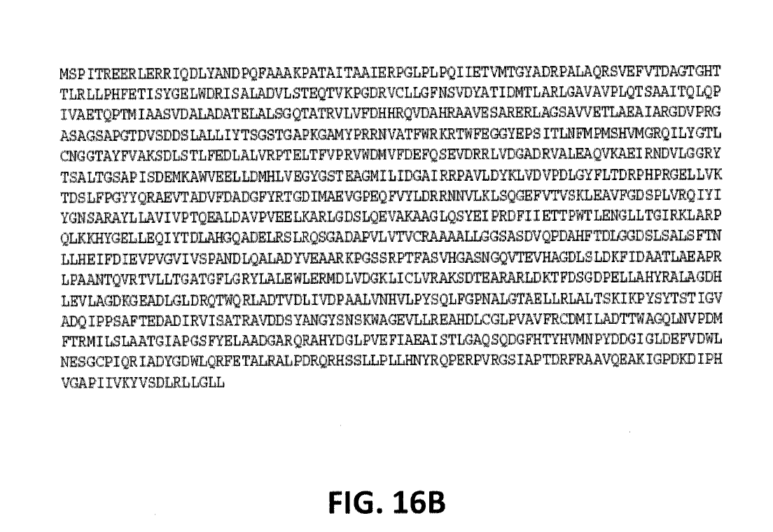

The car gene (GNM_{—}720) was cloned by PCR from Nocardia genomic DNA. Its nucleic acid and protein sequences are shown in FIGS. 12A and 12B, respectively. A codon-optimized version of the npt gene (GNM_{—}721) was synthesized by GeneArt (Regensburg, Germany). Its nucleic acid and protein sequences are shown in FIGS. 13A and 13B, respectively. The nucleic acid and protein sequences for the Mycobacterium smegmatis mc(2)155 (designated 890), Mycobacterium avium subspecies paratuberculosis K-10 (designated 891) and Mycobacterium marinum M (designated 892) genes and enzymes can be found in FIGS. 14, 15, and 16, respectively. The plasmids are transformed into a host cell to express the proteins and enzymes required for cyclohexanone production.

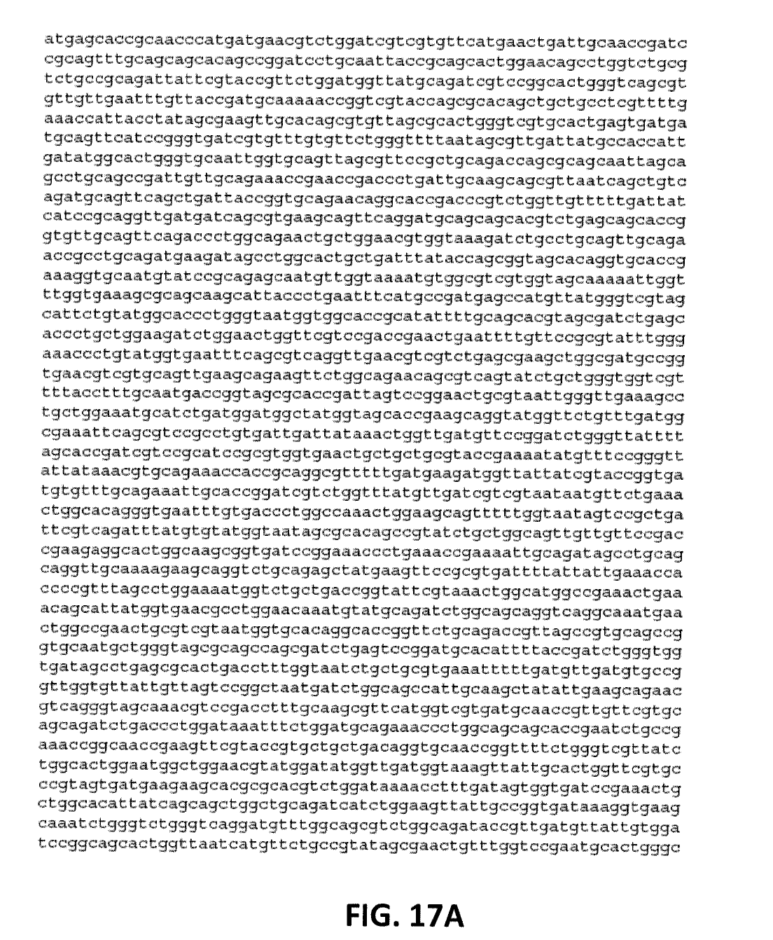

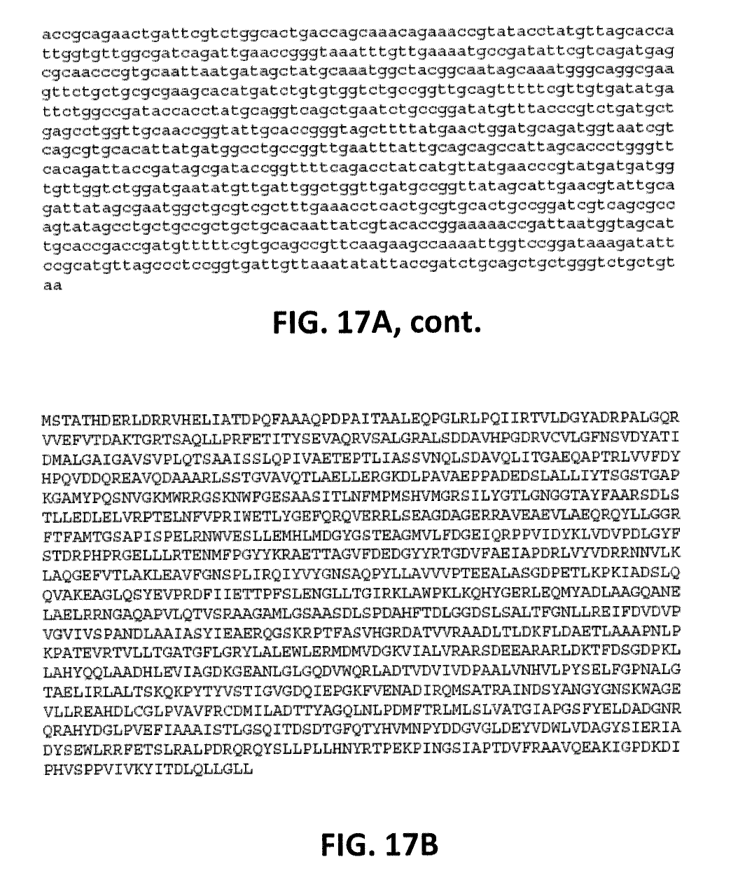

Additional CAR variants were generated. A codon optimized version of CAR 891 was generated and designated 891 GA. The nucleic acid and amino acid sequences of CAR 891GA are shown in FIGS. 17A and 17B, respectively. Over 2000 CAR variants were generated. In particular, all 20 amino acid combinations were made at positions V295, M296, G297, G391, G421, D413, G414, Y415, G416, and 5417, and additional variants were tested as well. Exemplary CAR variants include: E16K; Q95L; L100M; A1011T; K823E; T941S; H15Q; D198E; G446C; S392N; F699L; V883I; F467S; T987S; R12H; V295G; V295A; V295S; V295T; V295C; V295V; V295L; V295I; V295M; V295P; V295F; V295Y; V295W; V295D; V295E; V295N; V295Q; V295H; V295K; V295R; M296G; M296A; M296S; M296T; M296C; M296V; M296L; M2961; M296M; M296P; M296F; M296Y; M296W; M296D; M296E; M296N; M296Q; M296H; M296K; M296R; G297G; G297A; G297S; G297T; G297C; G297V; G297L; G2971; G297M; G297P; G297F; G297Y; G297W; G297D; G297E; G297N; G297Q; G297H; G297K; G297R; G391G; G391A; G391S; G391T; G391C; G391V; G391L; G3911; G391M; G391P; G391F; G391Y; G391W; G391D; G391E; G391N; G391Q; G391H; G391K; G391R; G421G; G421A; G421S; G421T; G421C; G421V; G421L; G421I G421M; G421P; G421F; G421Y; G421W; G421D; G421E; G421N; G421Q; G421H; G421K; G421R; D413G; D413A; D413S; D413T; D413C; D413V; D413L; D413I; D413M; D413P; D413F; D413Y; D413W; D413D; D413E; D413N; D413Q; D413H; D413K; D413R; G414G; G414A; G414S; G414T; G414C; G414V; G414L; G414I; G414M; G414P; G414F; G414Y; G414W; G414D; G414E; G414N; G414Q; G414H; G414K; G414R; Y415G; Y415A; Y415S; Y415T; Y415C; Y415V; Y415L; Y415I; Y415M; Y415P; Y415F; Y415Y; Y415W; Y415D; Y415E; Y415N; Y415Q; Y415H; Y415K; Y415R; G416G; G416A; G416S; G416T; G416C; G416V; G416L; G416I; G416M; G416P; G416F; G416Y; G416W; G416D; G416E; G416N; G416Q; G416H; G416K; G416R; S417G; S417A; S417S; S417T; S417C; S417V S417L; S4171; S417M; S417P; S417F; S417Y; S417W; S417D; S417E; S417N; S417Q; S417H; S417K; and S417R.

The CAR variants were screened for activity, and numerous CAR variants were found to exhibit CAR activity. This example describes the use of CAR for converting carboxylic acids to aldehydes.

### SEQUENCE LISTING

The present specification is being filed with a computer readable form (CRF) copy of the Sequence Listing. The CRF entitled 12956-140_SEQLIST.txt, which was created on Jun. 17, 2012 and is 77,766 bytes in size, is identical to the paper copy of the Sequence Listing and is incorporated herein by reference in its entirety.

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples and embodiments provided above, it should be understood that various modifications can be made without departing from the spirit of the invention.

What is claimed is:

1. A non-naturally occurring microbial organism having a cyclohexanone pathway, wherein said microbial organism comprises at least one exogenous nucleic acid encoding a cyclohexanone pathway enzyme expressed in a sufficient amount to produce cyclohexanone; said non-naturally occurring microbial organism further comprising:
(i) a reductive TCA pathway, wherein said microbial organism comprises at least one exogenous nucleic acid encoding a reductive TCA pathway enzyme selected from the group consisting of an ATP-citrate lyase, citrate lyase, a fumarate reductase, and an alpha-ketoglutarate:ferredoxin oxidoreductase;
(ii) a reductive TCA pathway, wherein said microbial organism comprises at least one exogenous nucleic acid encoding a reductive TCA pathway enzyme selected from the group consisting of a pyruvate:ferredoxin oxidoreductase, a phosphoenolpyruvate carboxylase, a phosphoenolpyruvate carboxykinase, a CO dehydrogenase, and an H₂ hydrogenase; or
(iii) at least one exogenous nucleic acid encodes an enzyme selected from a CO dehydrogenase, an H₂ hydrogenase, and combinations thereof;
wherein said cyclohexanone pathway comprises a pathway selected from the group consisting of:
(a) a PEP carboxykinase; a 2-ketocyclohexane-1-carboxyl-CoA hydrolase (acting on C—C bond); a 2-ketocyclohexane-1-carboxylate decarboxylase; and a 2-ketocyclohexane-1-carboxyl-CoA hydrolase (acting on thioester), 2-ketocyclohexane-1-carboxyl-CoA transferase, or 2-ketocyclohexane-1-carboxyl-CoA synthetase;
(b) a PEP carboxykinase; a 6-ketocyclohex-1-ene-1-carboxyl-CoA hydrolase (acting on C—C bond); a 6-ketocyclohex-1-ene-1-carboxyl-CoA synthetase; a 6-ketocyclohex-1-ene-1-carboxyl-CoA hydrolase (acting on thioester); a 6-ketocyclohex-1-ene-1-carboxyl-CoA transferase; a 6-ketocyclohex-1-ene-1-carboxyl-CoA reductase; a 6-ketocyclohex-1-ene-1-carboxylate decarboxylase; a 6-ketocyclohex-1-ene-1-carboxylate reductase; a 2-ketocyclohexane-1-carboxyl-CoA synthetase; a 2-ketocyclohexane-1-carboxyl-CoA transferase; a 2-ketocyclohexane-1-carboxyl-CoA hydrolase (acting on thioester); a 2-ketocyclohexane-1-carboxylate decarboxylase; and a cyclohexanone dehydrogenase;
(c) a PEP carboxykinase; a 6-ketocyclohex-1-ene-1-carboxyl-CoA hydrolase (acting on C—C bond); a 6-ketocyclohex-1-ene-1-carboxylate decarboxylase; a cyclohexanone dehydrogenase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;
(d) a PEP carboxykinase; a 6-ketocyclohex-1-ene-1-carboxyl-CoA hydrolase (acting on C—C bond); a 6-ketocyclohex-1-ene-1-carboxylate reductase; a 2-ketocyclohexane-1-carboxylate decarboxylase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;
(e) a PEP carboxykinase; a 6-ketocyclohex-1-ene-1-carboxyl-CoA hydrolase (acting on C—C bond); a 6-ketocyclohex-1-ene-1-carboxyl-CoA reductase; a 2-ketocyclohexane-1-carboxylate decarboxylase, and a 2-ketocyclohexane-1-carboxyl-CoA synthetase, 2-ketocyclohexane-1-carboxyl-CoA transferase, or 2-ketocyclohexane-1-carboxyl-CoA hydrolase (acting on thioester);
(f) a PEP carboxykinase; an adipate semialdehyde dehydratase; a cyclohexane-1,2-diol dehydrogenase; and a cyclohexane-1,2-diol dehydratase; and
(g) a PEP carboxykinase; a 3-oxopimelate decarboxylase; a 4-acetylbutyrate dehydratase; a 3-hydroxycyclohexanone dehydrogenase; a 2-cyclohexenone hydratase; a cyclohexanone dehydrogenase; and a 3-oxopimeloyl-CoA synthetase, 3-oxopimeloyl-CoA hydrolase (acting on thioester), or a 3-oxopimeloyl-coA transferase.

2. The non-naturally occurring microbial organism of claim 1, wherein the microbial organism has a cyclohexanone pathway comprising at least one exogenous nucleic acid encoding a cyclohexanone pathway enzyme from (a); and wherein the microbial organism further comprises a pimeloyl-CoA pathway comprising at least one exogenous nucleic acid encoding a pimeloyl-CoA pathway enzyme expressed in a sufficient amount to produce pimeloyl-CoA, said pimeloyl-CoA pathway comprising an acetoacetyl-CoA reductase, a 3-hydroxybutyryl-CoA dehydratase, a glutaryl-CoA dehydrogenase, a oxopimeloyl-CoA:glutaryl-CoA acyltransferase, a 3-hydroxypimeloyl-CoA dehydrogenase, a 3-hydroxypimeloyl-CoA dehydratase, and a pimeloyl-CoA dehydrogenase.

3. The non-naturally occurring microbial organism of claim 1, wherein the microbial organism has a cyclohexanone pathway comprising at least one exogenous nucleic acid encoding a cyclohexanone pathway enzyme from (b), and wherein said microbial organism has a native 3-hydroxypimeloyl-CoA pathway.

4. The non-naturally occurring microbial organism of claim 1, wherein the microbial organism has a cyclohexanone pathway comprising at least one exogenous nucleic acid encoding a cyclohexanone pathway enzyme from (b), and wherein the microbial organism further comprises a 3-hydroxypimeloyl-CoA pathway comprising at least one exogenous nucleic acid encoding a 3-hydroxypimeloyl-CoA pathway enzyme expressed in a sufficient amount to produce 3-hydroxypimeloyl-CoA, said 3-hydroxypimeloyl-CoA pathway comprising a acetoacetyl-CoA reductase, a 3-hydroxybutyryl-CoA dehydratase, a glutaryl-CoA dehydrogenase, a oxopimeloyl-CoA:glutaryl-CoA acyltransferase, and a 3-hydroxypimeloyl-CoA dehydrogenase.

5. The non-naturally occurring microbial organism of claim 1, wherein said microbial organism comprising (i) further comprises an exogenous nucleic acid encoding an enzyme selected from the group consisting of a pyruvate:ferredoxin oxidoreductase, an aconitase, an isocitrate dehydrogenase, a succinyl-CoA synthetase, a succinyl-CoA transferase, a fumarase, a malate dehydrogenase, an acetate kinase, a phosphotransacetylase, an acetyl-CoA synthetase, an NAD(P)H:ferredoxin oxidoreductase, ferredoxin, and combinations thereof.

6. The non-naturally occurring microbial organism of claim 1, wherein said microbial organism comprising (ii) further comprises an exogenous nucleic acid encoding an enzyme selected from the group consisting of an aconitase, an isocitrate dehydrogenase, a succinyl-CoA synthetase, a succinyl-CoA transferase, a fumarase, a malate dehydrogenase, and combinations thereof.

7. The non-naturally occurring microbial organism of claim 1, wherein said microbial organism comprises two, three, four, five, six or seven exogenous nucleic acids, each encoding a cyclohexanone pathway enzyme.

8. The non-naturally occurring microbial organism of claim 7, wherein said microbial organism comprises exogenous nucleic acids encoding each of the enzymes of a cyclohexanone pathway selected from the group consisting of
(a) a PEP carboxykinase; a 2-ketocyclohexane-1-carboxyl-CoA hydrolase (acting on C—C bond); a 2-ketocyclohexane-1-carboxylate decarboxylase; and a 2-ketocyclohexane-1-carboxyl-CoA hydrolase (acting on thioester), 2-ketocyclohexane-1-carboxyl-CoA transferase, or 2-ketocyclohexane-1-carboxyl-CoA synthetase;
(b) a PEP carboxykinase; a 6-ketocyclohex-1-ene-1-carboxyl-CoA hydrolase (acting on C—C bond); a 6-ketocyclohex-1-ene-1-carboxyl-CoA synthetase; a 6-ketocyclohex-1-ene-1-carboxyl-CoA hydrolase (acting on thioester); a 6-ketocyclohex-1-ene-1-carboxyl-CoA transferase; a 6-ketocyclohex-1-ene-1-carboxyl-CoA reductase; a 6-ketocyclohex-1-ene-1-carboxylate decarboxylase; a 6-ketocyclohex-1-ene-1-carboxylate reductase; a 2-ketocyclohexane-1-carboxyl-CoA synthetase; a 2-ketocyclohexane-1-carboxyl-CoA transferase; a 2-ketocyclohexane-1-carboxyl-CoA hydrolase (acting on thioester); a 2-ketocyclohexane-1-carboxylate decarboxylase; and a cyclohexanone dehydrogenase;
(c) a PEP carboxykinase; a 6-ketocyclohex-1-ene-1-carboxyl-CoA hydrolase (acting on C—C bond); a 6-ketocyclohex-1-ene-1-carboxylate decarboxylase; a cyclohexanone dehydrogenase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;
(d) a PEP carboxykinase; a 6-ketocyclohex-1-ene-1-carboxyl-CoA hydrolase (acting on C—C bond); a 6-ketocyclohex-1-ene-1-carboxylate reductase; a 2-ketocyclohexane-1-carboxylate decarboxylase; and a 6-ketocyclohex-1-ene-1-carboxyl-CoA synthetase, 6-ketocyclohex-1-ene-1-carboxyl-CoA hydrolase (acting on thioester), or 6-ketocyclohex-1-ene-1-carboxyl-CoA transferase;
(e) a PEP carboxykinase; a 6-ketocyclohex-1-ene-1-carboxyl-CoA hydrolase (acting on C—C bond); a 6-ketocyclohex-1-ene-1-carboxyl-CoA reductase; a 2-ketocyclohexane-1-carboxylate decarboxylase, and a 2-ketocyclohexane-1-carboxyl-CoA synthetase, 2-ketocyclohexane-1-carboxyl-CoA transferase, or 2-ketocyclohexane-1-carboxyl-CoA hydrolase (acting on thioester);
(f) a PEP carboxykinase; an adipate semialdehyde dehydratase; a cyclohexane-1,2-diol dehydrogenase; and a cyclohexane-1,2-diol dehydratase; and
(g) a PEP carboxykinase; a 3-oxopimelate decarboxylase; a 4-acetylbutyrate dehydratase; a 3-hydroxycyclohexanone dehydrogenase; a 2-cyclohexenone hydratase; a cyclohexanone dehydrogenase; and a 3-oxopimeloyl-CoA synthetase, 3-oxopimeloyl-CoA hydrolase (acting on thioester), or a 3-oxopimeloyl-coA transferase.

9. The non-naturally occurring microbial organism of claim 1, wherein said microbial organism comprises two, three, four or five exogenous nucleic acids each encoding enzymes of (i), (ii) or (iii).

10. The non-naturally occurring microbial organism of claim 9, wherein said microbial organism comprising (i) comprises four exogenous nucleic acids encoding ATP-citrate lyase, citrate lyase, a fumarate reductase, and an alpha-ketoglutarate:ferredoxin oxidoreductase;
wherein said microbial organism comprising (ii) comprises five exogenous nucleic acids encoding pyruvate:ferredoxin oxidoreductase, a phosphoenolpyruvate carboxylase, a phosphoenolpyruvate carboxykinase, a CO dehydrogenase, and an H₂ hydrogenase; or
wherein said microbial organism comprising (iii) comprises two exogenous nucleic acids encoding CO dehydrogenase and H₂ hydrogenase.

11. The non-naturally occurring microbial organism of claim 1, wherein said at least one exogenous nucleic acid is a heterologous nucleic acid.

12. The non-naturally occurring microbial organism of claim 1, wherein said non-naturally occurring microbial organism is in a substantially anaerobic culture medium.

13. A method for producing cyclohexanone, comprising culturing the non-naturally occurring microbial organism of claim 1 under conditions and for a sufficient period of time to produce cyclohexanone.

14. A method for producing cyclohexanone, comprising culturing the non-naturally occurring microbial organism of claim 8 under conditions and for a sufficient period of time to produce cyclohexanone.


SEQUENCE LISTING (12 sequences)

SEQ ID NO: 1 - DNA, 3525 bp, Nocardia iowensis
atggcagtggattcaccggatgagcggctacagcgccgcattgcacagttgtttgcagaagatgagcaggtcaaggccgcacgtccgctcgaagcggtgagcgcggcggtgagcgcgcccggtatgcggctggcgcagatcgccgccactgttatggcgggttacgccgaccgcccggccgccgggcagcgtgcgttcgaactgaacaccgacgacgcgacgggccgcacctcgctgcggttacttccccgattcgagaccatcacctatcgcgaactgtggcagcgagtcggcgaggttgccgcggcctggcatcatgatcccgagaaccccttgcgcgcaggtgatttcgtcgccctgctcggcttcaccagcatcgactacgccaccctcgacctggccgatatccacctcggcgcggttaccgtgccgttgcaggccagcgcggcggtgtcccagctgatcgctatcctcaccgagacttcgccgcggctgctcgcctcgaccccggagcacctcgatgcggcggtcgagtgcctactcgcgggcaccacaccggaacgactggtggtcttcgactaccaccccgaggacgacgaccagcgtgcggccttcgaatccgcccgccgccgccttgccgacgcgggcagcttggtgatcgtcgaaacgctcgatgccgtgcgtgcccggggccgcgacttaccggccgcgccactgttcgttcccgacaccgacgacgacccgctggccctgctgatctacacctccggcagcaccggaacgccgaagggcgcgatgtacaccaatcggttggccgccacgatgtggcaggggaactcgatgctgcaggggaactcgcaacgggtcgggatcaatctcaactacatgccgatgagccacatcgccggtcgcatatcgctgttcggcgtgctcgctcgcggtggcaccgcatacttcgcggccaagagcgacatgtcgacactgttcgaagacatcggcttggtacgtcccaccgagatcttcttcgtcccgcgcgtgtgcgacatggtcttccagcgctatcagagcgagctggaccggcgctcggtggcgggcgccgacctggacacgctcgatcgggaagtgaaagccgacctccggcagaactacctcggtgggcgcttcctggtggcggtcgtcggcagcgcgccgctggccgcggagatgaagacgttcatggagtccgtcctcgatctgccactgcacgacgggtacgggtcgaccgaggcgggcgcaagcgtgctgctcgacaaccagatccagcggccgccggtgctcgattacaagctcgtcgacgtgcccgaactgggttacttccgcaccgaccggccgcatccgcgcggtgagctgttgttgaaggcggagaccacgattccgggctactacaagcggcccgaggtcaccgcggagatcttcgacgaggacggcttctacaagaccggcgatatcgtggccgagctcgagcacgatcggctggtctatgtcgaccgtcgcaacaatgtgctcaaactgtcgcagggcgagttcgtgaccgtcgcccatctcgaggccgtgttcgccagcagcccgctgatccggcagatcttcatctacggcagcagcgaacgttcctatctgctcgcggtgatcgtccccaccgacgacgcgctgcgcggccgcgacaccgccaccttgaaatcggcactggccgaatcgattcagcgcatcgccaaggacgcgaacctgcagccctacgagattccgcgcgatttcctgatcgagaccgagccgttcaccatcgccaacggactgctctccggcatcgcgaagctgctgcgccccaatctgaaggaacgctacggcgctcagctggagcagatgtacaccgatctcgcgacaggccaggccgatgagctgctcgccctgcgccgcgaagccgccgacctgccggtgctcgaaaccgtcagccgggcagcgaaagcgatgctcggcgtcgcctccgccgatatgcgtcccgacgcgcacttcaccgacctgggcggcgattccctttccgcgctgtcgttctcgaacctgctgcacgagatcttcggggtcgaggtgccggtgggtgtcgtcgtcagcccggcgaacgagctgcgcgatctggcgaattacattgaggcggaacgcaactcgggcgcgaagcgtcccaccttcacctcggtgcacggcggcggttccgagatccgcgccgccgatctgaccctcgacaagttcatcgatgcccgcaccctggccgccgccgacagcattccgcacgcgccggtgccagcgcagacggtgctgctgaccggcgcgaacggctacctcggccggttcctgtgcctggaatggctggagcggctggacaagacgggtggcacgctgatctgcgtcgtgcgcggtagtgacgcggccgcggcccgtaaacggctggactcggcgttcgacagcggcgatcccggcctgctcgagcactaccagcaactggccgcacggaccctggaagtcctcgccggtgatatcggcgacccgaatctcggtctggacgacgcgacttggcagcggttggccgaaaccgtcgacctgatcgtccatcccgccgcgttggtcaaccacgtccttccctacacccagctgttcggccccaatgtcgtcggcaccgccgaaatcgtccggttggcgatcacggcgcggcgcaagccggtcacctacctgtcgaccgtcggagtggccgaccaggtcgacccggcggagtatcaggaggacagcgacgtccgcgagatgagcgcggtgcgcgtcgtgcgcgagagttacgccaacggctacggcaacagcaagtgggcgggggaggtcctgctgcgcgaagcacacgatctgtgtggcttgccggtcgcggtgttccgttcggacatgatcctggcgcacagccggtacgcgggtcagctcaacgtccaggacgtgttcacccggctgatcctcagcctggtcgccaccggcatcgcgccgtactcgttctaccgaaccgacgcggacggcaaccggcagcgggcccactatgacggcttgccggcggacttcacggcggcggcgatcaccgcgctcggcatccaagccaccgaaggcttccggacctacgacgtgctcaatccgtacgacgatggcatctccctcgatgaattcgtcgactggctcgtcgaatccggccacccgatccagcgcatcaccgactacagcgactggttccaccgtttcgagacggcgatccgcgcgctgccggaaaagcaacgccaggcctcggtgctgccgttgctggacgcctaccgcaacccctgcccggcggtccgcggcgcgatactcccggccaaggagttccaagcggcggtgcaaacagccaaaatcggtccggaacaggacatcccgcatttgtccgcgccactgatcgataagtacgtcagcgatctggaactgcttcagctgctctaa

SEQ ID NO: 2 - protein, 1174 aa, Nocardia iowensis
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 3 - DNA, 669 bp, Artificial Sequence
  Description of Artificial Sequence Synthetic codon optimized phosphpantetheine transferase polynucleotide
atgattgaaaccattctgcctgcaggcgttgaaagcgcagaactgctggaatatccggaagatctgaaagcacatccggcagaagaacatctgattgccaaaagcgttgaaaaacgtcgtcgtgattttattggtgcacgtcattgtgcacgtctggcactggcagaactgggtgaacctccggttgcaattggtaaaggtgaacgtggtgcaccgatttggcctcgtggtgttgttggtagcctgacccattgtgatggttatcgtgcagcagcagttgcacataaaatgcgctttcgcagcattggtattgatgcagaaccgcatgcaaccctgccggaaggtgttctggatagcgttagcctgccgccggaacgtgaatggctgaaaaccaccgatagcgcactgcatctggatcgtctgctgttttgtgcaaaagaagccacctataaagcctggtggccgctgacagcacgttggctgggttttgaagaagcccatattacctttgaaattgaagatggtagcgcagatagcggtaatggcacctttcatagcgaactgctggttccgggtcagaccaatgatggtggtacaccgctgctgagctttgatggtcgttggctgattgcagatggttttattctgaccgcaattgcctatgcctaa

SEQ ID NO: 4 - protein, 222 aa, Artificial Sequence
  Description of Artificial Sequence Synthetic codon optimized phosphpantetheine transferase polypeptide
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 5 - DNA, 3522 bp, Mycobacterium smegmatis
atgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctaa

SEQ ID NO: 6 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 7 - DNA, 3522 bp, Mycobacterium avium
atgtcgactgccacccatgacgaacgactcgaccgtcgcgtccacgaactcatcgccaccgacccgcaattcgccgccgcccaacccgacccggcgatcaccgccgccctcgaacagcccgggctgcggctgccgcagatcatccgcaccgtgctcgacggctacgccgaccggccggcgctgggacagcgcgtggtggagttcgtcacggacgccaagaccgggcgcacgtcggcgcagctgctcccccgcttcgagaccatcacgtacagcgaagtagcgcagcgtgtttcggcgctgggccgcgccctgtccgacgacgcggtgcaccccggcgaccgggtgtgcgtgctgggcttcaacagcgtcgactacgccaccatcgacatggcgctgggcgccatcggcgccgtctcggtgccgctgcagaccagcgcggcaatcagctcgctgcagccgatcgtggccgagaccgagcccaccctgatcgcgtccagcgtgaaccagctgtccgacgcggtgcagctgatcaccggcgccgagcaggcgcccacccggctggtggtgttcgactaccacccgcaggtcgacgaccagcgcgaggccgtccaggacgccgcggcgcggctgtccagcaccggcgtggccgtccagacgctggccgagctgctggagcgcggcaaggacctgcccgccgtcgcggagccgcccgccgacgaggactcgctggccctgctgatctacacctccgggtccaccggcgcccccaagggcgcgatgtacccacagagcaacgtcggcaagatgtggcgccgcggcagcaagaactggttcggcgagagcgccgcgtcgatcaccctgaacttcatgccgatgagccacgtgatgggccgaagcatcctctacggcacgctgggcaacggcggcaccgcctacttcgccgcccgcagcgacctgtccaccctgcttgaggacctcgagctggtgcggcccaccgagctcaacttcgtcccgcggatctgggagacgctgtacggcgaattccagcgtcaggtcgagcggcggctctccgaggccggggacgccggcgaacgtcgcgccgtcgaggccgaggtgctggccgagcagcgccagtacctgctgggcgggcggttcaccttcgcgatgacgggctcggcgcccatctcgccggagctgcgcaactgggtcgagtcgctgctcgaaatgcacctgatggacggctacggctccaccgaggccggaatggtgttgttcgacggggagattcagcgcccgccggtgatcgactacaagctggtcgacgtgccggacctgggctacttcagcaccgaccggccgcatccgcgcggcgagctgctgctgcgcaccgagaacatgttcccgggctactacaagcgggccgaaaccaccgcgggcgtcttcgacgaggacggctactaccgcaccggcgacgtgttcgccgagatcgccccggaccggctggtctacgtcgaccgccgcaacaacgtgctcaagctggcgcagggcgaattcgtcacgctggccaagctggaggcggtgttcggcaacagcccgctgatccgccagatctacgtctacggcaacagcgcccagccctacctgctggcggtcgtggtgcccaccgaggaggcgctggcctcgggtgaccccgagacgctcaagcccaagatcgccgactcgctgcagcaggtcgccaaggaggccggcctgcagtcctacgaggtgccgcgcgacttcatcatcgagaccaccccgttcagcctggaaaacggtctgctgaccgggatccggaagctggcgtggccgaaactgaagcagcactacggggaacggctggagcagatgtacgccgacctggccgccggacaggccaacgagctggccgagctgcgccgcaacggtgcccaggcgccggtgttgcagaccgtgagccgcgccgcgggcgccatgctgggttcggccgcctccgacctgtcccccgacgcccacttcaccgatctgggcggagactcgttgtcggcgttgacattcggcaacctgctgcgcgagatcttcgacgtcgacgtgccggtaggcgtgatcgtcagcccggccaacgacctggcggccatcgcgagctacatcgaggccgagcggcagggcagcaagcgcccgacgttcgcctcggtgcacggccgggacgcgaccgtggtgcgcgccgccgacctgacgctggacaagttcctcgacgccgagacgctggccgccgcgccgaacctgcccaagccggccaccgaggtgcgcaccgtgctgctgaccggcgccaccggcttcctgggccgctacctggccctggaatggctggagcggatggacatggtggacggcaaggtcatcgccctggtccgggcccgctccgacgaggaggcacgcgcccggctggacaagaccttcgacagcggcgacccgaaactgctcgcgcactaccagcagctggccgccgatcacctggaggtcatcgccggcgacaagggcgaggccaatctgggcctgggccaagacgtttggcaacgactggccgacacggtcgacgtgatcgtcgaccccgccgcgctggtcaaccacgtgttgccgtacagcgagctgttcgggcccaacgccctgggcaccgcggagctgatccggctggcgctgacgtccaagcagaagccgtacacctacgtgtccaccatcggcgtgggcgaccagatcgagccgggcaagttcgtcgagaacgccgacatccggcagatgagcgccacccgggcgatcaacgacagctacgccaacggctatggcaacagcaagtgggccggcgaggtgctgctgcgcgaggcgcacgacctgtgcgggctgcccgtcgcggtgttccgctgcgacatgatcctggccgacaccacgtatgccgggcagctcaacctgccggacatgttcacccggctgatgctgagcctggtggccaccgggatcgcgcccggctcgttctacgagctcgacgccgacggcaaccggcagcgggcgcactacgacggcctgccggtcgagttcatcgccgcggcgatctcgacgctgggttcgcagatcaccgacagcgacaccggcttccagacctaccacgtgatgaacccctacgatgacggcgtcggtctggacgagtacgtcgattggctggtggacgccggctattcgatcgagcggatcgccgactactccgaatggctgcggcggttcgagacctcgctgcgggccctgccggaccggcagcgccagtactcgctgctgccgctgctgcacaactaccgcacgccggagaagccgatcaacgggtcgatagctcccaccgacgtgttccgggcagcggtgcaggaggcgaaaatcggccccgacaaagacattccgcacgtgtcgccgccggtcatcgtcaagtacatcaccgacctgcagctgctcgggctgctctaa

SEQ ID NO: 8 - protein, 1173 aa, Mycobacterium avium
MSTATHDERLDRRVHELIATDPQFAAAQPDPAITAALEQPGLRLPQIIRTVLDGYADRPALGQRVVEFVTDAKTGRTSAQLLPRFETITYSEVAQRVSALGRALSDDAVHPGDRVCVLGFNSVDYATIDMALGAIGAVSVPLQTSAAISSLQPIVAETEPTLIASSVNQLSDAVQLITGAEQAPTRLVVFDYHPQVDDQREAVQDAAARLSSTGVAVQTLAELLERGKDLPAVAEPPADEDSLALLIYTSGSTGAPKGAMYPQSNVGKMWRRGSKNWFGESAASITLNFMPMSHVMGRSILYGTLGNGGTAYFAARSDLSTLLEDLELVRPTELNFVPRIWETLYGEFQRQVERRLSEAGDAGERRAVEAEVLAEQRQYLLGGRFTFAMTGSAPISPELRNWVESLLEMHLMDGYGSTEAGMVLFDGEIQRPPVIDYKLVDVPDLGYFSTDRPHPRGELLLRTENMFPGYYKRAETTAGVFDEDGYYRTGDVFAEIAPDRLVYVDRRNNVLKLAQGEFVTLAKLEAVFGNSPLIRQIYVYGNSAQPYLLAVVVPTEEALASGDPETLKPKIADSLQQVAKEAGLQSYEVPRDFIIETTPFSLENGLLTGIRKLAWPKLKQHYGERLEQMYADLAAGQANELAELRRNGAQAPVLQTVSRAAGAMLGSAASDLSPDAHFTDLGGDSLSALTFGNLLREIFDVDVPVGVIVSPANDLAAIASYIEAERQGSKRPTFASVHGRDATVVRAADLTLDKFLDAETLAAAPNLPKPATEVRTVLLTGATGFLGRYLALEWLERMDMVDGKVIALVRARSDEEARARLDKTFDSGDPKLLAHYQQLAADHLEVIAGDKGEANLGLGQDVWQRLADTVDVIVDPAALVNHVLPYSELFGPNALGTAELIRLALTSKQKPYTYVSTIGVGDQIEPGKFVENADIRQMSATRAINDSYANGYGNSKWAGEVLLREAHDLCGLPVAVFRCDMILADTTYAGQLNLPDMFTRLMLSLVATGIAPGSFYELDADGNRQRAHYDGLPVEFIAAAISTLGSQITDSDTGFQTYHVMNPYDDGVGLDEYVDWLVDAGYSIERIADYSEWLRRFETSLRALPDRQRQYSLLPLLHNYRTPEKPINGSIAPTDVFRAAVQEAKIGPDKDIPHVSPPVIVKYITDLQLLGLL

SEQ ID NO: 9 - DNA, 3525 bp, Mycobacterium marinum
atgtcgccaatcacgcgtgaagagcggctcgagcgccgcatccaggacctctacgccaacgacccgcagttcgccgccgccaaacccgccacggcgatcaccgcagcaatcgagcggccgggtctaccgctaccccagatcatcgagaccgtcatgaccggatacgccgatcggccggctctcgctcagcgctcggtcgaattcgtgaccgacgccggcaccggccacaccacgctgcgactgctcccccacttcgaaaccatcagctacggcgagctttgggaccgcatcagcgcactggccgacgtgctcagcaccgaacagacggtgaaaccgggcgaccgggtctgcttgttgggcttcaacagcgtcgactacgccacgatcgacatgactttggcgcggctgggcgcggtggccgtaccactgcagaccagcgcggcgataacccagctgcagccgatcgtcgccgagacccagcccaccatgatcgcggccagcgtcgacgcactcgctgacgccaccgaattggctctgtccggtcagaccgctacccgagtcctggtgttcgaccaccaccggcaggttgacgcacaccgcgcagcggtcgaatccgcccgggagcgcctggccggctcggcggtcgtcgaaaccctggccgaggccatcgcgcgcggcgacgtgccccgcggtgcgtccgccggctcggcgcccggcaccgatgtgtccgacgactcgctcgcgctactgatctacacctcgggcagcacgggtgcgcccaagggcgcgatgtacccccgacgcaacgttgcgaccttctggcgcaagcgcacctggttcgaaggcggctacgagccgtcgatcacgctgaacttcatgccaatgagccacgtcatgggccgccaaatcctgtacggcacgctgtgcaatggcggcaccgcctacttcgtggcgaaaagcgatctctccaccttgttcgaagacctggcgctggtgcggcccaccgagctgaccttcgtgccgcgcgtgtgggacatggtgttcgacgagtttcagagtgaggtcgaccgccgcctggtcgacggcgccgaccgggtcgcgctcgaagcccaggtcaaggccgagatacgcaacgacgtgctcggtggacggtataccagcgcactgaccggctccgcccctatctccgacgagatgaaggcgtgggtcgaggagctgctcgacatgcatctggtcgagggctacggctccaccgaggccgggatgatcctgatcgacggagccattcggcgcccggcggtactcgactacaagctggtcgatgttcccgacctgggttacttcctgaccgaccggccacatccgcggggcgagttgctggtcaagaccgatagtttgttcccgggctactaccagcgagccgaagtcaccgccgacgtgttcgatgctgacggcttctaccggaccggcgacatcatggccgaggtcggccccgaacagttcgtgtacctcgaccgccgcaacaacgtgttgaagctgtcgcagggcgagttcgtcaccgtctccaaactcgaagcggtgtttggcgacagcccactggtacggcagatctacatctacggcaacagcgcccgtgcctacctgttggcggtgatcgtccccacccaggaggcgctggacgccgtgcctgtcgaggagctcaaggcgcggctgggcgactcgctgcaagaggtcgcaaaggccgccggcctgcagtcctacgagatcccgcgcgacttcatcatcgaaacaacaccatggacgctggagaacggcctgctcaccggcatccgcaagttggccaggccgcagctgaaaaagcattacggcgagcttctcgagcagatctacacggacctggcacacggccaggccgacgaactgcgctcgctgcgccaaagcggtgccgatgcgccggtgctggtgacggtgtgccgtgcggcggccgcgctgttgggcggcagcgcctctgacgtccagcccgatgcgcacttcaccgatttgggcggcgactcgctgtcggcgctgtcgttcaccaacctgctgcacgagatcttcgacatcgaagtgccggtgggcgtcatcgtcagccccgccaacgacttgcaggccctggccgactacgtcgaggcggctcgcaaacccggctcgtcacggccgaccttcgcctcggtccacggcgcctcgaatgggcaggtcaccgaggtgcatgccggtgacctgtccctggacaaattcatcgatgccgcaaccctggccgaagctccccggctgcccgccgcaaacacccaagtgcgcaccgtgctgctgaccggcgccaccggcttcctcgggcgctacctggccctggaatggctggagcggatggacctggtcgacggcaaactgatctgcctggtccgggccaagtccgacaccgaagcacgggcgcggctggacaagacgttcgacagcggcgaccccgaactgctggcccactaccgcgcactggccggcgaccacctcgaggtgctcgccggtgacaagggcgaagccgacctcggactggaccggcagacctggcaacgcctggccgacacggtcgacctgatcgtcgaccccgcggccctggtcaaccacgtactgccatacagccagctgttcgggcccaacgcgctgggcaccgccgagctgctgcggctggcgctcacctccaagatcaagccctacagctacacctcgacaatcggtgtcgccgaccagatcccgccgtcggcgttcaccgaggacgccgacatccgggtcatcagcgccacccgcgcggtcgacgacagctacgccaatggctactcgaacagcaagtgggccggcgaggtgctgttgcgcgaggcgcatgacctgtgtggcctgccggttgcggtgttccgctgcgacatgatcctggccgacaccacatgggcgggacagctcaatgtgccggacatgttcacccggatgatcctgagcctggcggccaccggtatcgcgccgggttcgttctatgagcttgcggccgacggcgcccggcaacgcgcccactatgacggtctgcccgtcgagttcatcgccgaggcgatttcgactttgggtgcgcagagccaggatggtttccacacgtatcacgtgatgaacccctacgacgacggcatcggactcgacgagttcgtcgactggctcaacgagtccggttgccccatccagcgcatcgctgactatggcgactggctgcagcgcttcgaaaccgcactgcgcgcactgcccgatcggcagcggcacagctcactgctgccgctgttgcacaactatcggcagccggagcggcccgtccgcgggtcgatcgcccctaccgatcgcttccgggcagcggtgcaagaggccaagatcggccccgacaaagacattccgcacgtcggcgcgccgatcatcgtgaagtacgtcagcgacctgcgcctactcggcctgctctaa

SEQ ID NO: 10 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 11 - DNA, 3522 bp, Artificial Sequence
  Description of Artificial Sequence Synthetic carboxylic acid reductase polynucleotide designated 891GA
atgagcaccgcaacccatgatgaacgtctggatcgtcgtgttcatgaactgattgcaaccgatccgcagtttgcagcagcacagccggatcctgcaattaccgcagcactggaacagcctggtctgcgtctgccgcagattattcgtaccgttctggatggttatgcagatcgtccggcactgggtcagcgtgttgttgaatttgttaccgatgcaaaaaccggtcgtaccagcgcacagctgctgcctcgttttgaaaccattacctatagcgaagttgcacagcgtgttagcgcactgggtcgtgcactgagtgatgatgcagttcatccgggtgatcgtgtttgtgttctgggttttaatagcgttgattatgccaccattgatatggcactgggtgcaattggtgcagttagcgttccgctgcagaccagcgcagcaattagcagcctgcagccgattgttgcagaaaccgaaccgaccctgattgcaagcagcgttaatcagctgtcagatgcagttcagctgattaccggtgcagaacaggcaccgacccgtctggttgtttttgattatcatccgcaggttgatgatcagcgtgaagcagttcaggatgcagcagcacgtctgagcagcaccggtgttgcagttcagaccctggcagaactgctggaacgtggtaaagatctgcctgcagttgcagaaccgcctgcagatgaagatagcctggcactgctgatttataccagcggtagcacaggtgcaccgaaaggtgcaatgtatccgcagagcaatgttggtaaaatgtggcgtcgtggtagcaaaaattggtttggtgaaagcgcagcaagcattaccctgaatttcatgccgatgagccatgttatgggtcgtagcattctgtatggcaccctgggtaatggtggcaccgcatattttgcagcacgtagcgatctgagcaccctgctggaagatctggaactggttcgtccgaccgaactgaattttgttccgcgtatttgggaaaccctgtatggtgaatttcagcgtcaggttgaacgtcgtctgagcgaagctggcgatgccggtgaacgtcgtgcagttgaagcagaagttctggcagaacagcgtcagtatctgctgggtggtcgttttacctttgcaatgaccggtagcgcaccgattagtccggaactgcgtaattgggttgaaagcctgctggaaatgcatctgatggatggctatggtagcaccgaagcaggtatggttctgtttgatggcgaaattcagcgtccgcctgtgattgattataaactggttgatgttccggatctgggttattttagcaccgatcgtccgcatccgcgtggtgaactgctgctgcgtaccgaaaatatgtttccgggttattataaacgtgcagaaaccaccgcaggcgtttttgatgaagatggttattatcgtaccggtgatgtgtttgcagaaattgcaccggatcgtctggtttatgttgatcgtcgtaataatgttctgaaactggcacagggtgaatttgtgaccctggccaaactggaagcagtttttggtaatagtccgctgattcgtcagatttatgtgtatggtaatagcgcacagccgtatctgctggcagttgttgttccgaccgaagaggcactggcaagcggtgatccggaaaccctgaaaccgaaaattgcagatagcctgcagcaggttgcaaaagaagcaggtctgcagagctatgaagttccgcgtgattttattattgaaaccaccccgtttagcctggaaaatggtctgctgaccggtattcgtaaactggcatggccgaaactgaaacagcattatggtgaacgcctggaacaaatgtatgcagatctggcagcaggtcaggcaaatgaactggccgaactgcgtcgtaatggtgcacaggcaccggttctgcagaccgttagccgtgcagccggtgcaatgctgggtagcgcagccagcgatctgagtccggatgcacattttaccgatctgggtggtgatagcctgagcgcactgacctttggtaatctgctgcgtgaaatttttgatgttgatgtgccggttggtgttattgttagtccggctaatgatctggcagccattgcaagctatattgaagcagaacgtcagggtagcaaacgtccgacctttgcaagcgttcatggtcgtgatgcaaccgttgttcgtgcagcagatctgaccctggataaatttctggatgcagaaaccctggcagcagcaccgaatctgccgaaaccggcaaccgaagttcgtaccgtgctgctgacaggtgcaaccggttttctgggtcgttatctggcactggaatggctggaacgtatggatatggttgatggtaaagttattgcactggttcgtgcccgtagtgatgaagaagcacgcgcacgtctggataaaacctttgatagtggtgatccgaaactgctggcacattatcagcagctggctgcagatcatctggaagttattgccggtgataaaggtgaagcaaatctgggtctgggtcaggatgtttggcagcgtctggcagataccgttgatgttattgtggatccggcagcactggttaatcatgttctgccgtatagcgaactgtttggtccgaatgcactgggcaccgcagaactgattcgtctggcactgaccagcaaacagaaaccgtatacctatgttagcaccattggtgttggcgatcagattgaaccgggtaaatttgttgaaaatgccgatattcgtcagatgagcgcaacccgtgcaattaatgatagctatgcaaatggctacggcaatagcaaatgggcaggcgaagttctgctgcgcgaagcacatgatctgtgtggtctgccggttgcagtttttcgttgtgatatgattctggccgataccacctatgcaggtcagctgaatctgccggatatgtttacccgtctgatgctgagcctggttgcaaccggtattgcaccgggtagcttttatgaactggatgcagatggtaatcgtcagcgtgcacattatgatggcctgccggttgaatttattgcagcagccattagcaccctgggttcacagattaccgatagcgataccggttttcagacctatcatgttatgaacccgtatgatgatggtgttggtctggatgaatatgttgattggctggttgatgccggttatagcattgaacgtattgcagattatagcgaatggctgcgtcgctttgaaacctcactgcgtgcactgccggatcgtcagcgccagtatagcctgctgccgctgctgcacaattatcgtacaccggaaaaaccgattaatggtagcattgcaccgaccgatgtttttcgtgcagccgttcaagaagccaaaattggtccggataaagatattccgcatgttagccctccggtgattgttaaatatattaccgatctgcagctgctgggtctgctgtaa

SEQ ID NO: 12 - protein, 1173 aa, Artificial Sequence; identical to SEQ ID NO: 8
  Description of Artificial Sequence Synthetic carboxylic acid reductase polypeptide designated 891GA